SEQUENCE LISTING (175 sequences)

SEQ ID NO: 1 - DNA, 6923 bp, Homo sapiens
gagaggggcagggggcggagctggagggggtggttcggcgtgggggccgttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttcta

SEQ ID NO: 2 - DNA, 54000 bp, Homo sapiens
attaattcatttgaaagacatcaactacaatttaaaaaaacaaaacaaaaacagaaaaggtggtttgcaatttttttcacttcatgagactataatttatttccctgggtaggccacacaaatccttaagaacatggactttcccccatacaaggctgtgaaccgtgcacacagagatgctcgataaatggtaccgaatcctctccttcagcacctggtcctctctggcagtctccattctcttacctctcactgccccctctacttctctgaacctgcagtccccaaagtcaccaatggtctccatagcaacaaaatttagtgggctcttatacgaccttatattgcttgacttcttggcagcaactgacactgactaccccttgaagcatcccctgcccaggccttccacaatgtcacactctcttgcttctcctccctcctccttggttattgcttctcctatcctcacggctcagctctagccctcttctcaatcacttccttttccctaaaatgattttattcactcctatggtttcagttacttttttttttttttttttttgagacacagtttcactctgttgtccaggctggagtgcaatagcacgatcttggctcactgcaacctctacctcccggttcaagtgattctcctgcctcagcctcctgagtagctgggattataggcatgagccaccacgcccggctaatttttgtatttttgtagagatgaggtttcaccatgttggccagcctggtcttgaactcctggcctcaagagatctgcctgcctcagcctcccaaagtgctgggattacaggcatgcaccaccacaatggccttagctatctgttttttaatgacaattcctcattgagttcaaagcagcctcctgaacttcagactcggtatccaaccattactcaacataattactcaaatgtccatagatatttcaaattcagtatctccaaaatgtaaactctatcttattctcttcccaaaaccttgctgctcctcccatgtttctgctctcaataccactcctgccacagtggctgcccaggtcacaaacccagaagtcaccttcacatttctgtctcctaccttccccaatccctcattgtggtaggagttattaagaaattattttaggcagatagagaggaaaaggggtccttgaaaagtttttgtctcttttgaagcagctcctgagacgtttcctgtctagcataaaagccctgacttttagacccgagctggcaacgtttgatatgcaaatgccggcagttagaaactgggtccaccaacacggtgattcccaccatcgtcctcttgcccttgttctcacaagtgcctggcaacatggccacccccacatatccccacatgagtagaacatcatagcgccctgcatttgcatattaaaaggctagggtggaatggccagttttattagggggctacatgaatgacatgcgtggtcaaaccagtcccctgagccctatgcaaatcagacaccgcctcctgcatcctcctcctataactggctggtatctcccacgcactctgggtctcctctctcagctttggagcacccatccctctgtctctgtacaggggagctgcttctttctttcttctcccttccttcttgcttattaaactctccgctccttaaaaccactccacgtttgtccatgtcgttttatgtaattcgactcaagacgaaaaaggctagtattcctccactcggtatcatcaccacattctgttgagtctctttacgtccaaagaatctctcaaaatgggctatttccatctttaagacactctcttaaaccaccatcgtctctcccaagattccttcaacagcaactgcttttttctgcagccagtgatctttctaaaaggcaaacatgaccacatgtcttcttttaaaatatttatatagctccccacatcctttaaaaggtacactttggccaggtgcggtggctcacgcctgtaattccaggactttgggaggccaaggcaggtggatcacctgaggctgggggttcgagaccagcctggccaacatggtacaacctcttctctactaagaatacaaaaatagttggacttcgtggtgcacgcctgtaatcccagctgtttgggaggctgaggcaagagaatcacttgaacccaggaagcagaagttgcagtgagcggggagattgtgccactgcactccagcctgggggacagagagagggccgtctcaaaaaaaaaaaaaaaaaaagtacaccttaatctggtttcaaggtgcaagatctgcacctatccttccatttaagtttcatcaaaccgctgctacctccccgtctcccacacaatttatgggacttctaagttccctctaaagggtccgaacacctacactggtaacaagccacctggatttgaatcctggcaagacaacttactatctgaccttggacattgtgctgttcttaacctctccgtgcctcggtttcctcatgtgtatgaataacatcaacacctacatcaaagtttgctgtattaaatttgataatatatgcaaagcatttagaaaagtgcctagctcatagaaagccttatgtaaatattaactatcattttttttcttttttggggtggtgggggaggggtttcgcttttgttgcccaggctggcgtgcaatggcacgatctcggctcaccacaacctcggcctcccgggttcaagcgattctcctgcctcagcctcccgagtacctgggattacaggcatgcaccaccacgcccgggtaattttgtatttttagtggagacggggtttctccatgttggtcaggctgatctcaaactcccgacctcaggtgatccgcccgcctcggcctcccaaagtgctgggattacaagcgtgaagcacctcgcccggcctagctatcatttttatacaagtgctgggttttgggagaatgtaatgatggcttttttcttactaaactttcagtgcaggaggaggagaaagaaagtaaatagttatatgaacacagtagaaagtcaaagtggaaaacaaaaagaacatagaacccaggtgagcggtccagacctccccccagaaacctaagaatccatagaaatgggtgggaagcggagaagatcctccagacagcaggtggcgatgtagcatcccccagaaggcccgctaacagaagctaggaggacgcgctaccaaggtcacgtgtccccggcgttcactcgctcttcgcttcacgacactcgcatcctcacgggtgattggtctgcgtgcggcacgtgggcggggtaccggggcgggccggggaggggcggggtgggcggaggagaggggcagggggcggagctggagggggtggttcggcgtgggggccgttggctccagacaaataaacatggagtccatcttccacgagaaagtgagtgtccgcgttcggtggggagctgtctgccgcgcggtggcgggcgtggagcgcggcatcaccgcctctcggagggctgggtggggcccgagtcgcccccatgccgatctcgcccggcgaggggcgacgccgcagcctcccgcctcctcggctcgaggaggggagcatcacctacgcccctacttcccccgcggcccccgccctgggagccgggagggagtatgggcggggccgggggcgtctcgggacacgggagtggggtggcgcccagtgggtttgcttctgcctttctccgtcactttccatcgcttttcggaggattccttcacccctccccaatccttccctctccctagggtctagctagagtcatctctgggacacctccctcaacccctcctaccctaatcctggcagaattaacttttcctcctccggactgctcaattctatattggagtcttccctacacgtagatctttggggtcttgttcgtgtctttcccctgcactaggtccgcgagcctcccgagggaggagaccttggctcgcccactgtagggcctgacatttaggaagtgaagtaggaaacccggcgtgcccctaaacagggaagtcgtcacaagagtttttattacgggatgtttgggtttggtttcttttggtactcccatctttccggagcaggcggccagctttgtttttaggtattaggagtggactgggatgattttgttgtagtctgcctagcctgctgtccctttaactcttccgtgaccatgcacttgaagatactgtttgtgatatgtaaagaaactcctcgtttctctcatactattatccagccatttgtgtgtgagtgaagccttccccaggacagctttggcacatggtatcatgtttcataatagtttcgtgtttggaaagagttgctggtaaggctgttatttaataggaggagcaaagggtttttgttttattaaatacttataaatgatcatttatcccagacatttaaaattcacacacacacaacaaataaagcaaagacaaaagaatacatttaccaaatgtaaatctgtagcataaattttttttaatttttattttaaagatggggtctcattctgtcacccaggcaggtgtgcaatggagagatcatggctcactgcagccttgatctcctaggcacaagcgatcctcccgcctctgcctccagagtagctgggactacaggtgcatatcgccagggccaggtaatgtttttgggagagacggggtctcgctgtgttgcccaggctggtctcgaactcctggactcaggtgattctcccacctcggcctctcgaagtgctgtgattacaggcgtgagccactgtgcctggaacaaattgttaagtacaatgcttttcattgtagaaaacatctcggaaacttttgaaataggctgatgttcagtgggggaggaaggactcagtcgtatagttgtcactaattttttgacttgattgacatgactcgtaaatcatagacaatagagatttggttgcttggctgagtagagtgcgtgaaaaatacacacgtacttttttttttttttttttgagatggagtttggctcttgtcacccaggctggagtgcaatggcgccatcatggctcactgcaacctccgcctccccgttcaagcgattctcctgcctcagtctccccagtagctgagattacaggcgcccgccaccacgcccagctaatttttgtatttttagtagagacagggtttcaccatgttggccaggctggtctccaactcctgacaggtggtccgcccgcctcggcctcccaaagtgctgggattacaggcgtgagccaccgcacccggccatatttttgttattaattttcaaaggctttggtgtgggaccacatttcaacatggaaggccttaaacatgttccacactacttcctgagaattagacaagatttttaacaatattgttacctagttgggacacatttgtactgacccatgggatgaaaaaaagctgagtgctagcctagtgaaaatctacttacccgaaagaaatccctcttagtctgggtgcagtggctcacaccagtgctttgggaggcccagacgggcggatcatgaggtcagtagtttgagaccagcctggccaacatggtgaaaccccgtctctactaaaaatacaaaaaattagccaggtgtggtggcaggcgcctgtaatcccaggtactctggaggctgaggcaggagaattgcttgaacccgagaggcagaggttgcagtgagccgagaccgtgccactgcacttcagcctgggcaacagagcgagactccgtctcaaaaaaaagaaaaggaaaaaagagtccctcttaattatcagcatgtgtataggcctacagatacttcaggaatacctttaccattatcatcaacttgtatctacatagcatgtgaagattcaacaatttagttttttgggcgtcctcaagagtacgcacctataaccatatggcccaattgttaatctcctatacagtccattctgggaatgtttgggcttactgtgccatttttccgttcactgccttcccctctgcaatatacctttaacccttgctaggtcctgggtttggagagccagagaaccaactttggccctaaagaagctgtgtaggtagcaatatctgcctacgaagggccttgcaaccatttcctcttggaaccttggtttcctctttctgagtagtcactttgagtaccctttattaagttagaatgtaaaaacagtttctcactgatatatctgcagtgcctgagagagggcctggcacagagtaagtactcaataaatatttgaatggggccgggcgtggtgagacctgtctctacaagaatgaacaaaattagctgggcgtgttagcacatgcctgtagacttgggaggctgaggtgggaggattgcatgagtctgggaggtcgaggctgtagtgagccatgatcgcaccactgcactccagcctaggggacagagcaagatcctgtctcaaaagaaaaaaatgtatatatttgaatggataaagagatggctttgagtttctgagatatatatggtgctgtttatctaaagtaaacaagttttctgtaaatattttaaggctttgcaggccagctgtagtctctgtcacacattcttatttgtgcatgtttttcccaaccatgtaaaaatgtaaagtgcattcttagctactggggcaggttgaatttggcccatgggctagagtttgccaacccctaacttaaacctttgtactaactttatgaccactactggatttttgttgttgtttgttttagttctggtgcctgctttgtttttttttttttttttaatcctcttgctgatgtttcttggtgcagttactgtgccatttgtattggtgcttttaatgtaatgcaaactggtaataatatctaaacttgctggggttgtacataaaattattgaaaagattgaaaagatgctgagcattgactctgtggcattcattatgcccttttgtgattgctggattttagccatctttaggacatttgagctttaggagaagccaaattctgtataaatgacttgaagtgctaatagcacaggttttgaaacctctgcctgggtttgagtctcagctctgccttttactacctgtgtgatcctgagcaagttacttagtatccctgtcctctagtttcctcctctgtagtgtggggataataacatagacataacctgagagttagagtgtagagaaggctccctggcagatagtgctgtagaagtactggccattgccattactcaggtgcttgtgtttgctgaacctcatagtaagggctcggagagcactaagaggaggtgagaaatgctgctagattgacagcttgtccccagatagcccattcccgagagcaccttaggtttatacctgatttgtgttgtagttagtagtgtctctggtaatttgaactagtttcaggttggtcttgaaaacctggggaggttgggggtaaatgatttggtagcagttctcttttgtgattttatacattatctttgtagaactgcagtttgctaattctctgagcccaacacaatgaagtctgggcctaaaatcatagaatttcttttatttttttttttgtttttaatttatttattccctccctccctcctttcttcctttcttccttttctttctttctttccttccttccttccttctttcttttctttctttcttttctttctttggagtctcactctgtcaccaggctggagtgcagtggcacgaactttcttcagagtctcactttgtcaccaggctggagtgcagtggcgcgaactcagctcactgcaacctccgtctcctgagttcaagagattctcctgcctcagcctcccgagtagctgggactataggcatgtgccaccatgcccagctaattttcttatttttagtagagacgaggtttcaccatgttggccaggatggtcttgatctcttgacctcgtgatccacctgcctcagcctcccaaagtgcggggattacaggcgtgagctaccacgcccagcctattttttattttttgaggcagagtctcactctgtcacccaggctggagtgcagtggtgcaatctcagctcactgcaacctccgcctcctgggttcaggtgattctcctgccttagcctcctgagcacctgggactacaggcgcctgccaccacacctggctaattcttatatttttagtagaggcggggtttcaccatgttggccaggctggtctcgaactcctgatctcaagtgatcaacctgccttggcctcccaaagtgctggaattacagccatgagccaccatgcccagccaaatcatgagatttcaataccgctgaactttgattatggcaaagtgaacttctgctttgattaaagcttgatgagagaggtggctggggatagtttgagataagggcaaggcaggaaaatgcataatcttacgtgggctcattgtcattgtacaattcttttggtccatgtggaatttgatccgtcctatgacttaagttatgtttatttttgtttttatttttatttattttgtgtctttttgagagacatgatgttgctctgtcacctgggccagaatacagtggcacaatcttagctccgtgtagccttgaactcctgggctcaagtgatcctcccacctcagcccctcaaacagttgagattatagtatgaaccactgtgcctagccttaagtgatttttaaatttgtactgaacagtttgtcctttccttccattaaatcatattagaagtacagaacttgatatttcctgtagcaatacagtttttctttgatgaagtttgatttcaagtacttatttttcataatttaaagctattttttatagagagaattttaatcaaatatttggatgtcactattgctatatatggtattaagtatggtgaccatagtttgtaaactccaaactgacagcaagacaggaaatttgtgttagcaaaggcttttttcttactgtttgaattttttaaaaattagatacaatacagagaggagcacacaaatcattaagagtacagctcagcgaattttcacacagtgaacatgtgtaaacagcaagtaacaaaagatttacctgcatcctataacctcccattattcccttttctaggtactgtctctccactgcattcccaccaaatataaccactatgctgaattctgacatcataaatgagttttgcctgattttgagcttttgtgactggaagtgtacagtgtatataccctttcgattctgtcctctttagtttaccattgtttgagaaatttatccatactgttccagaattaactactgttaattattgttaattaactactgttgtagttaattcatcctcattgttatctagtattcttttgtgagtaaacacaatttccattctactgtgatcccagctatccatttgggtcgtttccagtttggggtccattacaaatagtaatgctatctgtaatgctattttgtattactacaaatagtaatgctatttgtggcacaaaaatactgcttttgtgaacattcttatacatgtcttttgatgaatgtatgtttgcattgctgttgtttacattatgtacctagtaatggaattgctagatcataggagatgtatatattaagctttagtggatgcattacataattattagttattattggttataccaatttatcctctcatcagtagtatacaacagtttctgtatctctaatctccaacattttagccattttagagtttgtgtactaacacattgtggttttaatttacatttccctgatgactaataaagttgagtacctcttttgtgttctttatagccatttgactgtcttgtgaagtgcttgtttgtcttgcctatttttcttttctttctttctttttcttccttccttcctttctttctttcttctttctttccttccttcttttctttctttctgtctttctttcttgtctttcttgtctttctgtctttcttggtcttgccctgtcacccatgctggagtgcagtggtgcagtctcagcttactgtagcctcgacctttttggggctcaagttatcctcctttctcagcctcccaagaagctggactacaagcacgcaccaccatgctcagttaattttttattttttgtagaaatggggtttcaccatgttgtccaggctggtctcaaacttctgggctcaagtaatcctcctgccttggcctcccaaaatgctgggattacaggcatgagccaccgcagccagccttggctatttttcaaaaggatataagtagaacatctgtatatcccttcaatttgcatattattcagtaagagttgcactctggtagtagaaatatataaggaggagaaagaagtggaaacaaaaagtctattctcatgagaagacttgggggatagtgttctctctagctccaagctacttattccttacgaaaagttgaagataaacttatctcagactgaggctgtctcaatgttgtcttcctattccattatacacatataacccatatttttttcaccagctgaattttgctcctagaaaattgattcatcaggaaaaatatccgtcttgcaaggtggttctctttagagtctgctgtgtgacatagctcaggacaaattgtgtgatgtcagataggttgggttaaggaatagaccttattggggaaagagagaacttggagggccaaggttagcaggagaaggaaatgttctctcatctgccgtcaattcagggaggggcaaacctggtgtctgtgttcacagggagggatccatccatctgtgattctcccttcttatcaggtagcatgggaaagctacactgttgcggggaggagggtcacacgcaggctacttagtaccaggcaccctggacttggattcaggttgccagttgtgtgagaaactgcccagcacctgaaggccctgaacccatgagaagttgtacctacctcccatgaggaggaatcctgtcatcccatgggagctgagcttgggtgcagtccctcttgctggcttgtccaggagtgagctccagggttgtttgggacagttctgctcattgctttacactgtgtatacattatctgtagagttccatgaagagaacttcagcactgtaactgcaagttttaacatggaacagaatttttctcacctgtattaattcttaagatttgaagttctatcaacaagcatttagattgtgtggagatttttttatttttatttttggagacagagtcttgctctgttacccagactggagtggcagtggcatggtcttggctcactgcaggctctacttcctgggttcaagcgattctcatgcctcagtgtcctgattagctaggactacaggtacacaccaccatgctggctaatttttgtatttttagtagagacgaggtttcaccgtattggtcaggctggtctcgaactcccagcctcaagcagtccacccacctcggcctcccaaactgctgggattacaggtgtgagccaccatgcttgactgacatcatcatgttaaaagaataaatgttctagggagctgggcacagtgtcatgtttctgtagttctagctgctcgggaggctgaggcaggaagatcccttgagccctggagttcaagtccagcctgggcaacatagtgagatctctttttttaaataaataaataactgttctagggactaaaatttcctttcaccattagtaatttactgtagaatctccaagaatgaacttattttaggtactgaaaatgagggagactaaatgttttatacagtagtttttagtaaaatatgagatttgatgcatttgatagatgatgtttgtttaaaataattcttaaatttttgatcatgtaattatagtttcattaatggtagatttgtaaaataaatgttaccaaatgaaaatgcatgtacctatgttaattatccttatctaaagctgaaagttcagttcaactatgttaaaacatagtaggggcctggcagggtggctcttgcctgtaatcccagaacttagggaggccaaggtgggcagatcacgaggtcaggagatcgagaccatcctggctaacattgtgaaaccgtatcgctactaaaaatacaaaaaattagccgggcatggcggtgggcacctgtagtcgcagctacttggtaggctgaggcaggagaatggcgtgaactcaggaggcagagcttacagtgagccgagatcatgccactgcactccaggctgggtgacagagcaagactccatctcaaaaaaaaaaaaaaagttggccaggtgtggcggctcacacctgtaatcccagcacttttggaggccgaggcaggcggatcacaagatcaggagtttgagaccagcctggctaacagagtgaaaccctgtatatactaaaaatacaaaaattagccaggcatggtggtgcatgcctgtagtcccagctacttgagaggctgaggcaggagaatcacttgaacccgggaggcggaggttgtggtaagctgagattgctccactgcactccagcctggacaacagagcaagactctgtctcaaaaaaaaaaaaaattaatgattaaattatttaggggagccgggcgcagtggctcacgcctgtaatcccagcactttgggaggccaaggcgggcggatcacgaggtcaggagatcaagaccatcctggctaacacaggatgaaaccccgtctctactaaaaatacaaaaatttagccgggcgtggtggcgggtgcctgtagtaccagctactcgggaggctgaggcaggagaatggcatgaacccgggtggcggagcttgcagtgagccaagatagcgccactgcactccggcctgggtgaaagagtgagactccgtctcaaaaaaaaaaaaaaattatttaggggaagatactatacaattctgtttaacaagtcacattttaattttttcttttggaaatattagcaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacaggtactgattttaaactcactaagtcacatttcttttttttttttttttttgagacggagtctcgccctgttgcccatgctggagtgcaatggcgcgatctcggctcactgcaacctctgcctcccgggttcaagcgattctcctgcctcagcctcccaagtagctgggattacaggcacacggcactatgcccggctaattttttgtatctttgttagagatggggtttcaccatgttggtcaggttggtctcaaactcctgaccttatgatccacctgtcttggcctcccaaagtgctgggattataggtgtgagccaccacacccggcttacatttcttttaaaaatgtggataccatttagaaaaggatgggccattcttcctatagggatctgactggtgaattataactgtgctgttaactttggaaatgggaatgcacaagatattgttttaaatatgcacgctaatgacagtttgtatccttctttccccacccccacccttgcttcaactacctgtcaaaattaacagcagccttctggaaatatggatgacagtggttttttctctattcaggtaagtagtcacaagcatgtactatgtgttgcttacatcccaggcaccgtttcacagcctttcaatagtcactgtaacaaggcgaccttcggaagttcttctgtctacagagtatagattatactctagagtactagattttttttttcttgagacagagtctcgttctgtcacctaggctggagtgcagtggcgtgatcttggctcactgtagcctctgcctcccgggttcaagcgatcctcctgcctcagcctcccaagtagctgggattacaggcacccgccaccacaccagttaatatttgtatttttagtagagatagtggggtttcaccgtgttggccagtctggtctccaactcctgacctcagcctcccaaagtgctgggattacaggtgtgagccactgcacctggccaactagagtactagatttttatatagataaacatgaaaggattgtagaatcttcatattagagtggggcatttaaaaattccttcttgagaaagattaatttgcatctggatgctaataataaccttaattctggccgggcgcggtggctcacacctgtaatcccagcactttggggaggccgaggtgggcggatcacgaggtcaggagattgagaccatcctggctaacatggtgaaaccccgtctctactaaaaatacaaaaattagctggacgtggtgacacgtgcctgtaatcccagctactcgggaggctgaggcaggagaatcgcttgaaccagggagtcgtaggttgcagtgagccaagatcgcgccactgcactctagcctggtgacagagcgagactccatctcaaagaaaaaaagaaatccttaattctaataagtcacaatgtctcaaacttaccatctgttgggtaaatttgagaaaatgcaataccttgctaccatccttttaaatcagcctaccagactggatttccttattatggtttgtggcttttgattttttttttttaatgtatagctctctttgaattctttggtggttatatatatatgtactcgcaagattcttttatctgtgggtctttcattctttttctaacactgtgagttgtatccagagtactttcggaacctctcctgagcgacctatctctgcagatatctttgtttatgtttcccttgtactgccctcctggactcttcctcatccaccagcatttccatctagtgctttaccgtgccactgctaacaggtaatggctactgcagggctgaaatcagaggccagagtaggcccagcacttggcgtttcctatttgtgccttgctgctcttggtgcctgttcatgtgtgcccactaccttgcactcaatttctgtctttgctggtacctggctcacttgcttctttgttggctaccttggagggcagatagtgaattttcagaaatttccctttttttgtcagacagattgaaataaacaggtttgcattttgttttttctacaagcggcaagcccatgaccctagaagtctgacatctatggaaccttcagtttaaatgcccagggagaacttattttggtagatatgatttctgacattgcaggtagcaagttgaatataatttttctaaagtagcacccacagcagccaaattatcagatgtatatagtagactagttttaagaaaagcacttatgggtagaatatacatctggatttttgaggcagttttatttaggaattgtgtggttttctggaacatctcagagacctggtatgaaaagcactcttctaatatatatgtgtttttttttatggatttagtgatatatctatacacacacactttttaaaacctatagccggctgggcgtggtggctcatgcctgtaatcccagtactttgggaggcccaggcgggtggatcacaaggtcaggagattgagaccagcctggccaacaaggtgaaaccctgtctctactaaaaatacaaaaatagctgggtgtggtggcgtgtgcttgtaatcccagctactcgggagcctcaggaggagaatcgcttgaacctgggaggcggaggttgcagcgagccgagatcgtgccactatactccagcctgggcgacagagcaagactctgtcacaaaaaaaaaaaaaaaaacctatagccttctagagaaatttatatatgaagtacacaactaacatagctacacttcctaaatttggaatggagtggtttagcttatgaaaagttgctatttttcttaacaggttataagcaatgccttgaaagtttggggtttagaactaatcctgttcaacagtccagagtatcagaggctcaggatcgatcctatgtaagattctgttttgcatttcatacatttcttttcccaaatttgatttttaaagttgtaatttcttaaagaagagaaatacattttgaatacttttgttttgatgttccctgtttcattcactcagactttcctatttcacctttgtgatgtccatgagcatctgccctgtagccttcctggcaccccagtgtctgtggcagcacagagctgaccccataagtggtgcatgaggccatcttgtggcacagcatcactaagctgctgcagagacgttcatatggttgtgtgatcttttaaaaacatcagtgacacttaactataaatataatcttaaattatcacaaattttatataatatttgccagtagacaacataaatatgaattcaatatttcaagttaatattgtctgttttcttttttagaaatgaaagatcatttatatgcaattataaggaacactggtttacagttagaaaattaggaaaacaggtaacatttcttacccttccttgtctttttttcttatattgtaccccatttaaaactaaaatgtgggccaggtgtggtggctcatgccaacagtttgggaggctgaggtggggggatcacttgaagccaggagtttgagaccagcctgggcaacaaagggaggtcctgtctcttaaaaaaaaaataaaaataaaaataaaaataaataaaaaaaaaaacaaagagccaggcatggtggctcacatctgtaattccagcttacttggaaggctgagtcagaaggatcacttgagctcaggagtttgaggctgcagtgaactatgattttgtcactgtaccccagcctgggtgacagagtaagactgttctataaaacataaaaataaaaaaaatatatttaaaaattaaaaaaaaaaaaggattgctgactttaaaattaggaaactgaccagtaatgtgtgtgtgtgtagcatggtttatccttcttgatagatagaaattgtcattttaaaagataatatcagttttccttataaatttatttgtgacaagtatatgcaatttaactatatcataagaaaaattctatattaaagataatacaaatgtggttacttttaagtgggtttttatgtgatgactatgttctgtcagttaattattacatttatagatttgtatttagcatagtgctgtcacaaagcctgaaatagtgtcaagcatgaataaagcattcaattatgtttgctttagtgtaagattattcattatgattccaaaagccatgtaatacgtacgtctacagaaaatcacttctattttttaaataaaacatgaaatatgtcttgagcaagctattttaagaaacaatcatttaacgtccttgttattagaattttgaatctttgaaagagggttattgaaaaccagctaggacagtaaaaaagaataaactagtgatacatgcagcaatatggatgaatctcaaaataattatgctgaaagaataacccacaaacaaaatactacctgctgtatggtatcatttattaaaagtctagaaaagtgcagattcatctgtagtgatggaaagcagattgaccagcggttgcctggggacgagaaggctatggaggagtgagaggggagggttacagagaggcacgggaaacatggcaatgaggaatgtgttcactatcttggttgtagtaatggtttcatgggagtacagtatacaaatgtgaaaacatttcagaggccagatgcagtggctcatgcctgtaatcccagcacttttggaggccaaggcaggaggattgcttgagctcaaggagttcaggaccagcctgggcaatggcacaagaccccatctctaaaaaaaaaatgaaagaaaaaaaaattggctaggcgtggtgatgcatggccgtagtcccaggtgctagggaggctgaggagggagcacagaggtcaagcctgcagtgaatcatgatcgtgctactgcactccagcttgggtgacagaaggagatcctgtctcaaaaaaaaagtttcaaattatacactttaaatatgtgcagtttattatatgtcacttataccccaataaatctgttttttttaaaatgtaaatacaagccaaaaaaggtataagtcaagaaaatatattgaattaaatctgtaagagataattcaaaaacaaaaaccctattgttatcttttaagtcacccaaatcaaatttgggaaaagtcacctacttagcttcatcctaagttggttctttctttctttctttccttcttttgagacggattcttgctctatcgcccaggctggattgcagtggcgggatcttggctccctgcaacctccgccacctgggttcaagcaattctcttgtctcagcctcccaaatagctgtgtctacagccacgcaccaccacacccagctaatttttgtatttttagtagagacggggtttcgccatgttggtcaggctggtcttgaactcctgacctcaggtgatccgtccgtctctgcctctcaaagtgctggggttacaggcgtgagccaccatgccgagccctaagttggttctttcttaaagttcttcctgaggagccaagagcaagttaaggagatgtaacctagaagcttacagtggaggctagctgggtgcagtggttcacgcctgtaatcccagcactttaggaggctgaggcagggagatcactgaggccaggagcttgagagcagcttggcccaacacagtgacaccttgtctctacaaaaaaaaaaaaaaaaaaaggcagcttacagcagtagaggctgatgcgagtgggaatcacctctaggtaaaaaccagtgtagcgtactgctgagattatttaacctctgggttttatttatgtgtttttaaaaattatgatccagtattttttacttttttttgtataaagtaagcactgaatttttaaggttgtattaatttgcaaataaatgtctatcttattattttgagagatttaaaaaattttagttcttcaaaattgcattttcacattttgaattacgttatctttgacaaatacagaagatgtcaaattttggtttattttctttggttctaatttatatttttgtttaaaactatatttttcactatagactctttctgtctctcgaggtccctgtataatgaaaaagaaggctggaaaaagtattaacattgtcaaaatccaggaaaagtagttggtcatgatattgatcgttaactttagaaactttttgtatcttgtgggttaaattaggattactatgtggtagtgataaatgatgttaattagggccgagtgcagtggctaacacctgtaattccagcatgtagggaggctgaggtgggaggatgtcttgaatccaggagtttgagaccagcctgtacaacatagtgtaagaccccttctccacacaaaaaaattagaaaatttgtcaagcatcttggtgcacacctgtagtcccagctgcttgggaggatgaagcgagagaatcacttaagcccaggtgttcgaggctgcagtgagctatgattgcaccactgcactccagactagatgaccatctcttttaaaaaaatgtgtttatatgttatatgtgatagtgctttttaaaaacatttttaaattatagagacagggtctcactatgttacagcccaggctggtctcaaattcctgggctcaagcaatcctcccaccttagctaacctcccaaagtgctcggattataggcatgagctgcatgcccagctaatttagtgatttttaaaaactgagctggtaattataaattctcttcctggaacttctgactttctcacaattggaatcttttgacaaaaattatcagtaatgggaaaactttgtgtagttgtcatttttcctcccatcagtgtgatagatatgattggagttatgttggactgatattttgaaaaaagatttaattatagctattaataaagacatttaaactactgactatgcatttttattcttttgggagggtttaatgtttatagtttaaagcaaactgttgtttttaaaaaagtatctaacagggccgggcgcggtggctcacacctgtaatcccagcactttgggaggcctaggcgggcggatcacaaggtcaagagatcaagaccatcctggctaacatggtgaaaccctgtctctactaaaaatacaaaaaaatagctgggtgtggcggcgtgcgcctgtagtcccagctactcgggaggctgaggcaggaggatggcatgaacccgggaggcggagcttgcagtgagccgagatcgcgccactgcactccagcctgggcgacagagcaatactctgtctaaaaaaaaaaaaaaaaaaaaaaaaagagtatttagcagaggccaggtgcagtggctcatgtttgtaatcccagaactttgggaggctgaggcgggcggatcatttgaggtcaggagtttgagaccagcctggccaatgtggcaaatgtgctgtctctaactaaaaatacaaaaattagctgggtgtggtggtgcagacctgtagtcccagctacttgggaggctgaggcaggagaatcacttgaacctgggaggcagaggttgcagtgatccgagatcatgccactgcactccagcctgggttacagagtgagactcttctcaaaaaaaaaaaaaagtatttaatagtgataaatctgcagtattctcttgtagtttttaagatcatattattcagtcaaagaaaagagctcaacttgaaatatttccagagtttaaacaatcttactaagctttgatgggttgtatctattcttaacatgtgaaacttccttattacctataatatacactaacttaaatattgacaatttttttccagtggtttaacttgaattctctcttgacgggtccagaattaatatcagatacatatcttgcacttttcttggctcaattacaacaggaaggtaagtaacggctgaacattttgtaatgttacctttcgaagtagttaaataaccaggcacattagatgacagtgtgataaaactgtttttctggcagtggcagtgaaacaatctttagttttgacgtggtgataggctgtgatttgggtgacgctgttcagttagagttctcactgacacctggcccttcctcttctgaggatgctgctttctttgcagcccttctaagtaatggctttttcttttatacatcacatatcacacggctgagaggagggatagatgtttttcttctttgcctcttctaggccactgttcttccttataaactccagtttctttgaaatacatgcccctaacggctgggcacggtggctcacgcctgtaatcccagcactttgggaggctgaggcaggcggatcacgatgtcaggagatcgagaccatcctggctaacacggtgaaatcctgtctctactaaaaataacaaaaaattagccggggtgtggtggcggacgcctgtagtccgagctactcgggaggctgaggcaggagaatggcgtgaacccaggaggcggagcttgcagtgagctgagatcgcgccactgccctccagcctgggcgacagagcgagactccgtctcaaaaaaaaaaagaaaagaaaaaaaaaagaaatacatgcccctagattaaactatcccttgtccttttgcactcatccacaagtctcttttcatcagtgattttaggatctgactcgttgtctttttctctacttcaactacttttatcattcttaattatttctgtatcgtcaatcaatccagtacctgcctcttagtttcaaaatcacttactcttgcttagctattaccagtaatcataaccactgtcaaatctcaattgcaagcatattactctttaactaccacctcctatctttaaaccatgttttgtctgtttttttattccagccattctttaaaccctactgtggggcccaagcatttcctttatacgcattcttcctttcttctactgcttattttctgtaatccgtcatcataatcactccattgcattcttcaacgtgtttcccctctctccctccatcatacttgaatgacaaaaatctcaaccctggttaaaccacatcttggccttgtccattcctgtaccagagtagctggacgtggctaaaaaataacataaaacatgatgattggttttacttttttcttaaatgatctatccatccattcacccatccatctatcaaagtgactaggcctatttctgaagcccaggctggagtgcagcagcataatcacagctcattgcagctccaaactcctgggctcaagtgattctcttgccttagcctgttgagtagctgggactacaggcttgtgctaccacacctagctaaggttttactttaaatttattataatcacaaaattcagatgagcctttagtgctgtctgatatttctactatgttttcttagtgatgtaccaccctccaaggtgtttataaaaaattatgtaccactctccaagaagtttataaaaaataatgtgccaccctccaaggtgactaatttcacagcttatgtctttaaacctttaagcactttcctctcccttacacaccttccttgtggctttccgttacattctgctgagaacatagaagcaattaaaattatgttctttctaccagcaaatttatcaatttgcttatatcttcacctgtgctttgagcctatttaaatagatgaatggtcccctacctctaaccaaaaccagtccctcacttgtgggctggatcccagctcttctcacctactcaagatgttcctgctttcatctctccactctcttatataatcagttccccccccctttttttgtaatattcctataagcagtaaaataagctttttatttccattgattaaaaataaaaatcctctcttaattccatgaaactccagctgcctccccatttttattttttccttaggattgtctctagtgtgccttctccttttcttgaactctgcctcctgggttcaagcgattctcctgcctcaacctcccgagtagctgggattacaggcgtgcaccaccatgaccggctaattttttttttttttttttgagatggagtttccctcttgttgctccggctggagtgcaatggcgtgatctcggctcaccgtaacttctgcctcctgggttcaagcgattttcttgcctcagcctcccgagtagctggatttacaggcatgtgccaccatgcctggctaattttgtattttagtagagatggaaggggtttctccatgtttgttaggctggtctccaactcctgacctcaggtgagccgcccacctcggccccctaaagtgctgggattacaggcatgagccactgcgcctggccccggctaaattttttttttttttttttgtatttttagtagagacagggtttcaccatattggccaggttggtctcgaattcctggcctcgagtgatccacctgcctcagcctcccaaagtgctgggattacaggcgtgagtcaccttgcctagccatcttttagtaatggtatttggagatcacaatttgagtgctggcatgcttattgctgctgggtttgttatgtagttattgtgaattcacatttaggaatatagggtttttaattctttgattttagatacttgtatcttttttcttttatatttaaaaccttggttcctgatgatatcccttcttagaaaccctgtctacctttggccttcagcccaccatgctgtggttttcctaacttgctgcctgcacttttcagattcctttcatggatcttaaatatcatctgtaaataagatctatgtgtcaataattaccaaacttttatctttagtcttgacatctaccctgaacacctagctttgactaactcctagctttggcatctccacttggaaatccaaaaagtgtttcaaactgaacatgtctatgaaagacttatttttttctctctatccatgctatccatcaggttttccatttccataagggtgactcttgtactctggttcctatatattataccgacagagcagcccagagtgcttcttaaccagtgtaaggcctgttatgtcccaccctcactctttgtccttcagtggcttcccagcacacttagaataaaatctgaagtcttaggccgggcttggtggctcatgcctgcaatcccagcactttgggaggatgagggggcagatcacttgaggtcaggagttgatgagaccagcctggccaacatggtgaaaccctgtctctaccaaaaaatacaaaaattaactgggtgtggtgttgtgcacctgtagtcccagctactcgggaggctgagataggagaatcacttgaacccgggaggcagaggttacagcgagccaagatcataccactgcactccagcctgggtgacagaacgagactctcaaaaaaaaattaaaaaaaaaaaatatgtgaagtcttgaataaaacccaagatctttaccatggcccctgaacagggcagagtatccattcttcagacactcttcatagaataccatggtgagctggcatatttattatacaatacagaaacaattttactggcagaaaacacattaaaccgtctaaactctgaatacagttgtcctcataaaaaatgttcaacatactattttgaggttttccattaatagttcttataatctttgtcccattatgtgttaatccaacaaaggatatccaataacaaacaccaaagtttaagaaaaatgtgctaggcgcggtggctcacacctgtaatcccagcactttgggaggccgaggtgggcagatcacctgaggtcaggagttcgagaccagcccagccaacatggtgaaaccctgcctctcctaaaaatacaaacattaactgggtgtggtggtgggtgcctgtaatcccagctactcaggaggctgaggcaggagaatcgcttgaacctcctgggaggcagaggttgcagtgagctaatattgcaccactgcactccagcctgggtgacagagtgagactccatctcaaattaaaaaaaaaaaaaaattaatgatagagaaacttaaatcagttagattgttttaggtatagcccatccttggtttttgtgtgtagcatctagcttggggaaaccctggatttctggaatcatatttagacacagtcacactagactaatgtaattcttttgggatgcaaaccacacgtttgacaccttaaatagcttttaggtatttggcttcccagcccctatttttagttacaaggggtgtacatgtgtgggtcagggtgggggtagctctttccgcagatgattagttttagccatgttactagttattgcacacattatctgtgtcctcacagcagccctgtgagtaagtgtattagggttctctagagggacagaactaataaggtagatgtatatatgaagggtaatgtattaaggagtatcgactcgtatgatcacaaggtgaagtcccacaataggctctctgcaggctgaggaaccaggaagccagtccaagtcccaaaacctcaaaagtagggaagctgacagtgcagccttcagtctgtggcaaaaggcctgagagcccctggcaaaccactggtgtaagttcaagagtccaaaagatgaagaacttggagtctgatgtttgagggcaggaagcatccagcatgggagaaagatgaaggctcagcaagtctagtacttccacactcttatttctgcctgctttattctagctgagctggcagctgattagatggtgaccacccagtttgagggtgggtctacctctcccagttcactggcttaaatgttaatctcctttggcaacaccctcgcagacacacccagaaacaataatttgtagccttcaatccaatcaagttgataatattaaccatcacaggaaggtactagtatcatatgtttaacagtagaaaccaagacaaatgcagctaggaagtgggagaactgggatcagatgcaggcagtctgattctaaatcagttgctgttacccactctgacaacagtaagtgagtagcctgctcagtcaagtactatattagtagggccctttacagacatatttatttctcacagtcactcaatgagacggctcttccagtcttacaatggagaaagtgaggctcagagactttaagtaacttaccttagacgactttactagtaagtataagaatcattatttggactaaagtctttctgaatcctcagcttgtatttttttccagtgttctgtgctgcctttttatctactagtgttttacatcaattttgaatctctttactaactggttaggttgatttttgccttttttttttaggttattctatatttgtcgttaagggtgatctgccagattgcgaagctgaccaactcctgcagatgattagggtccaacagatgcatcgaccaaaacttattggagaagaattagcacaactaaaagagcaaaggtaaaaatgaggcctgcagtatggaatatatggtagtatttcattatgagaattaaattttcatgcttagattgaatatgtggtccttgtgttgttggcgactctattttggaccttatattttagtgaagtttattagtttaaacttgaatcaactctttgaaatacttaaatatattaacttagttagctggtatggtatattcctagcacttcgggaggctgaggcaggctgattgcttcaacccaggagttcgagaccagcctgggcaacatggcaaaacctcatctctacaaatagtacaaaaattagccagatgtggtggtgtatgcctatagtcccagctacttgggaggcagaggaagaaggatcacctgaaactggggaggtagagactacagtgagccataatcacactaccgcactccagcctggtcgagagagtcagaccctgtctcaaaaaaaaaaaaaaaaagaaacggaaaaaaaaaacttagttggattcaaattgcaacacaatcattatattactagagcttatttgccagaaaacattttaagttttgacttacttaaagcctttacattacaaatgcctttatgttatgtctaaaatagaagattggttgcagttattaccagtgcttttgttctttagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtaaagacattctgatgtgtgttgtattcattgctgaagaattgattccaattattcttagatttcatggaagttaatgtactcttagaggtgttttgacaattactgcagaagcaatagctatatagtgggctttccctttagatttcttataatggaaatcactttttacaacctatattttattaggagtagttatatttttactcctggttattttatttggtttcaacactgtactaacacaatagtaaattgtggttttaatctttgtgggtatcagttgacccttatccaaatcagctgttacataaatatgtgccattagacactatggaagggcctggacagggaatataaactgattttacaaaaacccaacatttattggctatgcaacttaaaccgtaagcccactttggtgggcccagttttttagtgatataaactatcaatagagaaaagcgaaaacatatcccctagacaatctaggcaaagaaaaatgttaagacatagctcaaagtagcttaattaaaagtttgaagtgggttttttgttttatttttttctaactcatatgtatttgcttctactttctaatgaaattatttatcagttgatttccttagatatctaaataaaattgaaatttcattaatgggaagattatttttatcctgaacttttcttgcctctatgcatgcctctgagtactccatatggtgtgcaatcccatttttgattaatagagtcctgctggattagcagggacagaaatcagctttagatttctttcttttttttttttctttctttttttttttttttttttttgagtcagagtctcactgtcgcccagcctggagtgcagtgatcttggctcactgcaacccctgcctccgaggttcaagcgattctcctgcctcagcctcctgagtagctgggactacaggcgcctaccaccacgcccagctaattttttgtacttttagtagagatagggttttgcccttttggccaggctggtcttgaactcctgacctcaggtgatccacctgccttggcctcccaaagtgctgggattacatgtgtgagccaccacgcccagccagaagagtagaatattcttaaagagaaaacgttttaaaggcttactcaaatgagtataaacaaacatattgttgcttgaattggtaaatacagtgattggtttttgttgtgttgtgttttgttttcaggtagttccagaaacatatctcaagatatgacacagacatcaggtacaaatcttacttcagaagagcttcggaagagacgagaagcctactttgaaaagtaaagtagttggtacaagttaaagtagcatgtttaatatttgctttggctattttgtctatttgtaaatggttactgcctgaatcctgtgaatatttgaatgtattttttaaaaatttacagcaaataggacgggcacggtggcttacgcctgtgatgctagcagtttgggaggccaaggcgggcagattgcctgaggtcaggagttcgagaccagcctgggcaacacagtgaaaccccatctctactaaaaatacaaaagaatcagctgggcatggaagcgtgcgcctgtagtcccagctgcttgggaggctgagccaggagaattgcttgaacccgggacgtggaggttgcagtgagccgagatcgcaccactgccctccagactgggtgacagagtgagactccgtctccaaaaatatatgtatatatatataaataaaaataaaaatttacggcaaataacatgaaacaaaaaaaccttgccccaatactggataaattttttaaactgagtgaaggaaaccttataaaatttcatttattaaaagaaaaatgaaattaggacaagacaagaagaatgccaattgatcctttggatgtacttcttgcttacctgattaaccctgcaaaattcctctaccaatcagtacgaaaaacagctttggaggtatgggagcgcattcccaaatagacgtggtagttcatttagctgctcatggccgcttcaggcagtcctgtaagcctgttagcatcaggggaatggatgcaaaccataaatctggatcaactcctaaaaccttaccttgtgcccagccttgtaagtgcttgctaaataggaattccaccatatgaaaatacattcttttcaagtaactatcattcagacttttgtcccccactttttttttttaaagaaaaataaaaggctgggcacggtggcttacgtctgtaatcccaccattttaggaggccaaggcaggtggatcacctgaggtcaggaattcaagaccagcctgaccaacatggtgaaacctcatctctactaaaaatacaaaaattagccgggcatggtggtgggtgcctgtaatcccagctacttgggaggctcagacaggagaatcgcttgaatctgggaggcagaagttgcagtgagctgagataacgccattgcactccagcctgggggacaagagcgagacttcgtctcaaaaaaaaagagaaagaaaacttcatgttaaagattacaagataaataatcagacccactgatcctaggtcagaaaacagagtcatagctcaatctgacttactatttgctgtatttcatccattctgagatgcacatagtttcacatttcaatgtctctgaaattgagaagcatcttacagtcataattgacagtatattagcagcacctataaatattggctcattttacatttgatggtataatgaagaaaatatttacctttttttctgttttgtttttaagtcacaactcagaagtagatgaaggaaaattctgatcagctgacatcctcttaatgtgagatatttctagtctttattcagtatagattaatggctaattatatgttaaatttcaaagtagtgcttattagtgctttttacttttaagtttcaaaattaacttttttattataataaactccaaatttatacaaaagtagaaaaactagcatactcctgtttatgacccagattcaacaaatactagcacacggccaatcttgcttttttttttttttttttttgagatggagtcttgctctgttgcccaggctggagtgcaatggcacaatttctgctcactgcaacctctgcctcctgagttcaagcgattctcccacttcagcctcccaagtagctgggattacaggtacacaccaccatgcctggctaattcttgtatttttagtagacacgggatttcaccatgtcgtccaggctggccttaaactcctgacctcaagtgatccacctgcctcggcctcccagagtgctgggattacaggcatgagccactgagcccggcccaatctcgttttataatactcccatctcccattctttccactgtcccacctgcaagtttggattattttgtaacaaatctcaatcatcatattattctataaccattttaatatgtgtctctaaaatatattagctttatttttaacatagttaaatgctattgtcataaaataataatcataataattaattgtaattctatatcatcaattatctagttaatgtaaaaaataaatctaaggccaggcgcggtggctcacacctgtaatcccagcactttgggaggctgaggtgggcagatcacctgagatcaggagttcaagaccagcctgaccaacatggagaaaccccatctctactaaaaatacaaaaaattagccaggcgtggtggcgcatgcttgtaatcccagctacttgagaggctgaggcaggagaatcacttgaacccgggaggcgaggttgcggtgagccgagatcgtgccattgcactctagcctgggcaaaaagagtgaaactccatctcaaataaataaataaataaataataaaaaataacttaaatctacttaattagaaaaactaacattctaaaaattttattttaagaaatatcaaaattggctgggcacggtggctcacgcctctaatccctgcactttggaaggctgaggtgggcggatcacctgaggtcaggagggtcaggagtacaagaccagcctggccaacatggcgaaaccctgtctccactaaaaatacaaaaattagccaggcatgatgatgggcacctgtaatcccagctactcaggaggctgagacagaagaatcgcttgaacccaggaggtagaggttgcagtgagctgagatcaccccactgcactccagcctgggtgacagagtgaaactccgcctcaaaaaaaaaaaaaagagaaaagaaatatagaaattaaagcatacatggccaggcgtagtggctcatgtctgtaatcccagcactttgggaggctgaggcaggcagatcacttgaggccatgagttcaagaccaacctggccaacatggcgaaagcctgtctctactaaaaatacaaaaaaattagttgggcatggtggtgcacacctgtaatcacagctactttggaggctgaggcaggagaatcgtttgaacccagaggtggaggttgcagtgagccgagattgtgccactgcactctatcctgggtgacagagcgagatactgtctcaaaaagaaaaaaaaaaggctgggcgcggtagttcatgcctgcaatcccagcactttgggaggccgaggcaggcagattacgaagtcaggagatggagaccatcctggctaatacagtgaaaccccgtctctactaaaaaatacacaaaaattagctgggtgtggtggcaggcacctgtagtcccagctactctggaggctgaggcaggagaatggcatgaacccgggaggtggagcttgcagtgagcagagatcacaccactgcactccagtctgggcgacagagcgaggctctgtctcaaaaaaaaaaaagaaagcatactctcacctccttcagtgactgatgttagtattttggcacattctttttctgtgacatatacacacttaccttgtaagtgttgtactcatttcctatgacagtaaatagtctttgtaacaggctgcatgatatttcataaaatgaatggatgtggcataatttatatgtgagccttttgaattctgctattataattaatattgcaatgaacaattcttatattgcctctacacctcaaatgtcttatcatttcttctagtttttctgaggatgtcagattattgggttaaaggatatgaacatttttaaggccttggaacagatttctaaattgctttccagaataattcccatgtgatactttcaccatgtttatttcagacttttttttttttttttttttgagacgaaatctcactctgtcacccaggctggagtgtagtggcatgatctcggctcactgcaacctccgcctcctgagtttaagcgattattctgcctcagcctcccaagtagctgcggttacaggcaagtgcctccatgcctggctaatttttgtgtcttttgtagacatggggtttcaccatgttgcccaggctggtttcgaactcctgagctcaggcaatctgcctacctcggcctcccaaagttctgggattacaggcgtgcaccaccgcgcccagccatcagagtcttttttgtcaaaataaaatggtctaaagacatacatcatagagaaactataatacaaaatttacaggtatatctaagaaaagaaaagtatatttaaagcataaaaataaactgctcttttacttaaaattttttaaaaactggattaaaaatatgaaacttccaacaaattgagcttttttttttttttttttcttttttgagacgaggtctcgcttttgtcacccagtctggagtgcagtggcgcgatctcggctcactgcaacctccacctccctggttcaagcaattcccctgcctcagcctcccaagtagctgggattacaggcgcatgccaccacgtcgggctaatttttttgtatttttagtagagagggggtttcaccatgttggccagactggtctcgaactcctgatctcaggcaatctgccagcctgggtctcccaacatgctgggattacaggcatgagccactgcactcggcctgaactttttatagtagtaacgataattcagtaatgtccaataatgactaagtaagttataacaagtacaatgtcagcaataactagtgctttttagtaaacagggtcaggcaaccttgtacccttttaaaaatgttcgaatatcgatatacctccttcctacttggtggaggattgattgaggaggaaagtgtgcagtgatggttaccagcttcagcctcttggcttgactttgcaaatactggtgagaatttggaaagagcttgagaatatcttacatagtcacatgttgctgagaagagttaagaactaacttcttgatgttcatttttaacaatggcttgcattcaaaaccttgtagagctcattagtaggagctaagaagctaatatttgcctttcactaaaattcctgattacttagcctaggtagttcgttgtctctctaggttctgtctttgggagcttgggtctaaggttatcaagctaactctttcttccctctcacccttcccaaattgaccctggtgctgatttgttattcatacgattttctagtttttcttttccctttttgagtatttgaagcttcatactgaatatagtaatcatagtattcatgcataaagaaaatcataaagtaattgcataaatgcataaagtaatcatagttttcatgcattaaaaaaactagttttggctgggcgctatggctcacgcttgtaatcccagcactttcggaggccaaggcaggcgaatcatctgaggtcaggagttcgagactagcctggccaacatggcgaaacctcttctctactaaaaatacaaaaaaattagccgagtatggtggcgggcgcctgtaatcctagctatttggcaggctgaggcaggagaatcacttgaacctgggaggcagaggttgcagtgagccgaggttgtgccattgcactacagcctaggcgacaagagcaagactccatctcaaaaaaaaaaaaaaaaaaaaaaaaactccctattacagattcataatttatgagtcattaaataatattttcaagccatgacattttttccagcagtagtctctaaatctgttttaccatcataaaaccccaagcaaaactctactacatcagctgtgtcactgtaaaacctgccttaactcacagaagcatgaaattaagcaatgtgtgtgaaactattttataaactgtaaagtattccatacatacatgttggcagttattaatgtcttctctaggtgtggctttgaaatggatgcagatgctttctgttacaaaaaacataagttgcaaatgttctataacaaggagagacacaaatatcttcatggacatggattgctatgagtgtttgattgcctaatacttgagccaccacttcagtgatatggtataatttatcaaacagtgttgagaaacagaaactactggggatgttttaaagaggaaaatacttaatatagaaattaggggtttacataatcttaagaaaggatgaaggtgcagctcttagccaggcctccacagtaccacaaaccaacttgcaggaagagctgtaaccactgccccagttgggacaatgggtaatgaggatattaaatttaagaacatactgctatagcaatgatccttggcatagaaagctgccaccacaattgcctagagatgggaacatgaagtctggcccccattgcaacagcagtgaagcagaattttgggactggcatctcccaaatggctttgcttgccaccagagaacaaccaaagtggagggagatggctaggcctcatttctgcctattttattttattttttgagacggagtcttgtctgtcgcccaggctggagtgcagtagtgtgatctcggctcactgcagcctccgcctcccagcttcaaacaattctcctgcctcagcctcctgagtagctgggattacaggcacccgccactgtgcccagccaattttcttatttttagtagaggtggggttttgccacgttggccaggctggtcttgaactcctgacctcaggtgatctgcccgcctcagcctcccaaagtgttgtgattacaggtatgagccaccatgcctggcccatttctcccttttttttttttttttttttttgaggtggagtctcactctgttgcccagactggagtgcagtggtgcaatcttggcgcattgcaacctctgcctcccagtttcaagcaattcttctgcttcagcctcctgagtagctgggactacaggtgtgtagcaccacacctggctaatttttgtttttgttttgttttttttgagacagagtctcactctgtcacccaggctggagtgtagtggcatgatctgggctcactacaacctccgcctcccgggttcaagcaattctcctgcctcagcctccagagtagctgggattacaggtgtgcgccaacacacctggctaatttttttgtatttttaatagagatggggtttcaccatgttggccaggctggtctcgaactcctgacctcgtgatccgcccgcctcggcctcccaaagtgctgggattacaggcatgagccaccgtgcccagacaaggtttgtatttttagtagagacagttttgccatgttggccaggctggtcttgaactcctcacctcaggtgatccgcctgccttggcctcccaaagtgctgggattacaggcgcaagccactgtgcctgacccgtttctgctttttaaagctcatgtgagcacttaatttgtaaccagaatcctacttgtaaaataatctaagacatgtagcttttagctttgtaacctctataatattgatggcacagtgggagtggatgctgagtaccacttgaacatgttccacctcagtgtcttcacagctggaaggtgtctacattgtttcaaggtggacaattgatttacttctcatttttcataaactaaaagtagaataaaggctattcctctaaaattgctatctcacctgtcactcccttgcattctcacataccttcttgagtggaggggcagagggcatggagtgatagcagatgtgccaggaattctccataactcagtccgtccctcttgtgctatgttgcagcatcaggatttgctaatgggaggatactgcccttacgtgcatcattagccatgcacactaaggtcttacacctacacacaggtcagtattctggctcagagaccaacagggagaaattgcagttctcattagttgaactttctttattgttcacagttttaaaacacaaaattgagaggaactctataaaaaatgtgccattctattaataattgttgctggtaatttaaaaatccttgttccttttcaaattcttatataccttttttttttaaacacttgatcttagccaaaagaccgagaagcaatcttttttttttttttttttttttttaacctatagcttctcactgagattgtcagctgtttgtaagttttggtttttggttttctgtgtttgtatttacatatatgaaatacagattgagtatcccttatccaaaatgcttaagactggaagtgttttagatttggggttttttaggatttgtgaatatttgcactatacttaccagttaagcattccaaatccaaaatttcaaatctgaagtgttccactgagcacctcttttgagtatcatgttggtgctcaaaaagtttctgattttggagcatttggatttctgattctcggatttaggatgcttgacctgtaatttcagatttacataaaagcagaaatagtacacagagctccttatatccttcacccagattccccaattattggcctttctgaaccatttgggaataatatgcagatatgattttccattatgtctcagttgttcagtgtatattttctaagtacaagaatatattcctacatatttacatgataaccgtcatgtttaaacattttaaaatggggatttgtattacattgtttctctttttgaaaaaattacagaggagcttaatgcaatcagtattacttaaaatctgataatgtgtgttaaatagtagttttcatttatttcatttatcaggtgttcagtgaatgcttactatgtaacagcacagttatcagcactggggaaatagatgagtaagataagatttgcactttcattagcttacatgccataaagagggaaataaagagaacaccagatgatgataagtttatgctgagaattaaaatgaagtgatgaaataatgggaatgtcaggtggctacttttggtgggatggtcaggaaaggcatctctggggagataaattttaagctcagacctgagtgaaaagaatgagccagccatggaaacattatgttaactcacatggtagtttgaaatgctttatctgatcaaaggtacttatttttggtgactttcaacaatattaagggtctataaaccaacactcatttgcataagaataactaccagtgaatctttttgtatgataggttttttgtttgttgtttttttgagacagagtctcgctctgtcgcccaggctggagtgcagtggcgcgatcttggctcactgcaacctctacctccccggttcaagtgattctcctgcctcagcctcccaaagtagctgggattacaggtgcctgccaccacgcctggctaatttttgtatttttagtagagatggggtttcaccgtgttgtccaggctcgtgtcaaacttctgacctcaagccatccacccgcctcggcctcccaaagtgctgggattacaggtgtgagccaccactcctggccatgataggttattttgtgatgaaaatacctacctcttaatttgtctgataaatttaaattttatgtctagatttcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcagacagcagcaaaagcagcaacagcagcagcagcagcagcagcagggggacctatcaggacagagttcacatccatgtgaaaggccagccaccagttcaggagcacttgggagtgatctaggtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaagctagatgtggtggctcacatctgtaatcccagcactttgggaaaccaaggcaggagaggattgctagaacatttaatgaatactttaacataataatttaaacttcacagtaatttgtacagtctccaaaaattccttagacatcatggatatttttctttttttgagatggagtcttgctctgtcacccaggctggagtgcagtgtcgcgatctcggctcactgcaagctctgcttcctgggttcatggcattctcctgcctcagcctcctgagtagctgggactacaggcgcccgccacatcgcctggctaattttttgtatttttagtagagacagggtttcaccatgttagccaggatggtctcaatctcctgacctcatgatccgcccgcctcggcctcccaaagtgctgggattacaggcgtgagccatcacgtccggccagaaatcatgaatattagtaggtgaaaaataaacacattttaccacctggaaaatgaaaaatacttgagtataatctaaataacaatgggaagtgcagagttactttccaggtctcggtttaaatatgtcttaaactttggccaattagtagtagaagttgagagaaaaagtaactatctgacaaagaaattataagcagaatatataaagaactcttaaaactgaataatcagaaaacaactcaataaaaaggtgaaggatttgaaaagatatttcaccaaataagacatagggatgacaaataagcacatgaaaagactctcagcatcactagtcacagggaaatgcacgataaaaccacagtgagacaccatggcacccctgtaggtatggctttaatgaagaaataaaactgacaataccaagtgttggcaaggatccaagcagctgagactcatatactgttaatgggaatgtaaaagtgtacagctttggaaaacagtttggcatttttttgataaatgtatacttagccatgtgatccagcagtcccaatcatgtatatataaccaaaagaaaagaaaacttaggttcacataaaaacttatatcaaatgcttatagctgaccaggcatggtggcccatgcctataatcccagcactttgggaggccgaggttggcagatacctgaagtcaagtgttcgagaccagcctggccaacatggcaaaaccctgtctctacttaaaatacaaaaattagccaggcgtgatggcaggcacctgtagtccagctattcaggaggctgaggcaggagaatcacgtgaacccgggaggcagaggttgcagtgagccgagatcgtgccactatactccagcctgggtgacagagcaaaactctgtctcaaaaaaaaaaaaaaaaaaaagggctggacacggtggcttacgcctgttatcccggcactttgggaggccaaggctgatggatcacctgaggtcaggagttcaagaccagcctggccaacatggtgaaaccccatctctactaaaaatacaaaaatttgctgggcatggtggtgggcacctgtaatcccaggaggctgaggcaggagaatcacttgaacccgggaggcggagattgcagtgagccaagattgtgccattgaactccagcctgggtgacaagaccaaaactccttctcaaaaaaaaaaaagattatagcatctttattcatcattgcccaaaattacaaactgcctaaatgtagaccttcatttagttaatgaatgcacaaactgtggtatatccaaacaattgaataaaaaaaggaatgaactggtacttttttctattcctcctgtttaagtacagccaaaacacctcaacatttgtataaaacatgagctgggctgggtgcggtggctcacacgtgtaatcccagcactttgggaggctgaggcgggtggatcacctaaggttgggagttcaagaccggtctgaccaacatggagaaaccctgtctcaactaaaaatacaagattagtcgggcatggtggcgcatgcctgtaatcccagcttcttgggaggctgaggcaggagaattgcttgatcccgggaagcgaaggttgcagtaagctgagattgcaccattgcactccagcctgggcaacaagagcaaaactctgtctcaaaaagaaaaaaaaaaccattcagctgaatctcaaaggcagagagaagacagactggctagggaccttggaaccagaggagcagtgtggtggggagtggactggattttctttttgcctcatttatcctggacttggtgctggagaagctatgggttcagaccaagagaaaaccccatgaaaagcctgctctctctagccaaaagaggcaacctagcaagataaaaacctttagataataagcacttgactccagtcaaacaaaacagaataaactggccccattcacccctgtcagcaaaggccaagtgggagccaagatatgtaccccaacctggaagtcataaggtacacttctcccctttcccagccaaggtggtgttagagaaggctgactggggagctgggattctcattccctccaggaggtgataacactcctttcacatggtgtcagtggtcacagggaggctgaacttccacccagtaatacataggcatctctctggctcctatatgggtgatgttggagaagaggccgagtagagaatccagactgttgctgacacccagcagtaacaaggacacctccacaatgtccgtggaggccatgtggagatcagtaacaaggcactgctctccctcccagtcagagagatgtcagtggaggactagggggctagaactcccatgtgcgttcagcagtaatccccatgaccgccactccttgacatcacaggccttgaagaaacctggactttcactcccctctggttgtagcgaggtggcactcccttttccctgttgccagtgctgtgtcagtggaggcttgctaaattggaagatgtaaataagattcacattctcataacataataccccaaattttcaggatttaattgaaaatcactaagctgggcatggtggctcacacctgtaatcccagcactttgggaggccaaggtgggccaaacacttaaggtcaggaattcaagaccagcctggccagcatggtgaaaccctgtctctactaaaaatacaaaaattagctgggcgtggtggcacatgcctgtaatcccagctactgggaaggctaaggcaggaaaatcactggaacctgggagacggaggttgcagtgatccaagatcgcactagtgtactgcagcctgggcaacagagcaagactccatctaaatttgtgtcaggattcccagaaggagatgagaaagggtggggctgaaaaaaattgaggaagaagtcatggctgaaaatttcccaaatttggcaaaagtcagaaacctacagattgaaaaagctgaatgaagctcaaatatgataaactcaaagaagttcacacagagacacatcacagtcagatttctgaacactgcagacaaaaaatgaagatctcgaaattagcaagaaatgaccttacctaagcaatttgaatgacagcagatttcccatcagagatcataaaggccagaaggaaggggtacatacaacattttttctagtgctgaaagacaaaaactctaggctgggcacggtggcacacacctgtaatcccagcacttttggaggctgaggcaggcagatcacctgaagtcaggagttcgagaccagcctggccaacatggggaaaccctgtctctactaaaaatacaaaaattagccaggtgtggtggcacgcacctataatcctagctacttgggaggctgaggcaggggaatcgcttgaacctgggaggcgacggttgcagtgagccaaggtcgcgccactgcactccagcctgggcagttgagcgagactccatctcaaaaaaaaaaaaattatccaggcttggtggtgggcgcctatagtcccagctacttgggaggctgaggcaagagaattggttgaacccaggaggtggaggttgcagtgagccaagctcatgccactgtactccagcctgggtgacagagcgagaccttgtctcaaaaaaaaaaaaaaaaaaaaaaaacaagaaaaaaactctaaacccagagttacatatccagtgaaatatccttcaggagtgaagggaaaattaacgatttgtcttcaggagacctaccctaaaagaatggctaaaggaatttctctaaacagaaaagaaatgataaaagaagtaattttggaacatcaggaaggaagaaagaacaataaaaagagtaaaatatgggtaaacacaatagactttcccctccttttgaattttctaaattgtatgatggttgaagcaagaattatagcactgatttggttttcagtatatatattggaaatatttaaggcattatgttacagatgaaggagggtcaaaggatataaagggaggtaacctttctatatttcttttgtactgatgcaggcactttggaaaataatttcactatttgtttaaaaactgaacataccctgaccatatgacatagcatctatactcctgggcatttatcccagagaaacagaaatttatttattttttttttagtattacactccgtaagtgctgtaatactagcacttagggaggctgaggcaagcagattgcttgagcccaggagttcaagaccagcctgggcaatgctgcacagtcaaaaaagaaaaacaaacatttagaaaactattttaaaagtctttaattgctgaatgcctctttggctaatatttggaagatcattattattatttttcttttttaggcagagtcttgctctgtcactgaggctggagtgcagtggcgccatctcggcttactgcaacctctgcctcccgggttcacgccattctcctgcctcagcctcccgagtagctgggactacaggcgtgtgccaccatgcccggctaattttttgtgtttttagtagagatggggtttcactatgttagtcaggatggtctccatctcctaacctcgtgatccgcccacctcggcttcccaaaatgctgggattacaggcgtgagccactgtgcccagcctggaagatcattatttagtcctacaactgacacattgttccactgacgcaattgcccaggctggtcttgaactcctgggctcaagcaatctgcctgcctcggcctccctaagtgctagtattacaggcttgagccactgtgcccagccaaaaatagaaatttatattctcacaaaaacatgtacatgaatgtttatagcagctttacttgtcataatcaaaaactggaaacaaccaaaatgtcctacagtgaaacaaactgtagtacatccatagcatgtaatactctactgtcaggattaaaaagaaacccactgttggcacaggcagcaccgtggctggatctcaggggcattatgctgagtgcaaaaaagcctcaaagggtcttacactgtatgattccacttgttcaactaaaaatgacagctgtatagagatagagaacatattagtggtttccactagttagagaaagtgggtaaaagataggtgggtgggaatataaatcgatagcagggagatctttgtggtattataacacttctatgtcttgattgtagtggtggtggttacatgaatacacgtgtgataaaatgccatgtagaactacatataacgttgtgccaatgtcaatatctaggttttagtttgatctttagttacataagatgtaactattgggtgaaattgggcaaaagagtacacgaaacctctcttaaatatctttacaacttcctttgaattgacagtttttcaaaatagaaagttgggtttttgtaaatacatgaattgttgatatacacaacaaatctcaaatgcattatgctacgtgaaagaagccatattcaaaaggctacatacctactgatgccttttatatgacgtgcaggaaaagataaaactgtaggacagagaatatactggtggctatctgggattaggaaatggggatcgaccacaaaggggcagcatgggggaattttctggggcaatggaatggttgtgtatcttgatggtgtatttgtcaaaatatatagaactataaaagtaaattttgctttatatgtattaaatcaaaaaaagaaactcgtgctcaaatagaaatacattttctgagaacttgccttttgatgactttgagaattttctggaaattttaaagaaatgtggttttgtttcccaacaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttctatttccttttgaatttgtgtttattgttaattcatagctattcaaagtgtgattagagctgggcttggtggcttgcatctacagttccagctacccaggaggcagaagcaggaggattgcttgagcctaggagttcgaggctgcagtgagctatgatcctgccactgaattctagcctgggcgacaaaacaggaaaaaagtatggatggaggaccagcagcatctgtatcacctgtgagtctttcagaaatgcagagtttcaggctacactcggacctactgaatcagaacttgcactttttacaagatccccaggacactaaagtatagagtgaagcttgagaagcgctgttgtgtggattgttcttaaccagctgcagtgatgaatatgaataacgcaggccagcacagtccattgatattctattccagcttactgcctgccaaaaggtccattattactggatcctcagtcttttccaagagaagctaagaattccaaatttttatttgaaatatattttttaaatgtttgttcaactggcccagtgccagtggctcatgcctttaatcctagcactttgagaggccgaggtggaaggatcacttgaccccaggagtttgagaccagcctgggcaacataaagagaccccatctctattaaaaaaaaatagagacaatgctgccttaaaaaagtcaaataaatgtttgctcaactgatttttaatactgagggccaaacaaagcacatcaaatttttaagtgctgcttttcctcattttatccaactctggacaccagaatccaaatgtagtgattggaatccacctagactgattgaggaatatattgtcctcaaattttatgagggttgactattcattttaactttaattaggattgggcacaaattttgaaacataataacattacaggaccggggcccgatccagaccccaagagagggttcttggatcttgtgcaagaatgaattcagggcaagtccataaattgaaagctagttcattaagaaagtagaggaataaaagaatggctaccccataggtagagcagccctgagggctgctggttgccatgtccacccccgcgccccctccccccgctttgttgttgttgtttaagacagagcttcgctgtgtcatcaggctggagtgcagtggcatgatagctcactgcaacctctgcctcccgcgttcaagcaattctcctgcctcagcctcctgagtagctgggactacaggcgtgcaccaccacgcccagctaatttttgtatttttagtagagatgcggtttcaccatgttggccaggaaggtctcaatctcttgacttcgtgatccacccgccttggcctcccaaagtgctgggatgagccacctcgcctggctggttatttcttgatgatatgctaaacaaggggtggattattcatgcctgccctttttagaccatttagggtaacttcccggcattgccatggcatttgtcaactgtcatggtgccaatgggagtgtagcagtgaggaggaccagaggtcactttcatcaccattttggctttttcagccggcttctttactgcaacctgttttatcagcaaggtttttatgatctgtatcttgtgcagaccttctatctcatcctgtgacttagaatgccttaaccatctgggaatgcagcctagtaggtcttggcctcattttaccaaccccctattcaagatggagttgctctggttcaaatgcctctgatatttccctactcccttttataagaaaacccttaatcctaagggttgcagagggatgaagatccatcttctgtattcttcaggctgaataggggtgatgatattcctgcctatgagagtctcttgtattagggtagagaggagctcagtcagtcagtatggctccctatccttcctcccttccccagcccctgacaactaccattctactttgtctctatgattctaagtatctcctataagtggaatcattcagtatttgtagggtttttttgtgactagtgtatttcacttagcatgtcaaggttcatccatgttgtagcatacgtccaaattttcttctttttaaaaactgaataatattccattgtatgatatatgcacattttgcttattcatttatccatcaaagaaaaccagtagcttttttacataccggcagtaacctggttaaaaaaataagatggaaaataccaagcccgaaaatgtcaaattcttgaataccagacaggtactataatccaagctgagcagagaggaatggcccttgtctcagccctgagccacatgtcactacatgggccccaaggagctcttgtgcccagagtcctgctaaggctcgttcttcacccctctaaagtggctcaaactagcgtaggaggccaggagggagcagattctatgccacctccattgcttccgtgccttctccagatccattttttccaaaagctgtgtttgaattctgcacaggggcatctctgtctccaacactgaaacccc

SEQ ID NO: 3 - DNA, 72000 bp, Homo sapiens
attaattcatttgaaagacatcaactacaatttaaaaaaacaaaacaaaaacagaaaaggtggtttgcaatttttttcacttcatgagactataatttatttccctgggtaggccacacaaatccttaagaacatggactttcccccatacaaggctgtgaaccgtgcacacagagatgctcgataaatggtaccgaatcctctccttcagcacctggtcctctctggcagtctccattctcttacctctcactgccccctctacttctctgaacctgcagtccccaaagtcaccaatggtctccatagcaacaaaatttagtgggctcttatacgaccttatattgcttgacttcttggcagcaactgacactgactaccccttgaagcatcccctgcccaggccttccacaatgtcacactctcttgcttctcctccctcctccttggttattgcttctcctatcctcacggctcagctctagccctcttctcaatcacttccttttccctaaaatgattttattcactcctatggtttcagttacttttttttttttttttttttgagacacagtttcactctgttgtccaggctggagtgcaatagcacgatcttggctcactgcaacctctacctcccggttcaagtgattctcctgcctcagcctcctgagtagctgggattataggcatgagccaccacgcccggctaatttttgtatttttgtagagatgaggtttcaccatgttggccagcctggtcttgaactcctggcctcaagagatctgcctgcctcagcctcccaaagtgctgggattacaggcatgcaccaccacaatggccttagctatctgttttttaatgacaattcctcattgagttcaaagcagcctcctgaacttcagactcggtatccaaccattactcaacataattactcaaatgtccatagatatttcaaattcagtatctccaaaatgtaaactctatcttattctcttcccaaaaccttgctgctcctcccatgtttctgctctcaataccactcctgccacagtggctgcccaggtcacaaacccagaagtcaccttcacatttctgtctcctaccttccccaatccctcattgtggtaggagttattaagaaattattttaggcagatagagaggaaaaggggtccttgaaaagtttttgtctcttttgaagcagctcctgagacgtttcctgtctagcataaaagccctgacttttagacccgagctggcaacgtttgatatgcaaatgccggcagttagaaactgggtccaccaacacggtgattcccaccatcgtcctcttgcccttgttctcacaagtgcctggcaacatggccacccccacatatccccacatgagtagaacatcatagcgccctgcatttgcatattaaaaggctagggtggaatggccagttttattagggggctacatgaatgacatgcgtggtcaaaccagtcccctgagccctatgcaaatcagacaccgcctcctgcatcctcctcctataactggctggtatctcccacgcactctgggtctcctctctcagctttggagcacccatccctctgtctctgtacaggggagctgcttctttctttcttctcccttccttcttgcttattaaactctccgctccttaaaaccactccacgtttgtccatgtcgttttatgtaattcgactcaagacgaaaaaggctagtattcctccactcggtatcatcaccacattctgttgagtctctttacgtccaaagaatctctcaaaatgggctatttccatctttaagacactctcttaaaccaccatcgtctctcccaagattccttcaacagcaactgcttttttctgcagccagtgatctttctaaaaggcaaacatgaccacatgtcttcttttaaaatatttatatagctccccacatcctttaaaaggtacactttggccaggtgcggtggctcacgcctgtaattccaggactttgggaggccaaggcaggtggatcacctgaggctgggggttcgagaccagcctggccaacatggtacaacctcttctctactaagaatacaaaaatagttggacttcgtggtgcacgcctgtaatcccagctgtttgggaggctgaggcaagagaatcacttgaacccaggaagcagaagttgcagtgagcggggagattgtgccactgcactccagcctgggggacagagagagggccgtctcaaaaaaaaaaaaaaaaaaagtacaccttaatctggtttcaaggtgcaagatctgcacctatccttccatttaagtttcatcaaaccgctgctacctccccgtctcccacacaatttatgggacttctaagttccctctaaagggtccgaacacctacactggtaacaagccacctggatttgaatcctggcaagacaacttactatctgaccttggacattgtgctgttcttaacctctccgtgcctcggtttcctcatgtgtatgaataacatcaacacctacatcaaagtttgctgtattaaatttgataatatatgcaaagcatttagaaaagtgcctagctcatagaaagccttatgtaaatattaactatcattttttttcttttttggggtggtgggggaggggtttcgcttttgttgcccaggctggcgtgcaatggcacgatctcggctcaccacaacctcggcctcccgggttcaagcgattctcctgcctcagcctcccgagtacctgggattacaggcatgcaccaccacgcccgggtaattttgtatttttagtggagacggggtttctccatgttggtcaggctgatctcaaactcccgacctcaggtgatccgcccgcctcggcctcccaaagtgctgggattacaagcgtgaagcacctcgcccggcctagctatcatttttatacaagtgctgggttttgggagaatgtaatgatggcttttttcttactaaactttcagtgcaggaggaggagaaagaaagtaaatagttatatgaacacagtagaaagtcaaagtggaaaacaaaaagaacatagaacccaggtgagcggtccagacctccccccagaaacctaagaatccatagaaatgggtgggaagcggagaagatcctccagacagcaggtggcgatgtagcatcccccagaaggcccgctaacagaagctaggaggacgcgctaccaaggtcacgtgtccccggcgttcactcgctcttcgcttcacgacactcgcatcctcacgggtgattggtctgcgtgcggcacgtgggcggggtaccggggcgggccggggaggggcggggtgggcggaggagaggggcagggggcggagctggagggggtggttcggcgtgggggccgttggctccagacaaataaacatggagtccatcttccacgagaaagtgagtgtccgcgttcggtggggagctgtctgccgcgcggtggcgggcgtggagcgcggcatcaccgcctctcggagggctgggtggggcccgagtcgcccccatgccgatctcgcccggcgaggggcgacgccgcagcctcccgcctcctcggctcgaggaggggagcatcacctacgcccctacttcccccgcggcccccgccctgggagccgggagggagtatgggcggggccgggggcgtctcgggacacgggagtggggtggcgcccagtgggtttgcttctgcctttctccgtcactttccatcgcttttcggaggattccttcacccctccccaatccttccctctccctagggtctagctagagtcatctctgggacacctccctcaacccctcctaccctaatcctggcagaattaacttttcctcctccggactgctcaattctatattggagtcttccctacacgtagatctttggggtcttgttcgtgtctttcccctgcactaggtccgcgagcctcccgagggaggagaccttggctcgcccactgtagggcctgacatttaggaagtgaagtaggaaacccggcgtgcccctaaacagggaagtcgtcacaagagtttttattacgggatgtttgggtttggtttcttttggtactcccatctttccggagcaggcggccagctttgtttttaggtattaggagtggactgggatgattttgttgtagtctgcctagcctgctgtccctttaactcttccgtgaccatgcacttgaagatactgtttgtgatatgtaaagaaactcctcgtttctctcatactattatccagccatttgtgtgtgagtgaagccttccccaggacagctttggcacatggtatcatgtttcataatagtttcgtgtttggaaagagttgctggtaaggctgttatttaataggaggagcaaagggtttttgttttattaaatacttataaatgatcatttatcccagacatttaaaattcacacacacacaacaaataaagcaaagacaaaagaatacatttaccaaatgtaaatctgtagcataaattttttttaatttttattttaaagatggggtctcattctgtcacccaggcaggtgtgcaatggagagatcatggctcactgcagccttgatctcctaggcacaagcgatcctcccgcctctgcctccagagtagctgggactacaggtgcatatcgccagggccaggtaatgtttttgggagagacggggtctcgctgtgttgcccaggctggtctcgaactcctggactcaggtgattctcccacctcggcctctcgaagtgctgtgattacaggcgtgagccactgtgcctggaacaaattgttaagtacaatgcttttcattgtagaaaacatctcggaaacttttgaaataggctgatgttcagtgggggaggaaggactcagtcgtatagttgtcactaattttttgacttgattgacatgactcgtaaatcatagacaatagagatttggttgcttggctgagtagagtgcgtgaaaaatacacacgtacttttttttttttttttttgagatggagtttggctcttgtcacccaggctggagtgcaatggcgccatcatggctcactgcaacctccgcctccccgttcaagcgattctcctgcctcagtctccccagtagctgagattacaggcgcccgccaccacgcccagctaatttttgtatttttagtagagacagggtttcaccatgttggccaggctggtctccaactcctgacaggtggtccgcccgcctcggcctcccaaagtgctgggattacaggcgtgagccaccgcacccggccatatttttgttattaattttcaaaggctttggtgtgggaccacatttcaacatggaaggccttaaacatgttccacactacttcctgagaattagacaagatttttaacaatattgttacctagttgggacacatttgtactgacccatgggatgaaaaaaagctgagtgctagcctagtgaaaatctacttacccgaaagaaatccctcttagtctgggtgcagtggctcacaccagtgctttgggaggcccagacgggcggatcatgaggtcagtagtttgagaccagcctggccaacatggtgaaaccccgtctctactaaaaatacaaaaaattagccaggtgtggtggcaggcgcctgtaatcccaggtactctggaggctgaggcaggagaattgcttgaacccgagaggcagaggttgcagtgagccgagaccgtgccactgcacttcagcctgggcaacagagcgagactccgtctcaaaaaaaagaaaaggaaaaaagagtccctcttaattatcagcatgtgtataggcctacagatacttcaggaatacctttaccattatcatcaacttgtatctacatagcatgtgaagattcaacaatttagttttttgggcgtcctcaagagtacgcacctataaccatatggcccaattgttaatctcctatacagtccattctgggaatgtttgggcttactgtgccatttttccgttcactgccttcccctctgcaatatacctttaacccttgctaggtcctgggtttggagagccagagaaccaactttggccctaaagaagctgtgtaggtagcaatatctgcctacgaagggccttgcaaccatttcctcttggaaccttggtttcctctttctgagtagtcactttgagtaccctttattaagttagaatgtaaaaacagtttctcactgatatatctgcagtgcctgagagagggcctggcacagagtaagtactcaataaatatttgaatggggccgggcgtggtgagacctgtctctacaagaatgaacaaaattagctgggcgtgttagcacatgcctgtagacttgggaggctgaggtgggaggattgcatgagtctgggaggtcgaggctgtagtgagccatgatcgcaccactgcactccagcctaggggacagagcaagatcctgtctcaaaagaaaaaaatgtatatatttgaatggataaagagatggctttgagtttctgagatatatatggtgctgtttatctaaagtaaacaagttttctgtaaatattttaaggctttgcaggccagctgtagtctctgtcacacattcttatttgtgcatgtttttcccaaccatgtaaaaatgtaaagtgcattcttagctactggggcaggttgaatttggcccatgggctagagtttgccaacccctaacttaaacctttgtactaactttatgaccactactggatttttgttgttgtttgttttagttctggtgcctgctttgtttttttttttttttttaatcctcttgctgatgtttcttggtgcagttactgtgccatttgtattggtgcttttaatgtaatgcaaactggtaataatatctaaacttgctggggttgtacataaaattattgaaaagattgaaaagatgctgagcattgactctgtggcattcattatgcccttttgtgattgctggattttagccatctttaggacatttgagctttaggagaagccaaattctgtataaatgacttgaagtgctaatagcacaggttttgaaacctctgcctgggtttgagtctcagctctgccttttactacctgtgtgatcctgagcaagttacttagtatccctgtcctctagtttcctcctctgtagtgtggggataataacatagacataacctgagagttagagtgtagagaaggctccctggcagatagtgctgtagaagtactggccattgccattactcaggtgcttgtgtttgctgaacctcatagtaagggctcggagagcactaagaggaggtgagaaatgctgctagattgacagcttgtccccagatagcccattcccgagagcaccttaggtttatacctgatttgtgttgtagttagtagtgtctctggtaatttgaactagtttcaggttggtcttgaaaacctggggaggttgggggtaaatgatttggtagcagttctcttttgtgattttatacattatctttgtagaactgcagtttgctaattctctgagcccaacacaatgaagtctgggcctaaaatcatagaatttcttttatttttttttttgtttttaatttatttattccctccctccctcctttcttcctttcttccttttctttctttctttccttccttccttccttctttcttttctttctttcttttctttctttggagtctcactctgtcaccaggctggagtgcagtggcacgaactttcttcagagtctcactttgtcaccaggctggagtgcagtggcgcgaactcagctcactgcaacctccgtctcctgagttcaagagattctcctgcctcagcctcccgagtagctgggactataggcatgtgccaccatgcccagctaattttcttatttttagtagagacgaggtttcaccatgttggccaggatggtcttgatctcttgacctcgtgatccacctgcctcagcctcccaaagtgcggggattacaggcgtgagctaccacgcccagcctattttttattttttgaggcagagtctcactctgtcacccaggctggagtgcagtggtgcaatctcagctcactgcaacctccgcctcctgggttcaggtgattctcctgccttagcctcctgagcacctgggactacaggcgcctgccaccacacctggctaattcttatatttttagtagaggcggggtttcaccatgttggccaggctggtctcgaactcctgatctcaagtgatcaacctgccttggcctcccaaagtgctggaattacagccatgagccaccatgcccagccaaatcatgagatttcaataccgctgaactttgattatggcaaagtgaacttctgctttgattaaagcttgatgagagaggtggctggggatagtttgagataagggcaaggcaggaaaatgcataatcttacgtgggctcattgtcattgtacaattcttttggtccatgtggaatttgatccgtcctatgacttaagttatgtttatttttgtttttatttttatttattttgtgtctttttgagagacatgatgttgctctgtcacctgggccagaatacagtggcacaatcttagctccgtgtagccttgaactcctgggctcaagtgatcctcccacctcagcccctcaaacagttgagattatagtatgaaccactgtgcctagccttaagtgatttttaaatttgtactgaacagtttgtcctttccttccattaaatcatattagaagtacagaacttgatatttcctgtagcaatacagtttttctttgatgaagtttgatttcaagtacttatttttcataatttaaagctattttttatagagagaattttaatcaaatatttggatgtcactattgctatatatggtattaagtatggtgaccatagtttgtaaactccaaactgacagcaagacaggaaatttgtgttagcaaaggcttttttcttactgtttgaattttttaaaaattagatacaatacagagaggagcacacaaatcattaagagtacagctcagcgaattttcacacagtgaacatgtgtaaacagcaagtaacaaaagatttacctgcatcctataacctcccattattcccttttctaggtactgtctctccactgcattcccaccaaatataaccactatgctgaattctgacatcataaatgagttttgcctgattttgagcttttgtgactggaagtgtacagtgtatataccctttcgattctgtcctctttagtttaccattgtttgagaaatttatccatactgttccagaattaactactgttaattattgttaattaactactgttgtagttaattcatcctcattgttatctagtattcttttgtgagtaaacacaatttccattctactgtgatcccagctatccatttgggtcgtttccagtttggggtccattacaaatagtaatgctatctgtaatgctattttgtattactacaaatagtaatgctatttgtggcacaaaaatactgcttttgtgaacattcttatacatgtcttttgatgaatgtatgtttgcattgctgttgtttacattatgtacctagtaatggaattgctagatcataggagatgtatatattaagctttagtggatgcattacataattattagttattattggttataccaatttatcctctcatcagtagtatacaacagtttctgtatctctaatctccaacattttagccattttagagtttgtgtactaacacattgtggttttaatttacatttccctgatgactaataaagttgagtacctcttttgtgttctttatagccatttgactgtcttgtgaagtgcttgtttgtcttgcctatttttcttttctttctttctttttcttccttccttcctttctttctttcttctttctttccttccttcttttctttctttctgtctttctttcttgtctttcttgtctttctgtctttcttggtcttgccctgtcacccatgctggagtgcagtggtgcagtctcagcttactgtagcctcgacctttttggggctcaagttatcctcctttctcagcctcccaagaagctggactacaagcacgcaccaccatgctcagttaattttttattttttgtagaaatggggtttcaccatgttgtccaggctggtctcaaacttctgggctcaagtaatcctcctgccttggcctcccaaaatgctgggattacaggcatgagccaccgcagccagccttggctatttttcaaaaggatataagtagaacatctgtatatcccttcaatttgcatattattcagtaagagttgcactctggtagtagaaatatataaggaggagaaagaagtggaaacaaaaagtctattctcatgagaagacttgggggatagtgttctctctagctccaagctacttattccttacgaaaagttgaagataaacttatctcagactgaggctgtctcaatgttgtcttcctattccattatacacatataacccatatttttttcaccagctgaattttgctcctagaaaattgattcatcaggaaaaatatccgtcttgcaaggtggttctctttagagtctgctgtgtgacatagctcaggacaaattgtgtgatgtcagataggttgggttaaggaatagaccttattggggaaagagagaacttggagggccaaggttagcaggagaaggaaatgttctctcatctgccgtcaattcagggaggggcaaacctggtgtctgtgttcacagggagggatccatccatctgtgattctcccttcttatcaggtagcatgggaaagctacactgttgcggggaggagggtcacacgcaggctacttagtaccaggcaccctggacttggattcaggttgccagttgtgtgagaaactgcccagcacctgaaggccctgaacccatgagaagttgtacctacctcccatgaggaggaatcctgtcatcccatgggagctgagcttgggtgcagtccctcttgctggcttgtccaggagtgagctccagggttgtttgggacagttctgctcattgctttacactgtgtatacattatctgtagagttccatgaagagaacttcagcactgtaactgcaagttttaacatggaacagaatttttctcacctgtattaattcttaagatttgaagttctatcaacaagcatttagattgtgtggagatttttttatttttatttttggagacagagtcttgctctgttacccagactggagtggcagtggcatggtcttggctcactgcaggctctacttcctgggttcaagcgattctcatgcctcagtgtcctgattagctaggactacaggtacacaccaccatgctggctaatttttgtatttttagtagagacgaggtttcaccgtattggtcaggctggtctcgaactcccagcctcaagcagtccacccacctcggcctcccaaactgctgggattacaggtgtgagccaccatgcttgactgacatcatcatgttaaaagaataaatgttctagggagctgggcacagtgtcatgtttctgtagttctagctgctcgggaggctgaggcaggaagatcccttgagccctggagttcaagtccagcctgggcaacatagtgagatctctttttttaaataaataaataactgttctagggactaaaatttcctttcaccattagtaatttactgtagaatctccaagaatgaacttattttaggtactgaaaatgagggagactaaatgttttatacagtagtttttagtaaaatatgagatttgatgcatttgatagatgatgtttgtttaaaataattcttaaatttttgatcatgtaattatagtttcattaatggtagatttgtaaaataaatgttaccaaatgaaaatgcatgtacctatgttaattatccttatctaaagctgaaagttcagttcaactatgttaaaacatagtaggggcctggcagggtggctcttgcctgtaatcccagaacttagggaggccaaggtgggcagatcacgaggtcaggagatcgagaccatcctggctaacattgtgaaaccgtatcgctactaaaaatacaaaaaattagccgggcatggcggtgggcacctgtagtcgcagctacttggtaggctgaggcaggagaatggcgtgaactcaggaggcagagcttacagtgagccgagatcatgccactgcactccaggctgggtgacagagcaagactccatctcaaaaaaaaaaaaaaagttggccaggtgtggcggctcacacctgtaatcccagcacttttggaggccgaggcaggcggatcacaagatcaggagtttgagaccagcctggctaacagagtgaaaccctgtatatactaaaaatacaaaaattagccaggcatggtggtgcatgcctgtagtcccagctacttgagaggctgaggcaggagaatcacttgaacccgggaggcggaggttgtggtaagctgagattgctccactgcactccagcctggacaacagagcaagactctgtctcaaaaaaaaaaaaaattaatgattaaattatttaggggagccgggcgcagtggctcacgcctgtaatcccagcactttgggaggccaaggcgggcggatcacgaggtcaggagatcaagaccatcctggctaacacaggatgaaaccccgtctctactaaaaatacaaaaatttagccgggcgtggtggcgggtgcctgtagtaccagctactcgggaggctgaggcaggagaatggcatgaacccgggtggcggagcttgcagtgagccaagatagcgccactgcactccggcctgggtgaaagagtgagactccgtctcaaaaaaaaaaaaaaattatttaggggaagatactatacaattctgtttaacaagtcacattttaattttttcttttggaaatattagcaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacaggtactgattttaaactcactaagtcacatttcttttttttttttttttttgagacggagtctcgccctgttgcccatgctggagtgcaatggcgcgatctcggctcactgcaacctctgcctcccgggttcaagcgattctcctgcctcagcctcccaagtagctgggattacaggcacacggcactatgcccggctaattttttgtatctttgttagagatggggtttcaccatgttggtcaggttggtctcaaactcctgaccttatgatccacctgtcttggcctcccaaagtgctgggattataggtgtgagccaccacacccggcttacatttcttttaaaaatgtggataccatttagaaaaggatgggccattcttcctatagggatctgactggtgaattataactgtgctgttaactttggaaatgggaatgcacaagatattgttttaaatatgcacgctaatgacagtttgtatccttctttccccacccccacccttgcttcaactacctgtcaaaattaacagcagccttctggaaatatggatgacagtggttttttctctattcaggtaagtagtcacaagcatgtactatgtgttgcttacatcccaggcaccgtttcacagcctttcaatagtcactgtaacaaggcgaccttcggaagttcttctgtctacagagtatagattatactctagagtactagattttttttttcttgagacagagtctcgttctgtcacctaggctggagtgcagtggcgtgatcttggctcactgtagcctctgcctcccgggttcaagcgatcctcctgcctcagcctcccaagtagctgggattacaggcacccgccaccacaccagttaatatttgtatttttagtagagatagtggggtttcaccgtgttggccagtctggtctccaactcctgacctcagcctcccaaagtgctgggattacaggtgtgagccactgcacctggccaactagagtactagatttttatatagataaacatgaaaggattgtagaatcttcatattagagtggggcatttaaaaattccttcttgagaaagattaatttgcatctggatgctaataataaccttaattctggccgggcgcggtggctcacacctgtaatcccagcactttggggaggccgaggtgggcggatcacgaggtcaggagattgagaccatcctggctaacatggtgaaaccccgtctctactaaaaatacaaaaattagctggacgtggtgacacgtgcctgtaatcccagctactcgggaggctgaggcaggagaatcgcttgaaccagggagtcgtaggttgcagtgagccaagatcgcgccactgcactctagcctggtgacagagcgagactccatctcaaagaaaaaaagaaatccttaattctaataagtcacaatgtctcaaacttaccatctgttgggtaaatttgagaaaatgcaataccttgctaccatccttttaaatcagcctaccagactggatttccttattatggtttgtggcttttgattttttttttttaatgtatagctctctttgaattctttggtggttatatatatatgtactcgcaagattcttttatctgtgggtctttcattctttttctaacactgtgagttgtatccagagtactttcggaacctctcctgagcgacctatctctgcagatatctttgtttatgtttcccttgtactgccctcctggactcttcctcatccaccagcatttccatctagtgctttaccgtgccactgctaacaggtaatggctactgcagggctgaaatcagaggccagagtaggcccagcacttggcgtttcctatttgtgccttgctgctcttggtgcctgttcatgtgtgcccactaccttgcactcaatttctgtctttgctggtacctggctcacttgcttctttgttggctaccttggagggcagatagtgaattttcagaaatttccctttttttgtcagacagattgaaataaacaggtttgcattttgttttttctacaagcggcaagcccatgaccctagaagtctgacatctatggaaccttcagtttaaatgcccagggagaacttattttggtagatatgatttctgacattgcaggtagcaagttgaatataatttttctaaagtagcacccacagcagccaaattatcagatgtatatagtagactagttttaagaaaagcacttatgggtagaatatacatctggatttttgaggcagttttatttaggaattgtgtggttttctggaacatctcagagacctggtatgaaaagcactcttctaatatatatgtgtttttttttatggatttagtgatatatctatacacacacactttttaaaacctatagccggctgggcgtggtggctcatgcctgtaatcccagtactttgggaggcccaggcgggtggatcacaaggtcaggagattgagaccagcctggccaacaaggtgaaaccctgtctctactaaaaatacaaaaatagctgggtgtggtggcgtgtgcttgtaatcccagctactcgggagcctcaggaggagaatcgcttgaacctgggaggcggaggttgcagcgagccgagatcgtgccactatactccagcctgggcgacagagcaagactctgtcacaaaaaaaaaaaaaaaaacctatagccttctagagaaatttatatatgaagtacacaactaacatagctacacttcctaaatttggaatggagtggtttagcttatgaaaagttgctatttttcttaacaggttataagcaatgccttgaaagtttggggtttagaactaatcctgttcaacagtccagagtatcagaggctcaggatcgatcctatgtaagattctgttttgcatttcatacatttcttttcccaaatttgatttttaaagttgtaatttcttaaagaagagaaatacattttgaatacttttgttttgatgttccctgtttcattcactcagactttcctatttcacctttgtgatgtccatgagcatctgccctgtagccttcctggcaccccagtgtctgtggcagcacagagctgaccccataagtggtgcatgaggccatcttgtggcacagcatcactaagctgctgcagagacgttcatatggttgtgtgatcttttaaaaacatcagtgacacttaactataaatataatcttaaattatcacaaattttatataatatttgccagtagacaacataaatatgaattcaatatttcaagttaatattgtctgttttcttttttagaaatgaaagatcatttatatgcaattataaggaacactggtttacagttagaaaattaggaaaacaggtaacatttcttacccttccttgtctttttttcttatattgtaccccatttaaaactaaaatgtgggccaggtgtggtggctcatgccaacagtttgggaggctgaggtggggggatcacttgaagccaggagtttgagaccagcctgggcaacaaagggaggtcctgtctcttaaaaaaaaaataaaaataaaaataaaaataaataaaaaaaaaaacaaagagccaggcatggtggctcacatctgtaattccagcttacttggaaggctgagtcagaaggatcacttgagctcaggagtttgaggctgcagtgaactatgattttgtcactgtaccccagcctgggtgacagagtaagactgttctataaaacataaaaataaaaaaaatatatttaaaaattaaaaaaaaaaaaggattgctgactttaaaattaggaaactgaccagtaatgtgtgtgtgtgtagcatggtttatccttcttgatagatagaaattgtcattttaaaagataatatcagttttccttataaatttatttgtgacaagtatatgcaatttaactatatcataagaaaaattctatattaaagataatacaaatgtggttacttttaagtgggtttttatgtgatgactatgttctgtcagttaattattacatttatagatttgtatttagcatagtgctgtcacaaagcctgaaatagtgtcaagcatgaataaagcattcaattatgtttgctttagtgtaagattattcattatgattccaaaagccatgtaatacgtacgtctacagaaaatcacttctattttttaaataaaacatgaaatatgtcttgagcaagctattttaagaaacaatcatttaacgtccttgttattagaattttgaatctttgaaagagggttattgaaaaccagctaggacagtaaaaaagaataaactagtgatacatgcagcaatatggatgaatctcaaaataattatgctgaaagaataacccacaaacaaaatactacctgctgtatggtatcatttattaaaagtctagaaaagtgcagattcatctgtagtgatggaaagcagattgaccagcggttgcctggggacgagaaggctatggaggagtgagaggggagggttacagagaggcacgggaaacatggcaatgaggaatgtgttcactatcttggttgtagtaatggtttcatgggagtacagtatacaaatgtgaaaacatttcagaggccagatgcagtggctcatgcctgtaatcccagcacttttggaggccaaggcaggaggattgcttgagctcaaggagttcaggaccagcctgggcaatggcacaagaccccatctctaaaaaaaaaatgaaagaaaaaaaaattggctaggcgtggtgatgcatggccgtagtcccaggtgctagggaggctgaggagggagcacagaggtcaagcctgcagtgaatcatgatcgtgctactgcactccagcttgggtgacagaaggagatcctgtctcaaaaaaaaagtttcaaattatacactttaaatatgtgcagtttattatatgtcacttataccccaataaatctgttttttttaaaatgtaaatacaagccaaaaaaggtataagtcaagaaaatatattgaattaaatctgtaagagataattcaaaaacaaaaaccctattgttatcttttaagtcacccaaatcaaatttgggaaaagtcacctacttagcttcatcctaagttggttctttctttctttctttccttcttttgagacggattcttgctctatcgcccaggctggattgcagtggcgggatcttggctccctgcaacctccgccacctgggttcaagcaattctcttgtctcagcctcccaaatagctgtgtctacagccacgcaccaccacacccagctaatttttgtatttttagtagagacggggtttcgccatgttggtcaggctggtcttgaactcctgacctcaggtgatccgtccgtctctgcctctcaaagtgctggggttacaggcgtgagccaccatgccgagccctaagttggttctttcttaaagttcttcctgaggagccaagagcaagttaaggagatgtaacctagaagcttacagtggaggctagctgggtgcagtggttcacgcctgtaatcccagcactttaggaggctgaggcagggagatcactgaggccaggagcttgagagcagcttggcccaacacagtgacaccttgtctctacaaaaaaaaaaaaaaaaaaaggcagcttacagcagtagaggctgatgcgagtgggaatcacctctaggtaaaaaccagtgtagcgtactgctgagattatttaacctctgggttttatttatgtgtttttaaaaattatgatccagtattttttacttttttttgtataaagtaagcactgaatttttaaggttgtattaatttgcaaataaatgtctatcttattattttgagagatttaaaaaattttagttcttcaaaattgcattttcacattttgaattacgttatctttgacaaatacagaagatgtcaaattttggtttattttctttggttctaatttatatttttgtttaaaactatatttttcactatagactctttctgtctctcgaggtccctgtataatgaaaaagaaggctggaaaaagtattaacattgtcaaaatccaggaaaagtagttggtcatgatattgatcgttaactttagaaactttttgtatcttgtgggttaaattaggattactatgtggtagtgataaatgatgttaattagggccgagtgcagtggctaacacctgtaattccagcatgtagggaggctgaggtgggaggatgtcttgaatccaggagtttgagaccagcctgtacaacatagtgtaagaccccttctccacacaaaaaaattagaaaatttgtcaagcatcttggtgcacacctgtagtcccagctgcttgggaggatgaagcgagagaatcacttaagcccaggtgttcgaggctgcagtgagctatgattgcaccactgcactccagactagatgaccatctcttttaaaaaaatgtgtttatatgttatatgtgatagtgctttttaaaaacatttttaaattatagagacagggtctcactatgttacagcccaggctggtctcaaattcctgggctcaagcaatcctcccaccttagctaacctcccaaagtgctcggattataggcatgagctgcatgcccagctaatttagtgatttttaaaaactgagctggtaattataaattctcttcctggaacttctgactttctcacaattggaatcttttgacaaaaattatcagtaatgggaaaactttgtgtagttgtcatttttcctcccatcagtgtgatagatatgattggagttatgttggactgatattttgaaaaaagatttaattatagctattaataaagacatttaaactactgactatgcatttttattcttttgggagggtttaatgtttatagtttaaagcaaactgttgtttttaaaaaagtatctaacagggccgggcgcggtggctcacacctgtaatcccagcactttgggaggcctaggcgggcggatcacaaggtcaagagatcaagaccatcctggctaacatggtgaaaccctgtctctactaaaaatacaaaaaaatagctgggtgtggcggcgtgcgcctgtagtcccagctactcgggaggctgaggcaggaggatggcatgaacccgggaggcggagcttgcagtgagccgagatcgcgccactgcactccagcctgggcgacagagcaatactctgtctaaaaaaaaaaaaaaaaaaaaaaaaagagtatttagcagaggccaggtgcagtggctcatgtttgtaatcccagaactttgggaggctgaggcgggcggatcatttgaggtcaggagtttgagaccagcctggccaatgtggcaaatgtgctgtctctaactaaaaatacaaaaattagctgggtgtggtggtgcagacctgtagtcccagctacttgggaggctgaggcaggagaatcacttgaacctgggaggcagaggttgcagtgatccgagatcatgccactgcactccagcctgggttacagagtgagactcttctcaaaaaaaaaaaaaagtatttaatagtgataaatctgcagtattctcttgtagtttttaagatcatattattcagtcaaagaaaagagctcaacttgaaatatttccagagtttaaacaatcttactaagctttgatgggttgtatctattcttaacatgtgaaacttccttattacctataatatacactaacttaaatattgacaatttttttccagtggtttaacttgaattctctcttgacgggtccagaattaatatcagatacatatcttgcacttttcttggctcaattacaacaggaaggtaagtaacggctgaacattttgtaatgttacctttcgaagtagttaaataaccaggcacattagatgacagtgtgataaaactgtttttctggcagtggcagtgaaacaatctttagttttgacgtggtgataggctgtgatttgggtgacgctgttcagttagagttctcactgacacctggcccttcctcttctgaggatgctgctttctttgcagcccttctaagtaatggctttttcttttatacatcacatatcacacggctgagaggagggatagatgtttttcttctttgcctcttctaggccactgttcttccttataaactccagtttctttgaaatacatgcccctaacggctgggcacggtggctcacgcctgtaatcccagcactttgggaggctgaggcaggcggatcacgatgtcaggagatcgagaccatcctggctaacacggtgaaatcctgtctctactaaaaataacaaaaaattagccggggtgtggtggcggacgcctgtagtccgagctactcgggaggctgaggcaggagaatggcgtgaacccaggaggcggagcttgcagtgagctgagatcgcgccactgccctccagcctgggcgacagagcgagactccgtctcaaaaaaaaaaagaaaagaaaaaaaaaagaaatacatgcccctagattaaactatcccttgtccttttgcactcatccacaagtctcttttcatcagtgattttaggatctgactcgttgtctttttctctacttcaactacttttatcattcttaattatttctgtatcgtcaatcaatccagtacctgcctcttagtttcaaaatcacttactcttgcttagctattaccagtaatcataaccactgtcaaatctcaattgcaagcatattactctttaactaccacctcctatctttaaaccatgttttgtctgtttttttattccagccattctttaaaccctactgtggggcccaagcatttcctttatacgcattcttcctttcttctactgcttattttctgtaatccgtcatcataatcactccattgcattcttcaacgtgtttcccctctctccctccatcatacttgaatgacaaaaatctcaaccctggttaaaccacatcttggccttgtccattcctgtaccagagtagctggacgtggctaaaaaataacataaaacatgatgattggttttacttttttcttaaatgatctatccatccattcacccatccatctatcaaagtgactaggcctatttctgaagcccaggctggagtgcagcagcataatcacagctcattgcagctccaaactcctgggctcaagtgattctcttgccttagcctgttgagtagctgggactacaggcttgtgctaccacacctagctaaggttttactttaaatttattataatcacaaaattcagatgagcctttagtgctgtctgatatttctactatgttttcttagtgatgtaccaccctccaaggtgtttataaaaaattatgtaccactctccaagaagtttataaaaaataatgtgccaccctccaaggtgactaatttcacagcttatgtctttaaacctttaagcactttcctctcccttacacaccttccttgtggctttccgttacattctgctgagaacatagaagcaattaaaattatgttctttctaccagcaaatttatcaatttgcttatatcttcacctgtgctttgagcctatttaaatagatgaatggtcccctacctctaaccaaaaccagtccctcacttgtgggctggatcccagctcttctcacctactcaagatgttcctgctttcatctctccactctcttatataatcagttccccccccctttttttgtaatattcctataagcagtaaaataagctttttatttccattgattaaaaataaaaatcctctcttaattccatgaaactccagctgcctccccatttttattttttccttaggattgtctctagtgtgccttctccttttcttgaactctgcctcctgggttcaagcgattctcctgcctcaacctcccgagtagctgggattacaggcgtgcaccaccatgaccggctaattttttttttttttttttgagatggagtttccctcttgttgctccggctggagtgcaatggcgtgatctcggctcaccgtaacttctgcctcctgggttcaagcgattttcttgcctcagcctcccgagtagctggatttacaggcatgtgccaccatgcctggctaattttgtattttagtagagatggaaggggtttctccatgtttgttaggctggtctccaactcctgacctcaggtgagccgcccacctcggccccctaaagtgctgggattacaggcatgagccactgcgcctggccccggctaaattttttttttttttttttgtatttttagtagagacagggtttcaccatattggccaggttggtctcgaattcctggcctcgagtgatccacctgcctcagcctcccaaagtgctgggattacaggcgtgagtcaccttgcctagccatcttttagtaatggtatttggagatcacaatttgagtgctggcatgcttattgctgctgggtttgttatgtagttattgtgaattcacatttaggaatatagggtttttaattctttgattttagatacttgtatcttttttcttttatatttaaaaccttggttcctgatgatatcccttcttagaaaccctgtctacctttggccttcagcccaccatgctgtggttttcctaacttgctgcctgcacttttcagattcctttcatggatcttaaatatcatctgtaaataagatctatgtgtcaataattaccaaacttttatctttagtcttgacatctaccctgaacacctagctttgactaactcctagctttggcatctccacttggaaatccaaaaagtgtttcaaactgaacatgtctatgaaagacttatttttttctctctatccatgctatccatcaggttttccatttccataagggtgactcttgtactctggttcctatatattataccgacagagcagcccagagtgcttcttaaccagtgtaaggcctgttatgtcccaccctcactctttgtccttcagtggcttcccagcacacttagaataaaatctgaagtcttaggccgggcttggtggctcatgcctgcaatcccagcactttgggaggatgagggggcagatcacttgaggtcaggagttgatgagaccagcctggccaacatggtgaaaccctgtctctaccaaaaaatacaaaaattaactgggtgtggtgttgtgcacctgtagtcccagctactcgggaggctgagataggagaatcacttgaacccgggaggcagaggttacagcgagccaagatcataccactgcactccagcctgggtgacagaacgagactctcaaaaaaaaattaaaaaaaaaaaatatgtgaagtcttgaataaaacccaagatctttaccatggcccctgaacagggcagagtatccattcttcagacactcttcatagaataccatggtgagctggcatatttattatacaatacagaaacaattttactggcagaaaacacattaaaccgtctaaactctgaatacagttgtcctcataaaaaatgttcaacatactattttgaggttttccattaatagttcttataatctttgtcccattatgtgttaatccaacaaaggatatccaataacaaacaccaaagtttaagaaaaatgtgctaggcgcggtggctcacacctgtaatcccagcactttgggaggccgaggtgggcagatcacctgaggtcaggagttcgagaccagcccagccaacatggtgaaaccctgcctctcctaaaaatacaaacattaactgggtgtggtggtgggtgcctgtaatcccagctactcaggaggctgaggcaggagaatcgcttgaacctcctgggaggcagaggttgcagtgagctaatattgcaccactgcactccagcctgggtgacagagtgagactccatctcaaattaaaaaaaaaaaaaaattaatgatagagaaacttaaatcagttagattgttttaggtatagcccatccttggtttttgtgtgtagcatctagcttggggaaaccctggatttctggaatcatatttagacacagtcacactagactaatgtaattcttttgggatgcaaaccacacgtttgacaccttaaatagcttttaggtatttggcttcccagcccctatttttagttacaaggggtgtacatgtgtgggtcagggtgggggtagctctttccgcagatgattagttttagccatgttactagttattgcacacattatctgtgtcctcacagcagccctgtgagtaagtgtattagggttctctagagggacagaactaataaggtagatgtatatatgaagggtaatgtattaaggagtatcgactcgtatgatcacaaggtgaagtcccacaataggctctctgcaggctgaggaaccaggaagccagtccaagtcccaaaacctcaaaagtagggaagctgacagtgcagccttcagtctgtggcaaaaggcctgagagcccctggcaaaccactggtgtaagttcaagagtccaaaagatgaagaacttggagtctgatgtttgagggcaggaagcatccagcatgggagaaagatgaaggctcagcaagtctagtacttccacactcttatttctgcctgctttattctagctgagctggcagctgattagatggtgaccacccagtttgagggtgggtctacctctcccagttcactggcttaaatgttaatctcctttggcaacaccctcgcagacacacccagaaacaataatttgtagccttcaatccaatcaagttgataatattaaccatcacaggaaggtactagtatcatatgtttaacagtagaaaccaagacaaatgcagctaggaagtgggagaactgggatcagatgcaggcagtctgattctaaatcagttgctgttacccactctgacaacagtaagtgagtagcctgctcagtcaagtactatattagtagggccctttacagacatatttatttctcacagtcactcaatgagacggctcttccagtcttacaatggagaaagtgaggctcagagactttaagtaacttaccttagacgactttactagtaagtataagaatcattatttggactaaagtctttctgaatcctcagcttgtatttttttccagtgttctgtgctgcctttttatctactagtgttttacatcaattttgaatctctttactaactggttaggttgatttttgccttttttttttaggttattctatatttgtcgttaagggtgatctgccagattgcgaagctgaccaactcctgcagatgattagggtccaacagatgcatcgaccaaaacttattggagaagaattagcacaactaaaagagcaaaggtaaaaatgaggcctgcagtatggaatatatggtagtatttcattatgagaattaaattttcatgcttagattgaatatgtggtccttgtgttgttggcgactctattttggaccttatattttagtgaagtttattagtttaaacttgaatcaactctttgaaatacttaaatatattaacttagttagctggtatggtatattcctagcacttcgggaggctgaggcaggctgattgcttcaacccaggagttcgagaccagcctgggcaacatggcaaaacctcatctctacaaatagtacaaaaattagccagatgtggtggtgtatgcctatagtcccagctacttgggaggcagaggaagaaggatcacctgaaactggggaggtagagactacagtgagccataatcacactaccgcactccagcctggtcgagagagtcagaccctgtctcaaaaaaaaaaaaaaaaagaaacggaaaaaaaaaacttagttggattcaaattgcaacacaatcattatattactagagcttatttgccagaaaacattttaagttttgacttacttaaagcctttacattacaaatgcctttatgttatgtctaaaatagaagattggttgcagttattaccagtgcttttgttctttagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtaaagacattctgatgtgtgttgtattcattgctgaagaattgattccaattattcttagatttcatggaagttaatgtactcttagaggtgttttgacaattactgcagaagcaatagctatatagtgggctttccctttagatttcttataatggaaatcactttttacaacctatattttattaggagtagttatatttttactcctggttattttatttggtttcaacactgtactaacacaatagtaaattgtggttttaatctttgtgggtatcagttgacccttatccaaatcagctgttacataaatatgtgccattagacactatggaagggcctggacagggaatataaactgattttacaaaaacccaacatttattggctatgcaacttaaaccgtaagcccactttggtgggcccagttttttagtgatataaactatcaatagagaaaagcgaaaacatatcccctagacaatctaggcaaagaaaaatgttaagacatagctcaaagtagcttaattaaaagtttgaagtgggttttttgttttatttttttctaactcatatgtatttgcttctactttctaatgaaattatttatcagttgatttccttagatatctaaataaaattgaaatttcattaatgggaagattatttttatcctgaacttttcttgcctctatgcatgcctctgagtactccatatggtgtgcaatcccatttttgattaatagagtcctgctggattagcagggacagaaatcagctttagatttctttcttttttttttttctttctttttttttttttttttttttgagtcagagtctcactgtcgcccagcctggagtgcagtgatcttggctcactgcaacccctgcctccgaggttcaagcgattctcctgcctcagcctcctgagtagctgggactacaggcgcctaccaccacgcccagctaattttttgtacttttagtagagatagggttttgcccttttggccaggctggtcttgaactcctgacctcaggtgatccacctgccttggcctcccaaagtgctgggattacatgtgtgagccaccacgcccagccagaagagtagaatattcttaaagagaaaacgttttaaaggcttactcaaatgagtataaacaaacatattgttgcttgaattggtaaatacagtgattggtttttgttgtgttgtgttttgttttcaggtagttccagaaacatatctcaagatatgacacagacatcaggtacaaatcttacttcagaagagcttcggaagagacgagaagcctactttgaaaagtaaagtagttggtacaagttaaagtagcatgtttaatatttgctttggctattttgtctatttgtaaatggttactgcctgaatcctgtgaatatttgaatgtattttttaaaaatttacagcaaataggacgggcacggtggcttacgcctgtgatgctagcagtttgggaggccaaggcgggcagattgcctgaggtcaggagttcgagaccagcctgggcaacacagtgaaaccccatctctactaaaaatacaaaagaatcagctgggcatggaagcgtgcgcctgtagtcccagctgcttgggaggctgagccaggagaattgcttgaacccgggacgtggaggttgcagtgagccgagatcgcaccactgccctccagactgggtgacagagtgagactccgtctccaaaaatatatgtatatatatataaataaaaataaaaatttacggcaaataacatgaaacaaaaaaaccttgccccaatactggataaattttttaaactgagtgaaggaaaccttataaaatttcatttattaaaagaaaaatgaaattaggacaagacaagaagaatgccaattgatcctttggatgtacttcttgcttacctgattaaccctgcaaaattcctctaccaatcagtacgaaaaacagctttggaggtatgggagcgcattcccaaatagacgtggtagttcatttagctgctcatggccgcttcaggcagtcctgtaagcctgttagcatcaggggaatggatgcaaaccataaatctggatcaactcctaaaaccttaccttgtgcccagccttgtaagtgcttgctaaataggaattccaccatatgaaaatacattcttttcaagtaactatcattcagacttttgtcccccactttttttttttaaagaaaaataaaaggctgggcacggtggcttacgtctgtaatcccaccattttaggaggccaaggcaggtggatcacctgaggtcaggaattcaagaccagcctgaccaacatggtgaaacctcatctctactaaaaatacaaaaattagccgggcatggtggtgggtgcctgtaatcccagctacttgggaggctcagacaggagaatcgcttgaatctgggaggcagaagttgcagtgagctgagataacgccattgcactccagcctgggggacaagagcgagacttcgtctcaaaaaaaaagagaaagaaaacttcatgttaaagattacaagataaataatcagacccactgatcctaggtcagaaaacagagtcatagctcaatctgacttactatttgctgtatttcatccattctgagatgcacatagtttcacatttcaatgtctctgaaattgagaagcatcttacagtcataattgacagtatattagcagcacctataaatattggctcattttacatttgatggtataatgaagaaaatatttacctttttttctgttttgtttttaagtcacaactcagaagtagatgaaggaaaattctgatcagctgacatcctcttaatgtgagatatttctagtctttattcagtatagattaatggctaattatatgttaaatttcaaagtagtgcttattagtgctttttacttttaagtttcaaaattaacttttttattataataaactccaaatttatacaaaagtagaaaaactagcatactcctgtttatgacccagattcaacaaatactagcacacggccaatcttgcttttttttttttttttttttgagatggagtcttgctctgttgcccaggctggagtgcaatggcacaatttctgctcactgcaacctctgcctcctgagttcaagcgattctcccacttcagcctcccaagtagctgggattacaggtacacaccaccatgcctggctaattcttgtatttttagtagacacgggatttcaccatgtcgtccaggctggccttaaactcctgacctcaagtgatccacctgcctcggcctcccagagtgctgggattacaggcatgagccactgagcccggcccaatctcgttttataatactcccatctcccattctttccactgtcccacctgcaagtttggattattttgtaacaaatctcaatcatcatattattctataaccattttaatatgtgtctctaaaatatattagctttatttttaacatagttaaatgctattgtcataaaataataatcataataattaattgtaattctatatcatcaattatctagttaatgtaaaaaataaatctaaggccaggcgcggtggctcacacctgtaatcccagcactttgggaggctgaggtgggcagatcacctgagatcaggagttcaagaccagcctgaccaacatggagaaaccccatctctactaaaaatacaaaaaattagccaggcgtggtggcgcatgcttgtaatcccagctacttgagaggctgaggcaggagaatcacttgaacccgggaggcgaggttgcggtgagccgagatcgtgccattgcactctagcctgggcaaaaagagtgaaactccatctcaaataaataaataaataaataataaaaaataacttaaatctacttaattagaaaaactaacattctaaaaattttattttaagaaatatcaaaattggctgggcacggtggctcacgcctctaatccctgcactttggaaggctgaggtgggcggatcacctgaggtcaggagggtcaggagtacaagaccagcctggccaacatggcgaaaccctgtctccactaaaaatacaaaaattagccaggcatgatgatgggcacctgtaatcccagctactcaggaggctgagacagaagaatcgcttgaacccaggaggtagaggttgcagtgagctgagatcaccccactgcactccagcctgggtgacagagtgaaactccgcctcaaaaaaaaaaaaaagagaaaagaaatatagaaattaaagcatacatggccaggcgtagtggctcatgtctgtaatcccagcactttgggaggctgaggcaggcagatcacttgaggccatgagttcaagaccaacctggccaacatggcgaaagcctgtctctactaaaaatacaaaaaaattagttgggcatggtggtgcacacctgtaatcacagctactttggaggctgaggcaggagaatcgtttgaacccagaggtggaggttgcagtgagccgagattgtgccactgcactctatcctgggtgacagagcgagatactgtctcaaaaagaaaaaaaaaaggctgggcgcggtagttcatgcctgcaatcccagcactttgggaggccgaggcaggcagattacgaagtcaggagatggagaccatcctggctaatacagtgaaaccccgtctctactaaaaaatacacaaaaattagctgggtgtggtggcaggcacctgtagtcccagctactctggaggctgaggcaggagaatggcatgaacccgggaggtggagcttgcagtgagcagagatcacaccactgcactccagtctgggcgacagagcgaggctctgtctcaaaaaaaaaaaagaaagcatactctcacctccttcagtgactgatgttagtattttggcacattctttttctgtgacatatacacacttaccttgtaagtgttgtactcatttcctatgacagtaaatagtctttgtaacaggctgcatgatatttcataaaatgaatggatgtggcataatttatatgtgagccttttgaattctgctattataattaatattgcaatgaacaattcttatattgcctctacacctcaaatgtcttatcatttcttctagtttttctgaggatgtcagattattgggttaaaggatatgaacatttttaaggccttggaacagatttctaaattgctttccagaataattcccatgtgatactttcaccatgtttatttcagacttttttttttttttttttttgagacgaaatctcactctgtcacccaggctggagtgtagtggcatgatctcggctcactgcaacctccgcctcctgagtttaagcgattattctgcctcagcctcccaagtagctgcggttacaggcaagtgcctccatgcctggctaatttttgtgtcttttgtagacatggggtttcaccatgttgcccaggctggtttcgaactcctgagctcaggcaatctgcctacctcggcctcccaaagttctgggattacaggcgtgcaccaccgcgcccagccatcagagtcttttttgtcaaaataaaatggtctaaagacatacatcatagagaaactataatacaaaatttacaggtatatctaagaaaagaaaagtatatttaaagcataaaaataaactgctcttttacttaaaattttttaaaaactggattaaaaatatgaaacttccaacaaattgagcttttttttttttttttttcttttttgagacgaggtctcgcttttgtcacccagtctggagtgcagtggcgcgatctcggctcactgcaacctccacctccctggttcaagcaattcccctgcctcagcctcccaagtagctgggattacaggcgcatgccaccacgtcgggctaatttttttgtatttttagtagagagggggtttcaccatgttggccagactggtctcgaactcctgatctcaggcaatctgccagcctgggtctcccaacatgctgggattacaggcatgagccactgcactcggcctgaactttttatagtagtaacgataattcagtaatgtccaataatgactaagtaagttataacaagtacaatgtcagcaataactagtgctttttagtaaacagggtcaggcaaccttgtacccttttaaaaatgttcgaatatcgatatacctccttcctacttggtggaggattgattgaggaggaaagtgtgcagtgatggttaccagcttcagcctcttggcttgactttgcaaatactggtgagaatttggaaagagcttgagaatatcttacatagtcacatgttgctgagaagagttaagaactaacttcttgatgttcatttttaacaatggcttgcattcaaaaccttgtagagctcattagtaggagctaagaagctaatatttgcctttcactaaaattcctgattacttagcctaggtagttcgttgtctctctaggttctgtctttgggagcttgggtctaaggttatcaagctaactctttcttccctctcacccttcccaaattgaccctggtgctgatttgttattcatacgattttctagtttttcttttccctttttgagtatttgaagcttcatactgaatatagtaatcatagtattcatgcataaagaaaatcataaagtaattgcataaatgcataaagtaatcatagttttcatgcattaaaaaaactagttttggctgggcgctatggctcacgcttgtaatcccagcactttcggaggccaaggcaggcgaatcatctgaggtcaggagttcgagactagcctggccaacatggcgaaacctcttctctactaaaaatacaaaaaaattagccgagtatggtggcgggcgcctgtaatcctagctatttggcaggctgaggcaggagaatcacttgaacctgggaggcagaggttgcagtgagccgaggttgtgccattgcactacagcctaggcgacaagagcaagactccatctcaaaaaaaaaaaaaaaaaaaaaaaaactccctattacagattcataatttatgagtcattaaataatattttcaagccatgacattttttccagcagtagtctctaaatctgttttaccatcataaaaccccaagcaaaactctactacatcagctgtgtcactgtaaaacctgccttaactcacagaagcatgaaattaagcaatgtgtgtgaaactattttataaactgtaaagtattccatacatacatgttggcagttattaatgtcttctctaggtgtggctttgaaatggatgcagatgctttctgttacaaaaaacataagttgcaaatgttctataacaaggagagacacaaatatcttcatggacatggattgctatgagtgtttgattgcctaatacttgagccaccacttcagtgatatggtataatttatcaaacagtgttgagaaacagaaactactggggatgttttaaagaggaaaatacttaatatagaaattaggggtttacataatcttaagaaaggatgaaggtgcagctcttagccaggcctccacagtaccacaaaccaacttgcaggaagagctgtaaccactgccccagttgggacaatgggtaatgaggatattaaatttaagaacatactgctatagcaatgatccttggcatagaaagctgccaccacaattgcctagagatgggaacatgaagtctggcccccattgcaacagcagtgaagcagaattttgggactggcatctcccaaatggctttgcttgccaccagagaacaaccaaagtggagggagatggctaggcctcatttctgcctattttattttattttttgagacggagtcttgtctgtcgcccaggctggagtgcagtagtgtgatctcggctcactgcagcctccgcctcccagcttcaaacaattctcctgcctcagcctcctgagtagctgggattacaggcacccgccactgtgcccagccaattttcttatttttagtagaggtggggttttgccacgttggccaggctggtcttgaactcctgacctcaggtgatctgcccgcctcagcctcccaaagtgttgtgattacaggtatgagccaccatgcctggcccatttctcccttttttttttttttttttttttgaggtggagtctcactctgttgcccagactggagtgcagtggtgcaatcttggcgcattgcaacctctgcctcccagtttcaagcaattcttctgcttcagcctcctgagtagctgggactacaggtgtgtagcaccacacctggctaatttttgtttttgttttgttttttttgagacagagtctcactctgtcacccaggctggagtgtagtggcatgatctgggctcactacaacctccgcctcccgggttcaagcaattctcctgcctcagcctccagagtagctgggattacaggtgtgcgccaacacacctggctaatttttttgtatttttaatagagatggggtttcaccatgttggccaggctggtctcgaactcctgacctcgtgatccgcccgcctcggcctcccaaagtgctgggattacaggcatgagccaccgtgcccagacaaggtttgtatttttagtagagacagttttgccatgttggccaggctggtcttgaactcctcacctcaggtgatccgcctgccttggcctcccaaagtgctgggattacaggcgcaagccactgtgcctgacccgtttctgctttttaaagctcatgtgagcacttaatttgtaaccagaatcctacttgtaaaataatctaagacatgtagcttttagctttgtaacctctataatattgatggcacagtgggagtggatgctgagtaccacttgaacatgttccacctcagtgtcttcacagctggaaggtgtctacattgtttcaaggtggacaattgatttacttctcatttttcataaactaaaagtagaataaaggctattcctctaaaattgctatctcacctgtcactcccttgcattctcacataccttcttgagtggaggggcagagggcatggagtgatagcagatgtgccaggaattctccataactcagtccgtccctcttgtgctatgttgcagcatcaggatttgctaatgggaggatactgcccttacgtgcatcattagccatgcacactaaggtcttacacctacacacaggtcagtattctggctcagagaccaacagggagaaattgcagttctcattagttgaactttctttattgttcacagttttaaaacacaaaattgagaggaactctataaaaaatgtgccattctattaataattgttgctggtaatttaaaaatccttgttccttttcaaattcttatataccttttttttttaaacacttgatcttagccaaaagaccgagaagcaatcttttttttttttttttttttttttaacctatagcttctcactgagattgtcagctgtttgtaagttttggtttttggttttctgtgtttgtatttacatatatgaaatacagattgagtatcccttatccaaaatgcttaagactggaagtgttttagatttggggttttttaggatttgtgaatatttgcactatacttaccagttaagcattccaaatccaaaatttcaaatctgaagtgttccactgagcacctcttttgagtatcatgttggtgctcaaaaagtttctgattttggagcatttggatttctgattctcggatttaggatgcttgacctgtaatttcagatttacataaaagcagaaatagtacacagagctccttatatccttcacccagattccccaattattggcctttctgaaccatttgggaataatatgcagatatgattttccattatgtctcagttgttcagtgtatattttctaagtacaagaatatattcctacatatttacatgataaccgtcatgtttaaacattttaaaatggggatttgtattacattgtttctctttttgaaaaaattacagaggagcttaatgcaatcagtattacttaaaatctgataatgtgtgttaaatagtagttttcatttatttcatttatcaggtgttcagtgaatgcttactatgtaacagcacagttatcagcactggggaaatagatgagtaagataagatttgcactttcattagcttacatgccataaagagggaaataaagagaacaccagatgatgataagtttatgctgagaattaaaatgaagtgatgaaataatgggaatgtcaggtggctacttttggtgggatggtcaggaaaggcatctctggggagataaattttaagctcagacctgagtgaaaagaatgagccagccatggaaacattatgttaactcacatggtagtttgaaatgctttatctgatcaaaggtacttatttttggtgactttcaacaatattaagggtctataaaccaacactcatttgcataagaataactaccagtgaatctttttgtatgataggttttttgtttgttgtttttttgagacagagtctcgctctgtcgcccaggctggagtgcagtggcgcgatcttggctcactgcaacctctacctccccggttcaagtgattctcctgcctcagcctcccaaagtagctgggattacaggtgcctgccaccacgcctggctaatttttgtatttttagtagagatggggtttcaccgtgttgtccaggctcgtgtcaaacttctgacctcaagccatccacccgcctcggcctcccaaagtgctgggattacaggtgtgagccaccactcctggccatgataggttattttgtgatgaaaatacctacctcttaatttgtctgataaatttaaattttatgtctagatttcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcagacagcagcaaaagcagcaacagcagcagcagcagcagcagcagggggacctatcaggacagagttcacatccatgtgaaaggccagccaccagttcaggagcacttgggagtgatctaggtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaagctagatgtggtggctcacatctgtaatcccagcactttgggaaaccaaggcaggagaggattgctagaacatttaatgaatactttaacataataatttaaacttcacagtaatttgtacagtctccaaaaattccttagacatcatggatatttttctttttttgagatggagtcttgctctgtcacccaggctggagtgcagtgtcgcgatctcggctcactgcaagctctgcttcctgggttcatggcattctcctgcctcagcctcctgagtagctgggactacaggcgcccgccacatcgcctggctaattttttgtatttttagtagagacagggtttcaccatgttagccaggatggtctcaatctcctgacctcatgatccgcccgcctcggcctcccaaagtgctgggattacaggcgtgagccatcacgtccggccagaaatcatgaatattagtaggtgaaaaataaacacattttaccacctggaaaatgaaaaatacttgagtataatctaaataacaatgggaagtgcagagttactttccaggtctcggtttaaatatgtcttaaactttggccaattagtagtagaagttgagagaaaaagtaactatctgacaaagaaattataagcagaatatataaagaactcttaaaactgaataatcagaaaacaactcaataaaaaggtgaaggatttgaaaagatatttcaccaaataagacatagggatgacaaataagcacatgaaaagactctcagcatcactagtcacagggaaatgcacgataaaaccacagtgagacaccatggcacccctgtaggtatggctttaatgaagaaataaaactgacaataccaagtgttggcaaggatccaagcagctgagactcatatactgttaatgggaatgtaaaagtgtacagctttggaaaacagtttggcatttttttgataaatgtatacttagccatgtgatccagcagtcccaatcatgtatatataaccaaaagaaaagaaaacttaggttcacataaaaacttatatcaaatgcttatagctgaccaggcatggtggcccatgcctataatcccagcactttgggaggccgaggttggcagatacctgaagtcaagtgttcgagaccagcctggccaacatggcaaaaccctgtctctacttaaaatacaaaaattagccaggcgtgatggcaggcacctgtagtccagctattcaggaggctgaggcaggagaatcacgtgaacccgggaggcagaggttgcagtgagccgagatcgtgccactatactccagcctgggtgacagagcaaaactctgtctcaaaaaaaaaaaaaaaaaaaagggctggacacggtggcttacgcctgttatcccggcactttgggaggccaaggctgatggatcacctgaggtcaggagttcaagaccagcctggccaacatggtgaaaccccatctctactaaaaatacaaaaatttgctgggcatggtggtgggcacctgtaatcccaggaggctgaggcaggagaatcacttgaacccgggaggcggagattgcagtgagccaagattgtgccattgaactccagcctgggtgacaagaccaaaactccttctcaaaaaaaaaaaagattatagcatctttattcatcattgcccaaaattacaaactgcctaaatgtagaccttcatttagttaatgaatgcacaaactgtggtatatccaaacaattgaataaaaaaaggaatgaactggtacttttttctattcctcctgtttaagtacagccaaaacacctcaacatttgtataaaacatgagctgggctgggtgcggtggctcacacgtgtaatcccagcactttgggaggctgaggcgggtggatcacctaaggttgggagttcaagaccggtctgaccaacatggagaaaccctgtctcaactaaaaatacaagattagtcgggcatggtggcgcatgcctgtaatcccagcttcttgggaggctgaggcaggagaattgcttgatcccgggaagcgaaggttgcagtaagctgagattgcaccattgcactccagcctgggcaacaagagcaaaactctgtctcaaaaagaaaaaaaaaaccattcagctgaatctcaaaggcagagagaagacagactggctagggaccttggaaccagaggagcagtgtggtggggagtggactggattttctttttgcctcatttatcctggacttggtgctggagaagctatgggttcagaccaagagaaaaccccatgaaaagcctgctctctctagccaaaagaggcaacctagcaagataaaaacctttagataataagcacttgactccagtcaaacaaaacagaataaactggccccattcacccctgtcagcaaaggccaagtgggagccaagatatgtaccccaacctggaagtcataaggtacacttctcccctttcccagccaaggtggtgttagagaaggctgactggggagctgggattctcattccctccaggaggtgataacactcctttcacatggtgtcagtggtcacagggaggctgaacttccacccagtaatacataggcatctctctggctcctatatgggtgatgttggagaagaggccgagtagagaatccagactgttgctgacacccagcagtaacaaggacacctccacaatgtccgtggaggccatgtggagatcagtaacaaggcactgctctccctcccagtcagagagatgtcagtggaggactagggggctagaactcccatgtgcgttcagcagtaatccccatgaccgccactccttgacatcacaggccttgaagaaacctggactttcactcccctctggttgtagcgaggtggcactcccttttccctgttgccagtgctgtgtcagtggaggcttgctaaattggaagatgtaaataagattcacattctcataacataataccccaaattttcaggatttaattgaaaatcactaagctgggcatggtggctcacacctgtaatcccagcactttgggaggccaaggtgggccaaacacttaaggtcaggaattcaagaccagcctggccagcatggtgaaaccctgtctctactaaaaatacaaaaattagctgggcgtggtggcacatgcctgtaatcccagctactgggaaggctaaggcaggaaaatcactggaacctgggagacggaggttgcagtgatccaagatcgcactagtgtactgcagcctgggcaacagagcaagactccatctaaatttgtgtcaggattcccagaaggagatgagaaagggtggggctgaaaaaaattgaggaagaagtcatggctgaaaatttcccaaatttggcaaaagtcagaaacctacagattgaaaaagctgaatgaagctcaaatatgataaactcaaagaagttcacacagagacacatcacagtcagatttctgaacactgcagacaaaaaatgaagatctcgaaattagcaagaaatgaccttacctaagcaatttgaatgacagcagatttcccatcagagatcataaaggccagaaggaaggggtacatacaacattttttctagtgctgaaagacaaaaactctaggctgggcacggtggcacacacctgtaatcccagcacttttggaggctgaggcaggcagatcacctgaagtcaggagttcgagaccagcctggccaacatggggaaaccctgtctctactaaaaatacaaaaattagccaggtgtggtggcacgcacctataatcctagctacttgggaggctgaggcaggggaatcgcttgaacctgggaggcgacggttgcagtgagccaaggtcgcgccactgcactccagcctgggcagttgagcgagactccatctcaaaaaaaaaaaaattatccaggcttggtggtgggcgcctatagtcccagctacttgggaggctgaggcaagagaattggttgaacccaggaggtggaggttgcagtgagccaagctcatgccactgtactccagcctgggtgacagagcgagaccttgtctcaaaaaaaaaaaaaaaaaaaaaaaacaagaaaaaaactctaaacccagagttacatatccagtgaaatatccttcaggagtgaagggaaaattaacgatttgtcttcaggagacctaccctaaaagaatggctaaaggaatttctctaaacagaaaagaaatgataaaagaagtaattttggaacatcaggaaggaagaaagaacaataaaaagagtaaaatatgggtaaacacaatagactttcccctccttttgaattttctaaattgtatgatggttgaagcaagaattatagcactgatttggttttcagtatatatattggaaatatttaaggcattatgttacagatgaaggagggtcaaaggatataaagggaggtaacctttctatatttcttttgtactgatgcaggcactttggaaaataatttcactatttgtttaaaaactgaacataccctgaccatatgacatagcatctatactcctgggcatttatcccagagaaacagaaatttatttattttttttttagtattacactccgtaagtgctgtaatactagcacttagggaggctgaggcaagcagattgcttgagcccaggagttcaagaccagcctgggcaatgctgcacagtcaaaaaagaaaaacaaacatttagaaaactattttaaaagtctttaattgctgaatgcctctttggctaatatttggaagatcattattattatttttcttttttaggcagagtcttgctctgtcactgaggctggagtgcagtggcgccatctcggcttactgcaacctctgcctcccgggttcacgccattctcctgcctcagcctcccgagtagctgggactacaggcgtgtgccaccatgcccggctaattttttgtgtttttagtagagatggggtttcactatgttagtcaggatggtctccatctcctaacctcgtgatccgcccacctcggcttcccaaaatgctgggattacaggcgtgagccactgtgcccagcctggaagatcattatttagtcctacaactgacacattgttccactgacgcaattgcccaggctggtcttgaactcctgggctcaagcaatctgcctgcctcggcctccctaagtgctagtattacaggcttgagccactgtgcccagccaaaaatagaaatttatattctcacaaaaacatgtacatgaatgtttatagcagctttacttgtcataatcaaaaactggaaacaaccaaaatgtcctacagtgaaacaaactgtagtacatccatagcatgtaatactctactgtcaggattaaaaagaaacccactgttggcacaggcagcaccgtggctggatctcaggggcattatgctgagtgcaaaaaagcctcaaagggtcttacactgtatgattccacttgttcaactaaaaatgacagctgtatagagatagagaacatattagtggtttccactagttagagaaagtgggtaaaagataggtgggtgggaatataaatcgatagcagggagatctttgtggtattataacacttctatgtcttgattgtagtggtggtggttacatgaatacacgtgtgataaaatgccatgtagaactacatataacgttgtgccaatgtcaatatctaggttttagtttgatctttagttacataagatgtaactattgggtgaaattgggcaaaagagtacacgaaacctctcttaaatatctttacaacttcctttgaattgacagtttttcaaaatagaaagttgggtttttgtaaatacatgaattgttgatatacacaacaaatctcaaatgcattatgctacgtgaaagaagccatattcaaaaggctacatacctactgatgccttttatatgacgtgcaggaaaagataaaactgtaggacagagaatatactggtggctatctgggattaggaaatggggatcgaccacaaaggggcagcatgggggaattttctggggcaatggaatggttgtgtatcttgatggtgtatttgtcaaaatatatagaactataaaagtaaattttgctttatatgtattaaatcaaaaaaagaaactcgtgctcaaatagaaatacattttctgagaacttgccttttgatgactttgagaattttctggaaattttaaagaaatgtggttttgtttcccaacaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttctatttccttttgaatttgtgtttattgttaattcatagctattcaaagtgtgattagagctgggcttggtggcttgcatctacagttccagctacccaggaggcagaagcaggaggattgcttgagcctaggagttcgaggctgcagtgagctatgatcctgccactgaattctagcctgggcgacaaaacaggaaaaaagtatggatggaggaccagcagcatctgtatcacctgtgagtctttcagaaatgcagagtttcaggctacactcggacctactgaatcagaacttgcactttttacaagatccccaggacactaaagtatagagtgaagcttgagaagcgctgttgtgtggattgttcttaaccagctgcagtgatgaatatgaataacgcaggccagcacagtccattgatattctattccagcttactgcctgccaaaaggtccattattactggatcctcagtcttttccaagagaagctaagaattccaaatttttatttgaaatatattttttaaatgtttgttcaactggcccagtgccagtggctcatgcctttaatcctagcactttgagaggccgaggtggaaggatcacttgaccccaggagtttgagaccagcctgggcaacataaagagaccccatctctattaaaaaaaaatagagacaatgctgccttaaaaaagtcaaataaatgtttgctcaactgatttttaatactgagggccaaacaaagcacatcaaatttttaagtgctgcttttcctcattttatccaactctggacaccagaatccaaatgtagtgattggaatccacctagactgattgaggaatatattgtcctcaaattttatgagggttgactattcattttaactttaattaggattgggcacaaattttgaaacataataacattacaggaccggggcccgatccagaccccaagagagggttcttggatcttgtgcaagaatgaattcagggcaagtccataaattgaaagctagttcattaagaaagtagaggaataaaagaatggctaccccataggtagagcagccctgagggctgctggttgccatgtccacccccgcgccccctccccccgctttgttgttgttgtttaagacagagcttcgctgtgtcatcaggctggagtgcagtggcatgatagctcactgcaacctctgcctcccgcgttcaagcaattctcctgcctcagcctcctgagtagctgggactacaggcgtgcaccaccacgcccagctaatttttgtatttttagtagagatgcggtttcaccatgttggccaggaaggtctcaatctcttgacttcgtgatccacccgccttggcctcccaaagtgctgggatgagccacctcgcctggctggttatttcttgatgatatgctaaacaaggggtggattattcatgcctgccctttttagaccatttagggtaacttcccggcattgccatggcatttgtcaactgtcatggtgccaatgggagtgtagcagtgaggaggaccagaggtcactttcatcaccattttggctttttcagccggcttctttactgcaacctgttttatcagcaaggtttttatgatctgtatcttgtgcagaccttctatctcatcctgtgacttagaatgccttaaccatctgggaatgcagcctagtaggtcttggcctcattttaccaaccccctattcaagatggagttgctctggttcaaatgcctctgatatttccctactcccttttataagaaaacccttaatcctaagggttgcagagggatgaagatccatcttctgtattcttcaggctgaataggggtgatgatattcctgcctatgagagtctcttgtattagggtagagaggagctcagtcagtcagtatggctccctatccttcctcccttccccagcccctgacaactaccattctactttgtctctatgattctaagtatctcctataagtggaatcattcagtatttgtagggtttttttgtgactagtgtatttcacttagcatgtcaaggttcatccatgttgtagcatacgtccaaattttcttctttttaaaaactgaataatattccattgtatgatatatgcacattttgcttattcatttatccatcaaagaaaaccagtagcttttttacataccggcagtaacctggttaaaaaaataagatggaaaataccaagcccgaaaatgtcaaattcttgaataccagacaggtactataatccaagctgagcagagaggaatggcccttgtctcagccctgagccacatgtcactacatgggccccaaggagctcttgtgcccagagtcctgctaaggctcgttcttcacccctctaaagtggctcaaactagcgtaggaggccaggagggagcagattctatgccacctccattgcttccgtgccttctccagatccattttttccaaaagctgtgtttgaattctgcacaggggcatctctgtctccaacactgaaaccccttttgccagacccagagaagatttaactgcctttttttttgagacagggcctcgttctgtcacctaagctggagtgcagtggcacgatcttggctcacggcaacctctgcctcctgggctcaagcaattctcctgcctcagccacccgaatagctgggattataggcgcaggccaccacatccagctaagttttgtatttttagtagagacgcagttttgccatgttgcccaggctggtctagaacttctgagctcaggtgatccacccgcctcggcctcccaaagttctgggatgacaggcatgagccacctaacctggccagatttaactgctttgagccaccgtctcacacatcttccaggagcttctagtcaaatgcagaggattaatagtgtataattccatttacataagatctttggaatagtcaaactcatagagacagaaagtagaatggtggttgccgggggatggttgaggggaaaattaggagttgtttaacgagtatggagtttcagtttgggaagatgaaaaaagttctggagatggacagttgtaatggctgcacaacaacgtgaatgtacttaatgccggtgaaccgtataaccatttaacaactgaaaatggttacaaaattttgtgttatatatatcttatcacaataaaaaatatagaggattgatcactatatttgctcccttccaaaatctcacctaggttaaagtaaagaaaaatatataaacccacaagaacacagggaccaggagaggagatgcattcattggattacgtttggttgtaaaaaaaacaaaaaatcctccccggcacggtggctcacaccttgtaatcccagcactttgggaggctgaggcaggcggatcacgaggtcacgagtttgagaccagcctgaccaacatggtgaaacctcatctctactaaaaatacaaaaattagccgggtgtggtggcgtacgcctgtaatcccagctactgaagtggctgagacaggagaattgcttgaacccgggaggcggaggttgcagtgagccgagatcacgccactgcactccagattaggtgacagaacaagactccatctcaaacaaaaacaaaaacaaaaacaaactaatataacagtgactttacatgctatgggttttttttctcctctcatcttcaagaaatgtggagactgccagtccggggatgatacagcatcacagtgtcaggaatctgctctttctgtataattgttacaggaaaggggtccggatccagaccccaagagagggttcttgaattcagggcaagtccacagtgcaaagtgaaagcaagtttactaagaaagtaaaggaataaaaaaatcgctactccatagacagagcagccctgagggctgctggttgcccatttttatggttatttcttgatgatatgctaaacaaggggtggattattcatgcctcccctttttagaccatatagggtaacttcctgatgttgccatggcatttgcaaactgtcatggtgctggtgggggcgtagcaccaaggaggaccagaggtcactcttgtggcctttgtggttttggtgggttttggccagctcctttactgcaacctgttttatcggcaaggactttatgacctgtattttgtgttgaccttgtatctcatcctgtgacttagaatgccttaaccatctgggaatgcagcccagtaggtttcagcctcattttacccagctcctattcaagatggagttgcagccgggcacggtggttcatgcctgtaatcccagcactttgggaggttgaggcgggtggatcacaaggtcaagagttcaagaccagcctggccaacatggcaaaaccccatctctactaaaaatacaaaaattagccaggcgtggttggtgtgcgcctattatgccagctacttgggaggctgatgcaggagaatcacttggatctgggaggcggaggttgcagtaagcagatatcgcgccatgcactccagcctggatgacagagcaagactccgtctcgggggaaaaaaaaaccaaaatggagttgctctggtttacaggcctctgacataatggctttatgtacatggcttccatggctagggtcagctcatagatctaatatggctgtgttggtgctcatgttgcagcccagcaatcagaaagagaaaggaggctgggtttggtggcttacacctgtcatcccagcatttcgggaggccaaggcgggaggatcacatgagcccatgagtttgagaccagcttgagcaacacagggagacctcatctctacaagaaataaaaataaattagttgggtatggtggcatgcacctgtggtcccagctattcgggaggctgaggtgagaggataccttgagcctggaggtttgaggctgcagtgagctatgatcgcaccactgcactccagcctgggcaacagagtgagaccccatctcaaataattaaaaaaaaaaaaaagaaagaaagaaaaaaaaaaggagagcagaaaaaggctatgtaccttttctttcgggttaccttctcaaagttgtgcatactacttccgtttacattacattggccagaacttaacatgacaactactagctacaaggtggtttttattctgggttgccatgcatcttagcttaagtaccctacaggtcctgagataatgattcctatgaaaatgattatttacttatttaattaatttattttgagatggagtctcactctgtcacccaggctggagatcagtggcgtgatctcggctcactgcaacctctgcctcccaggttcaagcaattctcctgcttcagtctcccgagtagctgggactacaggcatgcgccaccatgcccagcttttttgtatttttagtagagacggggtttcactatgttggccaggctgatctcgaactcctgacctcaggtgatctgcctgcctcggcctcccaaagtgctgggattacaggcgtgagccactgtgcctggctgaaaatgattttttaaaagtgttccaggaggaaatggaaagggcataggggagtaagaaagtggaaataggaaaagaaggaagccaagcaagaggagcctatcaagcaaagcctcagtcctgcagacagtctggaggcaggatattcacatcttagagttgtccaaaacagaacaggcaagctgcataattttcaactctggctgaataaggttgcatttcagctctctgggcacctggaaagtatgggctccagtaacctggaggcagtctgctgactgctggctggtgtgcacaaaagtggtaaagggatgagggagtgcaatatgggcactggcatcttggctgtcatgcatccaggtaaaaagtggaggctcaattactatatatgaaggggagaacagactttggggaacaattagcagtcatttccaggtgagacaacaacagaagagaaatgctaatttttttttttttctttgagatggagtttcgctcttgttgcccaggctggagtgcaatggcgctatatccagctcaccgcaacctccgcctcctgggttcaagtgattctcctgcctcagcctcccgagcagctgagattacaggccaccacacccagctaattttgtatttttagtagagacggggtttctccatgttggtcagggtggtctcaaactcccaacctcaggtgatccgcccgcctcagcctcccaaagtgctgggattacaggcgtgaaccaccgcacccggcgagagatgcgaacattttttttgttttttggaggatgacaagttgatgcaggaatgagcagaaccaggaggctgaatactgagtgttaaagagttcctagcaatatgcaggaaaggaaagcaacaagtatcctgagaggtgggggttagatgtagactgaaatcaggaggtgtggttaaaactctttgaaagaggctgggtgcggtggctcacataatcccagcactttgggaggccaaggcgggtgcatcatgaggtcaggagttcgagaccagcctgaccaacatgtgaaaccccgtctctactaaaaatacaaaaattagccgggcatggtggcgcatgcctgtaatcccagctactaaggaggctaaggcaggagaaccgcttgaacctgggaggcgaaggttgcagtgagccgagatcatgccattggactccagcccaggcgacagagagagatcccatctcaaaaaaaaaaaaaaaaaaaaaaaaaaagaaaggaaaagaagaaaagaaaaaagaaacagcagattccctagagaggagacattcatcactaaaaaaaaaaaaaaaaattaaaataaatcgcagaagtaaaagccctttttgaaagggctcagggtacccagcttaatgtatgaaacaaaagcaaacccacatcaaagcacaccattattaatttcagagcatcagaaataaagagaagatctgacaagcttcttggaagaaaacaaggcatagagaataatcagagtagagtagagcaggaaaacagagtcctgggtagagcgaactgagcctagccaatcgatagaattcttgatgttttgttgaaaacagaagcactttacaattctcttggtaagttttagaaagattctgtagtaatggaaaattgtacaaatttaaaaaatggaatctgttaacttcaggaagaacaaaaggtagaaaaatcaaggcaatataatcttggtacaacatttgacttagctgcaatgaataagacttacatagtcatagaaggtaaacactgattacttactttaccaaaaattatgatatcatcatattgaaagcccagaaggaagggaaataaagggaaatacaagagtgctaaatcttttatttttcaataagttggtaatgtctaaaattaataagaaaatggcaaccataagcctaatgcttagaaattcagaaaagtatcagactaaattttaaagtctactttaaactgagtctttgctttatagttgcataaataaattcattttgatggttttctcagaacagatgaactggctgggtgcggtggttcatgcctgtaatcttagcactttgggaggccaaggccgatggctcacttcaagtcaggagtttgagaccagccaaaaaattagccgggtgtggtggtgcacgtttgtaataccagctactcaggaggctgaggcaggagaatcgcttgaacctgggaggcggaggttgcagtgagccaagatggaacgactgcactccagcctgggtgacacagtgagactccgtctcaaaaaaacacaaagaacagatgaactgttttcctttgaggaatattgatgatcccactgcttgagtaatggaaaccagcttgcagctgatagttattttttgtcttttttcctgttctatccactgtcctaggtttggaatttcagctgggcagcttcgatcccaccacctgtagccactgaggttcacaggctctgtttgaggtgttgtgttagattgtgctgggttggttttgctgcttattgagtgatgaagagcaacatattagctactgagatattttgcagaaaaaggaagagatgtcttcagctcaaatgctatcaaacttgtatgtcttttaactttttattataaaaaatttcaaacatatgcaaaagcaaaagactagtttaatgaaccttatgtacccaccacccagtttcaacaattatcagttcctgacaattcttgtttcacctatcccacctctattatacacacatacaaacacacagacaccacacacacacacacacacacacacacgagagcaaagtcaacttccattattttgaagcacatccaagacatcacatcacattatttatcacattatttattaagtttcttcagtcacagtccatctcacagaaagaaatcccccatgagaaacttttaggacatagcttttatgttaattacttaatttctgcactagccatccttgcctgccttctaattttctgcactagctatccttaccttccttctaattttctaacctgccaaggattctgctgcctcaaggtatttgtttctttctgcctggattgctctttcgcagatacttacatgtctcattctttttcacccaggtgaaataaacagcgttgttgctcacacaaagcctgtttggtagtctcttcacatggacgcgtgtgacatttggtgctgaagacccgggacaggaggactccttcaggagacgggtcccctgtccttgccctcactccgtgaggagatccacctacgacctcgggtcctcagaccaaccagcccaaggaacatctcatgaatttcaaatcgggtaagcggtcttttccatcctctcttgctacccttcaatctccctctctcgctacccttcaatctccctgtccttccaattccagttctttttcctctctagtagagacaaaggagacacattttatccgtggacccaaaattccggcgctggtcacggactcaagaagacagtcttctcttggtgtttaatcactgcagggacgactgcctgattattcacccacactccattggtgtctgatcaccgtgggggtgcctgtcttggtcattcacccacattcccttggtggcaagtcaattgcggggacgcctgctttggctgctcacccacccccttctccgtgtctctacctttctctttaaacttacttccttcactatgggcaacattccgtcctccattcccccttctactcccttagcctgtgttctcaagaacttaaaacctcttcaactcacacctgacctaaaacctaaaagccttattttcttctgcaataccgcttggccccaatacaaactcaacagtagttccaagtggccagagaatagcactttggatttgtctgtcctacaggatctagataatttttgttgaaaaatgggcaaatggtctgaggtgcctcagatccaggcattcttttacacactggtccctccctagtctctgctcccagtgcgactcatcccaaatctttcttctttctctcctgtctgttccttcagtctccaccccaagctccgagtcctttgaatcctccttttctatggactcatctgacctctcccctcttccccaggctgctcctcaccaggctgagccaggtcccaattcttcctcagcctccggtcccccaccctataatccttttatcaccccctcctcacaccgtgtctggcttacagtttctttctgtgactagccctcccctacctgcccaacaatttcttcttaaagaggtggctggagctgaaggcatagccaagggtaatgctcctttttctttatctgacctctcccaaatcagttagcgtttaggctctttttcatcaaatataaaaactcaacccagttcatggcctgtttggcaacaacccttggacactttaccgccctagacccagaatggccagaaggccgtcttattctcaacatgcattttattacccaacccactcccgacattagaaaaagctccaaaaattagattccggccctcaaaccccacaacaggacttaattaacatcaccttcaaggtgtacaataatagagtagaggcagccaagtagtaacgtatttctgagttgcaattccttgcctccactgtgagacaaaccccagccacatctccagcacacaagaccttccaaatgcctgaactgcagcggccaggcattcctccaggactgcctaccccaggatcttgcttcaagtgccggaaatctggccactgggccaaggaatgcccccagcccaggattcctcctaagccatgtcccatctgtgcaggaccccactggaaatcggactgtccaacccggcagccactcccagggcccctagaactctggccaaaggctctctgactgactccttcccagatcttctcagcttagcagctgaagactgatgctgcctgatcgccttggaagccccctggaccattacggatgctgagctttggataactcttacagtagagggtaggtccatcccctgtttaatcgatatgggagctacccactccacattaccttcttttcaagggcctgtttcctttgcccctataactgttgtgggtattgatggccaggattctaaaccccttaaaactcccccactctggcaccaacttggacaacattcttttatgtactcttttttagttatccccacctgcccagttcccttattaggccaagacattttaaccaaattatctgcttccctgattattcctggactacagccacatctcattgccacccttcttcccaacccaaagcctccttcgcctcttcctctcgtatcccccaaccttaacccacaagtatgggacacctccactccctccctggcaaccgatcaaacacccattactatcccattaaaacctaatcacccttacctggctcaatgccactattccatcccacaactggctttaagaggactgaagcctgttatcactcgcctgctacagcatgggcttctaaaacctataaactcttcttacaattctcccattttacctgttcaaaaaccggacaagtcttacaggttacctcaggatctggatcttatcaaccaaattgttttgcctatccaccctgtggtgcccaacccatacacttgtttgtcctcaataccttcctccacaactcactattccgttcttgatctcacagatgcttttttcactattccctacaccactcatcccagcctcttttgcttttacctggactgaccctgacacccatcagtcccagcagcttacctgggctgtactgccgcaaggcttcagggacagccctcattacttcagccaagctctttctcatgatatactttctttccatcccttcgcttctcaccttattcaatatattgatgaccttctactttgtagcccctcctttgaatcttctcaacaagatacttttcaacatttattctccaagggatatcggatgtccccctccaaagctcaaatttcttctccatccgttacctacctcagtataattcttcataaaaacacatgtgctctccctgctgatcatgtctgactgatctctcaaaccccaacaccttctacaaaacaacaactcctttccttcctgggcatggttggatacctttgcctttggatacctggttttgccatcctaacaaaaccattatataaactcacaaaagcaaacctagctgacctcataaatcctaaatcctttccccactcccctttccattccttaaaaaacagccctaaaagctgctcccacactagctctccctaactcatcccaacccttttttcattacacacagctgaaatgcagggctgtgcagttggaattcttacacaagagccaggaccatgccctgtagcctttctgtccaaacaacttgaccttactgttttaggctagcccccacattattcctgataccacacctgacccccatgactgtatctctctaatccacctggcattcactccatttccccatacttccttctttcctattcctcaccctgatcacacttggtttattgatggcagttccacctggcctaatcgccattcaccagcaaaggtgggctatgctatagtagtatcttccacatctatccttgaagctaccgctctgcccccctccactacctctcagcaagctgaactcattgccttaactcgagccctcgctcttgcaaaaggactacatgtcaatatttatactgactctaaatatgccttccatatcctgcaccacaatgctgttatatgggcagaaagaggtttcctcagtacgcaaaggtcctccatcattattgcctccttaataaaaactcttctcaaggccgctttacttccaaaggaagctggagtcattcactgcaagggccatcaaaaggcatcagatcccatcgctcagggcaatgcttatgctgataaggtagctaaagaagctgctagctttctaacttctgtccctcacggccagtttttcttcttctcatcagtcactcccacctattcaccgactgaaacttccacctatcaatctcttctcacacaaggcaaatggttcttagaccaaggaaaatatctccttccagcctcacaggcccattctattctgttgtcatttcataacctcttccatgtaggttacaagccactagcccacctcttagaacctctcatttcctttccatcatggaaatctatcctcaaggaaatcacttctcagtgttccatctgctattctactacccctcagggattattcaggccccctccctaccctatacatcaagttcagggatttgcccccacccaggactagcaaattgactctattcacatgccctgagttaggaaactaaaatacctcttggtctgggaagacactttcactggatgggtagaggcctttcccacagggtctgagaaggccaccacagtcatttcttcccttctgtcagaaataatttctcggtttggccttcccacctttatacagtccgataacggactggcctttactagtcaaatcacccaagcagtttctcaggctcttggtattcagtggaaccttcataccccttaccgtcctcaatcttcaggaaaggtagaacggactaatggtcttttaaagacatctcaccaagctcagcctccaacttaaaaaggactggacagtatttttacctcttgcccttcacagaattagagcccgtcctcgagaagctacagggtacagtccatttgaacttttatatggacgcactttcttgctcagccccaacctcgttccagacaccagccctctaggcgactatcttccagtcctctggcaggctagacaggaaatccaccaggctgctaatcttctcttgtctactccagattcccaactatatgaagacaccctagctggacgatcagttcttattaagaacctgacccctcaaactctacaacctcgatggactggaccctacttagtcatctatagtaccccaactgccgtccgcctgcaggatcctccccactgggttcaccgttccagaataaagctgtgcccatcggacaaccagcctaatctctcttcttcctcctggaagtcacaagtactccccactacttcccttaaagtcactctcatttctgaagaacagtaataacccttatgagcctaatacatcccttcattctattagctctattcatccttaccctactttttgcaacagggctttacacagtcaccccccctacttggactgaaccccaaaaacttgtcatccctactatcttctgtttactcatactcccattcactattctcaactactcataaatgccctaatcttgtttacattgcctgtttacactgtttctctaagccatcacagctggtatctcctggtgctatccccaaaccgccactcttaactccctcttagagtggatagatgatatttgctggcagggcaacctccaatattttcactctgatgaagttctattctttacttttatactcactcttattctcattcccattcttatgccaccctctacctctccccagctatctccaccacactatcaatcttactctctcctagccgtttctaatccctccttagcgaataattgctggctttgcatttccctttcttcctgcacctacacagctgcccccgccttatatacagactgggcaacatctcctgtctccctacacctccaaatttcctttaacagccctcatctttaccctcctgaagaacttcttcactttctagacaggtccagcaaggcctccccagacatttcacatcagcaagctgccgccctcctccacacttacttaaaaaacctttctccttatatcaactctactccccccacatttggatccctcacaacacaaactacttttcctgtggctgctcctttatgtatctctcagcaaagacccactggaattcccctgggtaacctttcaccttcttgatgttcattcactcttcatctccaaagcccaactacacacatcactgaaacaactggagccttccagctccgtattacagataagccttttatcaatacaggcaaacttaaaaacattagaagtaattattgcttaggaagacaccctgtatttcactccatccttggctaccttccccttgctcgtcagaccctcctcccaggccttcttgttatacccagccccgtaaataacagtgaaaggttgctcatagacactcgacgttttctcatacaccatgaaaattgaacctctccctctacacagttaccccatcagtccccattacaacctctgacggctgccgccctagctggatccctaggagtctgggtacaagacacccctttcagcactccttctcatctttttactttgcatttccagttttgcctggcacaagctctcttcttcctctgtggatcctctacctacatgtgtctacctgctaactggacaggcacatgcacactcattttccttaatcccaaaattcaatttgcaaatgggaccaaagagcttcctgttcctctcatgacaccgacatgacaaaaaagggttattccactaattcccttgcttgtcggtttaggactttctgcctcctctattgctctcggtactggaatagaaggcatttcaacctctgtcacaaccttccatagcctctctaatgacttctccactagcatcacagacatatcttaaactttatcagtacttcaggcccaagttgactctctggctgcagttgtcctccaaaactgccaaggccttgacttactcactgctgaaaaaggaaggcgctgtgtgtatgtgtgtgtgtatatatatatatatatatatatatatatattttttttttttttttttttttttttttttgagacggagtcttgctctgtcacccaggcagtggcgcatctcggctcactgctcactgcaagctccacctcctgggttcatgccattctcctgcctcagcctcccaagtagctgggactataggcgcctgccaccacgcccggctaattttttgtatttttttagtacagacagggtttcaccatgttagccaggatggtctcgatctcctgacctcatgatccgcctgcctcggcctcccaaagtgctggggttataggcgtgggccactgcgcccggccgggctctgtatatttttaaatgaagagtgttctttttacctaaatcaatctggcctggtgtatgacaatataaaaaaactcaaggacagagtccaaaaacttgccaaccaagcaagtaattatgctgaacccccttgggcactctctaatcggatgtcctgggtcctcccaattcttagtcctttaatacctgtttttctccttctcttatttggaccttgtgtcttccgtttggtttctcagttcatccaaaaccatatccaggccatcaccaatcattctatatgacaaatgctccttctaacaaccccacagtatcactccttaccacaaaatcttccttcagcttaatctctcccactctaggttcccacaccacccctaataccgctcgaagcagccctgaggaacatcgcccattatctctccatactaccccccaaaattttcaccactccaacacttcaacactattttgttttatttttcttattaatataagaagacaggaatgtctggcctctgagccaaggcctgcatgtatacatccagatggcctgaaggaactgaagaatcacaaaagaagtgaaaatggctgattcctgccttaactgatgacattaccctgtgaaattccttctcctggctcagaagctcccccaccaagcaccttgtgacccccccgcccctgcctgccagagaacaaccccctttgactgtagttttccactacccacccaaatcctataaaactgccccacccctaacaccctttgctgactctcttttgggactcagcccacctgcacccaggtgaaataaacagctttattgctcacacaaagcctgtttggaggtctcttcacacggatgcccgtgacaagtgcctgccaccacatcccactaatttttgtatttttagtagagatggggtttcaccatgttggccaggctggtcttgaacgcctgacctcaggtgatccacctgcctcggccttccaaagtgctgagattacaggcgtaagcccctgcacccggccctgttttttttttttttttttttttttacatcccggagtcacactggatatctgaatgtgcttaaaataaaattcacatctcattctggaagtgtttattaaatgctgtccctgcattcatgtgaacatcacagcccagcggttagatgtgcagactccaacgcctgactgcctgggtctgtcacttactagctgtatgaccttggatgggttacttaacatttctgggcactggtttctcattagtaaaatgaagaatgggcgcctgtagtcccagctacttgggagcctgaggcaggagaatcgctttaacccgggagacgaggttgcagtgagcctaggtcccgccattgcactccagcctgggccacagagcgaggctccatctcaaaaaataaaatttaaaaaaaaaaaattcagctttcacggagaattactactgcctggtgccatcagatcccaaatactggaggcattcccaatgttctgttcacaaactgtgcaatctgtgatagcccctcttgtgaatataatcaaagtgaaagatgccagtatacttattgctatgcttaaaagaactgcagatctatcatatccttttgaaaccaaaaaatgtggaagtctccctggctagatgctgtgtgtccacgtatcctgcaccaattgcttgaaagtcacatgacaacctttcttcctttagggaaggaagcaagcaaggtaggactctgtgtgttacagccagtgtgggtaactaaggactgagggaggtgtgggcacttgcatagctagtgccgccaataccataggagcgttgacactaaacggcacttctctattaagttgccctgctgtcttctaacgagtcattttgaaaaacggttgtccgtttttcaactacttgggtgagggcagcttcatgaaaaaagctcatcgtatgttaaagaaggtagggtttcaattgtgagctataaagccaccagaggagatttctcctatagaaggaaggtgtgtttcctgcttggacacctgaatttgcaacccatcctttcttttttcctttttccttttttttttttgcaacccatccgttctatctttgtagcctcagctcctaccatagtgcctgtcacggagcagctaatcgacacagtgacgcgtgtaagttatttgaagccacttcctgcgcagtggaacgtccccgccaagtctcccggcactgctgggtgtagtcccaattaaaacactaggtttcaacaacacaaggagggtccgcagggtccccggactacccaccaagggtcggctctgctttgaaacgcagctgcaggaggccgaggagcgccgcgatgccccagttatccaggccattccttccccggccgaagctggccgcctgcatgcacctaaccttgcgctaggcactggctgcggttaatccagcccgcgctcggtcctccgcgatcccagcctcccttgcgccaaggacacgtccggcggcttcgcggggcctgtctacgccaccgcggcgcttttctatgatccgctgccgctttccgagcgagtgtcatggcggccggcgtcgagttggcaggagtaacccacggaactgaggaaagtcattagagctgagaaagaagtggcccaatctggacggtgggaattcgtgggaatgagcagaaggccctccgtaggtgactgtgtcactagaggcgggcccctggtaaaattccaggccaggcctctgcgtttctaggcagaacctggagtcggccttgcctgagaacccagctttgtgttatcgtatcctgtctcgcgaaggcaggcgttcaaggatatttggtcggatcgcccggcggcgctaaacgttttcttttttccgagcggaccgggtcgttctctaaactcgccgcgatgtcgtcctggcttgggggcctcggctccggattgggccagtctctgggtcaagtcgggggcagcctggcttccctcactggccagatatcaaactttacaaaggatatgctgatggagggcacggaggaagtggaaggtaacagctggagcgagggaagggagggcttttctaagaccctgggaacgtccgtttggtccctacttccatcagtcaggagacgcggacaggtcgcaaaggaatgctgctcctcccttttttcttcacacacctcctcttgcctcttctcaacagattttctatgatgaaagaccactgggggccaggggaagagtcctggttctcctctctttaaatattctggatatctcaaagtacagtagagtgaaactgctcctacgttaagtcgagtttattttcgtttggttgatccacgcccaccactttgccctcagtttgggggctgatctcattcttaacatcttcctgtattctgacccagtttaaagaattgtatatctgttttctatttttgtaacagttgaaataactcataatttaaacacttggtgagttttgctcttttctgtcttgagtatgttatgaaacacggccatgtttttctatttaattattttttacttttccttattgtgatgatacattatagcgttccagctgagaatgtatttttgtgtataatcgcccatttcgcccatatttcgacagtagaatagggaagacaaccctattcttaaggtacccttagcaatcttcactcatttttgaagtcctcaatctcgttttactaatgaatagtgacccagagttacatggtaatcaatggctacattagttactatccattaattactattttgatttcttggcattttagtaagcaatgtttggatcaaaacagtttttaagtttcaatctaagtctgctccagggagataatgggctgttcttttgggtgagagctgctgctttgacaattgtatttcttataaataaatttaataaattagtattctggcttagtttttttttttaattgctaatttaacagatccttcatatttatttggtagaaatcgggaaagattgtttgatagaaagataatgcttatgttttgaatttctgtggggtctatttagatggaaggaagaatagactggggagctaccgaaatccattttaccgctttacgttccccatgccataaaggtgcatcagtgagacagctttggggatagaaatgaaagggcttctttatgtgtgggggaaagcatgggaccttgagggcagtcagaagcacagaagggctggataactatgggaaatgactggtcgctccttgtatcctagataggggaaaacggttggctgttccttagaccctaagccagagaatatgcatccctcaacctggatgtcttccaggtttaaggtggtgttcattttgttctcctgccaaatgtgctgttttctcattcttaatagcttcactctcagtttcctaaactgtaaatcttcgtgtccttgttttatgtcttttttcatctcctacttccagttggtcatgatatcttgtttatcctatgccagaattgtcctttgctttattactccttattctttagatccctataatctttcccagggactattcctctttca

SEQ ID NO: 4 - DNA, 24 bp, Artificial sequence
  Primer
tgacacagacatcaggtacaaatc

SEQ ID NO: 5 - DNA, 17 bp, Artificial sequence
  Primer
tgctgctgttgctgctt

SEQ ID NO: 6 - DNA, 25 bp, Artificial sequence
  Probe
agcttcggaagagacgagaagccta

SEQ ID NO: 7 - DNA, 15 bp, Artificial sequence
  Primer
tcgccgcttgctgca

SEQ ID NO: 8 - DNA, 17 bp, Artificial sequence
  Primer
atcggccgtgatgtcga

SEQ ID NO: 9 - DNA, 23 bp, Artificial sequence
  Probe
ccatggtcaaccccaccgtgttc

SEQ ID NO: 10 - DNA, 18 bp, Artificial sequence
  Synthetic oligonucleotide
gcatcttttcatactggc

SEQ ID NO: 11 - DNA, 20 bp, Artificial sequence
  Synthetic oligonucleotide
atagaatggcacatttttta

SEQ ID NO: 12 - DNA, 20 bp, Artificial sequence
  Synthetic oligonucleotide
aacccaataatctgacatcc

SEQ ID NO: 13 - DNA, 20 bp, Artificial sequence
  Synthetic oligonucleotide
aataatctgacatcctcaga

SEQ ID NO: 14 - DNA, 20 bp, Artificial sequence
  Synthetic oligonucleotide
aatagaatggcacatttttt

SEQ ID NO: 15 - DNA, 20 bp, Artificial sequence
  Synthetic oligonucleotide
ttttatagagttcctctcaa

SEQ ID NO: 16 - DNA, 20 bp, Artificial sequence
  Synthetic oligonucleotide
tttaacccaataatctgaca

SEQ ID NO: 17 - DNA, 20 bp, Artificial sequence
  Synthetic oligonucleotide
gcacattttttatagagttc

SEQ ID NO: 18 - DNA, 20 bp, Artificial sequence
  Synthetic oligonucleotide
agaatggcacattttttata

SEQ ID NO: 19 - DNA, 20 bp, Artificial sequence
  Synthetic oligonucleotide
ttttttatagagttcctctc

SEQ ID NO: 20 - DNA, 20 bp, Artificial sequence
  Synthetic oligonucleotide
ttaacccaataatctgacat

SEQ ID NO: 21 - DNA, 20 bp, Artificial sequence
  Synthetic oligonucleotide
aatggcacattttttataga

SEQ ID NO: 22 - DNA, 20 bp, Artificial sequence
  Synthetic oligonucleotide
ctttaacccaataatctgac

SEQ ID NO: 23 - DNA, 20 bp, Artificial sequence
  Synthetic oligonucleotide
atcctttaacccaataatct

SEQ ID NO: 24 - DNA, 20 bp, Artificial sequence
  Synthetic oligonucleotide
ttatagagttcctctcaatt

SEQ ID NO: 25 - DNA, 20 bp, Artificial sequence
  Synthetic oligonucleotide
atatcctttaacccaataat

SEQ ID NO: 26 - DNA, 20 bp, Artificial sequence
  Synthetic oligonucleotide
cacattttttatagagttcc

SEQ ID NO: 27 - DNA, 20 bp, Artificial sequence
  Synthetic oligonucleotide
ctcaagtacttgtgcaaggc

SEQ ID NO: 28 - DNA, 20 bp, Artificial sequence
  Synthetic oligonucleotide
tctcaagtacttgtgcaagg

SEQ ID NO: 29 - DNA, 20 bp, Artificial sequence
  Synthetic oligonucleotide
tggttttctcatttttatat

SEQ ID NO: 30 - DNA, 20 bp, Artificial sequence
  Synthetic oligonucleotide
tattctcaagtacttgtgca

SEQ ID NO: 31 - DNA, 20 bp, Artificial sequence
  Synthetic oligonucleotide
taaaaaatgctcatttattc

SEQ ID NO: 32 - DNA, 20 bp, Artificial sequence
  Synthetic oligonucleotide
tgctcatttattctcaagta

SEQ ID NO: 33 - DNA, 20 bp, Artificial sequence
  Synthetic oligonucleotide
taacccaataatctgacatc

SEQ ID NO: 34 - DNA, 20 bp, Artificial sequence
  Synthetic oligonucleotide
tatcctttaacccaataatc

SEQ ID NO: 35 - DNA, 20 bp, Artificial sequence
  Synthetic oligonucleotide
cctttaacccaataatctga

SEQ ID NO: 36 - DNA, 20 bp, Artificial sequence
  Synthetic oligonucleotide
ataatctgacatcctcagaa

SEQ ID NO: 37 - DNA, 20 bp, Artificial sequence
  Synthetic oligonucleotide
ggttttctcatttttatatt

SEQ ID NO: 38 - DNA, 20 bp, Artificial sequence
  Synthetic oligonucleotide
tgttcatatcctttaaccca

SEQ ID NO: 39 - DNA, 20 bp, Artificial sequence
  Synthetic oligonucleotide
tcaagtacttgtgcaaggct

SEQ ID NO: 40 - DNA, 20 bp, Artificial sequence
  Synthetic oligonucleotide
cattttttatagagttcctc

SEQ ID NO: 41 - DNA, 20 bp, Artificial sequence
  Synthetic oligonucleotide
cccaataatctgacatcctc

SEQ ID NO: 42 - DNA, 20 bp, Artificial sequence
  Synthetic oligonucleotide
acccaataatctgacatcct

SEQ ID NO: 43 - DNA, 20 bp, Artificial sequence
  Synthetic oligonucleotide
aatgttcatatcctttaacc

SEQ ID NO: 44 - DNA, 20 bp, Artificial sequence
  Synthetic oligonucleotide
ggcacattttttatagagtt

SEQ ID NO: 45 - DNA, 20 bp, Artificial sequence
  Synthetic oligonucleotide
aagtacttgtgcaaggctga

SEQ ID NO: 46 - DNA, 20 bp, Artificial sequence
  Synthetic oligonucleotide
atgttcatatcctttaaccc

SEQ ID NO: 47 - DNA, 20 bp, Artificial sequence
  Synthetic oligonucleotide
tttttatagagttcctctca

SEQ ID NO: 48 - DNA, 20 bp, Artificial sequence
  Synthetic oligonucleotide
acattttttatagagttcct

SEQ ID NO: 49 - DNA, 20 bp, Artificial sequence
  Synthetic oligonucleotide
aatctgacatcctcagaaaa

SEQ ID NO: 50 - DNA, 20 bp, Artificial sequence
  Synthetic oligonucleotide
gcatattggttttctcattt

SEQ ID NO: 51 - DNA, 19 bp, Artificial sequence
  Synthetic oligonucleotide
catattggttttctcattt

SEQ ID NO: 52 - DNA, 19 bp, Artificial sequence
  Synthetic oligonucleotide
gcatattggttttctcatt

SEQ ID NO: 53 - DNA, 20 bp, Artificial sequence
  Synthetic oligonucleotide
ttctcaagtacttgtgcaag

SEQ ID NO: 54 - DNA, 20 bp, Artificial sequence
  Synthetic oligonucleotide
ttattctcaagtacttgtgc

SEQ ID NO: 55 - DNA, 20 bp, Artificial sequence
  Synthetic oligonucleotide
gttttctcatttttatatta

SEQ ID NO: 56 - DNA, 20 bp, Artificial sequence
  Synthetic oligonucleotide
caagtacttgtgcaaggctg

SEQ ID NO: 57 - DNA, 20 bp, Artificial sequence
  Synthetic oligonucleotide
attctcaagtacttgtgcaa

SEQ ID NO: 58 - DNA, 20 bp, Artificial sequence
  Synthetic oligonucleotide
gctcatttattctcaagtac

SEQ ID NO: 59 - DNA, 20 bp, Artificial sequence
  Synthetic oligonucleotide
taatactttttccagccttc

SEQ ID NO: 60 - DNA, 20 bp, Artificial sequence
  Synthetic oligonucleotide
tggcacattttttatagagt

SEQ ID NO: 61 - DNA, 20 bp, Artificial sequence
  Synthetic oligonucleotide
gcaccatatatatctcagaa

SEQ ID NO: 62 - DNA, 20 bp, Artificial sequence
  Synthetic oligonucleotide
gttaatactttttccagcct

SEQ ID NO: 63 - DNA, 20 bp, Artificial sequence
  Synthetic oligonucleotide
gccaaaatactaacatcagt

SEQ ID NO: 64 - DNA, 20 bp, Artificial sequence
  Synthetic oligonucleotide
gtatagagtttacctgcagc

SEQ ID NO: 65 - DNA, 20 bp, Artificial sequence
  Synthetic oligonucleotide
tgagccaatatttataggtg

SEQ ID NO: 66 - DNA, 20 bp, Artificial sequence
  Synthetic oligonucleotide
atgttaatactttttccagc

SEQ ID NO: 67 - DNA, 20 bp, Artificial sequence
  Synthetic oligonucleotide
agaagagtgcttttcatacc

SEQ ID NO: 68 - DNA, 20 bp, Artificial sequence
  Synthetic oligonucleotide
gaagagtgcttttcatacca

SEQ ID NO: 69 - DNA, 20 bp, Artificial sequence
  Synthetic oligonucleotide
aagagtgcttttcataccag

SEQ ID NO: 70 - DNA, 20 bp, Artificial sequence
  Synthetic oligonucleotide
agagtgcttttcataccagg

SEQ ID NO: 71 - DNA, 20 bp, Artificial sequence
  Synthetic oligonucleotide
gtgcttttcataccaggtct

SEQ ID NO: 72 - DNA, 20 bp, Artificial sequence
  Synthetic oligonucleotide
tgcttttcataccaggtctc

SEQ ID NO: 73 - DNA, 20 bp, Artificial sequence
  Synthetic oligonucleotide
ttttcataccaggtctctga

SEQ ID NO: 74 - DNA, 20 bp, Artificial sequence
  Synthetic oligonucleotide
tcataccaggtctctgagat

SEQ ID NO: 75 - DNA, 20 bp, Artificial sequence
  Synthetic oligonucleotide
tgttaatactttttccagcc

SEQ ID NO: 76 - DNA, 20 bp, Artificial sequence
  Synthetic oligonucleotide
atactttttccagccttctt

SEQ ID NO: 77 - DNA, 20 bp, Artificial sequence
  Synthetic oligonucleotide
gataaacagcaccatatata

SEQ ID NO: 78 - DNA, 20 bp, Artificial sequence
  Synthetic oligonucleotide
taaacagcaccatatatatc

SEQ ID NO: 79 - DNA, 20 bp, Artificial sequence
  Synthetic oligonucleotide
ccaaaatactaacatcagtc

SEQ ID NO: 80 - DNA, 20 bp, Artificial sequence
  Synthetic oligonucleotide
aaaatactaacatcagtcac

SEQ ID NO: 81 - DNA, 20 bp, Artificial sequence
  Synthetic oligonucleotide
aaatactaacatcagtcact

SEQ ID NO: 82 - DNA, 20 bp, Artificial sequence
  Synthetic oligonucleotide
aatactaacatcagtcactg

SEQ ID NO: 83 - DNA, 20 bp, Artificial sequence
  Synthetic oligonucleotide
atactaacatcagtcactga

SEQ ID NO: 84 - DNA, 20 bp, Artificial sequence
  Synthetic oligonucleotide
aaacagcaccatatatatct

SEQ ID NO: 85 - DNA, 20 bp, Artificial sequence
  Synthetic oligonucleotide
acagcaccatatatatctca

SEQ ID NO: 86 - DNA, 20 bp, Artificial sequence
  Synthetic oligonucleotide
caccatatatatctcagaaa

SEQ ID NO: 87 - DNA, 20 bp, Artificial sequence
  Synthetic oligonucleotide
accatatatatctcagaaac

SEQ ID NO: 88 - DNA, 20 bp, Artificial sequence
  Synthetic oligonucleotide
acattactggtcagtttcct

SEQ ID NO: 89 - DNA, 20 bp, Artificial sequence
  Synthetic oligonucleotide
cattactggtcagtttccta

SEQ ID NO: 90 - DNA, 20 bp, Artificial sequence
  Synthetic oligonucleotide
attactggtcagtttcctaa

SEQ ID NO: 91 - DNA, 20 bp, Artificial sequence
  Synthetic oligonucleotide
tactggtcagtttcctaatt

SEQ ID NO: 92 - DNA, 20 bp, Artificial sequence
  Synthetic oligonucleotide
actggtcagtttcctaattt

SEQ ID NO: 93 - DNA, 20 bp, Artificial sequence
  Synthetic oligonucleotide
ctggtcagtttcctaatttt

SEQ ID NO: 94 - DNA, 20 bp, Artificial sequence
  Synthetic oligonucleotide
attttcatgttccagatcac

SEQ ID NO: 95 - DNA, 20 bp, Artificial sequence
  Synthetic oligonucleotide
catgttccagatcaccatct

SEQ ID NO: 96 - DNA, 20 bp, Artificial sequence
  Synthetic oligonucleotide
tccagatcaccatctttgac

SEQ ID NO: 97 - DNA, 20 bp, Artificial sequence
  Synthetic oligonucleotide
cagatcaccatctttgacaa

SEQ ID NO: 98 - DNA, 20 bp, Artificial sequence
  Synthetic oligonucleotide
agatcaccatctttgacaag

SEQ ID NO: 99 - DNA, 20 bp, Artificial sequence
  Synthetic oligonucleotide
gatcaccatctttgacaagc

SEQ ID NO: 100 - DNA, 20 bp, Artificial sequence
  Synthetic oligonucleotide
caccatctttgacaagctat

SEQ ID NO: 101 - DNA, 20 bp, Artificial sequence
  Synthetic oligonucleotide
accatctttgacaagctata

SEQ ID NO: 102 - DNA, 20 bp, Artificial sequence
  Synthetic oligonucleotide
tactaacatcagtcactgaa

SEQ ID NO: 103 - DNA, 20 bp, Artificial sequence
  Synthetic oligonucleotide
atcactgcacactttcctcc

SEQ ID NO: 104 - DNA, 20 bp, Artificial sequence
  Synthetic oligonucleotide
cactgcacactttcctcctc

SEQ ID NO: 105 - DNA, 20 bp, Artificial sequence
  Synthetic oligonucleotide
actgcacactttcctcctca

SEQ ID NO: 106 - DNA, 20 bp, Artificial sequence
  Synthetic oligonucleotide
ctgcacactttcctcctcaa

SEQ ID NO: 107 - DNA, 20 bp, Artificial sequence
  Synthetic oligonucleotide
tgcacactttcctcctcaat

SEQ ID NO: 108 - DNA, 20 bp, Artificial sequence
  Synthetic oligonucleotide
cacactttcctcctcaatca

SEQ ID NO: 109 - DNA, 20 bp, Artificial sequence
  Synthetic oligonucleotide
acactttcctcctcaatcaa

SEQ ID NO: 110 - DNA, 20 bp, Artificial sequence
  Synthetic oligonucleotide
cactttcctcctcaatcaat

SEQ ID NO: 111 - DNA, 20 bp, Artificial sequence
  Synthetic oligonucleotide
actttcctcctcaatcaatc

SEQ ID NO: 112 - DNA, 20 bp, Artificial sequence
  Synthetic oligonucleotide
ctttcctcctcaatcaatcc

SEQ ID NO: 113 - DNA, 20 bp, Artificial sequence
  Synthetic oligonucleotide
tttcctcctcaatcaatcct

SEQ ID NO: 114 - DNA, 20 bp, Artificial sequence
  Synthetic oligonucleotide
aaatgagccaatatttatag

SEQ ID NO: 115 - DNA, 20 bp, Artificial sequence
  Synthetic oligonucleotide
aatgagccaatatttatagg

SEQ ID NO: 116 - DNA, 20 bp, Artificial sequence
  Synthetic oligonucleotide
atgagccaatatttataggt

SEQ ID NO: 117 - DNA, 20 bp, Artificial sequence
  Synthetic oligonucleotide
agccaatatttataggtgct

SEQ ID NO: 118 - DNA, 20 bp, Artificial sequence
  Synthetic oligonucleotide
gccaatatttataggtgctg

SEQ ID NO: 119 - DNA, 20 bp, Artificial sequence
  Synthetic oligonucleotide
ccaatatttataggtgctgc

SEQ ID NO: 120 - DNA, 20 bp, Artificial sequence
  Synthetic oligonucleotide
caatatttataggtgctgct

SEQ ID NO: 121 - DNA, 20 bp, Artificial sequence
  Synthetic oligonucleotide
atatttataggtgctgctaa

SEQ ID NO: 122 - DNA, 20 bp, Artificial sequence
  Synthetic oligonucleotide
agtgcttttcataccaggtc

SEQ ID NO: 123 - DNA, 20 bp, Artificial sequence
  Synthetic oligonucleotide
gttcatatcctttaacccaa

SEQ ID NO: 124 - DNA, 20 bp, Artificial sequence
  Synthetic oligonucleotide
gcacactttcctcctcaatc

SEQ ID NO: 125 - DNA, 20 bp, Artificial sequence
  Synthetic oligonucleotide
cagcaccatatatatctcag

SEQ ID NO: 126 - DNA, 20 bp, Artificial sequence
  Synthetic oligonucleotide
ggtcagtttcctaattttaa

SEQ ID NO: 127 - DNA, 20 bp, Artificial sequence
  Synthetic oligonucleotide
aatgctcatttattctcaag

SEQ ID NO: 128 - DNA, 20 bp, Artificial sequence
  Synthetic oligonucleotide
atgctcatttattctcaagt

SEQ ID NO: 129 - DNA, 20 bp, Artificial sequence
  Synthetic oligonucleotide
tggaactaccttgcatactt

SEQ ID NO: 130 - DNA, 20 bp, Artificial sequence
  Synthetic oligonucleotide
actaccttgcatacttagct

SEQ ID NO: 131 - DNA, 20 bp, Artificial sequence
  Synthetic oligonucleotide
agtgctataattcttgcttc

SEQ ID NO: 132 - DNA, 20 bp, Artificial sequence
  Synthetic oligonucleotide
gtgctataattcttgcttca

SEQ ID NO: 133 - DNA, 20 bp, Artificial sequence
  Synthetic oligonucleotide
gctataattcttgcttcaac

SEQ ID NO: 134 - DNA, 20 bp, Artificial sequence
  Synthetic oligonucleotide
taattcttgcttcaaccatc

SEQ ID NO: 135 - DNA, 20 bp, Artificial sequence
  Synthetic oligonucleotide
aattcttgcttcaaccatca

SEQ ID NO: 136 - DNA, 20 bp, Artificial sequence
  Synthetic oligonucleotide
attcttgcttcaaccatcat

SEQ ID NO: 137 - DNA, 20 bp, Artificial sequence
  Synthetic oligonucleotide
gccattaatctatactgaat

SEQ ID NO: 138 - DNA, 20 bp, Artificial sequence
  Synthetic oligonucleotide
  misc_feature (2)..(3): bases at these positions are RNA
  misc_feature (5)..(7): bases at these positions are RNA
  misc_feature (12)..(17): bases at these positions are RNA
  misc_feature (20)..(20): bases at these positions are RNA
taatacuttttccagccttc

SEQ ID NO: 139 - DNA, 20 bp, Artificial sequence
  Synthetic oligonucleotide
  misc_feature (1)..(2): bases at these positions are RNA
  misc_feature (4)..(5): bases at these positions are RNA
  misc_feature (7)..(7): bases at these positions are RNA
  misc_feature (10)..(11): bases at these positions are RNA
  misc_feature (13)..(18): bases at these positions are RNA
  misc_feature (20)..(20): bases at these positions are RNA
agtgctuttcataccaggtc

SEQ ID NO: 140 - DNA, 20 bp, Artificial sequence
  Synthetic oligonucleotide
  misc_feature (2)..(3): bases at these positions are RNA
  misc_feature (5)..(6): bases at these positions are RNA
  misc_feature (9)..(9): bases at these positions are RNA
  misc_feature (12)..(17): bases at these positions are RNA
  misc_feature (20)..(20): bases at these positions are RNA
taatacttuttccagccttc

SEQ ID NO: 141 - DNA, 20 bp, Artificial sequence
  Synthetic oligonucleotide
tcaagtatttttcattttcc

SEQ ID NO: 142 - DNA, 20 bp, Artificial sequence
  Synthetic oligonucleotide
gctgaagacatctcttcctt

SEQ ID NO: 143 - DNA, 20 bp, Artificial sequence
  Synthetic oligonucleotide
tcttcattaaagccatacct

SEQ ID NO: 144 - DNA, 20 bp, Artificial sequence
  Synthetic oligonucleotide
ttctttatatattctgctta

SEQ ID NO: 145 - DNA, 20 bp, Artificial sequence
  Synthetic oligonucleotide
tcttttcaaatccttcacct

SEQ ID NO: 146 - DNA, 20 bp, Artificial sequence
  Synthetic oligonucleotide
tcagttttatttcttcatta

SEQ ID NO: 147 - DNA, 20 bp, Artificial sequence
  Synthetic oligonucleotide
tgtacacttttacattccca

SEQ ID NO: 148 - DNA, 20 bp, Artificial sequence
  Synthetic oligonucleotide
ctgtacacttttacattccc

SEQ ID NO: 149 - DNA, 20 bp, Artificial sequence
  Synthetic oligonucleotide
ccatgacttcttcctcaatt

SEQ ID NO: 150 - DNA, 20 bp, Artificial sequence
  Synthetic oligonucleotide
cctcaatttttttcagcccc

SEQ ID NO: 151 - DNA, 20 bp, Artificial sequence
  Synthetic oligonucleotide
gtacattaacttccatgaaa

SEQ ID NO: 152 - DNA, 20 bp, Artificial sequence
  Synthetic oligonucleotide
catattttactctttttatt

SEQ ID NO: 153 - DNA, 20 bp, Artificial sequence
  Synthetic oligonucleotide
gtcaccatacttaataccat

SEQ ID NO: 154 - DNA, 20 bp, Artificial sequence
  Synthetic oligonucleotide
tgtacaattttccattacta

SEQ ID NO: 155 - DNA, 20 bp, Artificial sequence
  Synthetic oligonucleotide
ccttatatatttctactacc

SEQ ID NO: 156 - DNA, 20 bp, Artificial sequence
  Synthetic oligonucleotide
cagtacctaaaataagttca

SEQ ID NO: 157 - DNA, 20 bp, Artificial sequence
  Synthetic oligonucleotide
ttgtacaattttccattact

SEQ ID NO: 158 - DNA, 20 bp, Artificial sequence
  Synthetic oligonucleotide
gcaatgaatacaacacacat

SEQ ID NO: 159 - DNA, 20 bp, Artificial sequence
  Synthetic oligonucleotide
cgtctaacattcctgagcca

SEQ ID NO: 160 - DNA, 20 bp, Artificial sequence
  Synthetic oligonucleotide
ccatccttttctaaatggta

SEQ ID NO: 161 - DNA, 20 bp, Artificial sequence
  Synthetic oligonucleotide
tcttttatcatttcttttct

SEQ ID NO: 162 - DNA, 20 bp, Artificial sequence
  Synthetic oligonucleotide
aaattacttcttttatcatt

SEQ ID NO: 163 - DNA, 20 bp, Artificial sequence
  Synthetic oligonucleotide
ttaattttcccttcactcct

SEQ ID NO: 164 - DNA, 20 bp, Artificial sequence
  Synthetic oligonucleotide
cctgatgttccaaaattact

SEQ ID NO: 165 - DNA, 20 bp, Artificial sequence
  Synthetic oligonucleotide
aatgcatattggttttctca

SEQ ID NO: 166 - DNA, 20 bp, Artificial sequence
  Synthetic oligonucleotide
ctataattcttgcttcaacc

SEQ ID NO: 167 - DNA, 20 bp, Artificial sequence
  Synthetic oligonucleotide
tttcatgttccagatcacca

SEQ ID NO: 168 - DNA, 20 bp, Artificial sequence
  Synthetic oligonucleotide
gttccagatcaccatctttg

SEQ ID NO: 169 - DNA, 20 bp, Artificial sequence
  Synthetic oligonucleotide
  misc_feature (2)..(3): bases at these positions are RNA
  misc_feature (5)..(6): bases at these positions are RNA
  misc_feature (9)..(9): bases at these positions are RNA
  misc_feature (12)..(17): bases at these positions are RNA
  misc_feature (20)..(20): bases at these positions are RNA
tgctttucataccaggtctc

SEQ ID NO: 170 - DNA, 20 bp, Artificial sequence
  Synthetic oligonucleotide
  misc_feature (1)..(1): bases at thes positions are RNA
  misc_feature (3)..(4): bases at thes positions are RNA
  misc_feature (7)..(7): bases at thes positions are RNA
  misc_feature (9)..(10): bases at thes positions are RNA
  misc_feature (12)..(17): bases at thes positions are RNA
  misc_feature (19)..(19): bases at thes positions are RNA
gtgcttutcataccaggtct

SEQ ID NO: 171 - DNA, 20 bp, Artificial sequence
  Synthetic oligonucleotide
ccccaaactttcaaggcatt

SEQ ID NO: 172 - DNA, 20 bp, Artificial sequence
  Synthetic oligonucleotide
gttcactttgccataatcaa

SEQ ID NO: 173 - DNA, 23 bp, Artificial sequence
  Primer
ctatcaggacagagttcacatcc

SEQ ID NO: 174 - DNA, 23 bp, Artificial sequence
  Primer
gtttctaaagacatggtcacagc

SEQ ID NO: 175 - DNA, 22 bp, Artificial sequence
  Probe
aaaggccagccaccagttcagg

(19) United States
(12) Patent Application Publication
(10) Pub. No.: US 2022/0195431 A1
(43) Pub. Date: Jun. 23, 2022

(54) COMPOUNDS AND METHODS FOR REDUCING ATXN3 EXPRESSION

(71) Applicant: Ionis Pharmaceuticals, Inc., Carlsbad, CA (US)

(72) Inventors: Hien Thuy Zhao, San Diego, CA (US); Holly Kordasiewicz, San Diego, CA (US); Ruben E. Valas, San Diego, CA (US)

(73) Assignee: Ionis Pharmaceuticals, Inc., Carlsbad, CA (US)

(21) Appl. No.: 17/432,237

(22) PCT Filed: Feb. 21, 2020
(86) PCT No.: PCT/US2020/019272

§ 371 Date: Aug. 19, 2021

Related U.S. Application Data
(60) Provisional application No. 62/809,492, filed on Feb. 22, 2019.

(51) Int. Cl. C12N 15/113; A61K 31/712; A61P 25/28

(52) U.S. Cl. CPC C12N 15/113; A61K 31/712; A61P 25/28; A01K 2217/054; A01K 2227/105; C12N 2310/346; C12N 2310/315; C12N 2310/322; C12N 2310/341; C12N 2310/345; A01K 2267/0306

(57) ABSTRACT

Provided are compounds, methods, and pharmaceutical compositions for reducing the amount or activity of ATXN3 RNA in a cell or animal, and in certain embodiments reducing the amount of ATXN3 protein in a cell or animal. Such compounds, methods, and pharmaceutical compositions are useful to ameliorate at least one symptom or hallmark of a neurodegenerative disease. Such symptoms and hallmarks include motor dysfunction, aggregation formation, and neuron death. Such neurodegenerative diseases include spinocerebellar ataxia type 3(SCA3).

### SEQUENCE LISTING

The present application is being filed along with a Sequence Listing in electronic format. The Sequence Listing is provided as a file entitled BIOL0354WOSEQ_ST25.txt, created on Feb. 20, 2019, which is 208 KB in size. The information in the electronic format of the sequence listing is incorporated herein by reference in its entirety.

### FIELD

Provided are compounds, methods, and pharmaceutical compositions for reducing the amount or activity of ATXN3 RNA in a cell or animal, and in certain instances reducing the amount of Ataxin-3 protein in a cell or animal Such compounds, methods, and pharmaceutical compositions are useful to ameliorate at least one symptom or hallmark of a neurodegenerative disease. Such symptoms and hallmarks include ataxia, neuropathy, and aggregate formation. Such neurodegenerative diseases include spinocerebellar ataxia type 3(SCA3).

### BACKGROUND

Spinocerebellar ataxia type 3 (SCA3), also known as Machado-Joseph disease (MJD), is caused by a mutation in the ATXN3 gene and is characterized by progressive cerebellar ataxia and variable findings including a dystonic-rigid syndrome, a parkinsonian syndrome, or a combined syndrome of dystonia and peripheral neuropathy. SCA3 is inherited in an autosomal dominant manner. Offspring of affected individuals have a 50% chance of inheriting the mutation. The diagnosis of SCA3 rests on the use of molecular genetic testing to detect an abnormal CAG trinucleotide repeat expansion in ATXN3. Affected individuals have alleles with 52 to 86 CAG trinucleotide repeats. Such testing detects 100% of affected individuals. Expanded CAG repeats in the ATXN3 gene are translated into expanded polyglutamine repeats (polyQ) in the ataxin-3 protein and this toxic ataxin-3 protein is associated with aggregates. The polyglutamine expanded ataxin-3 protein in these aggregates is ubiquinated and the aggregates contain other proteins, including heat shock proteins and transcription factors. Aggregates are frequently observed in the brain tissue of SCA3 patients. Management of SCA3 is supportive as no medication slows the course of disease; restless legs syndrome and extrapyramidal syndromes resembling parkinsonism may respond to levodopa or dopamine agonists; spasticity, drooling, and sleep problems respond variably to lioresal, atropine-like drugs, and hypnotic agents; botulinum toxin has been used for dystonia and spasticity; daytime fatigue may respond to psychostimulants such as modafinil; and accompanying depression should be treated. Riess, 0., Rill), U., Pastore, A. et al. Cerebellum (2008) 7: 125.

Currently there is a lack of acceptable options for treating neurodegenerative diseases such as SCA3. It is therefore an object herein to provide compounds, methods, and pharmaceutical compositions for the treatment of such diseases.

### SUMMARY OF THE INVENTION

Provided herein are compounds, methods, and pharmaceutical compositions for reducing the amount or activity of ATXN3 RNA, and in certain embodiments reducing the amount of Ataxin-3 protein in a cell or animal In certain embodiments, the animal has a neurodegenerative disease. In certain embodiments, the animal has SCA3. In certain embodiments, compounds useful for reducing expression of ATXN3 RNA are oligomeric compounds. In certain embodiments, the oligomeric compound comprises a modified oligonucleotide.

Also provided are methods useful for ameliorating at least one symptom or hallmark of a neurodegenerative disease. In certain embodiments, the neurodegenerative disease is SCA3. In certain embodiments symptoms and hallmarks include ataxia, neuropathy, and aggregate formation. In certain embodiments, amelioration of these symptoms results in improved motor function, reduced neuropathy, and reduction in number of aggregates.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “”2′-MOE” or “2′-MOE sugar moiety” means a 2′-OCH₂CH₂OCH₃ group in place of the 2′-OH group of a ribosyl sugar moiety. “MOE” means methoxyethyl. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “2′-OMe” or “2′-O-methyl sugar moiety” means a 2′-OCH₃ group in place of the 2′-OH group of a ribosyl sugar moiety.

Unless otherwise indicated, a 2′-OMe sugar moiety is in the β-D configuration. “OMe” means O-methyl.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, “administering” means providing a pharmaceutical agent to an animal

As used herein, “animal” means a human or non-human animal

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound or oligomeric duplex capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom or hallmark is ataxia, neuropathy, and aggregate formation. In certain embodiments, amelioration of these symptoms results in improved motor function, reduced neuropathy, or reduction in number of aggregates.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl moiety is a ribosyl moiety. In certain embodiments, the bicyclic sugar moiety does not comprise a furanosyl moiety.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cleavable moiety” means a bond or group of atoms that is cleaved under physiological conditions, for example, inside a cell, an animal, or a human

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Complementary oligonucleotides and/or nucleic acids need not have nucleobase complementarity at each nucleoside. Rather, some mismatches are tolerated. As used herein, “fully complementary” or “100% complementary” in reference to an oligonucleotide, or portion thereof, means that the oligonucleotide, or portion thereof, is complementary to another oligonucleotide or nucleic acid at each nucleobase of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moiety of each nucleoside of the gap is a 2′-β-D-deoxyribosyl sugar moiety. Thus, the term “MOE gapmer” indicates a gapmer having a gap comprising 2′-β-D-deoxynucleosides and wings comprising 2′-MOE nucleosides. An “altered gapmer” means a gapmer having one 2′-substituted nucleoside at position 1, 2, 3, 4, or 5 of the gap (from 5′ to 3′). Unless otherwise indicated, a gapmer and altered gapmer may comprise one or more modified internucleoside linkages and/or modified nucleobases and such modifications do not necessarily follow the gapmer pattern of the sugar modifications. The term “mixed gapmer” indicates a gapmer having a gap comprising 2′-β-D-deoxynucleosides and wings comprising modified nucleosides comprising at least two different sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of structure or function of neurons, including death of neurons. In certain embodiments, neurodegenerative disease is spinocerebellar ataxia type 3 (SCA3).

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within an animal or cells thereof. Typically, conversion of a prodrug within the animal is facilitated by the action of an enzyme (e.g., endogenous or viral enzyme) or chemicals present in cells or tissues and/or by physiologic conditions.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics In certain embodiments, an RNAi compound modulates the amount, activity, and/or splicing of a target nucleic acid. The term RNAi compound excludes antisense compounds that act through RNase H.

As used herein, “self-complementary” in reference to an oligonucleotide means an oligonucleotide that at least partially hybridizes to itself.

As used herein, “stereorandom” or “stereorandom chiral center” in the context of a population of molecules of identical molecular formula means a chiral center having a random stereochemical configuration. For example, in a population of molecules comprising a stereorandom chiral center, the number of molecules having the (5) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unless otherwise indicated, a 2′-OH(H) ribosyl sugar moiety or a 2′-H(H) deoxyribosyl sugar moiety is in the β-D configuration. “MOE” means O-methoxyethyl. Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “standard in vivo assay” means the assay described in Example 3 and reasonable variations thereof.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal For example, a therapeutically effective amount improves a symptom of a disease.

### Certain Embodiments

Embodiment 1. An oligomeric compound comprising a modified oligonucleotide consisting of 12 to 50 linked nucleosides wherein the nucleobase sequence of the modified oligonucleotide is at least 90% complementary to an equal length portion of an ATXN3 nucleic acid, and wherein the modified oligonucleotide comprises at least one modification selected from a modified sugar moiety and a modified internucleoside linkage.
Embodiment 2. An oligomeric compound comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 11-172, wherein the modified oligonucleotide comprises at least one modification selected from a modified sugar moiety and a modified internucleoside linkage.
Embodiment 3. The oligomeric compound of embodiment 1 or embodiment 2, wherein the modified oligonucleotide consists of 15, 16, 17, 18, 19, or 20 linked nucleosides and has a nucleobase sequence comprising at least 15, at least 16, at least 17, at least 18, at least 19, or 20 contiguous nucleobases of any of the nucleobase sequences of SEQ ID NOs: 11-172.
Embodiment 4. The oligomeric compound of embodiment 3, wherein the modified oligonucleotide consists of 18, 19, or 20 linked nucleosides.
Embodiment 5. The oligomeric compound of any of embodiments 1-4, wherein the modified oligonucleotide has a nucleobase sequence that is at least 90%, at least 95%, or 100% complementary to an equal length portion of an ATXN 3 nucleic acid when measured across the entire nucleobase sequence of the modified oligonucleotide.
Embodiment 6. The oligomeric compound of any of embodiments 1-5, wherein the modified oligonucleotide has a nucleobase sequence comprising a portion of at least 8, at least 9, at least 10, at least 11, at least 12, at least 13, at least 14, at least 15, at least 16, at least 17, at least 18, at least 19, or 20 contiguous nucleobases, wherein the portion is complementary to:
   an equal length portion of nucleobases 6,597-6,619 of SEQ ID NO: 2;
   an equal length portion of nucleobases 15,664-15,689 of SEQ ID NO: 2;
   an equal length portion of nucleobases 19,451-19,476 of SEQ ID NO: 2;
   an equal length portion of nucleobases 30,448-30,473 of SEQ ID NO: 2;
   an equal length portion of nucleobases 32,940-32,961 of SEQ ID NO: 2;
   an equal length portion of nucleobases 34,013-34,039 of SEQ ID NO: 2;
   an equal length portion of nucleobases 37,151-37,172 of SEQ ID NO: 2;
   an equal length portion of nucleobases 43,647-43,674 of SEQ ID NO: 2;
   an equal length portion of nucleobases 46,389-46,411 of SEQ ID NO: 2;
   an equal length portion of nucleobases 46,748-46,785 of SEQ ID NO: 2; or
   an equal length portion of nucleobases 47,594-47,619 of SEQ ID NO: 2.
Embodiment 7. The oligomeric compound of any one of embodiments 1-6, wherein the ATXN3 nucleic acid has the nucleobase sequence of any of SEQ ID NOs: 1, 2, or 3.
Embodiment 8. The oligomeric compound of any of embodiments 1-7, wherein the modified oligonucleotide comprises at least one modified sugar moiety.
Embodiment 9. The oligomeric compound of any of embodiments 8-10, wherein the modified oligonucleotide comprises at least one bicyclic sugar moiety.
Embodiment 10. The oligomeric compound of embodiment 9, wherein the bicyclic sugar moiety has a 4′-2′ bridge, wherein the 4′-2′ bridge is selected from —CH₂—O—; and —CH(CH₃)—O—.
Embodiment 11. The oligomeric compound of embodiment 8, wherein the modified oligonucleotide comprises at least one non-bicyclic modified sugar moiety.
Embodiment 12. The oligomeric compound of embodiment 11, wherein the non-bicyclic modified sugar moiety is any of a 2′-MOE sugar moiety or a 2′-OMe sugar moiety.
Embodiment 13. The oligomeric compound of embodiment 12, wherein each modified nucleoside of the modified oligonucleotide comprises a modified non-bicyclic sugar moiety comprising a 2′-MOE sugar moiety or a 2′-OMe sugar moiety.
Embodiment 14. The oligomeric compound of embodiment 12, wherein each modified sugar moiety is a 2′-MOE sugar moiety.
Embodiment 15. The oligomeric compound of any of embodiments 8-12, wherein the modified oligonucleotide comprises at least one sugar surrogate.
Embodiment 16. The oligomeric compound of embodiment 15, wherein the sugar surrogate is any of morpholino, modified morpholino, PNA, THP, and F-HNA.
Embodiment 17. The oligomeric compound of any of embodiments 1-12 and 15-16, wherein the modified oligonucleotide is a gapmer or an altered gapmer.
Embodiment 18. The oligomeric compound of any of embodiments 1-12 and 15-17, wherein the modified oligonucleotide has a sugar motif comprising:
   a 5′-region consisting of 1-6 linked 5′-nucleosides;
   a central region consisting of 6-10 linked central region nucleosides; and
   a 3′-region consisting of 1-5 linked 3′-nucleosides; wherein each of the 5′-region nucleosides and each of the 3′-region nucleosides comprises a modified sugar moiety and each of the central region nucleosides comprises a 2′-β-D--deoxyribosyl sugar moiety.
Embodiment 19. The oligomeric compound of embodiment 18, wherein the modified sugar moiety is a 2′-MOE sugar moiety.
Embodiment 20. The oligomeric compound of any of embodiments 1-12 and 15-17, wherein the modified oligonucleotide has a sugar motif comprising:
   a 5′-region consisting of 1-6 linked 5′-nucleosides, each comprising a 2′-MOE sugar moiety;
   a 3′-region consisting of 1-5 linked 3′-nucleosides, each comprising a 2′-MOE sugar moiety; and
   a central region consisting of 6-10 linked central region nucleosides, wherein one of the central region nucleosides comprises a 2′-O-methyl sugar moiety and the remainder of the central region nucleosides each comprise a 2′-β-D-deoxyribosyl sugar moiety.
Embodiment 21. The oligomeric compound of embodiment 20, wherein the central region has the following formula (5′-3′): (N_{d})(N_{y})(N_{d})ₙwherein N_{y} is a nucleoside comprising a 2′-O-methyl sugar moiety and each N_{d} is a nucleoside comprising a 2′-β-D-deoxyribosyl sugar moiety, and n is 10.
Embodiment 22. The oligomeric compound of any of embodiments 1-21, wherein the modified oligonucleotide comprises at least one modified internucleoside linkage.
Embodiment 23. The oligomeric compound of embodiment 22, wherein each internucleoside linkage of the modified oligonucleotide is a modified internucleoside linkage.
Embodiment 24. The oligomeric compound of embodiment 22 or embodiment 23, wherein at least one internucleoside linkage is a phosphorothioate internucleoside linkage.
Embodiment 25. The oligomeric compound of embodiment 22 or embodiment 24 wherein the modified oligonucleotide comprises at least one phosphodiester internucleoside linkage.
Embodiment 26. The oligomeric compound of any of embodiments 22 or 24-25, wherein each internucleoside linkage is either a phosphodiester internucleoside linkage or a phosphorothioate internucleoside linkage.
Embodiment 27. The oligomeric compound of embodiment 23, wherein each internucleoside linkage is a phosphorothioate internucleoside linkage.
Embodiment 28. The oligomeric compound of embodiments 1-22 or 24-25, wherein the modified oligonucleotide has an internucleoside linkage motif (5′ to 3′) selected from among: sooooossssssssssoss, soooossssssssssooos, soooossssssssssooss, sooosssssssssooss, sooossssssssssooss, sooosssssssssssooos, sooosssssssssssooss, sossssssssssssssoss, and ssoosssssssssssooss; wherein,
   s=a phosphorothioate internucleoside linkage, and
   o=a phosphodiester internucleoside linkage.
Embodiment 29. The oligomeric compound of any of embodiments 1-28, wherein the modified oligonucleotide comprises at least one modified nucleobase.
Embodiment 30. The oligomeric compound of embodiment 29, wherein the modified nucleobase is a 5-methyl cytosine.
Embodiment 31. The oligomeric compound of any one of embodiments 1-30, wherein the modified oligonucleotide consists of 12-22, 12-20, 14-20, 16-20, 18-20, or 18-22 linked nucleosides.
Embodiment 32. The oligomeric compound of any one of embodiments 1-30, wherein the modified oligonucleotide consists of 16, 17, 18, 19, or 20 linked nucleosides.
Embodiment 33. An oligomeric compound comprising a modified oligonucleotide according to the following chemical notation: AₑₛGₑₒ^{m}Cₑₒ^{m}CₑₒAₑₛA_{ds}T_{ds}A_{ds}T_{ds}T_{ds}T_{ds}A_{ds}T_{ds}A_{ds}G_{ds}GₑₒTₑₒGₑₛ^{m}CₑₛTₑ (SEQ ID NO: 117),
   wherein,
   A=an adenine nucleobase,
   mC=a 5-methyl cytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and
   o=a phosphodiester internucleoside linkage.
Embodiment 34. An oligomeric compound comprising a modified oligonucleotide according to the following chemical notation:
   Gₑₛ^{m}Cₑₒ^{m}CₑₒAₑₒTₑₒTₑₒA_{ds}A_{ds}T_{ds}^{m}C_{ds}T_{ds}A_{ds}T_{ds}A_{ds}^{m}C_{ds}T_{ds}GₑₒAₑₛAₑₛTₑ (SEQ ID NO: 137), wherein,
   A=an adenine nucleobase,
   mC=a 5-methyl cytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and
   o=a phosphodiester internucleoside linkage.
Embodiment 35. An oligomeric compound comprising a modified oligonucleotide according to the following chemical notation:
   Gₑₛ^{m}CₑₒAₑₒTₑₒAₑₒTₑₒT_{ds}G_{ds}G_{ds}T_{ds}T_{ds}T_{ds}T_{ds}^{m}C_{ds}T_{ds}^{m}C_{ds}AₑₒTₑₛTₑₛTₑ (SEQ ID NO: 50), wherein,
   A=an adenine nucleobase,
   mC=a 5-methyl cytosine nucleobase,
   G=a guanine nucleobase,
   T=a thymine nucleobase,
   e=a 2′-MOE sugar moiety,
   d=a 2′-β-D-deoxyribosyl sugar moiety,
   s=a phosphorothioate internucleoside linkage, and
   o=a phosphodiester internucleoside linkage.
Embodiment 36. The oligomeric compound of any of embodiments 1-35, wherein the oligomeric compound is a singled-stranded oligomeric compound.
Embodiment 37. The oligomeric compound of any of embodiments 1-36 consisting of the modified oligonucleotide.
Embodiment 38. The oligomeric compound of any of embodiments 1-37 comprising a conjugate group comprising a conjugate moiety and a conjugate linker.
Embodiment 39. The oligomeric compound of embodiment 38, wherein the conjugate group comprises a

GalNAc cluster comprising 1-3 GalNAc ligands
Embodiment 40. The oligomeric compound of embodiment 38 or embodiment 39, wherein the conjugate linker consists of a single bond.
Embodiment 41. The oligomeric compound of embodiment 38, wherein the conjugate linker is cleavable.
Embodiment 42. The oligomeric compound of embodiment 38, wherein the conjugate linker comprises 1-3 linker-nucleosides.
Embodiment 43. The oligomeric compound of any of embodiments 38-42, wherein the conjugate group is attached to the modified oligonucleotide at the 5′-end of the modified oligonucleotide.
Embodiment 44. The oligomeric compound of any of embodiments 38-42, wherein the conjugate group is attached to the modified oligonucleotide at the 3′-end of the modified oligonucleotide.
Embodiment 45. The oligomeric compound of any of embodiments 1-36 or 38-44 comprising a terminal group.
Embodiment 46. The oligomeric compound of any of embodiments 1-41 or 43-45, wherein the oligomeric compound does not comprise linker-nucleosides.
Embodiment 47. A modified oligonucleotide according to the following chemical structure:

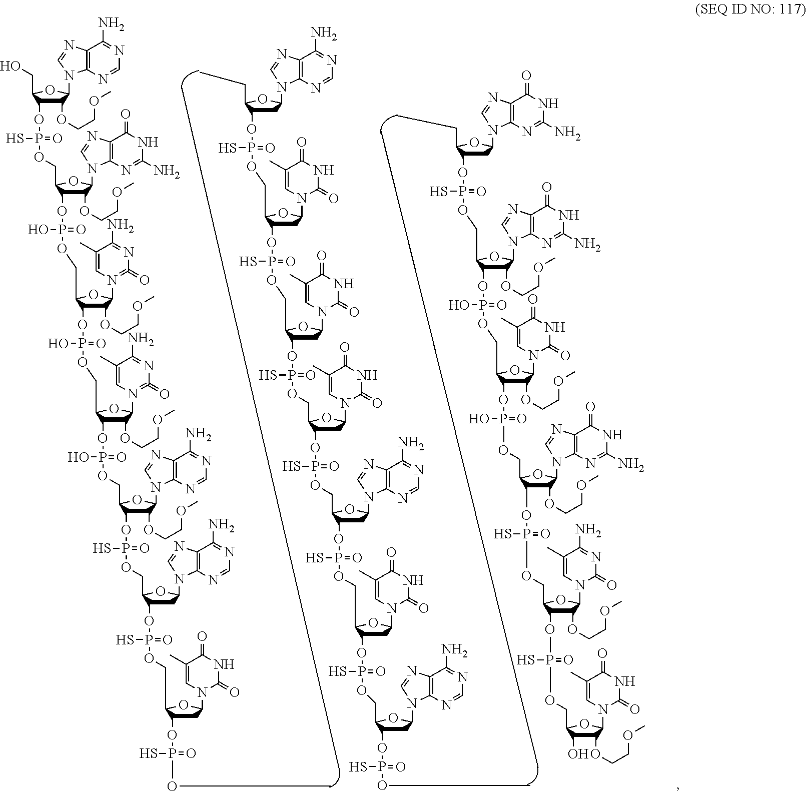

or a salt thereof.
Embodiment 48. The modified oligonucleotide of embodiment 47, which is the sodium salt or the potassium salt.
Embodiment 49. A modified oligonucleotide according to the following formula:

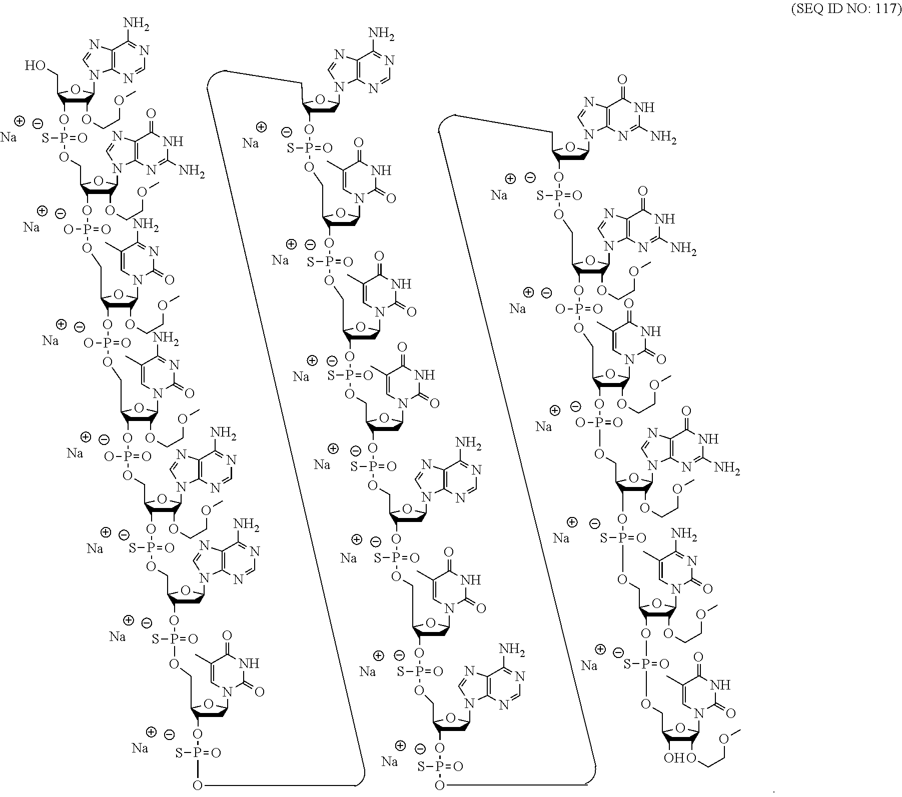

Embodiment 50. A modified oligonucleotide according to the following formula:

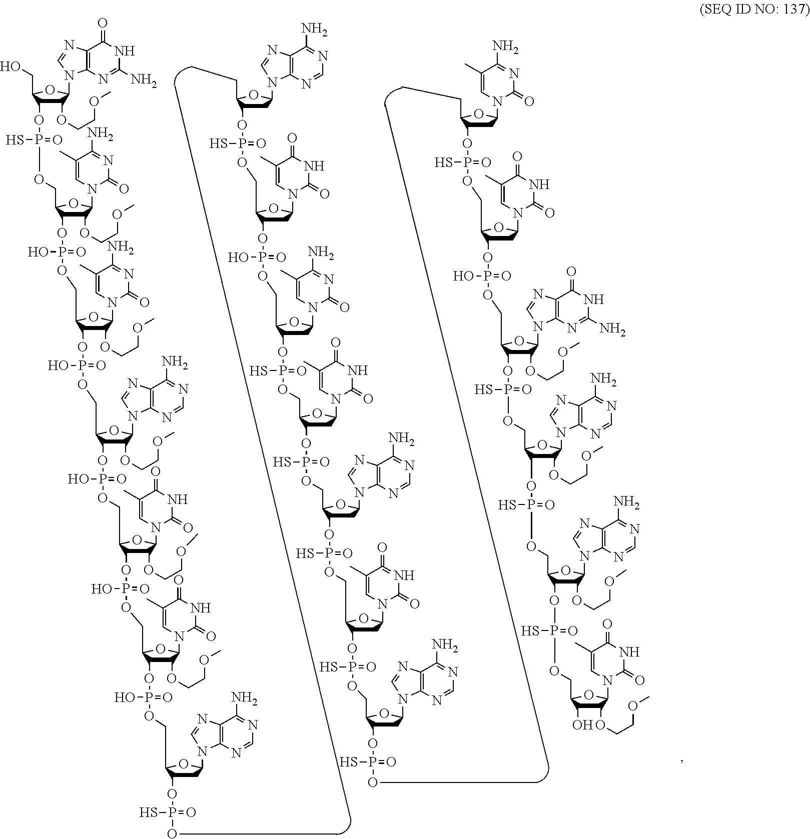

or a salt thereof.
Embodiment 51. The modified oligonucleotide of embodiment 50, which is the sodium salt or the potassium salt.
Embodiment 52. A modified oligonucleotide according to the following formula:

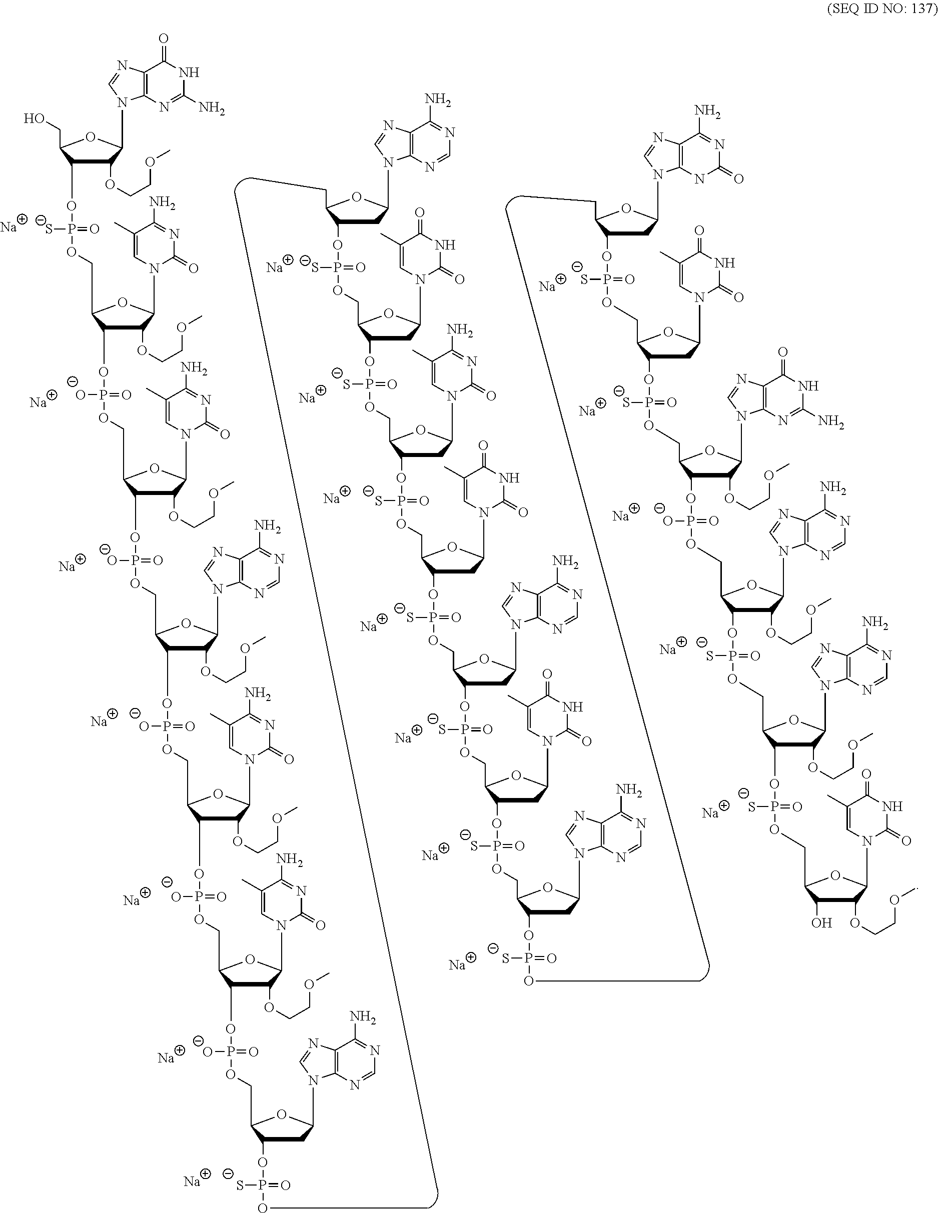

Embodiment 53. A modified oligonucleotide according to the following formula:

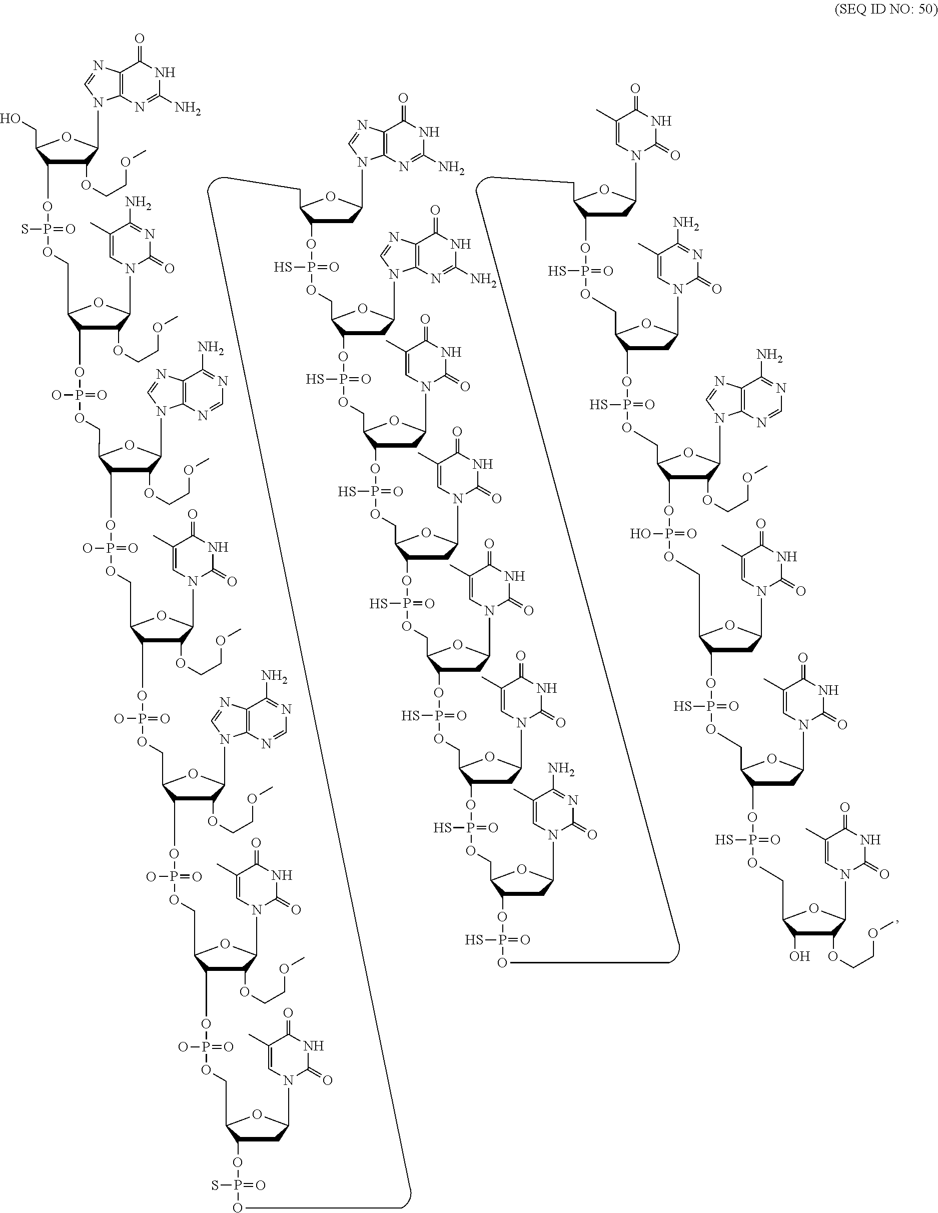

or a salt thereof.
Embodiment 54. The modified oligonucleotide of embodiment 53, which is the sodium salt or the potassium salt.
Embodiment 55. A modified oligonucleotide according to the following formula:

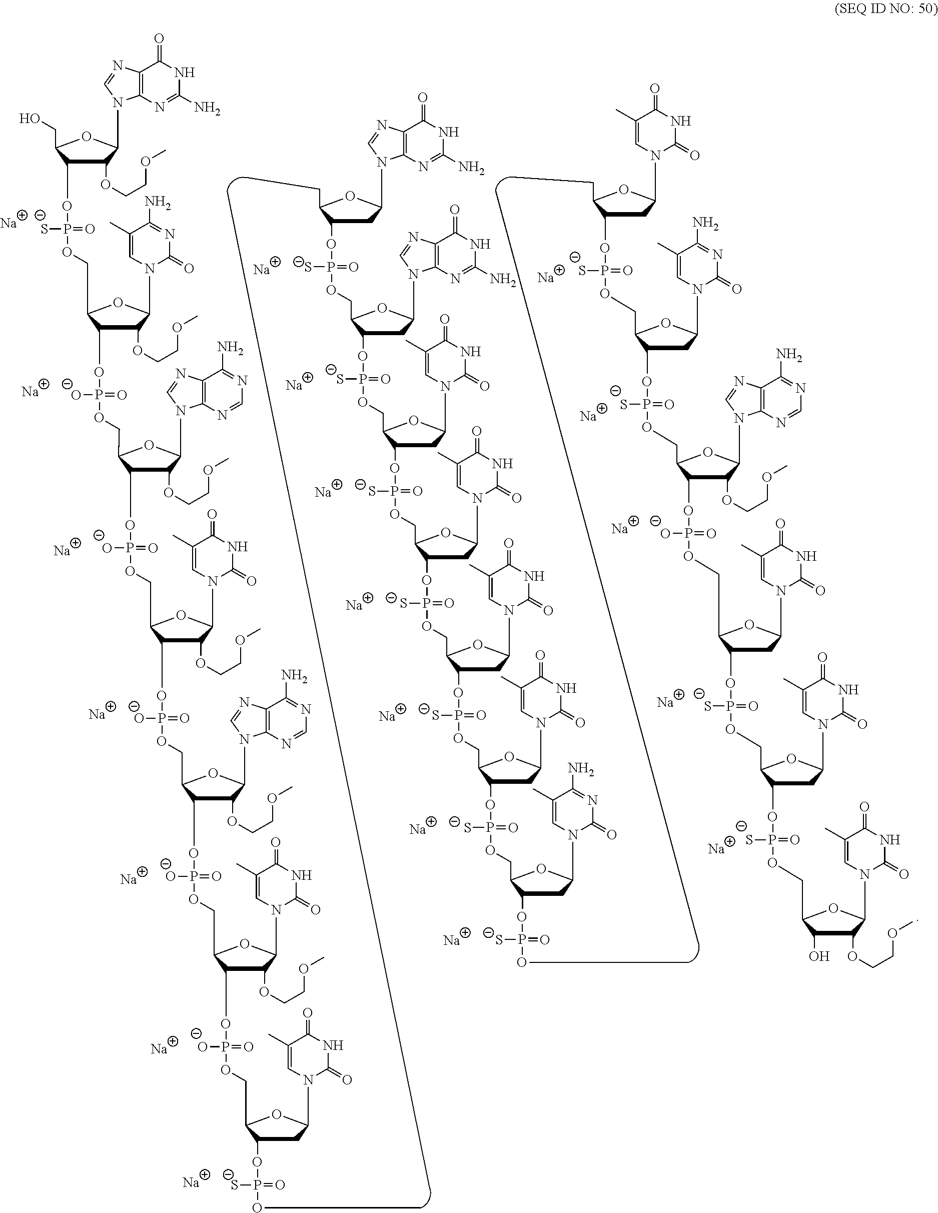

Embodiment 56. A pharmaceutical composition comprising the oligomeric compound of any of embodiments 1-46 or the modified oligonucleotide of any of embodiments 47-55, and a pharmaceutically acceptable diluent or carrier.
Embodiment 57. The pharmaceutical composition of embodiment 56, comprising a pharmaceutically acceptable diluent and wherein the pharmaceutically acceptable diluent is artificial CSF (aCSF) or PBS.
Embodiment 58. The pharmaceutical composition of embodiment 57, wherein the pharmaceutical composition consists essentially of the modified oligonucleotide and artificial CSF (aCSF).
Embodiment 59. The pharmaceutical composition of embodiment 57, wherein the pharmaceutical composition consists essentially of the modified oligonucleotide and PBS.
Embodiment 60. A chirally enriched population of modified oligonucleotides of any of embodiments 56-59, wherein the population is enriched for modified oligonucleotides comprising at least one particular phosphorothioate internucleoside linkage having a particular stereochemical configuration.
Embodiment 61. The chirally enriched population of embodiment 60, wherein the population is enriched for modified oligonucleotides comprising at least one particular phosphorothioate internucleoside linkage having the (Sp) configuration.
Embodiment 62. The chirally enriched population of embodiment 60, wherein the population is enriched for modified oligonucleotides comprising at least one particular phosphorothioate internucleoside linkage having the

(Rp) configuration.
Embodiment 63. The chirally enriched population of embodiment 60, wherein the population is enriched for modified oligonucleotides having a particular, independently selected stereochemical configuration at each phosphorothioate internucleoside linkage.
Embodiment 64. The chirally enriched population of embodiment 63, wherein the population is enriched for modified oligonucleotides having the (Sp) configuration at each phosphorothioate internucleoside linkage or for modified oligonucleotides having the (Rp) configuration at each phosphorothioate internucleoside linkage.
Embodiment 65. The chirally enriched population of embodiment 63, wherein the population is enriched for modified oligonucleotides having the (Rp) configuration at one particular phosphorothioate internucleoside linkage and the (Sp) configuration at each of the remaining phosphorothioate internucleoside linkages.
Embodiment 66. The chirally enriched population of embodiment 63, wherein the population is enriched for modified oligonucleotides having at least 3 contiguous phosphorothioate internucleoside linkages in the Sp, Sp, and Rp configurations, in the 5′ to 3′ direction.
Embodiment 67. A population of modified oligonucleotides of any of embodiments 47-55, wherein all of the phosphorothioate internucleoside linkages of the modified oligonucleotide are stereorandom.
Embodiment 68. A method of reducing expression of Ataxin 3 in a cell, comprising contacting the cell with an oligomeric compound of any of embodiments 1-46 or a modified oligonucleotide of any of embodiments 47-55.
Embodiment 69. The method of embodiment 68, wherein the level of Ataxin 3 RNA is reduced.
Embodiment 70. The method of any of embodiments 68-69, wherein the level of Ataxin 3 protein is reduced.
Embodiment 71. The method of any of embodiments 68-69, wherein the cell is in vitro.
Embodiment 72. The method of any of embodiments 68-69, wherein the cell is in an animal
Embodiment 73. A method comprising administering to an animal the pharmaceutical composition of any of embodiments 56-59.
Embodiment 74. The method of embodiment 73, wherein the animal is a human
Embodiment 75. A method of treating a disease associated with ATXN3 comprising administering to an individual having or at risk for developing a disease associated with ATXN3 a therapeutically effective amount of a pharmaceutical composition of embodiments 56-59, and thereby treating the disease associated with ATXN3.
Embodiment 76. The method of embodiment 75, wherein the disease associated with ATXN3 is a neurodegenerative disease.
Embodiment 77. The method of embodiment 76, wherein the neurodegenerative disease is SCA3.
Embodiment 78. The method of embodiment 76, wherein at least one symptom or hallmark of the neurodegenerative disease is ameliorated.
Embodiment 79. The method of embodiment 77, wherein the symptom or hallmark is ataxia, neuropathy, and aggregate formation.
Embodiment 80. The method of any of embodiments 73-79, wherein the pharmaceutical composition is administered to the central nervous system or systemically.
Embodiment 81. The method of embodiment 80, wherein the pharmaceutical composition is administered to the central nervous system and systemically.
Embodiment 82. The method of any of embodiment 73-79, wherein the pharmaceutical composition is administered any of intrathecally, systemically, subcutaneously, or intramuscularly.
Embodiment 83. Use of an oligomeric compound of any of embodiments 1-46 or a modified oligonucleotide of any of embodiments 47-55 for reducing Ataxin 3 expression in a cell.
Embodiment 84. The use of embodiment 83, wherein the level of Ataxin 3 RNA is reduced.
Embodiment 85. The use of embodiment 83, wherein the level of Ataxin 3 protein is reduced.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modifed sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the fumnosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂), ON(CH₃)₂, O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, P(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modifed sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-240 , 4′-(CH₂)₂-O-2′ (“ENA”), 4′-CH(CH₃)—O-2′ (referred to as “constrained ethyl” or “cEt”), 4′-CH₂—O—CH₂-22′, 4′-CH₂-N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂-O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem.,2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroalyl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂J₁), or sulfoxyl (S(=O)-J₁); and

each J₁ and J2 is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(=O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. P_at. No.6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No., 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

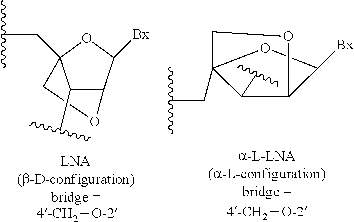

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

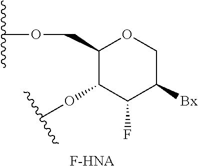

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3¹-fluoro tetrahydropyran), and nucleosides comprising additional modified THP compounds having the formula:

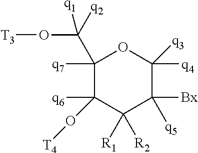

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

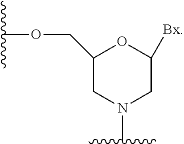

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp) Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. o. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond, P(O₂)═O, (also referred to as unmodified or naturally occurring linkages); phosphotriesters; methylphosphonates; methoxypropylphosphonates (“MOP”); phosphoramidates; phosphorothioates (P(O₂)═S); and phosphorodithioates (HS—P═S). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)-)—. Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al., JACS, 2003, 125, 8307, Wan et al. Nuc. Acid. Res., 2004, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

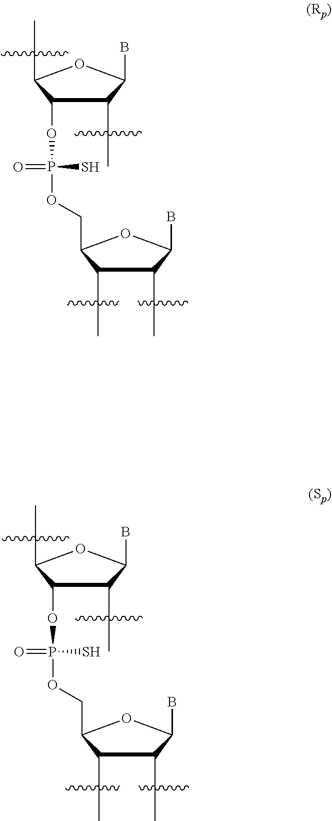

Unless otherwise indicated, chiral internucleoside linkages of modified oligonucleotides described herein can be stereorandom or in a particular stereochemical configuration.

In certain embodiments, modified oligonucleotides comprise an internucleoside motif of (5′ to 3′) sooosssssssssssssss. In certain embodiments, the particular stereochemical configuration of the modified oligonucleotides is (5′ to 3′) Sp-o-o-o-Sp-Sp-Sp-Rp-Sp-Sp-Rp-Sp-Sp-Sp-Sp-Sp-Sp-Sp-Sp or Sp-o-o-o-Sp-Sp-Sp-Rp-Sp-Sp-Sp-Sp-Sp-Sp-Sp-Sp-Sp-Sp-Sp; wherein each ‘Sp’ represents a phosphorothioate internucleoside linkage in the S configuration; Rp represents a phosphorothioate internucleoside linkage in the R configuration; and ‘o’ represents a phosphodiester internucleoside linkage.

Neutral internucleoside linkages include, without limitation, phosphotriesters, methylphosphonates, MMI (3′-CH₂—N(CH₃)—O-5′), amide-3 (3′-CH₂—C(═O—N(H)-5′), amide-4 (3′-CH₂-N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkages. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap.

Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, each nucleoside of each wing of a gapmer is a modified nucleoside. In certain embodiments, at least one nucleoside of each wing of a gapmer comprises a modified sugar moiety. In certain embodiments, at least two, at least three, at least four, at least five, or at least six nucleosides of each wing of a gapmer comprise a modified sugar moiety.

In certain embodiments, the gap of a gapmer comprises 7-12 nucleosides. In certain embodiments, each nucleoside of the gap of a gapmer is an unmodified 2′-deoxynucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside. In certain embodiments, at least one nucleoside of the gap of a gapmer comprises a modified sugar moiety and each remaining nucleoside comprises a 2′-deoxyribosyl sugar moiety.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleosides sugars. Thus, a 5-10-5 MOE gapmer consists of 5 linked 2′-MOE modified nucleosides in the 5′-wing, 10 linked 2′-deoxyribonucleosides in the gap, and 5 linked 2′-MOE nucleosides in the 3′-wing.

In certain embodiments, modified oligonucleotides are 5-10-5 MOE gapmers. In certain embodiments, modified oligonucleotides are 5-9-5 MOE gapmers. In certain embodiments, modified oligonucleotides are 6-10-4 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain such embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs. C. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24,20 to 25, 20 to 26,20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for (β-D ribosyl sugar moieties, and all of the phosphorothioate internucleoside linkages are stereorandom. In certain embodiments, the modified oligonucleotides of a chirally enriched population are enriched for both (β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

II. Certain Oliogomeric Compounds In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-l-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

B. Certain Terminal Groups

In certain embodiments, oligomeric compounds comprise one or more terminal groups. In certain such embodiments, oligomeric compounds comprise a stabilized 5′-phophate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit, modulate, or increase the amount or activity of a target nucleic acid by 25% or more in the standard in vivo assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal

### V. Certain Tar2et Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides. In certain embodiments, oligonucleotides are complementary to the target nucleic acid over the entire length of the oligonucleotide. In certain embodiments, oligonucleotides are at least 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length. In certain embodiments, the region of full complementarity is 6, 7, 8, 9, 10, 11, 12, 13, 14, 15, 16, 17, 18, 19, or 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

B. ATXN3

In certain embodiments, oligomeric compounds comprise or consist of an oligonucleotide comprising a region that is complementary to a target nucleic acid, wherein the target nucleic acid is ATXN3. In certain embodiments, ATXN3 nucleic acid has the sequence set forth in SEQ ID NO: 1 (GENBANK Accession No: NM_004993.5), SEQ ID NO: 2 (the complement of GENBANK Accession No NC_000014.9 truncated from nucleotides 92,056,001 to 92,110,000), or SEQ ID NO: 3 (the complement of GENBANK Accession No NC_000014.9 truncated from nucleotides 92038001 to 92110000).

In certain embodiments, contacting a cell with an oligomeric compound complementary to any of SEQ ID NOs: 1-3 reduces the amount of ATXN3 RNA, and in certain embodiments reduces the amount of Ataxin-3 protein. In certain embodiments, the oligomeric compound consists of a modified oligonucleotide. In certain embodiments, contacting a cell in an animal with an oligomeric compound complementary to any of SEQ ID NOs: 1-3 ameliorate one or more symptom or hallmark of a neurodegenerative disease. In certain embodiments, the symptom or hallmark is ataxia, neuropathy, and aggregate formation. In certain embodiments, contacting a cell in an animal with an oligonucleotide complementary to any of SEQ ID Nos: 1-3 results in improved motor function, reduced neuropathy, and/or reduction in number of aggregates. In certain embodiments, the oligomeric compound consists of a modified oligonucleotide.

C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS), including spinal cord, cortex, cerebellum, and brain stem.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1269455, Compound No. 1287621, and Compound No. 1287095 equals the number of fully protonated molecules that weighs 10 mg. This would be equivalent to 10.59 mg of solvent-free, sodium acetate-free, anhydrous sodiated Compound No. 1269455, 10.59 mg of solvent-free, sodium acetate-free, anhydrous sodiated Compound No. 1287621, and 10.59 mg of solvent-free, sodium acetate-free, anhydrous sodiated Compound No. 1287095. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### VII. Certain Compositions

1. Compound No. 1269455

In certain embodiments, Compound No. 1269455 is characterized as a 5-10-5 MOE gapmer having a sequence of (from 5′ to 3′) AGCCAATATTTATAGGTGCT (SEQ ID NO: 117), wherein each of nucleosides 1-5 and 16-20 (from 5′ to 3′) comprise a 2′-MOE sugar moiety and each of nucleosides 6-15 are 2′-β-D-deoxynucleosides, wherein the internucleoside linkages between nucleosides 2 to 3, 3 to 4, 4 to 5, 16 to 17, and 17 to 18 are phosphodiester internucleoside linkages and the internucleoside linkages between nucleosides 1 to 2, 5 to 6, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate internucleoside linkages, and wherein each cytosine is a 5-methyl cytosine.

In certain embodiments, Compound No. 1269455 is represented by the following chemical notation: AₑₛGₑₒ^{m}Cₑₒ^{m}CₑₒAₑₛA_{ds}T_{ds}A_{ds}T_{ds}T_{ds}T_{ds}A_{ds}T_{ds}A_{ds}G_{ds}GₑₒTₑₒGₑₛ^{m}CₑₛTₑ (SEQ ID NO: 117), wherein,

A=an adenine nucleobase,

mC=a 5-methyl cytosine nucleobase,

G=a guanine nucleobase,

T=a thymine nucleobase,

e=a 2′-MOE sugar moiety,

d=a 2′-β-D-deoxyribosyl sugar moiety,

s=a phosphorothioate internucleoside linkage, and

o=a phosphodiester internucleoside linkage.

In certain embodiments, Compound No. 1269455 is represented by the following chemical structure:

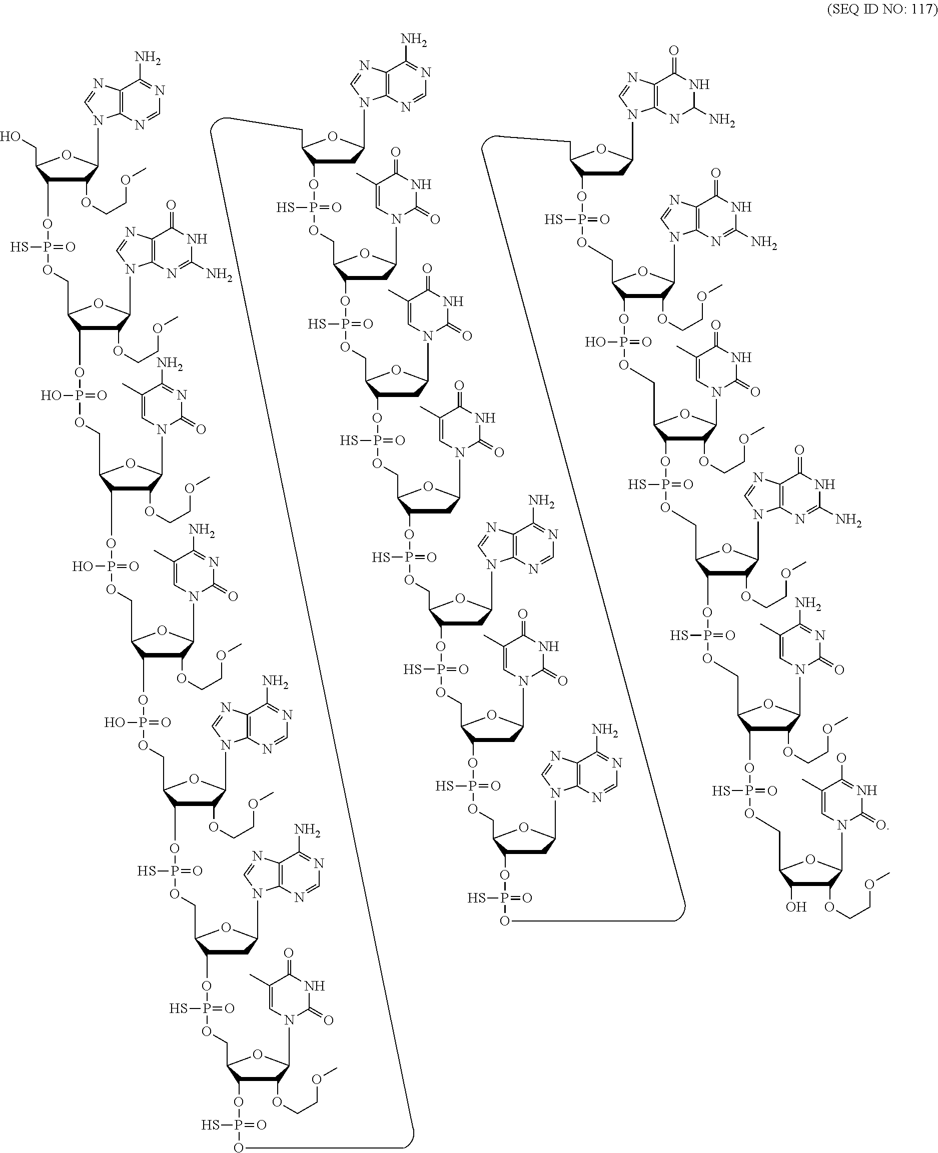

### Structure 1. Compound No. 1269455

In certain embodiments, the sodium salt of Compound No. 1269455 is represented by the following chemical structure:

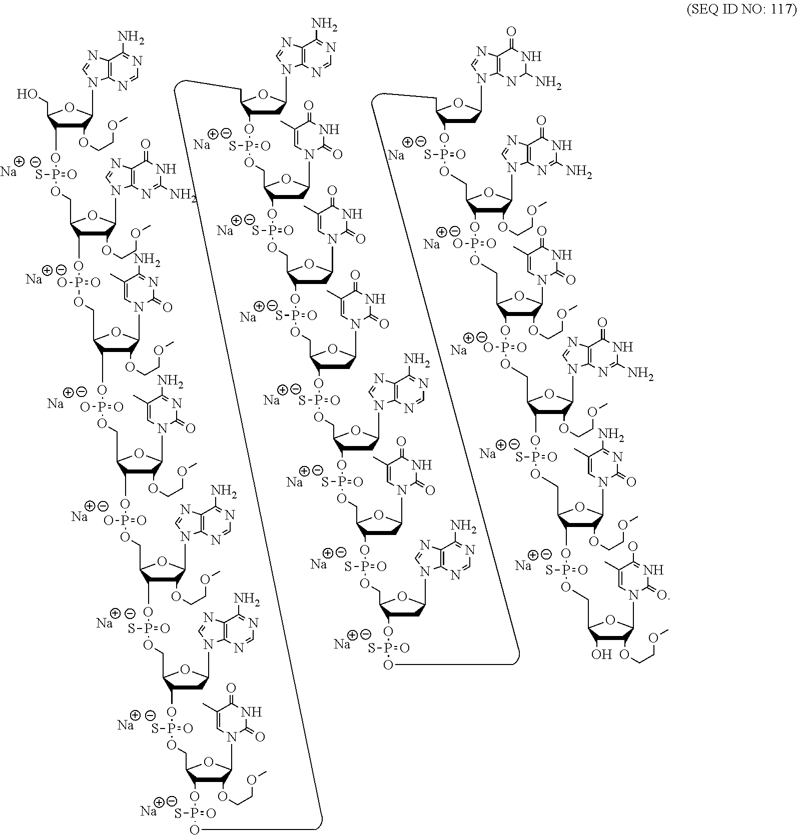

Structure 2. The sodium salt of Compound No. 1269455

2. Compound No. 1287621

In certain embodiments, Compound No. 1287621 is characterized as a 6-10-4 MOE gapmer having a sequence of (from 5′ to 3′) GCCATTAATCTATACTGAAT (SEQ ID NO: 137), wherein each of nucleosides 1-6 and 17-20 (from 5′ to 3′) comprise a 2′-MOE sugar moiety and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages and the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methyl cytosine.

In certain embodiments, Compound No. 1287621 is represented by the following chemical notation: Gₑₛ^{m}Cₑₒ^{m}CₑₒAₑₒTₑₒTₑₒA_{ds}A_{ds}T_{ds}^{m}C_{ds}T_{ds}A_{ds}T_{ds}A_{ds}^{m}C_{ds}T_{ds}GₑₒAₑₛAₑₛTₑ (SEQ ID NO: 137), wherein,

A=an adenine nucleobase,

mC=a 5-methyl cytosine nucleobase,

G=a guanine nucleobase,

T=a thymine nucleobase,

e=a 2′-MOE sugar moiety,

d=a 2′-β-D-deoxyribosyl sugar moiety,

s=a phosphorothioate internucleoside linkage, and

o=a phosphodiester internucleoside linkage.

In certain embodiments, Compound No. 1287621 is represented by the following chemical structure:

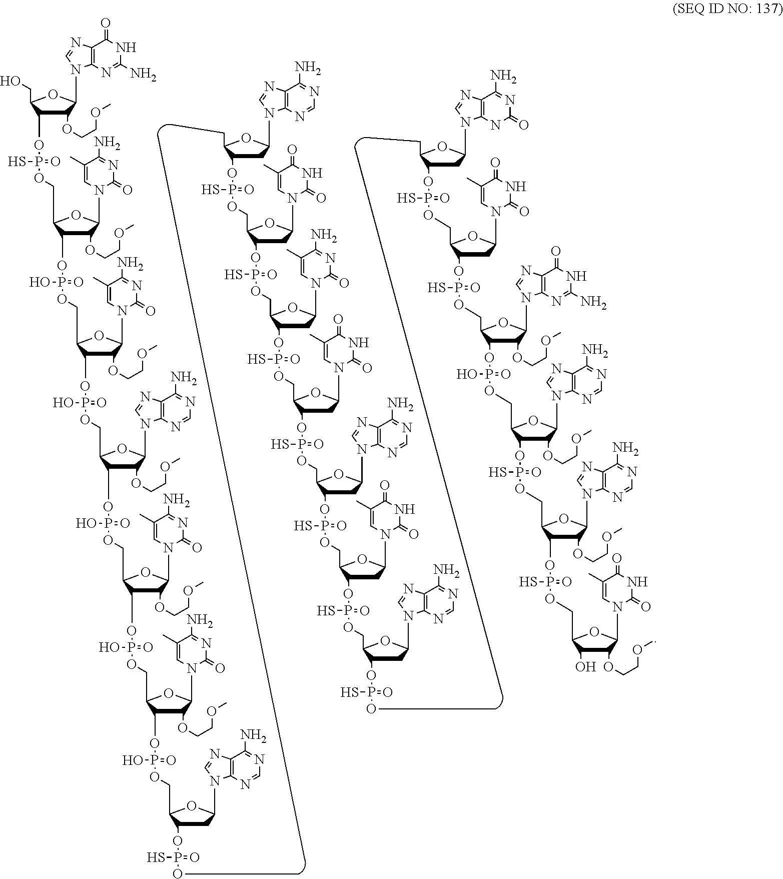

### Structure 3. Compound No. 1287621

In certain embodiments, the sodium salt of Compound No. 1287621 is represented by the following chemical structure:

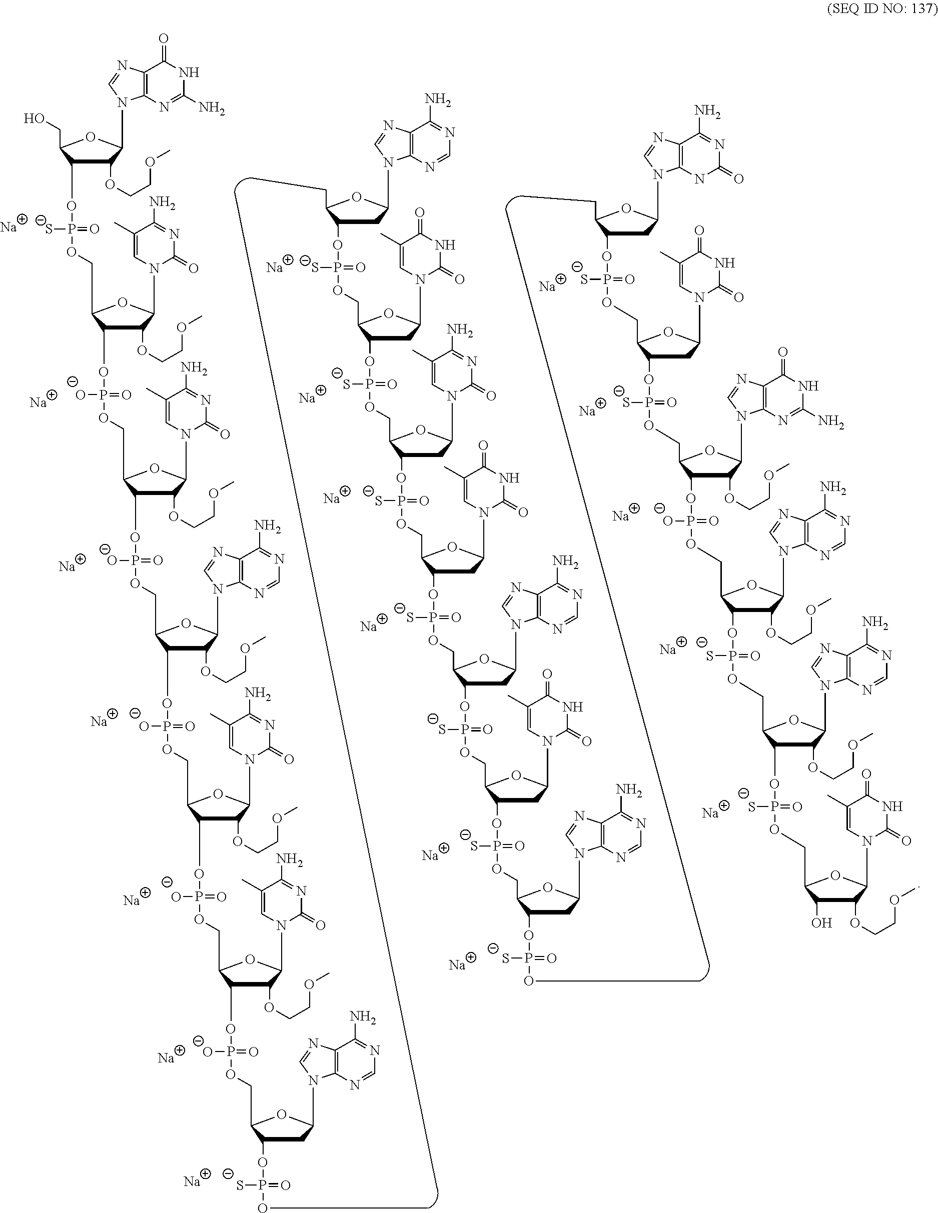

Structure 4. The sodium salt of Compound No. 1287621

3. Compound No. 1287095

In certain embodiments, Compound No. 1287095 is characterized as a 6-10-4 MOE gapmer having a sequence of (from 5′ to 3′) GCATATTGGTTTTCTCATTT (SEQ ID NO: 50), wherein each of nucleosides 1-6 and 17-20 (from 5′ to 3′) comprise a 2′-MOE sugar moiety and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages and the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methyl cytosine.

In certain embodiments, Compound No. 1287095 is represented by the following chemical notation: Gₑₛ^{m}CₑₒAₑₒTₑₒAₑₒTₑₒT_{ds}G_{ds}G_{ds}T_{ds}T_{ds}T_{ds}T_{ds}^{m}C_{ds}T_{ds}^{m}C_{ds}AₑₒTₑₛTₑₛTₑ (SEQ ID NO: 50), wherein,

A=an adenine nucleobase,

mC=a 5-methyl cytosine nucleobase,

G=a guanine nucleobase,

T=a thymine nucleobase,

e=a 2′-MOE sugar moiety,

d=a 2′-β-D-deoxyribosyl sugar moiety,

s=a phosphorothioate internucleoside linkage, and

o=a phosphodiester internucleoside linkage.

In certain embodiments, Compound No. 1287095 is represented by the following chemical structure:

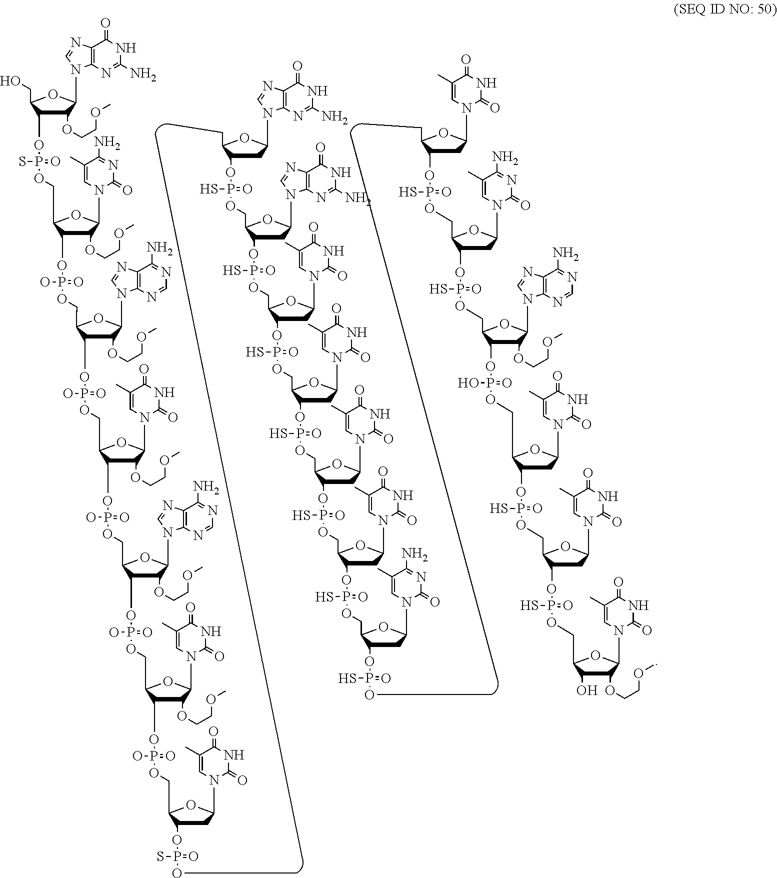

### Structure 5. Compound No. 1287095

In certain embodiments, the sodium salt of Compound No. 1287095 is represented by the following chemical structure:

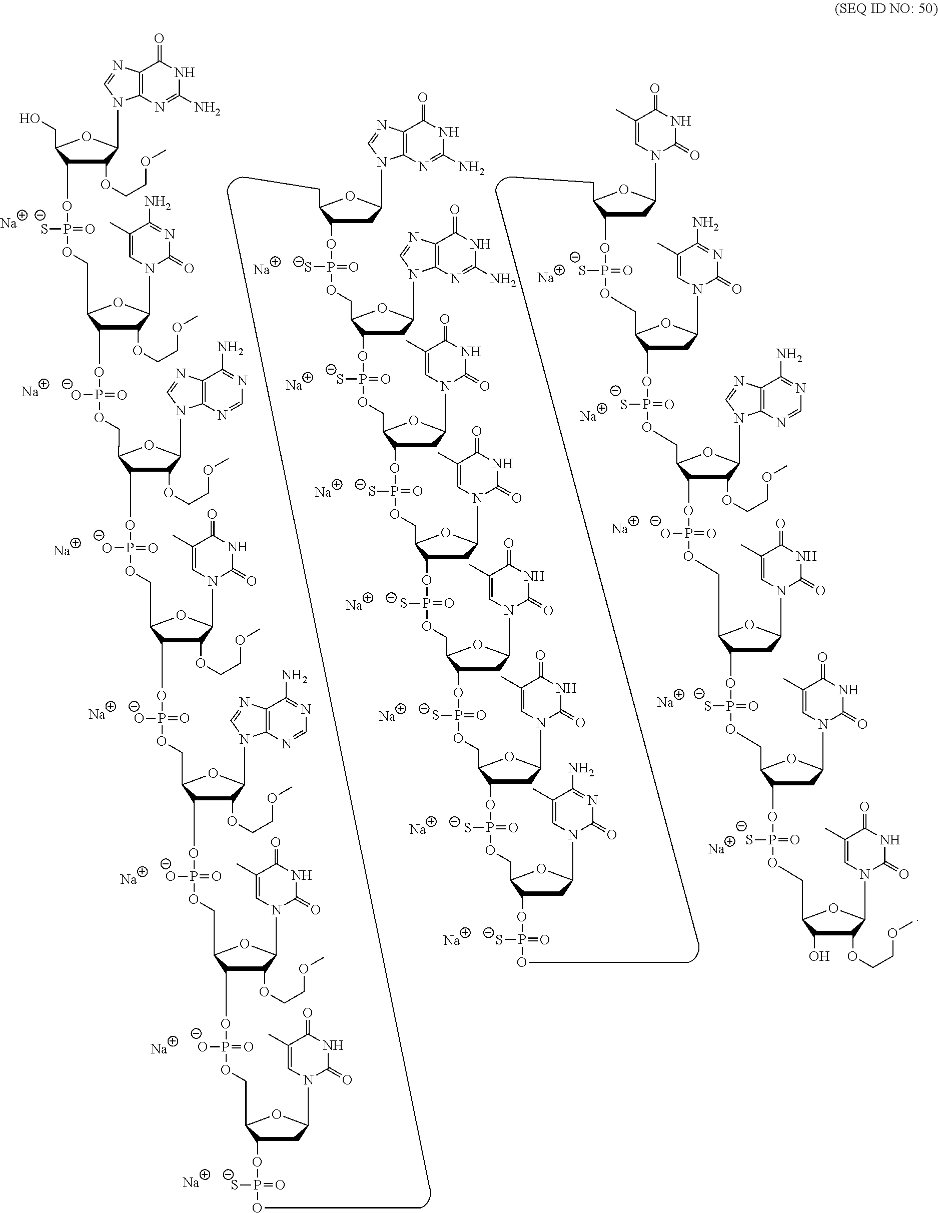

Structure 6. The sodium salt of Compound No. 1287095

### VIII. Certain Comparator Compositions

In certain embodiments, Compound No. 650528, which has been described in Moore, et al., Mol. Ther. Nucleic Acids, 2017, 7:200-210 (Moore, 2017) (”ASO-5″), WO 2018/089805, and McLoughlin et al., Ann. Neurol., 2018, 84:64-77 (McLoughlin, 2018) (each of which are incorporated herein by reference) was used as a comparator compound. Compound No. 650528 is a 5-8-5 MOE gapmer, having a sequence (from 5′ to 3′) GCATCTTTTCATACTGGC (SEQ ID NO: 10), wherein each cytosine is a 5-methylcytosine, each internucleoside linkage is either a phosphodiester internucleoside linkage or a phosphorothioate internucleoside linkage and the internucleoside linkage motif is sooosssssssssooss, wherein ‘s’ represents a phosphorothioate internucleoside linkage and ‘o’ represents a phosphodiester internucleoside linkage, and wherein each of nucleosides 1-5 and 14-18 comprise a 2′-MOE sugar moiety.

In certain embodiments, compounds described herein are superior relative to comparator Compound No. 650528, described in Moore, 2017, WO 2018/089805, and McLoughlin, 2018, because they demonstrate one or more improved properties, such as, potency and efficacy.

For example, as described herein, certain compounds, Compound No. 1269455, Compound No. 1287095, and Compound No. 1287621 are more potent than comparator Compound No. 650528 in vitro. See, e.g., Example 5, hereinbelow. For example, as described herein, certain compounds Compound No. 1269455, Compound No. 1287095, and Compound No. 1287621 achieved an ICso in Example 5, hereinbelow, of 0.09 μM, 0.02 μM, and 0.8 μM, respectively, whereas comparator Compound No. 650528 (“ASO-5”) achieved an IC₅₀ in Example 5, hereinbelow, of 2.03 μM. Therefore, certain compounds described herein are more potent than comparator Compound No. 650528 (“ASO-5”) in this assay.

For example, as described herein, certain compounds Compound No. 1269455, Compound No. 1287095, and Compound No. 1287621 are more efficacious than comparator Compound No. 650528 in vivo. See, e.g., Example 3, hereinbelow. For example, as provided in Table 10, Compound No. 1269455 achieved an average expression level (% control) of 18% in spinal cord, 20% in cortex, and 14% in brain stem of transgenic mice, whereas comparator Compound No. 650528 (“ASO-5”) achieved an average expression level (% control) of 38% in spinal cord, 39% in cortex, and 31% in brain stem of transgenic mice. For example, as provided in Table 11, certain compounds, Compound No. 1287095 and Compound No. 1287621, achieved an average expression level (% control) of 24% and 33%, respectively, in spinal cord of transgenic mice whereas comparator Compound No. 650528 (“ASO-5”) achieved an average expression level (% control) of 49% in spinal cord of transgenic mice. For example, as provided in Table 11, certain compounds, Compound No. 1287095 and Compound No. 1287621, achieved an average expression level (% control) of 17% and 27%, respectively, in cortex of transgenic mice whereas comparator Compound No. 650528 (“ASO-5”) achieved an average expression level (% control) of 49% in cortex of transgenic mice. For example, as provided in Table 11, certain compounds, Compound No. 1287095 and Compound No. 1287621, achieved an average expression level (% control) of 15% and 29%, respectively, in brain stem of transgenic mice whereas comparator Compound No. 650528 (“ASO-5”) achieved an average expression level (% control) of 45% in brain stem of transgenic mice. Therefore, certain compounds described herein are more efficacious than comparator Compound No. 650528 (“ASO-5”) in this assay.

### IX. Certain Hotspot Regions

1. Nucleobases 6,597-6,619 of SEQ ID NO: 2

In certain embodiments, nucleobases 6,597-6,619 of SEQ ID NO: 2 comprise a hotspot region. In certain embodiments, modified oligonucleotides are complementary to nucleobases 6,597-6,619 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers. In certain embodiments, the gapmers are 6-10-4 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sossssssssssssssoss, sooooossssssssssoss, or sooosssssssssssooss.

The nucleobase sequences of SEQ ID NOs: 61, 85, and 125 are complementary to nucleobases 6,597-6,619 of SEQ ID NO: 2.

In certain embodiments, modified oligonucleotides complementary to nucleobases 6,597-6,619 of SEQ ID NO: 2 achieve a minimum of 57% reduction of ATXN3 RNA in spinal cord tissue in the ATXN3 YAC transgenic mouse model. In certain embodiments, the modified oligonucleotides achieve an average of 70% reduction of ATXN3 RNA in spinal cord tissue. In certain embodiments, the modified oligonucleotides achieve a maximum of 78% reduction of ATXN3 RNA in spinal cord tissue. In certain embodiments, modified oligonucleotides complementary to nucleobases 6,597-6,619 of SEQ ID NO: 2 achieve a minimum of 48% reduction of ATXN3 RNA in cortex tissue in the ATXN3 YAC transgenic mouse model. In certain embodiments, the modified oligonucleotides achieve an average of 67% reduction of ATXN3 RNA in cortex tissue. In certain embodiments, the modified oligonucleotides achieve a maximum of 82% reduction of ATXN3 RNA in cortex tissue. In certain embodiments, modified oligonucleotides complementary to nucleobases 6,597-6,619 of SEQ ID NO: 2 achieve a minimum of 9% reduction of ATXN3 RNA in cerebellum tissue in the ATXN3 YAC transgenic mouse model. In certain embodiments, the modified oligonucleotides achieve an average of 51% reduction of ATXN3 RNA in cerebellum tissue. In certain embodiments, the modified oligonucleotides achieve a maximum of 78% reduction of ATXN3 RNA in cerebellum tissue.

In certain embodiments, modified oligonucleotides complementary to nucleobases 6,597-6,619 of SEQ ID NO: 2 achieve a minimum of 53% reduction of ATXN3 RNA in brain stem tissue in the ATXN3 YAC transgenic mouse model. In certain embodiments, the modified oligonucleotides achieve an average of 69% reduction of ATXN3 RNA in brain stem tissue. In certain embodiments, the modified oligonucleotides achieve a maximum of 77% reduction of ATXN3 RNA in brain stem tissue.

2. Nucleobases 15,664-15,689 of SEQ ID NO: 2

In certain embodiments, nucleobases 15,664-15,689 of SEQ ID NO: 2 comprise a hotspot region. In certain embodiments, modified oligonucleotides are complementary to nucleobases 15,664-15,689 of SEQ ID NO: 2. In certain embodiments, modified oligonucleotides are 20 nucleobases in length. In certain embodiments, modified oligonucleotides are gapmers. In certain embodiments, modified oligonucleotides are altered gapmers.

In certain embodiments, the gapmers are 5-10-5 MOE gapmers. In certain embodiments, the gapmers are 6-10-4 MOE gapmers. In certain embodiments, the altered gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′). In certain embodiments, the 2′-substituted nucleoside is at position 5 of the gap (5′ to 3′). In certain embodiments, the altered gapmers have the sugar motif in order from 5′ to 3′: eeeeedyddddddddeeeee or eeeeeddddydddddeeeee, wherein each “e” is nucleoside comprising a 2′-MOE sugar moiety, each “y” is a nucleoside comprising a 2′-OMe sugar moiety, and each “d” is a nucleoside comprising a 2′-β-D-deoxyribosyl sugar moiety.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss or sooooossssssssssoss.

The nucleobase sequences of SEQ ID NOs: 68, 69, 70, 71, 72, 122, and 139 are complementary to nucleobases 15,664-15,689 of SEQ ID NO: 2.

In certain embodiments, modified oligonucleotides complementary to nucleobases 15,664-15,689 of SEQ ID NO: 2 achieve a minimum of 56% reduction of ATXN3 RNA in spinal cord tissue in the ATXN3 YAC transgenic mouse model. In certain embodiments, the modified oligonucleotides achieve an average of 70% reduction of ATXN3 RNA in spinal cord tissue. In certain embodiments, the modified oligonucleotides achieve a maximum of 82% reduction of ATXN3 RNA in spinal cord tissue. In certain embodiments, modified oligonucleotides complementary to nucleobases 15,664-15,689 of SEQ ID NO: 2 achieve a minimum of 29% reduction of ATXN3 RNA in cortex tissue in the ATXN3 YAC transgenic mouse model. In certain embodiments, the modified oligonucleotides achieve an average of 62% reduction of ATXN3 RNA in cortex tissue. In certain embodiments, the modified oligonucleotides achieve a maximum of 86% reduction of ATXN3 RNA in cortex tissue.

In certain embodiments, modified oligonucleotides complementary to nucleobases 15,664-15,689 of SEQ ID NO: 2 achieve a minimum of 13% reduction of ATXN3 RNA in cerebellum tissue in the ATXN3 YAC transgenic mouse model. In certain embodiments, the modified oligonucleotides achieve an average of 36% reduction of ATXN3 RNA in cerebellum tissue. In certain embodiments, the modified oligonucleotides achieve a maximum of 73% reduction of ATXN3 RNA in cerebellum tissue.

In certain embodiments, modified oligonucleotides complementary to nucleobases 15,664-15,689 of SEQ ID NO: 2 achieve a minimum of 43% reduction of ATXN3 RNA in brain stem tissue in the ATXN3 YAC transgenic mouse model. In certain embodiments, the modified oligonucleotides achieve an average of 65% reduction of ATXN3 RNA in brain stem tissue. In certain embodiments, the modified oligonucleotides achieve a maximum of 86% reduction of ATXN3 RNA in brain stem tissue.

3. Nucleobases 19,451-19,476 of SEQ ID NO: 2

In certain embodiments, nucleobases 19,451-19,476 of SEQ ID NO: 2 comprise a hotspot region. In certain embodiments, modified oligonucleotides are complementary to nucleobases 19,451-19,476 of SEQ ID NO: 2. In certain embodiments, modified oligonucleotides are 20 nucleobases in length. In certain embodiments, modified oligonucleotides are gapmers. In certain embodiments, modified oligonucleotides are altered gapmers.

In certain embodiments, the gapmers are 5-10-5 MOE gapmers. In certain embodiments, the gapmers are 6-10-4 MOE gapmers. In certain embodiments, the altered gapmers comprise a 2′-substituted nucleoside in the gap. In certain embodiments, the 2′-substituted nucleoside comprises a 2′-OMe sugar moiety. In certain embodiments, the 2′-substituted nucleoside is at position 2 of the gap (5′ to 3′). In certain embodiments, the 2′-substituted nucleoside is at position 4 of the gap (5′ to 3′). In certain embodiments, the altered gapmers have the sugar motif in order from 5′ to 3′: eeeeedyddddddddeeeee or eeeeedddyddddddeeeee, wherein each “e” is nucleoside comprising a 2′-MOE sugar moiety, each “y” is a nucleoside comprising a 2′-OMe sugar moiety, and each “d” is a nucleoside comprising a 2′-β-D-deoxyribosyl sugar moiety.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss, sooooossssssssssoss, or sossssssssssssssoss.

The nucleobase sequences of SEQ ID NOs: 59, 62, 66, 75, 76, 138, and 140 are complementary to nucleobases 19,451-19,476 of SEQ ID NO: 2.

In certain embodiments, modified oligonucleotides complementary to nucleobases 19,451-19,476 of SEQ ID NO: 2 achieve a minimum of 42% reduction of ATXN3 RNA in spinal cord tissue in the ATXN3 YAC transgenic mouse model. In certain embodiments, the modified oligonucleotides achieve an average of 66% reduction of ATXN3 RNA in spinal cord tissue. In certain embodiments, the modified oligonucleotides achieve a maximum of 81% reduction of ATXN3 RNA in spinal cord tissue.

In certain embodiments, modified oligonucleotides complementary to nucleobases 19,451-19,476 of SEQ ID NO: 2 achieve a minimum of 50% reduction of ATXN3 RNA in cortex tissue in the ATXN3 YAC transgenic mouse model. In certain embodiments, the modified oligonucleotides achieve an average of 66% reduction of ATXN3 RNA in cortex tissue. In certain embodiments, the modified oligonucleotides achieve a maximum of 86% reduction of ATXN3 RNA in cortex tissue.

In certain embodiments, modified oligonucleotides complementary to nucleobases 19,451-19,476 of SEQ ID NO: 2 achieve a minimum of 18% reduction of ATXN3 RNA in cerebellum tissue in the ATXN3 YAC transgenic mouse model. In certain embodiments, the modified oligonucleotides achieve an average of 38% reduction of ATXN3 RNA in cerebellum tissue. In certain embodiments, the modified oligonucleotides achieve a maximum of 53% reduction of ATXN3 RNA in cerebellum tissue.

In certain embodiments, modified oligonucleotides complementary to nucleobases 19,451-19,476 of SEQ ID NO: 2 achieve a minimum of 29% reduction of ATXN3 RNA in brain stem tissue in the ATXN3 YAC transgenic mouse model. In certain embodiments, the modified oligonucleotides achieve an average of 64% reduction of ATXN3 RNA in brain stem tissue. In certain embodiments, the modified oligonucleotides achieve a maximum of 80% reduction of ATXN3 RNA in brain stem tissue.

4. Nucleobases 30,448-30,473 of SEQ ID NO: 2

In certain embodiments, nucleobases 30,448-30,473 of SEQ ID NO: 2 comprise a hotspot region. In certain embodiments, modified oligonucleotides are complementary to nucleobases 30,448-30,473 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss or sossssssssssssssoss.

The nucleobase sequences of SEQ ID NOs: 65, 116, 117, 118, 119, and 120 are complementary to nucleobases 30,448-30,473 of SEQ ID NO: 2.

In certain embodiments, modified oligonucleotides complementary to nucleobases 30,448-30,473 of SEQ ID NO: 2 achieve a minimum of 57% reduction of ATXN3 RNA in spinal cord tissue in the ATXN3 YAC transgenic mouse model. In certain embodiments, the modified oligonucleotides achieve an average of 70% reduction of ATXN3 RNA in spinal cord tissue. In certain embodiments, the modified oligonucleotides achieve a maximum of 83% reduction of ATXN3 RNA in spinal cord tissue.

In certain embodiments, modified oligonucleotides complementary to nucleobases 30,448-30,473 of SEQ ID NO: 2 achieve a minimum of 52% reduction of ATXN3 RNA in cortex tissue in the ATXN3 YAC transgenic mouse model. In certain embodiments, the modified oligonucleotides achieve an average of 71% reduction of ATXN3 RNA in cortex tissue. In certain embodiments, the modified oligonucleotides achieve a maximum of 85% reduction of ATXN3 RNA in cortex tissue.

In certain embodiments, modified oligonucleotides complementary to nucleobases 30,448-30,473 of SEQ ID NO: 2 achieve a minimum of 23% reduction of ATXN3 RNA in cerebellum tissue in the ATXN3 YAC transgenic mouse model. In certain embodiments, the modified oligonucleotides achieve an average of 33% reduction of ATXN3 RNA in cerebellum tissue. In certain embodiments, the modified oligonucleotides achieve a maximum of 45% reduction of ATXN3 RNA in cerebellum tissue.

In certain embodiments, modified oligonucleotides complementary to nucleobases 30,448-30,473 of SEQ ID NO: 2 achieve a minimum of 65% reduction of ATXN3 RNA in brain stem tissue in the ATXN3 YAC transgenic mouse model. In certain embodiments, the modified oligonucleotides achieve an average of 75% reduction of ATXN3 RNA in brain stem tissue. In certain embodiments, the modified oligonucleotides achieve a maximum of 86% reduction of ATXN3 RNA in brain stem tissue.

5. Nucleobases 32,940-32,961 of SEQ ID NO: 2

In certain embodiments, nucleobases 32,940-32,961 of SEQ ID NO: 2 comprise a hotspot region. In certain embodiments, modified oligonucleotides are complementary to nucleobases 32,940-32,961 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers. In certain embodiments, the gapmers are 6-10-4 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooooossssssssssoss or sooosssssssssssooss.

The nucleobase sequences of SEQ ID NOs: 38, 46, and 123 are complementary to nucleobases 32,940-32,961 of SEQ ID NO: 2.

In certain embodiments, modified oligonucleotides complementary to nucleobases 32,940-32,961 of SEQ ID NO: 2 achieve a minimum of 67% reduction of ATXN3 RNA in spinal cord tissue in the ATXN3 YAC transgenic mouse model. In certain embodiments, the modified oligonucleotides achieve an average of 73% reduction of ATXN3 RNA in spinal cord tissue. In certain embodiments, the modified oligonucleotides achieve a maximum of 77% reduction of ATXN3 RNA in spinal cord tissue.

In certain embodiments, modified oligonucleotides complementary to nucleobases 32,940-32,961 of SEQ ID NO: 2 achieve a minimum of 68% reduction of ATXN3 RNA in cortex tissue in the ATXN3 YAC transgenic mouse model. In certain embodiments, the modified oligonucleotides achieve an average of 76% reduction of ATXN3 RNA in cortex tissue. In certain embodiments, the modified oligonucleotides achieve a maximum of 86% reduction of ATXN3 RNA in cortex tissue.

In certain embodiments, modified oligonucleotides complementary to nucleobases 32,940-32,961 of SEQ ID NO: 2 achieve a minimum of 27% reduction of ATXN3 RNA in cerebellum tissue in the ATXN3 YAC transgenic mouse model. In certain embodiments, the modified oligonucleotides achieve an average of 49% reduction of ATXN3 RNA in cerebellum tissue. In certain embodiments, the modified oligonucleotides achieve a maximum of 72% reduction of ATXN3 RNA in cerebellum tissue.

In certain embodiments, modified oligonucleotides complementary to nucleobases 32,940-32,961 of SEQ ID NO: 2 achieve a minimum of 65% reduction of ATXN3 RNA in brain stem tissue in the ATXN3 YAC transgenic mouse model. In certain embodiments, the modified oligonucleotides achieve an average of 74% reduction of ATXN3 RNA in brain stem tissue. In certain embodiments, the modified oligonucleotides achieve a maximum of 79% reduction of ATXN3 RNA in bmin stem tissue.

6. Nucleobases 34,013-34,039 of SEQ ID NO: 2

In certain embodiments, nucleobases 34,013-34,039 of SEQ ID NO: 2 comprise a hotspot region. In certain embodiments, modified oligonucleotides are complementary to nucleobases 34,013-34,039 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers. In certain embodiments, the gapmers are 6-10-4 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss or sooooossssssssssoss.

The nucleobase sequences of SEQ ID NOs: 103, 104, 105, 106, 107, 108, and 124 are complementary to nucleobases 34,013-34,039 of SEQ ID NO: 2.

In certain embodiments, modified oligonucleotides complementary to nucleobases 34,013-34,039 of SEQ ID NO: 2 achieve a minimum of 39% reduction of ATXN3 RNA in spinal cord tissue in the ATXN3 YAC transgenic mouse model. In certain embodiments, the modified oligonucleotides achieve an average of 52% reduction of ATXN3 RNA in spinal cord tissue. In certain embodiments, the modified oligonucleotides achieve a maximum of 70% reduction of ATXN3 RNA in spinal cord tissue.

In certain embodiments, modified oligonucleotides complementary to nucleobases 34,013-34,039 of SEQ ID NO: 2 achieve a minimum of 54% reduction of ATXN3 RNA in cortex tissue in the ATXN3 YAC transgenic mouse model. In certain embodiments, the modified oligonucleotides achieve an average of 62% reduction of ATXN3 RNA in cortex tissue. In certain embodiments, the modified oligonucleotides achieve a maximum of 72% reduction of ATXN3 RNA in cortex tissue.

In certain embodiments, modified oligonucleotides complementary to nucleobases 34,013-34,039 of SEQ ID NO: 2 achieve a minimum of 34% reduction of ATXN3 RNA in cerebellum tissue in the ATXN3 YAC transgenic mouse model. In certain embodiments, the modified oligonucleotides achieve an average of 45% reduction of ATXN3 RNA in cerebellum tissue. In certain embodiments, the modified oligonucleotides achieve a maximum of 67% reduction of ATXN3 RNA in cerebellum tissue.

In certain embodiments, modified oligonucleotides complementary to nucleobases 34,013-34,039 of SEQ ID NO: 2 achieve a minimum of 46% reduction of ATXN3 RNA in brain stem tissue in the ATXN3 YAC transgenic mouse model. In certain embodiments, the modified oligonucleotides achieve an average of 54% reduction of ATXN3 RNA in brain stem tissue. In certain embodiments, the modified oligonucleotides achieve a maximum of 64% reduction of ATXN3 RNA in bmin stem tissue.

7. Nucleobases 37,151-37,172 of SEQ ID NO: 2

In certain embodiments, nucleobases 37,151-37,172 of SEQ ID NO: 2 comprise a hotspot region. In certain embodiments, modified oligonucleotides are complementary to nucleobases 37,151-37,172 of SEQ ID NO: 2. In certain embodiments, modified oligonucleotides are 20 nucleobases in length. In certain embodiments, modified oligonucleotides are gapmers. In certain embodiments, modified oligonucleotides are altered gapmers.

In certain embodiments, the gapmers are 5-10-5 MOE gapmers. In certain embodiments, the gapmers are 6-10-4 MOE gapmers. In certain embodiments, the altered gapmers comprise a 2′-substituted nucleoside in the gap. In certain embodiments, the 2′-substituted nucleoside comprises a 2′-OMe sugar moiety. In certain embodiments, the 2′-substituted nucleoside is at position 1 of the gap (5′ to 3′). In certain embodiments, the 2′-substituted nucleoside is at position 2 of the gap (5′ to 3′). In certain embodiments, the altered gapmers have the sugar motif in order from 5′ to 3′: eeeeeydddddddddeeeee or eeeeedyddddddddeeeee, wherein each “e” is nucleoside comprising a 2′-MOE sugar moiety, each “y” is a nucleoside comprising a 2′-OMe sugar moiety, and each “d” is a nucleoside comprising a 2′-β-D-deoxyribosyl sugar moiety.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss, sooooossssssssssoss, or sossssssssssssssoss.

The nucleobase sequences of SEQ ID NOs: 17, 44, and 60 are complementary to nucleobases 37,151-37,172 of SEQ ID NO: 2.

In certain embodiments, modified oligonucleotides complementary to nucleobases 37,151-37,172 of SEQ ID NO: 2 achieve a minimum of 54% reduction of ATXN3 RNA in spinal cord tissue in the ATXN3 YAC transgenic mouse model. In certain embodiments, the modified oligonucleotides achieve an average of 71% reduction of ATXN3 RNA in spinal cord tissue. In certain embodiments, the modified oligonucleotides achieve a maximum of 81% reduction of ATXN3 RNA in spinal cord tissue.

In certain embodiments, modified oligonucleotides complementary to nucleobases 37,151-37,172 of SEQ ID NO: 2 achieve a minimum of 50% reduction of ATXN3 RNA in cortex tissue in the ATXN3 YAC transgenic mouse model. In certain embodiments, the modified oligonucleotides achieve an average of 68% reduction of ATXN3 RNA in cortex tissue. In certain embodiments, the modified oligonucleotides achieve a maximum of 76% reduction of ATXN3 RNA in cortex tissue.

In certain embodiments, modified oligonucleotides complementary to nucleobases 37,151-37,172 of SEQ ID NO: 2 achieve a minimum of 18% reduction of ATXN3 RNA in cerebellum tissue in the ATXN3 YAC transgenic mouse model. In certain embodiments, the modified oligonucleotides achieve an average of 42% reduction of ATXN3 RNA in cerebellum tissue. In certain embodiments, the modified oligonucleotides achieve a maximum of 69% reduction of ATXN3 RNA in cerebellum tissue.

In certain embodiments, modified oligonucleotides complementary to nucleobases 37,151-37,172 of SEQ ID NO: 2 achieve a minimum of 53% reduction of ATXN3 RNA in brain stem tissue in the ATXN3 YAC transgenic mouse model. In certain embodiments, the modified oligonucleotides achieve an average of 71% reduction of ATXN3 RNA in brain stem tissue. In certain embodiments, the modified oligonucleotides achieve a maximum of 83% reduction of ATXN3 RNA in brain stem tissue.

8. Nucleobases 43,647-43,674 of SEQ ID NO: 2

In certain embodiments, nucleobases 43,647-43,674 of SEQ ID NO: 2 comprise a hotspot region. In certain embodiments, modified oligonucleotides are complementary to nucleobases 43,647-43,674 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss.

The nucleobase sequences of SEQ ID NOs: 131, 132, 133, 134, and 135 are complementary to nucleobases 43,647-43,674 of SEQ ID NO: 2.

In certain embodiments, modified oligonucleotides complementary to nucleobases 43,647-43,674 of SEQ ID NO: 2 achieve a minimum of 28% reduction of ATXN3 RNA in spinal cord tissue in the ATXN3 YAC transgenic mouse model. In certain embodiments, the modified oligonucleotides achieve an average of 39% reduction of ATXN3 RNA in spinal cord tissue. In certain embodiments, the modified oligonucleotides achieve a maximum of 54% reduction of ATXN3 RNA in spinal cord tissue.

In certain embodiments, modified oligonucleotides complementary to nucleobases 43,647-43,674 of SEQ ID NO: 2 achieve a minimum of 44% reduction of ATXN3 RNA in cortex tissue in the ATXN3 YAC transgenic mouse model. In certain embodiments, the modified oligonucleotides achieve an average of 55% reduction of ATXN3 RNA in cortex tissue. In certain embodiments, the modified oligonucleotides achieve a maximum of 74% reduction of ATXN3 RNA in cortex tissue.

In certain embodiments, modified oligonucleotides complementary to nucleobases 43,647-43,674 of SEQ ID NO: 2 achieve a minimum of 39% reduction of ATXN3 RNA in cerebellum tissue in the ATXN3 YAC transgenic mouse model. In certain embodiments, the modified oligonucleotides achieve an average of 60% reduction of ATXN3 RNA in cerebellum tissue. In certain embodiments, the modified oligonucleotides achieve a maximum of 72% reduction of ATXN3 RNA in cerebellum tissue.

In certain embodiments, modified oligonucleotides complementary to nucleobases 43,647-43,674 of SEQ ID NO: 2 achieve a minimum of 61% reduction of ATXN3 RNA in brain stem tissue in the ATXN3 YAC transgenic mouse model. In certain embodiments, the modified oligonucleotides achieve an average of 66% reduction of ATXN3 RNA in brain stem tissue. In certain embodiments, the modified oligonucleotides achieve a maximum of 79% reduction of ATXN3 RNA in brain stem tissue.

9. Nucleobases 46,389-46,411 of SEQ ID NO: 2

In certain embodiments, nucleobases 46,389-46,411 of SEQ ID NO: 2 comprise a hotspot region. In certain embodiments, modified oligonucleotides are complementary to nucleobases 46,389-46,411 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers. In certain embodiments, the gapmers are 6-10-4 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sossssssssssssssoss, sooooossssssssssoss, or sooosssssssssssooss.

The nucleobase sequences of SEQ ID NOs: 32, 58, 127, and 128 are complementary to nucleobases 46,389-46,411 of SEQ ID NO: 2.

In certain embodiments, modified oligonucleotides complementary to nucleobases 46,389-46,411 of SEQ ID NO: 2 achieve a minimum of 47% reduction of ATXN3 RNA in spinal cord tissue in the ATXN3 YAC transgenic mouse model. In certain embodiments, the modified oligonucleotides achieve an average of 72% reduction of ATXN3 RNA in spinal cord tissue. In certain embodiments, the modified oligonucleotides achieve a maximum of 84% reduction of ATXN3 RNA in spinal cord tissue.

In certain embodiments, modified oligonucleotides complementary to nucleobases 46,389-46,411 of SEQ ID NO: 2 achieve a minimum of 39% reduction of ATXN3 RNA in cortex tissue in the ATXN3 YAC transgenic mouse model. In certain embodiments, the modified oligonucleotides achieve an average of 73% reduction of ATXN3 RNA in cortex tissue. In certain embodiments, the modified oligonucleotides achieve a maximum of 89% reduction of ATXN3 RNA in cortex tissue.

In certain embodiments, modified oligonucleotides complementary to nucleobases 46,389-46,411 of SEQ ID NO: 2 achieve a minimum of 36% reduction of ATXN3 RNA in cerebellum tissue in the ATXN3 YAC transgenic mouse model. In certain embodiments, the modified oligonucleotides achieve an average of 61% reduction of ATXN3 RNA in cerebellum tissue. In certain embodiments, the modified oligonucleotides achieve a maximum of 78% reduction of ATXN3 RNA in cerebellum tissue.

In certain embodiments, modified oligonucleotides complementary to nucleobases 46,389-46,411 of SEQ ID NO: 2 achieve a minimum of 44% reduction of ATXN3 RNA in brain stem tissue in the ATXN3 YAC transgenic mouse model. In certain embodiments, the modified oligonucleotides achieve an average of 72% reduction of ATXN3 RNA in brain stem tissue. In certain embodiments, the modified oligonucleotides achieve a maximum of 85% reduction of ATXN3 RNA in brain stem tissue.

10. Nucleobases 46,748-46,785 of SEQ ID NO: 2

In certain embodiments, nucleobases 46,748-46,785 of SEQ ID NO: 2 comprise a hotspot region. In certain embodiments, modified oligonucleotides are complementary to nucleobases 46,748-46,785 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooosssssssssssooss.

The nucleobase sequences of SEQ ID NOs: 94, 95, 96, 97, 98, 99, 100, and 101 are complementary to nucleobases 46,748-46,785 of SEQ ID NO: 2.

In certain embodiments, modified oligonucleotides complementary to nucleobases 46,748-46,785 of SEQ ID NO: 2 achieve a minimum of 36% reduction of ATXN3 RNA in spinal cord tissue in the ATXN3 YAC transgenic mouse model. In certain embodiments, the modified oligonucleotides achieve an average of 51% reduction of ATXN3 RNA in spinal cord tissue. In certain embodiments, the modified oligonucleotides achieve a maximum of 62% reduction of ATXN3 RNA in spinal cord tissue.

In certain embodiments, modified oligonucleotides complementary to nucleobases 46,748-46,785 of SEQ ID NO: 2 achieve a minimum of 41% reduction of ATXN3 RNA in cortex tissue in the ATXN3 YAC transgenic mouse model. In certain embodiments, the modified oligonucleotides achieve an average of 58% reduction of ATXN3 RNA in cortex tissue. In certain embodiments, the modified oligonucleotides achieve a maximum of 72% reduction of ATXN3 RNA in cortex tissue.

In certain embodiments, modified oligonucleotides complementary to nucleobases 46,748-46,785 of SEQ ID NO: 2 achieve a minimum of 23% reduction of ATXN3 RNA in cerebellum tissue in the ATXN3 YAC transgenic mouse model. In certain embodiments, the modified oligonucleotides achieve an average of 36% reduction of ATXN3 RNA in cerebellum tissue. In certain embodiments, the modified oligonucleotides achieve a maximum of 50% reduction of ATXN3 RNA in cerebellum tissue.

In certain embodiments, modified oligonucleotides complementary to nucleobases 46,748-46,785 of SEQ ID NO: 2 achieve a minimum of 30% reduction of ATXN3 RNA in brain stem tissue in the ATXN3 YAC transgenic mouse model. In certain embodiments, the modified oligonucleotides achieve an average of 47% reduction of ATXN3 RNA in brain stem tissue. In certain embodiments, the modified oligonucleotides achieve a maximum of 57% reduction of ATXN3 RNA in brain stem tissue.

11. Nucleobases 47,594-47,619 of SEQ ID NO: 2

In certain embodiments, nucleobases 47,594-47,619 of SEQ ID NO: 2 comprise a hotspot region. In certain embodiments, modified oligonucleotides are complementary to nucleobases 47,594-47,619 of SEQ ID NO: 2. In certain embodiments, modified oligonucleotides are 20 nucleobases in length. In certain embodiments, modified oligonucleotides are gapmers.

In certain embodiments, the gapmers are 5-10-5 MOE gapmers. In certain embodiments, the gapmers are 6-10-4 MOE gapmers.

In certain embodiments, the internucleoside linkages of the modified oligonucleotides are phosphodiester (“o”) and phosphorothioate (“s”) internucleoside linkages. In certain embodiments, the phosphodiester (“o”) and phosphorothioate (“s”) internucleoside linkages are arranged in order from 5′ to 3′: sooooossssssssssoss, soooossssssssssooos, soooossssssssssooss, sooosssssssssssooos, or sooosssssssssssooss.

The nucleobase sequences of SEQ ID NOs: 29 and 50 are complementary to nucleobases 47,594-47,619 of SEQ ID NO: 2.

In certain embodiments, modified oligonucleotides complementary to nucleobases 47,594-47,619 of SEQ ID NO: 2 achieve a minimum of 71% reduction of ATXN3 RNA in spinal cord tissue in the ATXN3 YAC transgenic mouse model. In certain embodiments, the modified oligonucleotides achieve an average of 74% reduction of ATXN3 RNA in spinal cord tissue. In certain embodiments, the modified oligonucleotides achieve a maximum of 79% reduction of ATXN3 RNA in spinal cord tissue.

In certain embodiments, modified oligonucleotides complementary to nucleobases 47,594-47,619 of SEQ ID NO: 2 achieve a minimum of 64% reduction of ATXN3 RNA in cortex tissue in the ATXN3 YAC transgenic mouse model. In certain embodiments, the modified oligonucleotides achieve an average of 71% reduction of ATXN3 RNA in cortex tissue. In certain embodiments, the modified oligonucleotides achieve a maximum of 87% reduction of ATXN3 RNA in cortex tissue.

In certain embodiments, modified oligonucleotides complementary to nucleobases 47,594-47,619 of SEQ ID NO: 2 achieve a minimum of 42% reduction of ATXN3 RNA in cerebellum tissue in the ATXN3 YAC transgenic mouse model. In certain embodiments, the modified oligonucleotides achieve an average of 52% reduction of ATXN3 RNA in cerebellum tissue. In certain embodiments, the modified oligonucleotides achieve a maximum of 81% reduction of ATXN3 RNA in cerebellum tissue.

In certain embodiments, modified oligonucleotides complementary to nucleobases 47,594-47,619 of SEQ ID NO: 2 achieve a minimum of 71% reduction of ATXN3 RNA in brain stem tissue in the ATXN3 YAC transgenic mouse model. In certain embodiments, the modified oligonucleotides achieve an average of 74% reduction of ATXN3 RNA in brain stem tissue. In certain embodiments, the modified oligonucleotides achieve a maximum of 82% reduction of ATXN3 RNA in brain stem tissue.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1

### Design of Gapmers with Mixed PO/PS Internucleoside Linkages Complementary to Human ATXN3 RNA

Modified oligonucleotides complementary to a human ATXN3 nucleic acid were designed. The modified oligonucleotides in the table below are 5-10-5 MOE gapmers, 6-10-4 MOE gapmers, or 5-9-5 MOE gapmers. The gapmers have a central gap segment that comprises 2′-deoxynucleosides and is flanked by wing segments on both the 5′ end and on the 3′ end comprising 2′-MOE nucleosides. The internucleoside linkages throughout each gapmer are mixed phosphodiester internucleoside linkages and phosphorothioate internucleoside linkages. Internucleoside linkage motifs include, in order from 5′ to 3′: sooooossssssssssoss, soooo ssssssssssooos, soooossssssssssooss, sooo sssssssssooss, sooossssssssssooss, sooosssssssssssooos, sooosssssssssssooss, sossssssssssssssoss, and ssoosssssssssssooss. Each cytosine residue is a 5-methyl cytosine. The sequence and chemical notation column specifies the sequence, including 5-methyl cytosines, sugar chemistry, and the internucleoside linkage chemistry; wherein subscript ‘d’ represents a 2′-β-D-deoxyribosyl sugar moiety, subscript ‘e’ represents a 2′-MOE sugar moiety, subscript ‘o’ represents a phosphodiester internucleoside linkage, subscript ‘s’ refers represents to a phosphorothioate internucleoside linkage, and superscript ‘m’ before the cytosine residue represents a 5-methyl cytosine. “Start site” indicates the 5′-most nucleoside to which the gapmer is complementary in the human nucleic acid sequence. “Stop site” indicates the 3′-most nucleoside to which the gapmer is complementary in the human nucleic acid sequence.

Each modified oligonucleotide listed in the table below is complementary to human ATXN3 nucleic acid sequence SEQ ID NO: 1 (GENBANK Accession No: NM_004993.5), SEQ ID NO: 2 (the complement of GENBANK Accession No NC_000014.9 truncated from nucleotides 92,056,001 to 92,110,000), or SEQ ID NO: 3 (the complement of GENBANK Accession No NC 000014.9 truncated from nucleotides 92038001 to 92110000), as indicated. ‘N/A’ indicates that the modified oligonucleotide is not 100% complementary to that particular nucleic acid.

TABLE 1
MOE gapmers with mixed PO/PS internucleoside linkages complementary to human ATXN3

| | | | SEQ | SEQ | SEQ | SEQ | | |
|---|---|---|---|---|---|---|---|---|
| | | | ID | ID | ID | ID | | |
| | | | NO: 1 | NO: 1 | NO: 2 | NO: 2 | | SEQ |
| Compound | Sequence | Gapmer | Start | Stop | Start | Stop | Chemistry Notation | ID |
| Number | (5′ to 3′) | Motif | Site | Site | Site | Site | (5′ to 3′) | NO |
| 1248258 | ATAGAATGGC | 5-10-5 | N/A | N/A | 37159 | 37178 | AₑₛTₑₒAₑₒGₑₒAₑₛA_{ds}T_{ds}G_{ds}G_{ds}^{m}C_{ds}A_{ds}^{m} | 11 |
| | ACATTTTTTA | MOE | | | | | C_{ds}A_{ds}T_{ds}T_{ds}TₑₒTₑₒTₑₛTₑₛAₑ | |
| 1248259 | AACCCAATAA | 5-10-5 | N/A | N/A | 32927 | 32946 | AₑₛAₑₒ^{m}Cₑₒ^{m}Cₑₒ^{m}CₑₛA_{ds}A_{ds}T_{ds}A_{ds}A_{ds}T_{ds}^{m} | 12 |
| | TCTGACATCC | MOE | | | | | C_{ds}T_{ds}G_{ds}A_{ds}^{m}CₑₒAₑₒTₑₛ^{m}Cₑₛ^{m}Cₑ | |
| 1248261 | AATAATCTGA | 5-10-5 | N/A | N/A | 32922 | 32941 | AₑₛAₑₒTₑₒAₑₒAₑₛT_{ds}^{m}C_{ds}T_{ds}G_{ds}A_{ds}^{m}C_{ds} | 13 |
| | CATCCTCAGA | MOE | | | | | A_{ds}T_{ds}^{m}C_{ds}^{m}C_{ds}Tₑₒ^{m}CₑₒAₑₛGₑₛAₑ | |
| 1248262 | AATAGAATGG | 5-10-5 | N/A | N/A | 37160 | 37179 | AₑₛAₑₒTₑₒAₑₒGₑₛA_{ds}A_{ds}T_{ds}G_{ds}G_{ds}^{m}C_{ds} | 14 |
| | CACATTTTTT | MOE | | | | | A_{ds}^{m}C_{ds}A_{ds}T_{ds}TₑₒTₑₒTₑₛTₑₛTₑ | |
| 1248264 | TTTTATAGAGT | 5-10-5 | N/A | N/A | 37144 | 37163 | TₑₛTₑₒTₑₒTₑₒAₑₛT_{ds}A_{ds}G_{ds}A_{ds}G_{ds}T_{ds}T_{ds}^{m} | 15 |
| | TCCTCTCAA | MOE | | | | | C_{ds}^{m}C_{ds}T_{ds}^{m}CₑₒTₑₒ^{m}CₑₛAₑₛAₑ | |
| 1248265 | TTTAACCCAAT | 5-10-5 | N/A | N/A | 32930 | 32949 | TₑₛTₑₒTₑₒAₑₒAₑₛ^{m}C_{ds}^{m}C_{ds}^{m}C_{ds}A_{ds}A_{ds}T_{ds} | 16 |
| | AATCTGACA | MOE | | | | | A_{ds}A_{ds}T_{ds}^{m}C_{ds}TₑₒGₑₒAₑₛ^{m}CₑₛAₑ | |
| 1248266 | GCACATTTTTT | 5-10-5 | N/A | N/A | 37151 | 37170 | Gₑₛ^{m}CₑₒAₑₒ^{m}CₑₒAₑₛT_{ds}T_{ds}T_{ds}T_{ds}T_{ds}T_{ds} | 17 |
| | ATAGAGTTC | MOE | | | | | A_{ds}T_{ds}A_{ds}G_{ds}AₑₒGₑₒTₑₛTₑₛ^{m}Cₑ | |
| 1248267 | AGAATGGCAC | 5-10-5 | N/A | N/A | 37157 | 37176 | AₑₛGₑₒAₑₒAₑₒTₑₛG_{ds}G_{ds}^{m}C_{ds}A_{ds}^{m}C_{ds}A_{ds} | 18 |
| | ATTTTTTATA | MOE | | | | | T_{ds}T_{ds}T_{ds}T_{ds}TₑₒTₑₒAₑₛTₑₛAₑ | |
| 1248268 | TTTTTTATAGA | 5-10-5 | N/A | N/A | 37146 | 37165 | TₑₛTₑₒTₑₒTₑₒTₑₛT_{ds}A_{ds}T_{ds}A_{ds}G_{ds}A_{ds}G_{ds} | 19 |
| | GTTCCTCTC | MOE | | | | | T_{ds}T_{ds}^{m}C_{ds}^{m}CₑₒTₑₒ^{m}CₑₛTₑₛ^{m}Cₑ | |
| 1248269 | TTAACCCAAT | 5-10-5 | N/A | N/A | 32929 | 32948 | TₑₛTₑₒAₑₒAₑₒ^{m}Cₑₛ^{m}C_{ds}^{m}C_{ds}A_{ds}A_{ds}T_{ds}A_{ds} | 20 |
| | AATCTGACAT | MOE | | | | | A_{ds}T_{ds}^{m}C_{ds}T_{ds}GₑₒAₑₒ^{m}CₑₛAₑₛTₑ | |
| 1248271 | AATGGCACAT | 5-10-5 | N/A | N/A | 37155 | 37174 | AₑₛAₑₒTₑₒGₑₒGₑₛ^{m}C_{ds}A_{ds}^{m}C_{ds}A_{ds}T_{ds}T_{ds} | 21 |
| | TTTTTATAGA | MOE | | | | | T_{ds}T_{ds}T_{ds}T_{ds}AₑₒTₑₒAₑₛGₑₛAₑ | |
| 1248273 | CTTTAACCCAA | 5-10-5 | N/A | N/A | 32931 | 32950 | ^{m}CₑₛTₑₒTₑₒTₑₒAₑₛA_{ds}^{m}C_{ds}^{m}C_{ds}^{m}C_{ds}A_{ds} | 22 |
| | TAATCTGAC | MOE | | | | | A_{ds}T_{ds}A_{ds}A_{ds}T_{ds}^{m}CₑₒTₑₒGₑₛAₑₛ^{m}Cₑ | |
| 1248275 | ATCCTTTAACC | 5-10-5 | N/A | N/A | 32934 | 32953 | AₑₛTₑₒ^{m}Cₑₒ^{m}CₑₒTₑₛT_{ds}T_{ds}A_{ds}A_{ds}^{m}C_{ds}^{m} | 23 |
| | CAATAATCT | MOE | | | | | C_{ds}^{m}C_{ds}A_{ds}A_{ds}T_{ds}AₑₒAₑₒTₑₛ^{m}CₑₛTₑ | |
| 1248276 | TTATAGAGTTC | 5-10-5 | N/A | N/A | 37142 | 37161 | TₑₛTₑₒAₑₒTₑₒAₑₛG_{ds}A_{ds}G_{ds}T_{ds}T_{ds}^{m}C_{ds}^{m} | 24 |
| | CTCTCAATT | MOE | | | | | C_{ds}T_{ds}^{m}C_{ds}T_{ds}^{m}CₑₒAₑₒAₑₛTₑₛTₑ | |
| 1248277 | ATATCCTTTAA | 5-10-5 | N/A | N/A | 32936 | 32955 | AₑₛTₑₒAₑₒTₑₒ^{m}Cₑₛ^{m}C_{ds}T_{ds}T_{ds}T_{ds}A_{ds}A_{ds}^{m} | 25 |
| | CCCAATAAT | MOE | | | | | C_{ds}^{m}C_{ds}^{m}C_{ds}A_{ds}AₑₒTₑₒAₑₛAₑₛTₑ | |
| 1248278 | CACATTTTTTA | 5-10-5 | N/A | N/A | 37150 | 37169 | ^{m}CₑₛAₑₒ^{m}CₑₒAₑₒTₑₛT_{ds}T_{ds}T_{ds}T_{ds}T_{ds}A_{ds} | 26 |
| | TAGAGTTCC | MOE | | | | | T_{ds}A_{ds}G_{ds}A_{ds}GₑₒTₑₒTₑₛ^{m}Cₑₛ^{m}Cₑ | |
| 1248257 | CTCAAGTACTT | 5-10-5 | 1852 | 1871 | 46378 | 46397 | ^{m}CₑₛTₑₒ^{m}CₑₒAₑₒAₑₛG_{ds}T_{ds}A_{ds}^{m}C_{ds}T_{ds}T_{ds} | 27 |
| | GTGCAAGGC | MOE | | | | | G_{ds}T_{ds}G_{ds}^{m}C_{ds}AₑₒAₑₒGₑₛGₑₛ^{m}Cₑ | |
| 1248260 | TCTCAAGTACT | 5-10-5 | 1853 | 1872 | 46379 | 46398 | Tₑₛ^{m}CₑₒTₑₒ^{m}CₑₒAₑₛA_{ds}G_{ds}T_{ds}A_{ds}^{m}C_{ds}T_{ds} | 28 |
| | TGTGCAAGG | MOE | | | | | T_{ds}G_{ds}T_{ds}G_{ds}^{m}CₑₒAₑₒAₑₛGₑₛGₑ | |
| 1248263 | TGGTTTTCTCA | 5-10-5 | 3068 | 3087 | 47594 | 47613 | TₑₛGₑₒGₑₒTₑₒTₑₛT_{ds}T_{ds}^{m}C_{ds}T_{ds}^{m}C_{ds}A_{ds} | 29 |
| | TTTTTATAT | MOE | | | | | T_{ds}T_{ds}T_{ds}T_{ds}TₑₒAₑₒTₑₛAₑₛTₑ | |
| 1248270 | TATTCTCAAGT | 5-10-5 | 1856 | 1875 | 46382 | 46401 | TₑₛAₑₒTₑₒTₑₒ^{m}CₑₛT_{ds}^{m}C_{ds}A_{ds}A_{ds}G_{ds}T_{ds} | 30 |
| | ACTTGTGCA | MOE | | | | | A_{ds}^{m}C_{ds}T_{ds}T_{ds}GₑₒTₑₒGₑₛ^{m}CₑₛAₑ | |
| 1248272 | TAAAAAATGC | 5-10-5 | 1871 | 1890 | 46397 | 46416 | TₑₛAₑₒAₑₒAₑₒAₑₛA_{ds}A_{ds}T_{ds}G_{ds}^{m}C_{ds}T_{ds}^{m} | 31 |
| | TCATTTATTC | MOE | | | | | C_{ds}A_{ds}T_{ds}T_{ds}TₑₒAₑₒTₑₛTₑₛ^{m}Cₑ | |
| 1248274 | TGCTCATTTAT | 5-10-5 | 1864 | 1883 | 46390 | 46409 | TₑₛGₑₒ^{m}CₑₒTₑₒ^{m}CₑₛA_{ds}T_{ds}T_{ds}T_{ds}A_{ds}T_{ds} | 32 |
| | TCTCAAGTA | MOE | | | | | T_{ds}^{m}C_{ds}T_{ds}^{m}C_{ds}AₑₒAₑₒGₑₛTₑₛAₑ | |
| 1248279 | TAACCCAATA | 5-10-5 | N/A | N/A | 32928 | 32947 | TₑₛAₑₒAₑₒ^{m}Cₑₒ^{m}Cₑₛ^{m}C_{ds}A_{ds}A_{ds}T_{ds}A_{ds}A_{ds} | 33 |
| | ATCTGACATC | MOE | | | | | T_{ds}^{m}C_{ds}T_{ds}G_{ds}Aₑₒ^{m}CₑₒAₑₛTₑₛ^{m}Cₑ | |
| 1248280 | TATCCTTTAAC | 5-10-5 | N/A | N/A | 32935 | 32954 | TₑₛAₑₒTₑₒ^{m}Cₑₒ^{m}CₑₛT_{ds}T_{ds}T_{ds}A_{ds}A_{ds}^{m}C_{ds}^{m} | 34 |
| | CCAATAATC | MOE | | | | | C_{ds}^{m}C_{ds}A_{ds}A_{ds}TₑₒAₑₒAₑₛTₑₛ^{m}Cₑ | |
| 1248281 | CCTTTAACCCA | 5-10-5 | N/A | N/A | 32932 | 32951 | ^{m}Cₑₛ^{m}CₑₒTₑₒTₑₒTₑₛA_{ds}A_{ds}^{m}C_{ds}^{m}C_{ds}^{m}C_{ds} | 35 |
| | ATAATCTGA | MOE | | | | | A_{ds}A_{ds}T_{ds}A_{ds}A_{ds}Tₑₒ^{m}CₑₒTₑₛGₑₛAₑ | |
| 1248282 | ATAATCTGAC | 5-10-5 | N/A | N/A | 32921 | 32940 | AₑₛTₑₒAₑₒAₑₒTₑₛ^{m}C_{ds}T_{ds}G_{ds}A_{ds}^{m}C_{ds}A_{ds} | 36 |
| | ATCCTCAGAA | MOE | | | | | T_{ds}^{m}C_{ds}^{m}C_{ds}T_{ds}^{m}CₑₒAₑₒGₑₛAₑₛAₑ | |
| 1248283 | GGTTTTCTCAT | 5-10-5 | 3067 | 3086 | 47593 | 47612 | GₑₛGₑₒTₑₒTₑₒTₑₛT_{ds}^{m}C_{ds}T_{ds}^{m}C_{ds}A_{ds}T_{ds} | 37 |
| | TTTTATATT | MOE | | | | | T_{ds}T_{ds}T_{ds}T_{ds}AₑₒTₑₒAₑₛTₑₛTₑ | |
| 1248284 | TGTTCATATCC | 5-10-5 | N/A | N/A | 32941 | 32960 | TₑₛGₑₒTₑₒTₑₒ^{m}CₑₛA_{ds}T_{ds}A_{ds}T_{ds}^{m}C_{ds}^{m}C_{ds} | 38 |
| | TTTAACCCA | MOE | | | | | T_{ds}T_{ds}T_{ds}A_{ds}Aₑₒ^{m}Cₑₒ^{m}Cₑₛ^{m}CₑₛAₑ | |
| 1248285 | TCAAGTACTTG | 5-10-5 | 1851 | 1870 | 46377 | 46396 | Tₑₛ^{m}CₑₒAₑₒAₑₒGₑₛT_{ds}A_{ds}^{m}C_{ds}T_{ds}T_{ds}G_{ds} | 39 |
| | TGCAAGGCT | MOE | | | | | T_{ds}G_{ds}^{m}C_{ds}A_{ds}AₑₒGₑₒGₑₛ^{m}CₑₛTₑ | |
| 1248286 | CATTTTTTATA | 5-10-5 | N/A | N/A | 37148 | 37167 | ^{m}CₑₛAₑₒTₑₒTₑₒTₑₛT_{ds}T_{ds}T_{ds}A_{ds}T_{ds}A_{ds}G_{ds} | 40 |
| | GAGTTCCTC | MOE | | | | | A_{ds}G_{ds}T_{ds}Tₑₒ^{m}Cₑₒ^{m}CₑₛTₑₛ^{m}Cₑ | |
| 1248287 | CCCAATAATCT | 5-10-5 | N/A | N/A | 32925 | 32944 | ^{m}Cₑₛ^{m}Cₑₒ^{m}CₑₒAₑₒAₑₛT_{ds}A_{ds}A_{ds}T_{ds}^{m}C_{ds} | 41 |
| | GACATCCTC | MOE | | | | | T_{ds}G_{ds}A_{ds}^{m}C_{ds}A_{ds}Tₑₒ^{m}Cₑₒ^{m}CₑₛTₑₛ^{m}Cₑ | |
| 1248288 | ACCCAATAAT | 5-10-5 | N/A | N/A | 32926 | 32945 | Aₑₛ^{m}Cₑₒ^{m}Cₑₒ^{m}CₑₒAₑₛA_{ds}T_{ds}A_{ds}A_{ds}T_{ds}^{m} | 42 |
| | CTGACATCCT | MOE | | | | | C_{ds}T_{ds}G_{ds}A_{ds}^{m}C_{ds}AₑₒTₑₒ^{m}Cₑₛ^{m}CₑₛTₑ | |
| 1248289 | AATGTTCATAT | 5-10-5 | N/A | N/A | 32943 | 32962 | AₑₛAₑₒTₑₒGₑₒTₑₛT_{ds}^{m}C_{ds}A_{ds}T_{ds}A_{ds}T_{ds}^{m} | 43 |
| | CCTTTAACC | MOE | | | | | C_{ds}^{m}C_{ds}T_{ds}T_{ds}TₑₒAₑₒAₑₛ^{m}Cₑₛ^{m}Cₑ | |
| 1248290 | GGCACATTTTT | 5-10-5 | N/A | N/A | 37152 | 37171 | GₑₛGₑₒ^{m}CₑₒAₑₒ^{m}CₑₛA_{ds}T_{ds}T_{ds}T_{ds}T_{ds}T_{ds} | 44 |
| | TATAGAGTT | MOE | | | | | T_{ds}A_{ds}T_{ds}A_{ds}GₑₒAₑₒGₑₛTₑₛTₑ | |
| 1248291 | AAGTACTTGT | 5-10-5 | 1849 | 1868 | 46375 | 46394 | AₑₛAₑₒGₑₒTₑₒAₑₛ^{m}C_{ds}T_{ds}T_{ds}G_{ds}T_{ds}G_{ds}^{m} | 45 |
| | GCAAGGCTGA | MOE | | | | | C_{ds}A_{ds}A_{ds}G_{ds}Gₑₒ^{m}CₑₒTₑₛGₑₛAₑ | |
| 1248292 | ATGTTCATATC | 5-10-5 | N/A | N/A | 32942 | 32961 | AₑₛTₑₒGₑₒTₑₒTₑₛ^{m}C_{ds}A_{ds}T_{ds}A_{ds}T_{ds}^{m}C_{ds}^{m} | 46 |
| | CTTTAACCC | MOE | | | | | C_{ds}T_{ds}T_{ds}T_{ds}AₑₒAₑₒ^{m}Cₑₛ^{m}Cₑₛ^{m}Cₑ | |
| 1248293 | TTTTTATAGAG | 5-10-5 | N/A | N/A | 37145 | 37164 | TₑₛTₑₒTₑₒTₑₒTₑₛA_{ds}T_{ds}A_{ds}G_{ds}A_{ds}G_{ds}T_{ds} | 47 |
| | TTCCTCTCA | MOE | | | | | T_{ds}^{m}C_{ds}^{m}C_{ds}Tₑₒ^{m}CₑₒTₑₛ^{m}CₑₛAₑ | |
| 1248297 | ACATTTTTTAT | 5-10-5 | N/A | N/A | 37149 | 37168 | Aₑₛ^{m}CₑₒAₑₒTₑₒTₑₛT_{ds}T_{ds}T_{ds}T_{ds}A_{ds}T_{ds}A_{ds} | 48 |
| | AGAGTTCCT | MOE | | | | | G_{ds}A_{ds}G_{ds}TₑₒTₑₒ^{m}Cₑₛ^{m}CₑₛTₑ | |
| 1248298 | AATCTGACAT | 5-10-5 | N/A | N/A | 32919 | 32938 | AₑₛAₑₒTₑₒ^{m}CₑₒTₑₛG_{ds}A_{ds}^{m}C_{ds}A_{ds}T_{ds}^{m}C_{ds}^{m} | 49 |
| | CCTCAGAAAA | MOE | | | | | C_{ds}T_{ds}^{m}C_{ds}A_{ds}GₑₒAₑₒAₑₛAₑₛAₑ | |
| 1247564 | GCATATTGGTT | 5-10-5 | 3074 | 3093 | 49600 | 47619 | Gₑₛ^{m}CₑₒAₑₒTₑₒAₑₒT_{ds}T_{ds}G_{ds}G_{ds}T_{ds}T_{ds}T_{ds} | 50 |
| | TTCTCATTT | MOE | | | | | T_{ds}^{m}C_{ds}T_{ds}^{m}CₑₒAₑₒTₑₛTₑₛTₑ | |
| 1247565 | GCATATTGGTT | 5-10-5 | 3074 | 3093 | 47600 | 47619 | Gₑₛ^{m}CₑₒAₑₒTₑₒAₑₒT_{ds}T_{ds}G_{ds}G_{ds}T_{ds}T_{ds}T_{ds} | 50 |
| | TTCTCATTT | MOE | | | | | T_{ds}^{m}C_{ds}T_{ds}^{m}CₑₒAₑₒTₑₛTₑₛTₑ | |
| 1247566 | GCATATTGGTT | 5-10-5 | 3074 | 3093 | 47600 | 47619 | Gₑₛ^{m}CₑₒAₑₒTₑₒAₑₒT_{ds}T_{ds}G_{ds}G_{ds}T_{ds}T_{ds}T_{ds} | 50 |
| | TTCTCATTT | MOE | | | | | T_{ds}^{m}C_{ds}T_{ds}^{m}CₑₒAₑₒTₑₛTₑₛTₑ | |
| 1247567 | CATATTGGTTT | 5-10-5 | 3074 | 3092 | 47600 | 47618 | ^{m}CₑₛAₑₒTₑₒAₑₒTₑₛT_{ds}G_{ds}G_{ds}T_{ds}T_{ds}T_{ds}T_{ds}^{m} | 51 |
| | TCTCATTT | MOE | | | | | C_{ds}T_{ds}^{m}CₑₒAₑₒTₑₛTₑₛTₑ | |
| 1247568 | GCATATTGGTT | 5-10-5 | 3075 | 3093 | 47601 | 47619 | Gₑₛ^{m}CₑₒAₑₒTₑₒAₑₛT_{ds}T_{ds}G_{ds}G_{ds}T_{ds}T_{ds}T_{ds} | 52 |
| | TTCTCATT | MOE | | | | | T_{ds}^{m}C_{ds}Tₑₒ^{m}CₑₒAₑₛTₑₛTₑ | |
| 1248294 | TTCTCAAGTAC | 5-10-5 | 1854 | 1873 | 46380 | 46399 | TₑₛTₑₒ^{m}CₑₒTₑₒ^{m}CₑₛA_{ds}A_{ds}G_{ds}T_{ds}A_{ds}^{m}C_{ds} | 53 |
| | TTGTGCAAG | MOE | | | | | T_{ds}T_{ds}G_{ds}T_{ds}Gₑₒ^{m}CₑₒAₑₛAₑₛGₑ | |
| 1248295 | TTATTCTCAAG | 5-10-5 | 1857 | 1876 | 46383 | 46402 | TₑₛTₑₒAₑₒTₑₒTₑₛ^{m}C_{ds}T_{ds}^{m}C_{ds}A_{ds}A_{ds}G_{ds} | 54 |
| | TACTTGTGC | MOE | | | | | T_{ds}A_{ds}^{m}C_{ds}T_{ds}TₑₒGₑₒTₑₛGₑₛ^{m}Cₑ | |
| 1248296 | GTTTTCTCATT | 5-10-5 | 3066 | 3085 | 47592 | 47611 | GₑₛTₑₒTₑₒTₑₒTₑₛ^{m}C_{ds}T_{ds}^{m}C_{ds}A_{ds}T_{ds}T_{ds} | 55 |
| | TTTATATTA | MOE | | | | | T_{ds}T_{ds}T_{ds}A_{ds}TₑₒAₑₒTₑₛTₑₛAₑ | |
| 1248299 | CAAGTACTTGT | 5-10-5 | 1850 | 1869 | 46376 | 46395 | ^{m}CₑₛAₑₒAₑₒGₑₒTₑₛA_{ds}^{m}C_{ds}T_{ds}T_{ds}G_{ds}T_{ds} | 56 |
| | GCAAGGCTG | MOE | | | | | G_{ds}^{m}C_{ds}A_{ds}A_{ds}GₑₒGₑₒ^{m}CₑₛTₑₛGₑ | |
| 1248300 | ATTCTCAAGTA | 5-10-5 | 1855 | 1874 | 46381 | 46400 | AₑₛTₑₒTₑₒ^{m}CₑₒTₑₛ^{m}C_{ds}A_{ds}A_{ds}G_{ds}T_{ds}A_{ds}^{m} | 57 |
| | CTTGTGCAA | MOE | | | | | C_{ds}T_{ds}T_{ds}G_{ds}TₑₒGₑₒ^{m}CₑₛAₑₛAₑ | |
| 1269632 | GCTCATTTATT | 5-10-5 | 1863 | 1882 | 46389 | 46408 | Gₑₛ^{m}CₑₒTₑₛ^{m}CₑₛAₑₛT_{ds}T_{ds}T_{ds}A_{ds}T_{ds}T_{ds}^{m} | 58 |
| | CTCAAGTAC | MOE | | | | | C_{ds}T_{ds}^{m}C_{ds}A_{ds}AₑₛGₑₒTₑₛAₑₛ^{m}Cₑ | |
| 1269633 | TAATACTTTTT | 5-10-5 | N/A | N/A | 19453 | 19472 | TₑₛAₑₒAₑₛTₑₛAₑₛ^{m}C_{ds}T_{ds}T_{ds}T_{ds}T_{ds}T_{ds}^{m} | 59 |
| | CCAGCCTTC | MOE | | | | | C_{ds}^{m}C_{ds}A_{ds}G_{ds}^{m}Cₑₛ^{m}CₑₒTₑₛTₑₛ^{m}Cₑ | |
| 1269634 | TGGCACATTTT | 5-10-5 | N/A | N/A | 37153 | 37172 | TₑₛGₑₒGₑₛ^{m}CₑₛAₑₛ^{m}C_{ds}A_{ds}T_{ds}T_{ds}T_{ds}T_{ds} | 60 |
| | TTATAGAGT | MOE | | | | | T_{ds}T_{ds}A_{ds}T_{ds}AₑₛGₑₒAₑₛGₑₛTₑ | |
| 1269635 | GCACCATATA | 5-10-5 | N/A | N/A | 6597 | 6616 | Gₑₛ^{m}CₑₒAₑₛ^{m}Cₑₛ^{m}CₑₛA_{ds}T_{ds}A_{ds}T_{ds}A_{ds}T_{ds} | 61 |
| | TATCTCAGAA | MOE | | | | | A_{ds}T_{ds}^{m}C_{ds}T_{ds}^{m}CₑₛAₑₒGₑₛAₑₛAₑ | |
| 1269636 | GTTAATACTTT | 5-10-5 | N/A | N/A | 19455 | 19474 | GₑₛTₑₒTₑₛAₑₛAₑₛT_{ds}A_{ds}^{m}C_{ds}T_{ds}T_{ds}T_{ds}T_{ds} | 62 |
| | TTCCAGCCT | MOE | | | | | T_{ds}^{m}C_{ds}^{m}C_{ds}AₑₛGₑₒ^{m}Cₑₛ^{m}CₑₛTₑ | |
| 1269637 | GCCAAAATAC | 5-10-5 | N/A | N/A | 32676 | 32695 | Gₑₛ^{m}Cₑₒ^{m}CₑₛAₑₛAₑₛA_{ds}A_{ds}T_{ds}A_{ds}^{m}C_{ds}T_{ds} | 63 |
| | TAACATCAGT | MOE | | | | | A_{ds}A_{ds}^{m}C_{ds}A_{ds}Tₑₛ^{m}CₑₒAₑₛGₑₛTₑ | |
| 1269638 | GTATAGAGTTT | 5-10-5 | 4142 | 4161 | 48668 | 48687 | GₑₛTₑₒAₑₛTₑₛAₑₛG_{ds}A_{ds}G_{ds}T_{ds}T_{ds}T_{ds}A_{ds}^{m} | 64 |
| | ACCTGCAGC | MOE | | | | | C_{ds}^{m}C_{ds}T_{ds}Gₑₛ^{m}CₑₒAₑₛGₑₛ^{m}Cₑ | |
| 1269639 | TGAGCCAATA | 5-10-5 | N/A | N/A | 30453 | 30472 | TₑₛGₑₒAₑₛGₑₛ^{m}Cₑₛ^{m}C_{ds}A_{ds}A_{ds}T_{ds}A_{ds}T_{ds} | 65 |
| | TTTATAGGTG | MOE | | | | | T_{ds}T_{ds}A_{ds}T_{ds}AₑₛGₑₒGₑₛTₑₛGₑ | |
| 1269640 | ATGTTAATACT | 5-10-5 | N/A | N/A | 19457 | 19476 | AₑₛTₑₒGₑₛTₑₛTₑₛA_{ds}A_{ds}T_{ds}A_{ds}^{m}C_{ds}T_{ds}T_{ds} | 66 |
| | TTTTCCAGC | MOE | | | | | T_{ds}T_{ds}T_{ds}^{m}Cₑₛ^{m}CₑₒAₑₛGₑₛ^{m}Cₑ | |
| 1269481 | AGAAGAGTGC | 5-10-5 | N/A | N/A | 15671 | 15690 | AₑₛGₑₒAₑₒAₑₒGₑₛA_{ds}G_{ds}T_{ds}G_{ds}^{m}C_{ds}T_{ds} | 67 |
| | TTTTCATACC | MOE | | | | | T_{ds}T_{ds}T_{ds}^{m}C_{ds}AₑₒTₑₒAₑₛ^{m}Cₑₛ^{m}Cₑ | |
| 1269482 | GAAGAGTGCT | 5-10-5 | N/A | N/A | 15670 | 15689 | GₑₛAₑₒAₑₒGₑₒAₑₛG_{ds}T_{ds}G_{ds}^{m}C_{ds}T_{ds}T_{ds} | 68 |
| | TTTCATACCA | MOE | | | | | T_{ds}T_{ds}^{m}C_{ds}A_{ds}TₑₒAₑₒ^{m}Cₑₛ^{m}CₑₛAₑ | |
| 1269483 | AAGAGTGCTT | 5-10-5 | N/A | N/A | 15669 | 15688 | AₑₛAₑₒGₑₒAₑₒGₑₛT_{ds}G_{ds}^{m}C_{ds}T_{ds}T_{ds}T_{ds}T_{ds}^{m} | 69 |
| | TTCATACCAG | MOE | | | | | C_{ds}A_{ds}T_{ds}Aₑₒ^{m}Cₑₒ^{m}CₑₛAₑₛGₑ | |
| 1269484 | AGAGTGCTTTT | 5-10-5 | N/A | N/A | 15668 | 15687 | AₑₛGₑₒAₑₒGₑₒTₑₛG_{ds}^{m}C_{ds}T_{ds}T_{ds}T_{ds}T_{ds}^{m} | 70 |
| | CATACCAGG | MOE | | | | | C_{ds}A_{ds}T_{ds}A_{ds}^{m}Cₑₒ^{m}CₑₒAₑₛGₑₛGₑ | |
| 1269485 | GTGCTTTTCAT | 5-10-5 | N/A | N/A | 15665 | 15684 | GₑₛTₑₒGₑₒ^{m}CₑₒTₑₛT_{ds}T_{ds}T_{ds}^{m}C_{ds}A_{ds}T_{ds} | 71 |
| | ACCAGGTCT | MOE | | | | | A_{ds}^{m}C_{ds}^{m}C_{ds}A_{ds}GₑₒGₑₒTₑₛ^{m}CₑₛTₑ | |
| 1269486 | TGCTTTTCATA | 5-10-5 | N/A | N/A | 15664 | 15683 | TₑₛGₑₒ^{m}CₑₒTₑₒTₑₛT_{ds}T_{ds}^{m}C_{ds}A_{ds}T_{ds}A_{ds}^{m} | 72 |
| | CCAGGTCTC | MOE | | | | | C_{ds}^{m}C_{ds}A_{ds}G_{ds}GₑₒTₑₒ^{m}CₑₛTₑₛ^{m}Cₑ | |
| 1269487 | TTTTCATACCA | 5-10-5 | N/A | N/A | 15661 | 15680 | TₑₛTₑₒTₑₒTₑₒ^{m}CₑₛA_{ds}T_{ds}A_{ds}^{m}C_{ds}^{m}C_{ds}A_{ds} | 73 |
| | GGTCTCTGA | MOE | | | | | G_{ds}G_{ds}T_{ds}^{m}C_{ds}Tₑₒ^{m}CₑₒTₑₛGₑₛAₑ | |
| 1269488 | TCATACCAGG | 5-10-5 | N/A | N/A | 15658 | 15677 | Tₑₛ^{m}CₑₒAₑₒTₑₒAₑₛ^{m}C_{ds}^{m}C_{ds}A_{ds}G_{ds}G_{ds}T_{ds}^{m} | 74 |
| | TCTCTGAGAT | MOE | | | | | C_{ds}T_{ds}^{m}C_{ds}T_{ds}GₑₒAₑₒGₑₛAₑₛTₑ | |
| 1269495 | TGTTAATACTT | 5-10-5 | N/A | N/A | 19456 | 19475 | TₑₛGₑₒTₑₒTₑₒAₑₛA_{ds}T_{ds}A_{ds}^{m}C_{ds}T_{ds}T_{ds}T_{ds} | 75 |
| | TTTCCAGCC | MOE | | | | | T_{ds}T_{ds}^{m}C_{ds}^{m}CₑₒAₑₒGₑₛ^{m}Cₑₛ^{m}Cₑ | |
| 1269496 | ATACTTTTTCC | 5-10-5 | N/A | N/A | 19451 | 19470 | AₑₛTₑₒAₑₒ^{m}CₑₒTₑₛT_{ds}T_{ds}T_{ds}T_{ds}^{m}C_{ds}^{m}C_{ds} | 76 |
| | AGCCTTCTT | MOE | | | | | A_{ds}G_{ds}^{m}C_{ds}^{m}C_{ds}TₑₒTₑₒ^{m}CₑₛTₑₛTₑ | |
| 1269636 | GTTAATACTTT | 5-10-5 | N/A | N/A | 19455 | 19474 | GₑₛTₑₒTₑₛAₑₛAₑₛT_{ds}A_{ds}^{m}C_{ds}T_{ds}T_{ds}T_{ds}T_{ds} | 62 |
| | TTCCAGCCT | MOE | | | | | T_{ds}^{m}C_{ds}^{m}C_{ds}AₑₛGₑₒ^{m}Cₑₛ^{m}CₑₛTₑ | |
| 1269450 | GATAAACAGC | 5-10-5 | N/A | N/A | 6605 | 6624 | GₑₛAₑₒTₑₒAₑₒAₑₛA_{ds}^{m}C_{ds}A_{ds}G_{ds}^{m}C_{ds}A_{ds}^{m} | 77 |
| | ACCATATATA | MOE | | | | | C_{ds}^{m}C_{ds}A_{ds}T_{ds}AₑₒTₑₒAₑₛTₑₛAₑ | |
| 1269451 | TAAACAGCAC | 5-10-5 | N/A | N/A | 6603 | 6622 | TₑₛAₑₒAₑₒAₑₒ^{m}CₑₛA_{ds}G_{ds}^{m}C_{ds}A_{ds}^{m}C_{ds}^{m} | 78 |
| | CATATATATC | MOE | | | | | C_{ds}A_{ds}T_{ds}A_{ds}T_{ds}AₑₒTₑₒAₑₛTₑₛ^{m}Cₑ | |
| 1269460 | CCAAAATACT | 5-10-5 | N/A | N/A | 32675 | 32694 | ^{m}Cₑₛ^{m}CₑₒAₑₒAₑₒAₑₛA_{ds}T_{ds}A_{ds}^{m}C_{ds}T_{ds}A_{ds} | 79 |
| | AACATCAGTC | MOE | | | | | A_{ds}^{m}C_{ds}A_{ds}T_{ds}^{m}CₑₒAₑₒGₑₛTₑₛ^{m}Cₑ | |
| 1269461 | AAAATACTAA | 5-10-5 | N/A | N/A | 32673 | 32692 | AₑₛAₑₒAₑₒAₑₒTₑₛA_{ds}^{m}C_{ds}T_{ds}A_{ds}A_{ds}^{m}C_{ds} | 80 |
| | CATCAGTCAC | MOE | | | | | A_{ds}T_{ds}^{m}C_{ds}A_{ds}GₑₒTₑₒ^{m}CₑₛAₑₛ^{m}Cₑ | |
| 1269462 | AAATACTAAC | 5-10-5 | N/A | N/A | 32672 | 32691 | AₑₛAₑₒAₑₒTₑₒAₑₛ^{m}C_{ds}T_{ds}A_{ds}A_{ds}^{m}C_{ds}A_{ds} | 81 |
| | ATCAGTCACT | MOE | | | | | T_{ds}^{m}C_{ds}A_{ds}G_{ds}Tₑₒ^{m}CₑₒAₑₛ^{m}CₑₛTₑ | |
| 1269463 | AATACTAACA | 5-10-5 | N/A | N/A | 32671 | 32690 | AₑₛAₑₒTₑₒAₑₒ^{m}CₑₛT_{ds}A_{ds}A_{ds}^{m}C_{ds}A_{ds}T_{ds}^{m} | 82 |
| | TCAGTCACTG | MOE | | | | | C_{ds}A_{ds}G_{ds}T_{ds}^{m}CₑₒAₑₒ^{m}CₑₛTₑₛGₑ | |
| 1269464 | ATACTAACAT | 5-10-5 | N/A | N/A | 32670 | 32689 | AₑₛTₑₒAₑₒ^{m}CₑₒTₑₛA_{ds}A_{ds}^{m}C_{ds}A_{ds}T_{ds}^{m}C_{ds} | 83 |
| | CAGTCACTGA | MOE | | | | | A_{ds}G_{ds}T_{ds}^{m}C_{ds}Aₑₒ^{m}CₑₒTₑₛGₑₛAₑ | |
| 1269477 | AAACAGCACC | 5-10-5 | N/A | N/A | 6602 | 6621 | AₑₛAₑₒAₑₒ^{m}CₑₒAₑₛG_{ds}^{m}C_{ds}A_{ds}^{m}C_{ds}^{m}C_{ds} | 84 |
| | ATATATATCT | MOE | | | | | A_{ds}T_{ds}A_{ds}T_{ds}A_{ds}TₑₒAₑₒTₑₛ^{m}CₑₛTₑ | |
| 1269478 | ACAGCACCAT | 5-10-5 | N/A | N/A | 6600 | 6619 | Aₑₛ^{m}CₑₒAₑₒGₑₒ^{m}CₑₛA_{ds}^{m}C_{ds}^{m}C_{ds}A_{ds}T_{ds} | 85 |
| | ATATATCTCA | MOE | | | | | A_{ds}T_{ds}A_{ds}T_{ds}A_{ds}Tₑₒ^{m}CₑₒTₑₛ^{m}CₑₛAₑ | |
| 1269479 | CACCATATAT | 5-10-5 | N/A | N/A | 6596 | 6615 | ^{m}CₑₛAₑₒ^{m}Cₑₒ^{m}CₑₒAₑₛT_{ds}A_{ds}T_{ds}A_{ds}T_{ds}A_{ds} | 86 |
| | ATCTCAGAAA | MOE | | | | | T_{ds}^{m}C_{ds}T_{ds}^{m}C_{ds}AₑₒGₑₒAₑₛAₑₛAₑ | |
| 1269480 | ACCATATATAT | 5-10-5 | N/A | N/A | 6595 | 6614 | Aₑₛ^{m}Cₑₒ^{m}CₑₒAₑₒTₑₛA_{ds}T_{ds}A_{ds}T_{ds}A_{ds}T_{ds}^{m} | 87 |
| | CTCAGAAAC | MOE | | | | | C_{ds}T_{ds}^{m}C_{ds}A_{ds}GₑₒAₑₒAₑₛAₑₛ^{m}Cₑ | |
| 1269489 | ACATTACTGGT | 5-10-5 | N/A | N/A | 17188 | 17207 | Aₑₛ^{m}CₑₒAₑₒTₑₒTₑₛA_{ds}^{m}C_{ds}T_{ds}G_{ds}G_{ds}T_{ds}^{m} | 88 |
| | CAGTTTCCT | MOE | | | | | C_{ds}A_{ds}G_{ds}T_{ds}TₑₒTₑₒ^{m}Cₑₛ^{m}CₑₛTₑ | |
| 1269490 | CATTACTGGTC | 5-10-5 | N/A | N/A | 17187 | 17206 | ^{m}CₑₛAₑₒTₑₒTₑₒAₑₛ^{m}C_{ds}T_{ds}G_{ds}G_{ds}T_{ds}^{m}C_{ds} | 89 |
| | AGTTTCCTA | MOE | | | | | A_{ds}G_{ds}T_{ds}T_{ds}Tₑₒ^{m}Cₑₒ^{m}CₑₛTₑₛAₑ | |
| 1269491 | ATTACTGGTCA | 5-10-5 | N/A | N/A | 17186 | 17205 | AₑₛTₑₒTₑₒAₑₒ^{m}CₑₛT_{ds}G_{ds}G_{ds}T_{ds}^{m}C_{ds}A_{ds} | 90 |
| | GTTTCCTAA | MOE | | | | | G_{ds}T_{ds}T_{ds}T_{ds}^{m}Cₑₒ^{m}CₑₒTₑₛAₑₛAₑ | |
| 1269492 | TACTGGTCAGT | 5-10-5 | N/A | N/A | 17184 | 17203 | TₑₛAₑₒ^{m}CₑₒTₑₒGₑₛG_{ds}T_{ds}^{m}C_{ds}A_{ds}G_{ds}T_{ds} | 91 |
| | TTCCTAATT | MOE | | | | | T_{ds}T_{ds}^{m}C_{ds}^{m}C_{ds}TₑₒAₑₒAₑₛTₑₛTₑ | |
| 1269493 | ACTGGTCAGTT | 5-10-5 | N/A | N/A | 17183 | 17202 | Aₑₛ^{m}CₑₒTₑₒGₑₒGₑₛT_{ds}^{m}C_{ds}A_{ds}G_{ds}T_{ds}T_{ds} | 92 |
| | TCCTAATTT | MOE | | | | | T_{ds}^{m}C_{ds}^{m}C_{ds}T_{ds}AₑₒAₑₒTₑₛTₑₛTₑ | |
| 1269494 | CTGGTCAGTTT | 5-10-5 | N/A | N/A | 17182 | 17201 | ^{m}CₑₛTₑₒGₑₒGₑₒTₑₛ^{m}C_{ds}A_{ds}G_{ds}T_{ds}T_{ds}T_{ds}^{m} | 93 |
| | CCTAATTTT | MOE | | | | | C_{ds}^{m}C_{ds}T_{ds}A_{ds}AₑₒTₑₒTₑₛTₑₛTₑ | |
| 1269442 | ATTTTCATGTT | 5-10-5 | 2240 | 2259 | 46766 | 46785 | AₑₛTₑₒTₑₒTₑₒTₑₛ^{m}C_{ds}A_{ds}T_{ds}G_{ds}T_{ds}T_{ds}^{m} | 94 |
| | CCAGATCAC | MOE | | | | | C_{ds}^{m}C_{ds}A_{ds}G_{ds}AₑₒTₑₒ^{m}CₑₛAₑₛ^{m}Cₑ | |
| 1269443 | CATGTTCCAG | 5-10-5 | 2235 | 2254 | 46761 | 46780 | ^{m}CₑₛAₑₒTₑₒGₑₒTₑₛT_{ds}^{m}C_{ds}^{m}C_{ds}A_{ds}G_{ds}A_{ds} | 95 |
| | ATCACCATCT | MOE | | | | | T_{ds}^{m}C_{ds}A_{ds}^{m}C_{ds}^{m}CₑₒAₑₒTₑₛ^{m}CₑₛTₑ | |
| 1269444 | TCCAGATCAC | 5-10-5 | 2230 | 2249 | 46756 | 46775 | Tₑₛ^{m}Cₑₒ^{m}CₑₒAₑₒGₑₛA_{ds}T_{ds}^{m}C_{ds}A_{ds}^{m}C_{ds}^{m} | 96 |
| | CATCTTTGAC | MOE | | | | | C_{ds}A_{ds}T_{ds}^{m}C_{ds}T_{ds}TₑₒTₑₒGₑₛAₑₛ^{m}Cₑ | |
| 1269445 | CAGATCACCA | 5-10-5 | 2228 | 2247 | 46754 | 46773 | ^{m}CₑₛAₑₒGₑₒAₑₒTₑₛ^{m}C_{ds}A_{ds}^{m}C_{ds}^{m}C_{ds}A_{ds} | 97 |
| | TCTTTGACAA | MOE | | | | | T_{ds}^{m}C_{ds}T_{ds}T_{ds}T_{ds}GₑₒAₑₒ^{m}CₑₛAₑₛAₑ | |
| 1269446 | AGATCACCAT | 5-10-5 | 2227 | 2246 | 46753 | 46772 | AₑₛGₑₒAₑₒTₑₒ^{m}CₑₛA_{ds}^{m}C_{ds}^{m}C_{ds}A_{ds}T_{ds}^{m} | 98 |
| | CTTTGACAAG | MOE | | | | | C_{ds}T_{ds}T_{ds}T_{ds}G_{ds}Aₑₒ^{m}CₑₒAₑₛAₑₛGₑ | |
| 1269447 | GATCACCATCT | 5-10-5 | 2226 | 2245 | 46752 | 46771 | GₑₛAₑₒTₑₒ^{m}CₑₒAₑₛ^{m}C_{ds}^{m}C_{ds}A_{ds}T_{ds}^{m}C_{ds} | 99 |
| | TTGACAAGC | MOE | | | | | T_{ds}T_{ds}T_{ds}G_{ds}A_{ds}^{m}CₑₒAₑₒAₑₛGₑₛ^{m}Cₑ | |
| 1269448 | CACCATCTTTG | 5-10-5 | 2223 | 2242 | 46749 | 46768 | ^{m}CₑₛAₑₒ^{m}Cₑₒ^{m}CₑₒAₑₛT_{ds}^{m}C_{ds}T_{ds}T_{ds}T_{ds} | 100 |
| | ACAAGCTAT | MOE | | | | | G_{ds}A_{ds}^{m}C_{ds}A_{ds}A_{ds}Gₑₒ^{m}CₑₒTₑₛAₑₛTₑ | |
| 1269449 | ACCATCTTTGA | 5-10-5 | 2222 | 2241 | 46748 | 46767 | Aₑₛ^{m}Cₑₒ^{m}CₑₒAₑₒTₑₛ^{m}C_{ds}T_{ds}T_{ds}T_{ds}G_{ds}A_{ds}^{m} | 101 |
| | CAAGCTATA | MOE | | | | | C_{ds}A_{ds}A_{ds}G_{ds}^{m}CₑₒTₑₒAₑₛTₑₛAₑ | |
| 1269465 | TACTAACATC | 5-10-5 | N/A | N/A | 32669 | 32688 | TₑₛAₑₒ^{m}CₑₒTₑₒAₑₛA_{ds}^{m}C_{ds}A_{ds}T_{ds}^{m}C_{ds}A_{ds} | 102 |
| | AGTCACTGAA | MOE | | | | | G_{ds}T_{ds}^{m}C_{ds}A_{ds}^{m}CₑₒTₑₒGₑₛAₑₛAₑ | |
| 1269466 | ATCACTGCAC | 5-10-5 | N/A | N/A | 34020 | 34039 | AₑₛTₑₒ^{m}CₑₒAₑₒ^{m}CₑₛT_{ds}G_{ds}^{m}C_{ds}A_{ds}^{m}C_{ds} | 103 |
| | ACTTTCCTCC | MOE | | | | | A_{ds}^{m}C_{ds}T_{ds}T_{ds}T_{ds}^{m}Cₑₒ^{m}CₑₒTₑₛ^{m}Cₑₛ^{m}Cₑ | |
| 1269467 | CACTGCACAC | 5-10-5 | N/A | N/A | 34018 | 34037 | ^{m}CₑₛAₑₒ^{m}CₑₒTₑₒGₑₛ^{m}C_{ds}A_{ds}^{m}C_{ds}A_{ds}^{m}C_{ds} | 104 |
| | TTTCCTCCTC | MOE | | | | | T_{ds}T_{ds}T_{ds}^{m}C_{ds}^{m}C_{ds}Tₑₒ^{m}Cₑₒ^{m}CₑₛTₑₛ^{m}Cₑ | |
| 1269468 | ACTGCACACTT | 5-10-5 | N/A | N/A | 34017 | 34036 | Aₑₛ^{m}CₑₒTₑₒGₑₒ^{m}CₑₛA_{ds}^{m}C_{ds}A_{ds}^{m}C_{ds}T_{ds} | 105 |
| | TCCTCCTCA | MOE | | | | | T_{ds}T_{ds}^{m}C_{ds}^{m}C_{ds}T_{ds}^{m}Cₑₒ^{m}CₑₒTₑₛ^{m}CₑₛAₑ | |
| 1269469 | CTGCACACTTT | 5-10-5 | N/A | N/A | 34016 | 34035 | ^{m}CₑₛTₑₒGₑₒ^{m}CₑₒAₑₛ^{m}C_{ds}A_{ds}^{m}C_{ds}T_{ds}T_{ds} | 106 |
| | CCTCCTCAA | MOE | | | | | T_{ds}^{m}C_{ds}^{m}C_{ds}T_{ds}^{m}C_{ds}^{m}CₑₒTₑₒ^{m}CₑₛAₑₛAₑ | |
| 1269470 | TGCACACTTTC | 5-10-5 | N/A | N/A | 34015 | 34034 | TₑₛGₑₒ^{m}CₑₒAₑₒ^{m}CₑₛA_{ds}^{m}C_{ds}T_{ds}T_{ds}T_{ds}^{m} | 107 |
| | CTCCTCAAT | MOE | | | | | C_{ds}^{m}C_{ds}T_{ds}^{m}C_{ds}^{m}C_{ds}Tₑₒ^{m}CₑₒAₑₛAₑₛTₑ | |
| 1269471 | CACACTTTCCT | 5-10-5 | N/A | N/A | 34013 | 34032 | ^{m}CₑₛAₑₒ^{m}CₑₒAₑₒ^{m}CₑₛT_{ds}T_{ds}T_{ds}^{m}C_{ds}^{m}C_{ds} | 108 |
| | CCTCAATCA | MOE | | | | | T_{ds}^{m}C_{ds}^{m}C_{ds}T_{ds}^{m}C_{ds}AₑₒAₑₒTₑₛ^{m}CₑₛAₑ | |
| 1269472 | ACACTTTCCTC | 5-10-5 | N/A | N/A | 34012 | 34031 | Aₑₛ^{m}CₑₒAₑₒ^{m}CₑₒTₑₛT_{ds}T_{ds}^{m}C_{ds}^{m}C_{ds}T_{ds}^{m} | 109 |
| | CTCAATCAA | MOE | | | | | C_{ds}^{m}C_{ds}T_{ds}^{m}C_{ds}A_{ds}AₑₒTₑₒ^{m}CₑₛAₑₛAₑ | |
| 1269473 | CACTTTCCTCC | 5-10-5 | N/A | N/A | 34011 | 34030 | ^{m}CₑₛAₑₒ^{m}CₑₒTₑₒTₑₛtT_{ds}^{m}C_{ds}^{m}C_{ds}T_{ds}^{m}C_{ds}^{m} | 110 |
| | TCAATCAAT | MOE | | | | | C_{ds}T_{ds}^{m}C_{ds}A_{ds}A_{ds}Tₑₒ^{m}CₑₒAₑₛAₑₛTₑ | |
| 1269474 | ACTTTCCTCCT | 5-10-5 | N/A | N/A | 34010 | 34029 | Aₑₛ^{m}CₑₒTₑₒTₑₒTₑₛ^{m}C_{ds}^{m}C_{ds}T_{ds}^{m}C_{ds}^{m}C_{ds} | 111 |
| | CAATCAATC | MOE | | | | | T_{ds}^{m}C_{ds}A_{ds}A_{ds}T_{ds}^{m}CₑₒAₑₒAₑₛTₑₛ^{m}Cₑ | |
| 1269475 | CTTTCCTCCTC | 5-10-5 | N/A | N/A | 34009 | 34028 | ^{m}CₑₛTₑₒTₑₒTₑₒ^{m}Cₑₛ^{m}C_{ds}T_{ds}^{m}C_{ds}^{m}C_{ds}T_{ds}^{m} | 112 |
| | AATCAATCC | MOE | | | | | C_{ds}A_{ds}A_{ds}T_{ds}^{m}C_{ds}AₑₒAₑₒTₑₛ^{m}Cₑₛ^{m}Cₑ | |
| 1269476 | TTTCCTCCTCA | 5-10-5 | N/A | N/A | 34008 | 34027 | TₑₛTₑₒTₑₒ^{m}Cₑₒ^{m}CₑₛT_{ds}^{m}C_{ds}^{m}C_{ds}T_{ds}^{m}C_{ds} | 113 |
| | ATCAATCCT | MOE | | | | | A_{ds}A_{ds}T_{ds}^{m}C_{ds}A_{ds}AₑₒTₑₒ^{m}Cₑₛ^{m}CₑₛTₑ | |
| 1269452 | AAATGAGCCA | 5-10-5 | N/A | N/A | 30456 | 30475 | AₑₛAₑₒAₑₒTₑₒGₑₛA_{ds}G_{ds}^{m}C_{ds}^{m}C_{ds}A_{ds}A_{ds} | 114 |
| | ATATTTATAG | MOE | | | | | T_{ds}A_{ds}T_{ds}T_{ds}TₑₒAₑₒTₑₛAₑₛGₑ | |
| 1269453 | AATGAGCCAA | 5-10-5 | N/A | N/A | 30455 | 30474 | AₑₛAₑₒTₑₒGₑₒAₑₛG_{ds}^{m}C_{ds}^{m}C_{ds}A_{ds}A_{ds}T_{ds} | 115 |
| | TATTTATAGG | MOE | | | | | A_{ds}T_{ds}T_{ds}T_{ds}AₑₒTₑₒAₑₛGₑₛGₑ | |
| 1269454 | ATGAGCCAAT | 5-10-5 | N/A | N/A | 30454 | 30473 | AₑₛTₑₒGₑₒAₑₒGₑₛ^{m}C_{ds}^{m}C_{ds}A_{ds}A_{ds}T_{ds}A_{ds} | 116 |
| | ATTTATAGGT | MOE | | | | | T_{ds}T_{ds}T_{ds}A_{ds}TₑₒAₑₒGₑₛGₑₛTₑ | |
| 1269455 | AGCCAATATTT | 5-10-5 | N/A | N/A | 30451 | 30470 | AₑₛGₑₒ^{m}Cₑₒ^{m}CₑₒAₑₛA_{ds}T_{ds}A_{ds}T_{ds}T_{ds}T_{ds} | 117 |
| | ATAGGTGCT | MOE | | | | | A_{ds}T_{ds}A_{ds}G_{ds}GₑₒTₑₒGₑₛ^{m}CₑₛTₑ | |
| 1269456 | GCCAATATTTA | 5-10-5 | N/A | N/A | 30450 | 30469 | Gₑₛ^{m}Cₑₒ^{m}CₑₒAₑₒAₑₛT_{ds}A_{ds}T_{ds}T_{ds}T_{ds}A_{ds} | 118 |
| | TAGGTGCTG | MOE | | | | | T_{ds}A_{ds}G_{ds}G_{ds}TₑₒGₑₒ^{m}CₑₛTₑₛGₑ | |
| 1269457 | CCAATATTTAT | 5-10-5 | N/A | N/A | 30449 | 30468 | ^{m}Cₑₛ^{m}CₑₒAₑₒAₑₒTₑₛA_{ds}T_{ds}T_{ds}T_{ds}A_{ds}T_{ds} | 119 |
| | AGGTGCTGC | MOE | | | | | A_{ds}G_{ds}G_{ds}T_{ds}Gₑₒ^{m}CₑₒTₑₛGₑₛ^{m}Cₑ | |
| 1269458 | CAATATTTATA | 5-10-5 | N/A | N/A | 30448 | 30467 | ^{m}CₑₛAₑₒAₑₒTₑₒAₑₛT_{ds}T_{ds}T_{ds}A_{ds}T_{ds}A_{ds}G_{ds} | 120 |
| | GGTGCTGCT | MOE | | | | | G_{ds}T_{ds}G_{ds}^{m}CₑₒTₑₒGₑₛ^{m}CₑₛTₑ | |
| 1269459 | ATATTTATAGG | 5-10-5 | N/A | N/A | 30446 | 30465 | AₑₛTₑₒAₑₒTₑₒTₑₛT_{ds}A_{ds}T_{ds}A_{ds}G_{ds}G_{ds}T_{ds} | 121 |
| | TGCTGCTAA | MOE | | | | | G_{ds}^{m}C_{ds}T_{ds}Gₑₒ^{m}CₑₒTₑₛAₑₛAₑ | |
| 1287089 | AGTGCTTTTCA | 6-10-4 | N/A | N/A | 15666 | 15685 | AₑₛGₑₒTₑₒGₑₒ^{m}CₑₒTₑₒT_{ds}T_{ds}T_{ds}^{m}C_{ds}A_{ds} | 122 |
| | TACCAGGTC | MOE | | | | | T_{ds}A_{ds}^{m}C_{ds}^{m}C_{ds}A_{ds}GₑₒGₑₛTₑₛ^{m}Cₑ | |
| 1287090 | GCTCATTTATT | 6-10-4 | 1863 | 1882 | 46389 | 46408 | Gₑₛ^{m}CₑₒTₑₒ^{m}CₑₒAₑₒTₑₒT_{ds}T_{ds}A_{ds}T_{ds}T_{ds}^{m} | 58 |
| | CTCAAGTAC | MOE | | | | | C_{ds}T_{ds}^{m}C_{ds}A_{ds}A_{ds}GₑₒTₑₛAₑₛ^{m}Cₑ | |
| 1287091 | GTTCATATCCT | 6-10-4 | N/A | N/A | 32940 | 32959 | GₑₛTₑₒTₑₒ^{m}CₑₒAₑₒTₑₒA_{ds}T_{ds}^{m}C_{ds}^{m}C_{ds}T_{ds} | 123 |
| | TTAACCCAA | MOE | | | | | T_{ds}T_{ds}A_{ds}A_{ds}^{m}C_{ds}^{m}Cₑₒ^{m}CₑₛAₑₛAₑ | |
| 1287092 | TGGCACATTTT | 6-10-4 | N/A | N/A | 37153 | 37172 | TₑₛGₑₒGₑₒ^{m}CₑₒAₑₒ^{m}CₑₒA_{ds}T_{ds}T_{ds}T_{ds}T_{ds} | 60 |
| | TTATAGAGT | MOE | | | | | T_{ds}T_{ds}A_{ds}T_{ds}A_{ds}GₑₒAₑₛGₑₛTₑ | |
| 1287093 | GGCACATTTTT | 6-10-4 | N/A | N/A | 37152 | 37171 | GₑₛGₑₒ^{m}CₑₒAₑₒ^{m}CₑₒAₑₒT_{ds}T_{ds}T_{ds}T_{ds}T_{ds} | 44 |
| | TATAGAGTT | MOE | | | | | T_{ds}A_{ds}T_{ds}A_{ds}G_{ds}AₑₒGₑₛTₑₛTₑ | |
| 1287094 | GCACACTTTCC | 6-10-4 | N/A | N/A | 34014 | 34033 | Gₑₛ^{m}CₑₒAₑₒ^{m}CₑₒAₑₒ^{m}CₑₒT_{ds}T_{ds}T_{ds}^{m}C_{ds}^{m} | 124 |
| | TCCTCAATC | MOE | | | | | C_{ds}T_{ds}^{m}C_{ds}^{m}C_{ds}T_{ds}^{m}C_{ds}AₑₒAₑₛTₑₛ^{m}Cₑ | |
| 1287095 | GCATATTGGTT | 6-10-4 | 3074 | 3093 | 47600 | 47619 | Gₑₛ^{m}CₑₒAₑₒTₑₒAₑₒTₑₒT_{ds}G_{ds}G_{ds}T_{ds}T_{ds}T_{ds} | 50 |
| | TTCTCATTT | MOE | | | | | T_{ds}^{m}C_{ds}T_{ds}^{m}C_{ds}AₑₒTₑₛTₑₛTₑ | |
| 1287096 | GCACCATATA | 6-10-4 | N/A | N/A | 6597 | 6616 | Gₑₛ^{m}CₑₒAₑₒ^{m}Cₑₒ^{m}CₑₒAₑₒT_{ds}A_{ds}T_{ds}A_{ds}T_{ds} | 61 |
| | TATCTCAGAA | MOE | | | | | A_{ds}T_{ds}^{m}C_{ds}T_{ds}^{m}C_{ds}AₑₒGₑₛAₑₛAₑ | |
| 1287098 | GTTAATACTTT | 6-10-4 | N/A | N/A | 19455 | 19474 | GₑₛTₑₒTₑₒAₑₒAₑₒTₑₒA_{ds}^{m}C_{ds}T_{ds}T_{ds}T_{ds}T_{ds} | 62 |
| | TTCCAGCCT | MOE | | | | | T_{ds}^{m}C_{ds}^{m}C_{ds}A_{ds}Gₑₒ^{m}Cₑₛ^{m}CₑₛTₑ | |
| 1287099 | CAGCACCATA | 6-10-4 | N/A | N/A | 6599 | 6618 | ^{m}CₑₛAₑₒGₑₒ^{m}CₑₒAₑₒ^{m}Cₑₒ^{m}C_{ds}A_{ds}T_{ds}A_{ds} | 125 |
| | TATATCTCAG | MOE | | | | | T_{ds}A_{ds}T_{ds}A_{ds}T_{ds}^{m}C_{ds}Tₑₒ^{m}CₑₛAₑₛGₑ | |
| 1287100 | ATGTTCATATC | 6-10-4 | N/A | N/A | 32942 | 32961 | AₑₛTₑₒGₑₒTₑₒTₑₒ^{m}CₑₒA_{ds}T_{ds}A_{ds}T_{ds}^{m}C_{ds}^{m} | 46 |
| | CTTTAACCC | MOE | | | | | C_{ds}T_{ds}T_{ds}T_{ds}A_{ds}Aₑₒ^{m}Cₑₛ^{m}Cₑₛ^{m}Cₑ | |
| 1287101 | GGTCAGTTTCC | 6-10-4 | N/A | N/A | 17180 | 17199 | GₑₛGₑₒTₑₒ^{m}CₑₒAₑₒGₑₒT_{ds}T_{ds}T_{ds}^{m}C_{ds}^{m}C_{ds} | 126 |
| | TAATTTTAA | MOE | | | | | T_{ds}A_{ds}A_{ds}T_{ds}T_{ds}TₑₒTₑₛAₑₛAₑ | |
| 1287102 | TAATACTTTTT | 6-10-4 | N/A | N/A | 19453 | 19472 | TₑₛAₑₒAₑₒTₑₒAₑₒ^{m}CₑₒT_{ds}T_{ds}T_{ds}T_{ds}T_{ds}^{m} | 59 |
| | CCAGCCTTC | MOE | | | | | C_{ds}^{m}C_{ds}A_{ds}G_{ds}^{m}C_{ds}^{m}CₑₒTₑₛTₑₛ^{m}Cₑ | |
| 1287103 | TGCTCATTTAT | 6-10-4 | 1864 | 1883 | 46390 | 46409 | TₑₛGₑₒ^{m}CₑₒTₑₒ^{m}CₑₒAₑₒT_{ds}T_{ds}T_{ds}A_{ds}T_{ds} | 32 |
| | TCTCAAGTA | MOE | | | | | T_{ds}^{m}C_{ds}T_{ds}^{m}C_{ds}A_{ds}AₑₒGₑₛTₑₛAₑ | |
| 1287104 | GCACATTTTTT | 6-10-4 | N/A | N/A | 37151 | 37170 | Gₑₛ^{m}CₑₒAₑₒ^{m}CₑₒAₑₒTₑₒT_{ds}T_{ds}T_{ds}T_{ds}T_{ds} | 17 |
| | ATAGAGTTC | MOE | | | | | A_{ds}T_{ds}A_{ds}G_{ds}A_{ds}GₑₒTₑₛTₑₛ^{m}Cₑ | |
| 1287569 | AATGCTCATTT | 5-10-5 | 1866 | 1885 | 46392 | 46411 | AₑₛAₑₒTₑₒGₑₒ^{m}CₑₛT_{ds}^{m}C_{ds}A_{ds}T_{ds}T_{ds}T_{ds} | 127 |
| | ATTCTCAAG | MOE | | | | | A_{ds}T_{ds}T_{ds}^{m}C_{ds}Tₑₒ^{m}CₑₒAₑₛAₑₛGₑ | |
| 1287570 | ATGCTCATTTA | 5-10-5 | 1865 | 1884 | 46391 | 46410 | AₑₛTₑₒGₑₒ^{m}CₑₒTₑₛ^{m}C_{ds}A_{ds}T_{ds}T_{ds}T_{ds}A_{ds} | 128 |
| | ATTCTCAAG | MOE | | | | | T_{ds}T_{ds}^{m}C_{ds}T_{ds}^{m}CₑₒAₑₒAₑₛGₑₛTₑ | |
| 1287612 | TGGAACTACC | 5-10-5 | 834 | 853 | N/A | N/A | TₑₛGₑₒGₑₒAₑₒAₑₛ^{m}C_{ds}T_{ds}A_{ds}^{m}C_{ds}^{m}C_{ds}T_{ds} | 129 |
| | TTGCATACTT | MOE | | | | | T_{ds}G_{ds}^{m}C_{ds}A_{ds}TₑₒAₑₒ^{m}CₑₛTₑₛTₑ | |
| 1287613 | ACTACCTTGCA | 5-10-5 | 830 | 849 | N/A | N/A | Aₑₛ^{m}CₑₒTₑₒAₑₒ^{m}Cₑₛ^{m}C_{ds}T_{ds}T_{ds}G_{ds}^{m}C_{ds} | 130 |
| | TACTTAGCT | MOE | | | | | A_{ds}T_{ds}A_{ds}^{m}C_{ds}T_{ds}TₑₒAₑₒGₑₛ^{m}CₑₛTₑ | |
| 1287614 | AGTGCTATAA | 5-10-5 | N/A | N/A | 43655 | 43674 | AₑₛGₑₒTₑₒGₑₒ^{m}CₑₛT_{ds}A_{ds}T_{ds}A_{ds}A_{ds}T_{ds}T_{ds}^{m} | 131 |
| | TTCTTGCTTC | MOE | | | | | C_{ds}T_{ds}T_{ds}Gₑₒ^{m}CₑₒTₑₛTₑₛ^{m}Cₑ | |
| 1287615 | GTGCTATAATT | 5-10-5 | N/A | N/A | 43654 | 43673 | GₑₛTₑₒGₑₒ^{m}CₑₒTₑₛA_{ds}T_{ds}A_{ds}A_{ds}T_{ds}T_{ds}^{m} | 132 |
| | CTTGCTTCA | MOE | | | | | C_{ds}T_{ds}T_{ds}G_{ds}^{m}CₑₒTₑₒTₑₛ^{m}CₑₛAₑ | |
| 1287617 | GCTATAATTCT | 5-10-5 | N/A | N/A | 43652 | 43671 | Gₑₛ^{m}CₑₒTₑₒAₑₒTₑₛA_{ds}A_{ds}T_{ds}T_{ds}^{m}C_{ds}T_{ds} | 133 |
| | TGCTTCAAC | MOE | | | | | T_{ds}G_{ds}^{m}C_{ds}T_{ds}Tₑₒ^{m}CₑₒAₑₛAₑₛ^{m}Cₑ | |
| 1287618 | TAATTCTTGCT | 5-10-5 | N/A | N/A | 43648 | 43667 | TₑₛAₑₒAₑₒTₑₒTₑₛ^{m}C_{ds}T_{ds}T_{ds}G_{ds}^{m}C_{ds}T_{ds} | 134 |
| | TCAACCATC | MOE | | | | | T_{ds}^{m}C_{ds}A_{ds}A_{ds}^{m}Cₑₒ^{m}CₑₒAₑₛTₑₛ^{m}Cₑ | |
| 1287619 | AATTCTTGCTT | 5-10-5 | N/A | N/A | 43647 | 43666 | AₑₛAₑₒTₑₒTₑₒ^{m}CₑₛT_{ds}T_{ds}G_{ds}^{m}C_{ds}T_{ds}T_{ds}^{m} | 135 |
| | CAACCATCA | MOE | | | | | C_{ds}A_{ds}A_{ds}^{m}C_{ds}^{m}CₑₒAₑₒTₑₛ^{m}CₑₛAₑ | |
| 1287620 | ATTCTTGCTTC | 5-10-5 | N/A | N/A | 43646 | 43665 | AₑₛTₑₒTₑₒ^{m}CₑₒTₑₛT_{ds}G_{ds}^{m}C_{ds}T_{ds}T_{ds}^{m}C_{ds} | 136 |
| | AACCATCAT | MOE | | | | | A_{ds}A_{ds}^{m}C_{ds}^{m}C_{ds}AₑₒTₑₒ^{m}CₑₛAₑₛTₑ | |
| 1287621 | GCCATTAATCT | 6-10-4 | N/A | N/A | 30607 | 30626 | Gₑₛ^{m}Cₑₒ^{m}CₑₒAₑₒTₑₒTₑₒA_{ds}A_{ds}T_{ds}^{m}C_{ds}T_{ds} | 137 |
| | ATACTGAAT | MOE | | | | | A_{ds}T_{ds}A_{ds}^{m}C_{ds}T_{ds}GₑₒAₑₛAₑₛTₑ | |
| 1304855 | TCAAGTATTTT | 5-10-5 | N/A | N/A | 39752 | 39771 | Tₑₛ^{m}CₑₒAₑₒAₑₒGₑₛT_{ds}A_{ds}T_{ds}T_{ds}T_{ds}T_{ds}T_{ds}^{m} | 141 |
| | TCATTTTCC | MOE | | | | | C_{ds}A_{ds}T_{ds}TₑₒTₑₒTₑₛ^{m}Cₑₛ^{m}Cₑ | |
| 1304856 | GCTGAAGACA | 5-10-5 | N/A | N/A | 59623 | 59642 | Gₑₛ^{m}CₑₒTₑₒGₑₒAₑₛA_{ds}G_{ds}A_{ds}^{m}C_{ds}A_{ds}T_{ds}^{m} | 142 |
| | TCTCTTCCTT | MOE | | | | | C_{ds}T_{ds}^{m}C_{ds}T_{ds}Tₑₒ^{m}Cₑₒ^{m}CₑₛTₑₛTₑ | |
| 1304857 | TCTTCATTAAA | 5-10-5 | N/A | N/A | 40090 | 40109 | Tₑₛ^{m}CₑₒTₑₒTₑₒ^{m}CₑₛA_{ds}T_{ds}T_{ds}A_{ds}A_{ds}A_{ds} | 143 |
| | GCCATACCT | MOE | | | | | G_{ds}^{m}C_{ds}^{m}C_{ds}A_{ds}TₑₒAₑₒ^{m}Cₑₛ^{m}CₑₛTₑ | |
| 1304858 | TTCTTTATATA | 5-10-5 | N/A | N/A | 39897 | 39916 | TₑₛTₑₒ^{m}CₑₒTₑₒTₑₛT_{ds}A_{ds}T_{ds}A_{ds}T_{ds}A_{ds}T_{ds} | 144 |
| | TTCTGCTTA | MOE | | | | | T_{ds}^{m}C_{ds}T_{ds}Gₑₒ^{m}CₑₒTₑₛTₑₛAₑ | |
| 1304859 | TCTTTTCAAAT | 5-10-5 | N/A | N/A | 39955 | 39974 | Tₑₛ^{m}CₑₒTₑₒTₑₒTₑₛT_{ds}^{m}C_{ds}A_{ds}A_{ds}A_{ds}T_{ds}^{m} | 145 |
| | CCTTCACCT | MOE | | | | | C_{ds}^{m}C_{ds}T_{ds}T_{ds}^{m}CₑₒAₑₒ^{m}Cₑₛ^{m}CₑₛTₑ | |
| 1304860 | TCAGTTTTATT | 5-10-5 | N/A | N/A | 40101 | 40120 | Tₑₛ^{m}CₑₒAₑₒGₑₒTₑₛT_{ds}T_{ds}T_{ds}A_{ds}T_{ds}T_{ds}T_{ds}^{m} | 146 |
| | TCTTCATTA | MOE | | | | | C_{ds}T_{ds}T_{ds}^{m}CₑₒAₑₒTₑₛTₑₛAₑ | |
| 1304861 | TGTACACTTTT | 5-10-5 | N/A | N/A | 40173 | 40192 | TₑₛGₑₒTₑₒAₑₒ^{m}CₑₛA_{ds}^{m}C_{ds}T_{ds}T_{ds}T_{ds}T_{ds} | 147 |
| | ACATTCCCA | MOE | | | | | A_{ds}^{m}C_{ds}A_{ds}T_{ds}Tₑₒ^{m}Cₑₒ^{m}Cₑₛ^{m}CₑₛAₑ | |
| 1304862 | CTGTACACTTT | 5-10-5 | N/A | N/A | 40174 | 40193 | ^{m}CₑₛTₑₒGₑₒTₑₒAₑₛ^{m}C_{ds}A_{ds}^{m}C_{ds}T_{ds}T_{ds}T_{ds} | 148 |
| | TACATTCCC | MOE | | | | | T_{ds}A_{ds}^{m}C_{ds}A_{ds}TₑₒTₑₒ^{m}Cₑₛ^{m}Cₑₛ^{m}Cₑ | |
| 1304863 | CCATGACTTCT | 5-10-5 | N/A | N/A | 42638 | 42657 | ^{m}Cₑₛ^{m}CₑₒAₑₒTₑₒGₑₛA_{ds}^{m}C_{ds}T_{ds}T_{ds}^{m}C_{ds} | 149 |
| | TCCTCAATT | MOE | | | | | T_{ds}T_{ds}^{m}C_{ds}^{m}C_{ds}T_{ds}^{m}CₑₒAₑₒAₑₛTₑₛTₑ | |
| 1304864 | CCTCAATTTTT | 5-10-5 | N/A | N/A | 42626 | 42645 | ^{m}Cₑₛ^{m}CₑₒTₑₒ^{m}CₑₒAₑₛA_{ds}T_{ds}T_{ds}T_{ds}T_{ds} | 150 |
| | TTCAGCCCC | MOE | | | | | T_{ds}T_{ds}^{m}C_{ds}A_{ds}Gₑₒ^{m}Cₑₒ^{m}Cₑₛ^{m}Cₑₛ^{m}Cₑ | |
| 1304865 | GTACATTAACT | 5-10-5 | N/A | N/A | 27764 | 27783 | GₑₛTₑₒAₑₒ^{m}CₑₒAₑₛT_{ds}T_{ds}A_{ds}A_{ds}^{m}C_{ds}T_{ds} | 151 |
| | TCCATGAAA | MOE | | | | | T_{ds}^{m}C_{ds}^{m}C_{ds}A_{ds}TₑₒGₑₒAₑₛAₑₛAₑ | |
| 1304866 | CATATTTTACT | 5-10-5 | N/A | N/A | 43580 | 43599 | ^{m}CₑₛAₑₒTₑₒAₑₒTₑₛT_{ds}T_{ds}T_{ds}A_{ds}^{m}C_{ds}T_{ds}^{m} | 152 |
| | CTTTTTATT | MOE | | | | | C_{ds}T_{ds}T_{ds}T_{ds}TₑₒTₑₒAₑₛTₑₛTₑ | |
| 1304867 | GTCACCATACT | 5-10-5 | N/A | N/A | 9019 | 9038 | GₑₛTₑₒ^{m}CₑₒAₑₒ^{m}Cₑₛ^{m}C_{ds}A_{ds}T_{ds}A_{ds}^{m}C_{ds} | 153 |
| | TAATACCAT | MOE | | | | | T_{ds}T_{ds}A_{ds}A_{ds}T_{ds}Aₑₒ^{m}Cₑₒ^{m}CₑₛAₑₛTₑ | |
| 1304868 | TGTACAATTTT | 5-10-5 | N/A | N/A | 58670 | 58689 | TₑₛGₑₒTₑₒAₑₒ^{m}CₑₛA_{ds}A_{ds}T_{ds}T_{ds}T_{ds}T_{ds}^{m} | 154 |
| | CCATTACTA | MOE | | | | | C_{ds}^{m}C_{ds}A_{ds}T_{ds}TₑₒAₑₒ^{m}CₑₛTₑₛAₑ | |
| 1304869 | CCTTATATATT | 5-10-5 | N/A | N/A | 10496 | 10515 | ^{m}Cₑₛ^{m}CₑₒTₑₒTₑₒAₑₛT_{ds}A_{ds}T_{ds}A_{ds}T_{ds}T_{ds} | 155 |
| | TCTACTACC | MOE | | | | | T_{ds}^{m}C_{ds}T_{ds}A_{ds}^{m}CₑₒTₑₒAₑₛ^{m}Cₑₛ^{m}Cₑ | |
| 1304870 | CAGTACCTAA | 5-10-5 | N/A | N/A | 11923 | 11942 | ^{m}CₑₛAₑₒGₑₒTₑₒAₑₛ^{m}C_{ds}^{m}C_{ds}T_{ds}A_{ds}A_{ds}A_{ds} | 156 |
| | AATAAGTTCA | MOE | | | | | A_{ds}T_{ds}A_{ds}A_{ds}GₑₒTₑₒTₑₛ^{m}CₑₛAₑ | |
| 1304871 | TTGTACAATTT | 5-10-5 | N/A | N/A | 58671 | 58690 | TₑₛTₑₒGₑₒTₑₒAₑₛ^{m}C_{ds}A_{ds}A_{ds}T_{ds}T_{ds}T_{ds}T_{ds}^{m} | 157 |
| | TCCATTACT | MOE | | | | | C_{ds}^{m}C_{ds}A_{ds}TₑₒTₑₒAₑₛ^{m}CₑₛTₑ | |
| 1304872 | GCAATGAATA | 5-10-5 | N/A | N/A | 27716 | 27735 | Gₑₛ^{m}CₑₒAₑₒAₑₒTₑₛG_{ds}A_{ds}A_{ds}T_{ds}A_{ds}^{m}C_{ds} | 158 |
| | CAACACACAT | MOE | | | | | A_{ds}A_{ds}^{m}C_{ds}A_{ds}^{m}CₑₒAₑₒ^{m}CₑₛAₑₛTₑ | |
| 1304873 | CGTCTAACATT | 5-10-5 | 720 | 739 | 27577 | 27596 | ^{m}CₑₛGₑₒTₑₒ^{m}CₑₒTₑₛA_{ds}A_{ds}^{m}C_{ds}A_{ds}T_{ds}T_{ds}^{m} | 159 |
| | CCTGAGCCA | MOE | | | | | C_{ds}^{m}C_{ds}T_{ds}G_{ds}AₑₒGₑₒ^{m}Cₑₛ^{m}CₑₛAₑ | |
| 1304874 | CCATCCTTTTC | 5-10-5 | N/A | N/A | 13682 | 13701 | ^{m}Cₑₛ^{m}CₑₒAₑₒTₑₒ^{m}Cₑₛ^{m}C_{ds}T_{ds}T_{ds}T_{ds}T_{ds}^{m} | 160 |
| | TAAATGGTA | MOE | | | | | C_{ds}T_{ds}A_{ds}A_{ds}A_{ds}TₑₒGₑₒGₑₛTₑₛAₑ | |
| 1304875 | TCTTTTATCAT | 5-10-5 | N/A | N/A | 43526 | 43545 | Tₑₛ^{m}CₑₒTₑₒTₑₒTₑₛT_{ds}A_{ds}T_{ds}^{m}C_{ds}A_{ds}T_{ds} | 161 |
| | TTCTTTTCT | MOE | | | | | T_{ds}T_{ds}^{m}C_{ds}T_{ds}TₑₒTₑₒTₑₛ^{m}CₑₛTₑ | |
| 1304876 | AAATTACTTCT | 5-10-5 | N/A | N/A | 43534 | 43553 | AₑₛAₑₒAₑₒTₑₒTₑₛA_{ds}^{m}C_{ds}T_{ds}T_{ds}^{m}C_{ds}T_{ds} | 162 |
| | TTTATCATT | MOE | | | | | T_{ds}T_{ds}T_{ds}A_{ds}Tₑₒ^{m}CₑₒAₑₛTₑₛTₑ | |
| 1304877 | TTAATTTTCCC | 5-10-5 | N/A | N/A | 43450 | 43469 | TₑₛTₑₒAₑₒAₑₒTₑₛT_{ds}T_{ds}T_{ds}^{m}C_{ds}^{m}C_{ds}^{m}C_{ds} | 163 |
| | TTCACTCCT | MOE | | | | | T_{ds}T_{ds}^{m}C_{ds}A_{ds}^{m}CₑₒTₑₒ^{m}Cₑₛ^{m}CₑₛTₑ | |
| 1304878 | CCTGATGTTCC | 5-10-5 | N/A | N/A | 43546 | 43565 | ^{m}Cₑₛ^{m}CₑₒTₑₒGₑₒAₑₛT_{ds}G_{ds}T_{ds}T_{ds}^{m}C_{ds}^{m} | 164 |
| | AAAATTACT | MOE | | | | | C_{ds}A_{ds}A_{ds}A_{ds}A_{ds}TₑₒTₑₒAₑₛ^{m}CₑₛTₑ | |
| 1295851 | AATGCATATT | 5-10-5 | 3077 | 3096 | 47603 | 47622 | AₑₛAₑₒTₑₒGₑₒ^{m}CₑₒA_{ds}T_{ds}A_{ds}T_{ds}T_{ds}G_{ds} | 165 |
| | GGTTTTCTCA | MOE | | | | | G_{ds}T_{ds}T_{ds}T_{ds}Tₑₒ^{m}CₑₒTₑₛ^{m}CₑₛAₑ | |
| 1295852 | GTATAGAGTTT | 5-10-5 | 4142 | 4161 | 48668 | 48687 | GₑₛTₑₒAₑₒTₑₒAₑₒG_{ds}A_{ds}G_{ds}T_{ds}T_{ds}T_{ds}A_{ds}^{m} | 64 |
| | ACCTGCAGC | MOE | | | | | C_{ds}^{m}C_{ds}T_{ds}Gₑₒ^{m}CₑₒAₑₛGₑₛ^{m}Cₑ | |
| 1295853 | CTATAATTCTT | 5-10-5 | N/A | N/A | 43651 | 43670 | ^{m}CₑₛTₑₒAₑₒTₑₒAₑₒA_{ds}T_{ds}T_{ds}^{m}C_{ds}T_{ds}T_{ds} | 166 |
| | GCTTCAACC | MOE | | | | | G_{ds}^{m}C_{ds}T_{ds}T_{ds}^{m}CₑₒAₑₒAₑₛ^{m}Cₑₛ^{m}Cₑ | |
| 1295854 | TTTCATGTTCC | 5-10-5 | 2238 | 2257 | 46764 | 46783 | TₑₛTₑₒTₑₒ^{m}CₑₒAₑₒT_{ds}G_{ds}T_{ds}T_{ds}^{m}C_{ds}^{m}C_{ds} | 167 |
| | AGATCACCA | MOE | | | | | A_{ds}G_{ds}A_{ds}T_{ds}^{m}CₑₒAₑₒ^{m}Cₑₛ^{m}CₑₛAₑ | |
| 1295855 | CCAATATTTAT | 5-10-5 | N/A | N/A | 30449 | 30468 | ^{m}Cₑₛ^{m}CₑₒAₑₒAₑₒTₑₒA_{ds}T_{ds}T_{ds}T_{ds}A_{ds}T_{ds} | 119 |
| | AGGTGCTGC | MOE | | | | | A_{ds}G_{ds}G_{ds}T_{ds}Gₑₒ^{m}CₑₒTₑₛGₑₛ^{m}Cₑ | |
| 1295856 | CAATATTTATA | 5-10-5 | N/A | N/A | 30448 | 30467 | ^{m}CₑₛAₑₒAₑₒTₑₒAₑₒT_{ds}T_{ds}T_{ds}A_{ds}T_{ds}A_{ds} | 120 |
| | GGTGCTGCT | MOE | | | | | G_{ds}G_{ds}T_{ds}G_{ds}^{m}CₑₒTₑₒGₑₛ^{m}CₑₛTₑ | |
| 1295857 | GCCAAAATAC | 5-10-5 | N/A | N/A | 32676 | 32695 | Gₑₛ^{m}Cₑₒ^{m}CₑₒAₑₒAₑₒA_{ds}A_{ds}T_{ds}A_{ds}^{m}C_{ds}T_{ds} | 63 |
| | TAACATCAGT | MOE | | | | | A_{ds}A_{ds}^{m}C_{ds}A_{ds}Tₑₒ^{m}CₑₒAₑₛGₑₛTₑ | |
| 1295858 | GCCAATATTTA | 5-10-5 | N/A | N/A | 30450 | 30469 | Gₑₛ^{m}Cₑₒ^{m}CₑₒAₑₒAₑₒT_{ds}A_{ds}T_{ds}T_{ds}T_{ds}A_{ds} | 118 |
| | TAGGTGCTG | MOE | | | | | T_{ds}A_{ds}G_{ds}G_{ds}TₑₒGₑₒ^{m}CₑₛTₑₛGₑ | |
| 1295859 | GCACCATATA | 5-10-5 | N/A | N/A | 6597 | 6616 | Gₑₛ^{m}CₑₒAₑₒ^{m}Cₑₒ^{m}CₑₒA_{ds}T_{ds}A_{ds}T_{ds}A_{ds}T_{ds} | 61 |
| | TATCTCAGAA | MOE | | | | | A_{ds}T_{ds}^{m}C_{ds}T_{ds}^{m}CₑₒAₑₒGₑₛAₑₛAₑ | |
| 1295860 | AGCCAATATTT | 5-10-5 | N/A | N/A | 30451 | 30470 | AₑₛGₑₒ^{m}Cₑₒ^{m}CₑₒAₑₒA_{ds}T_{ds}A_{ds}T_{ds}T_{ds}T_{ds} | 117 |
| | ATAGGTGCT | MOE | | | | | A_{ds}T_{ds}A_{ds}G_{ds}GₑₒTₑₒGₑₛ^{m}CₑₛTₑ | |
| 1295861 | GTTAATACTTT | 5-10-5 | N/A | N/A | 19455 | 19474 | GₑₛTₑₒTₑₒAₑₒAₑₒT_{ds}A_{ds}^{m}C_{ds}T_{ds}T_{ds}T_{ds}T_{ds} | 62 |
| | TTCCAGCCT | MOE | | | | | T_{ds}^{m}C_{ds}^{m}C_{ds}AₑₒGₑₒ^{m}Cₑₛ^{m}CₑₛTₑ | |
| 1295862 | GTGCTTTTCAT | 5-10-5 | N/A | N/A | 15665 | 15684 | GₑₛTₑₒGₑₒ^{m}CₑₒTₑₒT_{ds}T_{ds}T_{ds}^{m}C_{ds}A_{ds}T_{ds} | 71 |
| | ACCAGGTCT | MOE | | | | | A_{ds}^{m}C_{ds}^{m}C_{ds}A_{ds}GₑₒGₑₒTₑₛ^{m}CₑₛTₑ | |
| 1295863 | TGCTTTTCATA | 5-10-5 | N/A | N/A | 15664 | 15683 | TₑₛGₑₒ^{m}CₑₒTₑₒTₑₒTₑₒT_{ds}T_{ds}^{m}C_{ds}A_{ds}T_{ds} | 72 |
| | CCAGGTCTC | MOE | | | | | A_{ds}^{m}C_{ds}^{m}C_{ds}A_{ds}G_{ds}GₑₒTₑₒ^{m}CₑₛTₑₛ^{m}Cₑ | |
| 1295864 | TGTTAATACTT | 5-10-5 | N/A | N/A | 19456 | 19475 | TₑₛGₑₒTₑₒTₑₒAₑₒA_{ds}T_{ds}A_{ds}^{m}C_{ds}T_{ds}T_{ds} | 75 |
| | TTTCCAGCC | MOE | | | | | T_{ds}T_{ds}T_{ds}^{m}C_{ds}^{m}CₑₒAₑₒGₑₛ^{m}Cₑₛ^{m}Cₑ | |
| 1295865 | TAATACTTTTT | 5-10-5 | N/A | N/A | 19453 | 19472 | TₑₛAₑₒAₑₒTₑₒAₑₒ^{m}C_{ds}T_{ds}T_{ds}T_{ds}T_{ds}T_{ds}^{m} | 59 |
| | CCAGCCTTC | MOE | | | | | C_{ds}^{m}C_{ds}A_{ds}G_{ds}^{m}Cₑₒ^{m}CₑₒTₑₛTₑₛ^{m}Cₑ | |
| 1295866 | GCCATTAATCT | 5-10-5 | N/A | N/A | 30607 | 30626 | Gₑₛ^{m}Cₑₒ^{m}CₑₒAₑₒTₑₒT_{ds}A_{ds}A_{ds}T_{ds}^{m}C_{ds} | 137 |
| | ATACTGAAT | MOE | | | | | T_{ds}A_{ds}T_{ds}A_{ds}^{m}C_{ds}TₑₒGₑₒAₑₛAₑₛTₑ | |
| 1295867 | GGCACATTTTT | 5-10-5 | N/A | N/A | 37152 | 37171 | GₑₛGₑₒ^{m}CₑₒAₑₒ^{m}CₑₒA_{ds}T_{ds}T_{ds}T_{ds}T_{ds}T_{ds} | 44 |
| | TATAGAGTT | MOE | | | | | T_{ds}A_{ds}T_{ds}A_{ds}GₑₒAₑₒGₑₛTₑₛTₑ | |
| 1295868 | GTTCCAGATC | 5-10-5 | 2232 | 2251 | 46758 | 46777 | GₑₛTₑₒTₑₒ^{m}Cₑₒ^{m}CₑₒA_{ds}G_{ds}A_{ds}T_{ds}^{m}C_{ds} | 168 |
| | ACCATCTTTG | MOE | | | | | A_{ds}^{m}C_{ds}^{m}C_{ds}A_{ds}T_{ds}^{m}CₑₒTₑₒTₑₛTₑₛGₑ | |
| 1295869 | ACCCAATAAT | 5-10-5 | N/A | N/A | 32926 | 32945 | Aₑₛ^{m}Cₑₒ^{m}Cₑₒ^{m}CₑₒAₑₒA_{ds}T_{ds}A_{ds}A_{ds}T_{ds}^{m} | 42 |
| | CTGACATCCT | MOE | | | | | C_{ds}T_{ds}G_{ds}A_{ds}^{m}C_{ds}AₑₒTₑₒ^{m}Cₑₛ^{m}CₑₛTₑ | |
| 1295870 | TGGCACATTTT | 5-10-5 | N/A | N/A | 37153 | 37172 | TₑₛGₑₒGₑₒ^{m}CₑₒAₑₒ^{m}C_{ds}A_{ds}T_{ds}T_{ds}T_{ds}T_{ds} | 60 |
| | TTATAGAGT | MOE | | | | | T_{ds}T_{ds}A_{ds}T_{ds}AₑₒGₑₒAₑₛGₑₛTₑ | |
| 1295871 | TGCTCATTTAT | 5-10-5 | 1864 | 1883 | 46390 | 46409 | TₑₛGₑₒ^{m}CₑₒTₑₒ^{m}CₑₒA_{ds}T_{ds}T_{ds}T_{ds}A_{ds}T_{ds} | 32 |
| | TCTCAAGTA | MOE | | | | | T_{ds}^{m}C_{ds}T_{ds}^{m}C_{ds}AₑₒAₑₒGₑₛTₑₛAₑ | |
| 1295872 | GCTCATTTATT | 5-10-5 | 1863 | 1882 | 46389 | 46408 | Gₑₛ^{m}CₑₒTₑₒ^{m}CₑₒAₑₒT_{ds}T_{ds}T_{ds}A_{ds}T_{ds}T_{ds}^{m} | 58 |
| | CTCAAGTAC | MOE | | | | | C_{ds}T_{ds}^{m}C_{ds}A_{ds}AₑₒGₑₒTₑₛAₑₛ^{m}Cₑ | |
| 1295873 | TGGCACATTTT | 5-10-5 | N/A | N/A | 37153 | 37172 | TₑₛGₑₛGₑₒ^{m}CₑₒAₑₛ^{m}C_{ds}A_{ds}T_{ds}T_{ds}T_{ds}T_{ds} | 60 |
| | TTATAGAGT | MOE | | | | | T_{ds}T_{ds}A_{ds}T_{ds}AₑₒGₑₒAₑₛGₑₛTₑ | |
| 1295874 | AGCCAATATTT | 5-10-5 | N/A | N/A | 30451 | 30470 | AₑₛGₑₛ^{m}Cₑₒ^{m}CₑₒAₑₛA_{ds}T_{ds}A_{ds}T_{ds}T_{ds}T_{ds} | 117 |
| | ATAGGTGCT | MOE | | | | | A_{ds}T_{ds}A_{ds}G_{ds}GₑₒTₑₒGₑₛ^{m}CₑₛTₑ | |
| 1295875 | GCACCATATA | 5-10-5 | N/A | N/A | 6597 | 6616 | Gₑₛ^{m}CₑₛAₑₒ^{m}Cₑₒ^{m}CₑₛA_{ds}T_{ds}A_{ds}T_{ds}A_{ds}T_{ds} | 61 |
| | TATCTCAGAA | MOE | | | | | A_{ds}T_{ds}^{m}C_{ds}T_{ds}^{m}CₑₒAₑₒGₑₛAₑₛAₑ | |
| 1295876 | TGTTAATACTT | 5-10-5 | N/A | N/A | 19456 | 19475 | TₑₛGₑₛTₑₒTₑₒAₑₛA_{ds}T_{ds}A_{ds}^{m}C_{ds}T_{ds}T_{ds}T_{ds} | 75 |
| | TTTCCAGCC | MOE | | | | | T_{ds}T_{ds}^{m}C_{ds}^{m}CₑₒAₑₒGₑₛ^{m}Cₑₛ^{m}Cₑ | |
| 1295877 | GTTAATACTTT | 5-10-5 | N/A | N/A | 19455 | 19474 | GₑₛTₑₛtTₑₒAₑₒAₑₛT_{ds}A_{ds}^{m}C_{ds}T_{ds}T_{ds}T_{ds}T_{ds} | 62 |
| | TTCCAGCCT | MOE | | | | | T_{ds}^{m}C_{ds}^{m}C_{ds}AₑₒGₑₒ^{m}Cₑₛ^{m}CₑₛTₑ | |
| 1295878 | TGCTCATTTAT | 5-10-5 | 1864 | 1883 | 46390 | 46409 | TₑₛGₑₛ^{m}CₑₒTₑₒ^{m}CₑₛA_{ds}T_{ds}T_{ds}T_{ds}A_{ds}T_{ds} | 32 |
| | TCTCAAGTA | MOE | | | | | T_{ds}^{m}C_{ds}T_{ds}^{m}C_{ds}AₑₒAₑₒGₑₛTₑₛAₑ | |
| 1295879 | TAATACTTTTT | 5-10-5 | N/A | N/A | 19453 | 19472 | TₑₛAₑₛAₑₒTₑₒAₑₛ^{m}C_{ds}T_{ds}T_{ds}T_{ds}T_{ds}T_{ds}^{m} | 59 |
| | CCAGCCTTC | MOE | | | | | C_{ds}^{m}C_{ds}A_{ds}G_{ds}^{m}Cₑₒ^{m}CₑₒTₑₛTₑₛ^{m}Cₑ | |
| 1295880 | ACCCAATAAT | 5-10-5 | N/A | N/A | 32926 | 32945 | Aₑₛ^{m}Cₑₛ^{m}Cₑₒ^{m}CₑₒAₑₛA_{ds}T_{ds}A_{ds}A_{ds}T_{ds}^{m} | 42 |
| | CTGACATCCT | MOE | | | | | C_{ds}T_{ds}G_{ds}A_{ds}^{m}C_{ds}AₑₒTₑₒ^{m}Cₑₛ^{m}CₑₛTₑ | |
| 1295881 | GCCATTAATCT | 5-10-5 | N/A | N/A | 30607 | 30626 | Gₑₛ^{m}Cₑₛ^{m}CₑₒAₑₒTₑₛT_{ds}A_{ds}A_{ds}T_{ds}^{m}C_{ds}T_{ds} | 137 |
| | ATACTGAAT | MOE | | | | | A_{ds}T_{ds}A_{ds}^{m}C_{ds}TₑₒGₑₒAₑₛAₑₛTₑ | |
| 1295882 | GCTCATTTATT | 5-10-5 | 1863 | 1882 | 46389 | 46408 | Gₑₛ^{m}CₑₛTₑₒ^{m}CₑₒAₑₛT_{ds}T_{ds}T_{ds}A_{ds}T_{ds}T_{ds}^{m} | 58 |
| | CTCAAGTAC | MOE | | | | | C_{ds}T_{ds}^{m}C_{ds}A_{ds}AₑₒGₑₒTₑₛAₑₛ^{m}Cₑ | |
| 1295883 | GGCACATTTTT | 5-10-5 | N/A | N/A | 37152 | 37171 | GₑₛGₑₛ^{m}CₑₒAₑ^{m}CₑₛA_{ds}T_{ds}T_{ds}T_{ds}T_{ds}T_{ds} | 44 |
| | TATAGAGTT | MOE | | | | | T_{ds}A_{ds}T_{ds}A_{ds}GₑₒAₑₒGₑₛTₑₛTₑ | |
| 1299093 | ACCATCTTTGA | 5-10-5 | 2222 | 2241 | 46748 | 46767 | Aₑₛ^{m}Cₑₒ^{m}CₑₒAₑₒTₑₒ^{m}C_{ds}T_{ds}T_{ds}T_{ds}G_{ds} | 101 |
| | CAAGCTATA | MOE | | | | | A_{ds}^{m}C_{ds}A_{ds}A_{ds}G_{ds}^{m}CₑₒTₑₒAₑₛTₑₛAₑ | |
| 1299091 | CCCCAAACTTT | 5-10-5 | 313 | 332 | 16179 | 16198 | ^{m}Cₑₛ^{m}Cₑₒ^{m}Cₑₒ^{m}CₑₒAₑₒA_{ds}A_{ds}^{m}C_{ds}T_{ds}T_{ds} | 171 |
| | CAAGGCATT | MOE | | | | | T_{ds}^{m}C_{ds}A_{ds}A_{ds}G_{ds}Gₑₒ^{m}CₑₒAₑₛTₑₛTₑ | |
| 1299092 | GTTCACTTTGC | 5-10-5 | N/A | N/A | 8455 | 8474 | GₑₛTₑₒTₑₒ^{m}CₑₒAₑₒ^{m}C_{ds}T_{ds}T_{ds}T_{ds}G_{ds}^{m}C_{ds}^{m} | 172 |
| | CATAATCAA | MOE | | | | | C_{ds}A_{ds}T_{ds}A_{ds}AₑₒTₑₒ^{m}CₑₛAₑₛAₑ | |

### Example 2

### Design of Altered Gapmers having a 2′-O-Methyl Nucleoside in the Gap and Mixed PO/PS Internucleoside Linkages Complementary to Human ATXN3 RNA

Modified oligonucleotides complementary to a human ATXN3 nucleic acid were designed. The modified oligonucleotides in the table below are 5-10-5 altered gapmers. The altered gapmers have a central gap segment that comprises 2′-deoxynucleosides and is flanked by wing segments on both the 5′ end and on the 3′ end comprising 2′-MOE nucleosides. The gap contains one 2′-O-methyl nucleoside. The internucleoside linkages throughout each gapmer are mixed phosphodiester internucleoside linkages and phosphorothioate internucleoside linkages. Internucleoside linkage motifs include, in order from 5′ to 3′: sooosssssssssssooss and sossssssssssssssoss. The sequence and chemical notation column specifies the sequence, including 5-methyl cytosines, sugar chemistry, and the internucleoside linkage chemistry; wherein subscript ‘d’ represents a 2′-β-D-deoxyribosyl sugar moiety, subscript ‘e’ represents a 2′-MOE sugar moiety, subscript ‘y’ represents a 2′-O-methyl sugar moiety, subscript ‘o’ represents a phosphodiester internucleoside linkage, subscript ‘s’ refers represents to a phosphorothioate internucleoside linkage, and superscript ‘m’ before the cytosine residue represents a 5-methyl cytosine. “Start site” indicates the 5′-most nucleoside to which the gapmer is complementary in the human nucleic acid sequence. “Stop site” indicates the 3′-most nucleoside to which the gapmer is complementary in the human nucleic acid sequence.

Each modified oligonucleotide listed in the table below is complementary to human ATXN3 nucleic acid sequence SEQ ID NO: 1 (GENBANK Accession No: NM_004993.5), SEQ ID NO: 2 (the complement of GENBANK Accession No NC_000014.9 truncated from nucleotides 92,056,001 to 92,110,000), or SEQ ID NO: 3 (the complement of GENBANK Accession No NC_000014.9 truncated from nucleotides 92038001 to 92110000), as indicated. ‘N/A’ indicates that the modified oligonucleotide is not 100% complementary to that particular nucleic acid.

TABLE 2
Altered gapmers having a 2′-O-methyl nucleoside in the gap and
mixed PO/PS internucleoside linkages complementary to human
ATXN3 RNA

| | | | SEQ | SEQ | SEQ | SEQ | | |
|---|---|---|---|---|---|---|---|---|
| | | | ID | ID | ID | ID | | |
| | | | NO: 1 | NO: 1 | NO: 2 | NO: 2 | | SEQ |
| Compound | Sequence | Gapmer | Start | Stop | Start | Stop | Chemistry Notation | ID |
| Number | (5′ to 3′) | Motif | Site | Site | Site | Site | (5′ to 3′) | NO |
| 1288220 | TAATACUTTTTC | 5-10-5 | N/A | N/A | 19453 | 19472 | TₑₛAₑₒAₑₒTₑₒAₑₛ^{m}C_{ds}U_{ys}T_{ds}T_{ds}T_{ds}T_{ds}^{m} | 138 |
| | CAGCCTTC | MOE | | | | | C_{ds}^{m}C_{ds}A_{ds}G_{ds}^{m}Cₑₒ^{m}CₑₒTₑₛTₑₛ^{m}Cₑ | |
| 1288221 | AGTGCTUTTCA | 5-10-5 | N/A | N/A | 15666 | 15685 | AₑₛGₑₒTₑₒGₑₒ^{m}CₑₛT_{ds}U_{ys}T_{ds}T_{ds}^{m}C_{ds}A_{ds} | 139 |
| | TACCAGGTC | MOE | | | | | T_{ds}A_{ds}^{m}C_{ds}^{m}C_{ds}AₑₒGₑₒGₑₛTₑₛ^{m}Cₑ | |
| 1288222 | GGTTTTCTCATT | 5-10-5 | 3067 | 3084 | 47593 | 47612 | GₑₛGₑₒTₑₒTₑₒTₑₛT_{ds}C_{ys}T_{ds}^{m}C_{ds}A_{ds}T_{ds}T_{ds} | 37 |
| | TTTATATT | MOE | | | | | T_{ds}T_{ds}T_{ds}AₑₒTₑₒAₑₛTₑₛTₑ | |
| 1288223 | TGGCACATTTTT | 5-10-5 | N/A | N/A | 37153 | 37172 | TₑₛGₑₒGₑₒ^{m}CₑₒAₑₛC_{ys}A_{ds}T_{ds}T_{ds}T_{ds}T_{ds}T_{ds} | 60 |
| | TATAGAGT | MOE | | | | | T_{ds}A_{ds}T_{ds}AₑₒGₑₒAₑₛGₑₛTₑ | |
| 1288287 | TAATACTTUTTC | 5-10-5 | N/A | N/A | 19453 | 19472 | TₑₛAₑₒAₑₒTₑₒAₑₛ^{m}C_{ds}T_{ds}T_{ds}U_{ys}T_{ds}T_{ds}^{m} | 140 |
| | CAGCCTTC | MOE | | | | | C_{ds}^{m}C_{ds}A_{ds}G_{ds}^{m}Cₑₒ^{m}CₑₒTₑₛTₑₛ^{m}Cₑ | |
| 1288288 | AGTGCTTTTCAT | 5-10-5 | N/A | N/A | 15666 | 15685 | AₑₛGₑₒTₑₒGₑₒ^{m}CₑₛT_{ds}T_{ds}T_{ds}T_{ds}C_{ys}A_{ds} | 122 |
| | ACCAGGTC | MOE | | | | | T_{ds}A_{ds}^{m}C_{ds}^{m}C_{ds}AₑₒGₑₒGₑₛTₑₛ^{m}Cₑ | |
| 1288289 | TGGCACATTTTT | 5-10-5 | N/A | N/A | 37153 | 37172 | TₑₛGₑₒGₑₒ^{m}CₑₒAₑₛ^{m}C_{ds}A_{ys}T_{ds}T_{ds}T_{ds}T_{ds} | 60 |
| | TATAGAGT | MOE | | | | | T_{ds}T_{ds}A_{ds}T_{ds}AₑₒGₑₒAₑₛGₑₛTₑ | |
| 1299087 | GTTAATACTTTT | 5-10-5 | N/A | N/A | 19455 | 19474 | GₑₛTₑₒTₑₛAₑₛAₑₛT_{ds}A_{ys}^{m}C_{ds}T_{ds}T_{ds}T_{ds}T_{ds} | 62 |
| | TCCAGCCT | MOE | | | | | T_{ds}^{m}C_{ds}^{m}C_{ds}AₑₛGₑₒ^{m}Cₑₛ^{m}CₑₛTₑ | |
| 1299090 | TAATACUTTTTC | 5-10-5 | N/A | N/A | 19453 | 19472 | TₑₛAₑₒAₑₛTₑₛAₑₛ^{m}C_{ds}U_{ys}T_{ds}T_{ds}T_{ds}T_{ds}^{m} | 138 |
| | CAGCCTTC | MOE | | | | | C_{ds}^{m}C_{ds}A_{ds}G_{ds}^{m}Cₑₛ^{m}CₑₒTₑₛTₑₛ^{m}Cₑ | |
| 1299089 | TGCTTTUCATA | 5-10-5 | N/A | N/A | 15664 | 15683 | TₑₛGₑₒ^{m}CₑₒTₑₒTₑₛT_{ds}U_{ys}^{m}C_{ds}A_{ds}T_{ds}A_{ds}^{m} | 169 |
| | CCAGGTCTC | MOE | | | | | C_{ds}^{m}C_{ds}A_{ds}G_{ds}GₑₒTₑₒ^{m}CₑₛTₑₛ^{m}Cₑ | |
| 1299088 | GTGCTTUTCAT | 5-10-5 | N/A | N/A | 15665 | 15684 | GₑₛTₑₒGₑₒ^{m}CₑₒTₑₛT_{ds}U_{ys}T_{ds}^{m}C_{ds}A_{ds}T_{ds} | 170 |
| | ACCAGGTCT | MOE | | | | | A_{ds}^{m}C_{ds}^{m}C_{ds}A_{ds}GₑₒGₑₒTₑₛ^{m}CₑₛTₑ | |

### Example 3

### Activity of Modified Oligonucleotides Complementary to Human ATXN3 RNA in Transgenic Mice

Modified oligonucleotides were tested in the ATXN3 YAC transgenic mouse model which contains the full-length human ATXN3 disease gene harboring an expanded CAG repeat (CAG₈₄, Q84). The hemizygous SCA3-Q84.2 mice are designated as wt/Q84 and were described in Costa Mdo C., et al., Toward RNAi Therapy for the Polyglutamine Disease Machado-Joseph Disease. Mol Ther, 2013. 21 (10): 1898-908.

The ATXN3 transgenic mice were divided into groups of 2 or 3 mice each. Mice in each group were given a single ICV bolus of oligonucleotide at a dose of 300 μg and sacrificed two weeks later. A group of 2 or 3 mice was injected with PBS and served as the control group to which oligonucleotide-treated groups were compared. After two weeks, mice were sacrificed, and RNA was extracted from various regions of the central nervous system. ATXN3 RNA levels were measured by quantitative real-time RTPCR using human primer probe set RTS43981 (forward sequence TGACACAGACATCAGGTACAAATC, designated herein as SEQ ID NO: 4; reverse sequence TGCTGCTGTTGCTGCTT, designated herein as SEQ ID NO: 5; probe sequence AGCTTCGGAAGAGACGAGAAGCCTA, designated herein as SEQ ID NO: 6). The expression level of ATXN3 RNA was normalized to that of the house keeping gene cyclophilin-A RNA using mouse primer probe set m_cyclo24 ((forward sequence TCGCCGCTTGCTGCA, designated herein as SEQ ID NO: 7; reverse sequence ATCGGCCGTGATGTCGA, designated herein as SEQ ID NO: 8; probe sequence CCATGGTCAACCCCACCGTGTTC, designated herein as SEQ ID NO: 9), and this was further normalized to the group mean of vehicle control (PBS) treated animals Expression data are reported as percent mean vehicle-treated control group. Comparator Compound No. 650528 was also tested in this assay. As shown in the tables below, human ATXN3 RNA was reduced in various tissues. Each of Tables 3-15 represents a different experiment.

TABLE 3
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 650528 | 29 | 47 | 80 | 37 |
| 1248258 | 71 | 85 | 104 | 87 |
| 1248259 | 64 | 67 | 79 | 60 |
| 1248261 | 74 | 98 | 97 | 85 |
| 1248262 | 73 | 93 | 102 | 86 |
| 1248264 | 73 | 90 | 99 | 92 |
| 1248265 | 85 | 93 | 105 | 102 |
| 1248266 | 29 | 50 | 66 | 30 |
| 1248267 | 80 | 108 | 99 | 84 |
| 1248268 | 82 | 92 | 97 | 90 |
| 1248269 | 73 | 101 | 89 | 84 |
| 1248271 | 66 | 82 | 83 | 76 |
| 1248273 | 62 | 66 | 99 | 63 |
| 1248275 | 59 | 72 | 90 | 70 |
| 1248276 | 98 | 85 | 99 | 97 |
| 1248277 | 72 | 78 | 99 | 79 |
| 1248278 | 49 | 54 | 93 | 47 |

TABLE 4
Reduction of human ATXN3 RNA in transgenic mice

| Compound | hATXN3 Expression (% control) | | | |
|---|---|---|---|---|
| Number | Spinal cord | Cortex | Cerebellum | Brain stem |
| PBS | 100 | 100 | 100 | 100 |
| 650528 | 30 | 42 | 67 | 34 |
| 1248257 | 45 | 48 | 64 | 46 |
| 1248260 | 42 | 41 | 66 | 44 |
| 1248263 | 29 | 33 | 52 | 28 |
| 1248270 | 59 | 61 | 68 | 47 |
| 1248272 | 90 | 93 | 86 | 90 |
| 1248274 | 28 | 29 | 60 | 29 |
| 1248279 | 69 | 86 | 74 | 60 |
| 1248280 | 66 | 83 | 72 | 57 |
| 1248281 | 60 | 53 | 67 | 43 |
| 1248282 | 84 | 76 | 87 | 67 |
| 1248283 | 25 | 20 | 54 | 23 |
| 1248284 | 29 | 32 | 73 | 35 |
| 1248285 | 65 | 66 | 74 | 65 |
| 1248286 | 65 | 75 | 89 | 76 |
| 1248287 | 46 | 44 | 63 | 43 |
| 1248288 | 44 | 29 | 67 | 40 |
| 1248289 | 58 | 50 | 79 | 52 |
| 1248290 | 23 | 34 | 60 | 20 |
| 1248291 | 52 | 72 | 84 | 68 |
| 1248292 | 33 | 31 | 70 | 27 |
| 1248293 | 75 | 83 | 90 | 79 |
| 1248297 | 62 | 66 | 65 | 62 |
| 1248298 | 80 | 84 | 79 | 74 |

TABLE 5
Reduction of human ATXN3 RNA in transgenic mice

| Compound | hATXN3 Expression (% control) | | | |
|---|---|---|---|---|
| Number | Spinal cord | Cortex | Cerebellum | Brain stem |
| PBS | 100 | 100 | 100 | 100 |
| 650528 | 39 | 61 | 81 | 38 |
| 1247564 | 27 | 36 | 58 | 29 |
| 1247565 | 28 | 32 | 57 | 28 |
| 1247566 | 23 | 33 | 53 | 26 |
| 1247567 | 59 | 68 | 79 | 62 |
| 1247568 | 30 | 27 | 51 | 29 |
| 1248294 | 62 | 93 | 86 | 79 |
| 1248295 | 58 | 84 | 83 | 55 |
| 1248296 | 66 | 73 | 81 | 70 |
| 1248299 | 84 | 93 | 94 | 84 |
| 1248300 | 70 | 86 | 82 | 70 |

TABLE 6
Reduction of human ATXN3 RNA in transgenic mice

| Compound | hATXN3 Expression (% control) | | | |
|---|---|---|---|---|
| Number | Spinal cord | Cortex | Cerebellum | Brain stem |
| PBS | 100 | 100 | 100 | 100 |
| 650528 | 36 | 47 | 81 | 44 |
| 1269632 | 16 | 11 | 64 | 18 |
| 1269633 | 25 | 14 | 62 | 28 |
| 1269634 | 26 | 30 | 74 | 26 |
| 1269635 | 26 | 21 | 59 | 25 |
| 1269636 | 21 | 16 | 59 | 22 |
| 1269637 | 27 | 34 | 68 | 32 |
| 1269638 | 49 | 27 | 79 | 51 |
| 1269639 | 33 | 35 | 68 | 35 |
| 1269640 | 42 | 34 | 69 | 41 |

TABLE 7
Reduction of human ATXN3 RNA in transgenic mice

| Compound | hATXN3 Expression (% control) | | | |
|---|---|---|---|---|
| Number | Spinal cord | Cortex | Cerebellum | Brain stem |
| PBS | 100 | 100 | 100 | 100 |
| 650528 | 28 | 43 | 76 | 32 |
| 1269481 | 61 | 75 | 84 | 62 |
| 1269482 | 43 | 71 | 80 | 52 |
| 1269483 | 37 | 63 | 87 | 46 |
| 1269484 | 44 | 69 | 83 | 57 |
| 1269485 | 18 | 14 | 54 | 14 |
| 1269486 | 24 | 26 | 62 | 23 |
| 1269487 | 67 | 61 | 95 | 60 |
| 1269488 | 65 | 75 | 109 | 60 |
| 1269495 | 26 | 21 | 57 | 26 |
| 1269496 | 47 | 38 | 82 | 44 |
| 1269633 | 19 | 14 | 59 | 23 |
| 1269636 | 22 | 15 | 60 | 20 |
| 1269640 | 37 | 34 | 73 | 41 |

TABLE 8
Reduction of human ATXN3 RNA in transgenic mice

| Compound | hATXN3 Expression (% control) | | | |
|---|---|---|---|---|
| Number | Spinal cord | Cortex | Cerebellum | Brain stem |
| PBS | 100 | 100 | 100 | 100 |
| 650528 | 40 | 33 | 78 | 44 |
| 1269450 | 68 | 62 | 89 | 70 |
| 1269451 | 70 | 74 | 101 | 85 |
| 1269460 | 51 | 56 | 106 | 63 |
| 1269461 | 67 | 81 | 125 | 77 |
| 1269462 | 59 | 61 | 98 | 67 |
| 1269463 | 61 | 65 | 100 | 72 |
| 1269464 | 76 | 79 | 117 | 95 |
| 1269477 | 71 | 60 | 75 | 66 |
| 1269478 | 43 | 52 | 91 | 47 |
| 1269479 | 51 | 47 | 83 | 58 |
| 1269480 | 46 | 49 | 78 | 54 |
| 1269489 | 53 | 55 | 82 | 66 |
| 1269490 | 53 | 55 | 96 | 63 |
| 1269491 | 50 | 58 | 91 | 62 |
| 1269492 | 47 | 53 | 83 | 57 |
| 1269493 | 42 | 45 | 95 | 43 |
| 1269494 | 42 | 35 | 79 | 42 |
| 1269635 | 26 | 18 | 58 | 28 |
| 1269637 | 25 | 24 | 81 | 30 |

TABLE 9
Reduction of human ATXN3 RNA in transgenic mice

| Compound | hATXN3 Expression (% control) | | | |
|---|---|---|---|---|
| Number | Spinal cord | Cortex | Cerebellum | Brain stem |
| PBS | 100 | 100 | 100 | 100 |
| 650528 | 28 | 49 | 68 | 40 |
| 1269442 | 41 | 28 | 63 | 46 |
| 1269443 | 58 | 48 | 66 | 51 |
| 1269444 | 51 | 43 | 65 | 49 |
| 1269445 | 44 | 49 | 58 | 57 |
| 1269446 | 64 | 59 | 72 | 70 |
| 1269447 | 56 | 48 | 77 | 62 |
| 1269448 | 40 | 35 | 60 | 46 |
| 1269449 | 38 | 28 | 50 | 43 |
| 1269465 | 106 | 87 | 54 | 85 |
| 1269466 | 56 | 39 | 61 | 54 |
| 1269467 | 61 | 41 | 57 | 53 |
| 1269468 | 41 | 28 | 59 | 36 |
| 1269469 | 44 | 32 | 54 | 40 |
| 1269470 | 44 | 42 | 66 | 49 |
| 1269471 | 57 | 46 | 53 | 51 |
| 1269472 | 90 | 60 | 65 | 69 |
| 1269473 | 79 | 64 | 66 | 73 |
| 1269474 | 63 | 60 | 71 | 65 |
| 1269475 | 101 | 81 | 71 | 77 |
| 1269476 | 131 | 74 | 64 | 95 |

TABLE 10
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 650528 | 38 | 39 | 80 | 31 |
| 1269452 | 85 | 84 | 94 | 69 |
| 1269453 | 64 | 74 | 87 | 50 |
| 1269454 | 43 | 48 | 75 | 33 |
| 1269455 | 18 | 20 | 58 | 14 |
| 1269456 | 17 | 15 | 55 | 14 |
| 1269457 | 30 | 27 | 70 | 27 |
| 1269458 | 40 | 31 | 77 | 29 |
| 1269459 | 58 | 61 | 95 | 47 |

TABLE 11
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 650528 | 49 | 49 | 56 | 45 |
| 1287089 | 27 | 24 | 38 | 14 |
| 1287090 | 12 | 12 | 32 | 12 |
| 1287091 | 24 | 17 | 39 | 19 |
| 1287092 | 26 | 36 | 44 | 19 |
| 1287093 | 37 | 30 | 53 | 14 |
| 1287094 | 32 | 41 | 47 | 32 |
| 1287095 | 24 | 17 | 27 | 15 |
| 1287096 | 31 | 34 | 31 | 19 |
| 1287098 | 38 | 35 | 67 | 29 |
| 1287099 | 32 | 38 | 34 | 21 |
| 1287100 | 49 | 28 | 49 | 17 |
| 1287101 | 28 | 46 | 43 | 29 |
| 1287102 | 50 | 61 | 72 | 59 |
| 1287103 | 22 | 19 | 37 | 21 |
| 1287104 | 40 | 33 | 57 | 22 |
| 1287569 | 69 | 68 | 50 | 46 |
| 1287570 | 26 | 34 | 42 | 23 |
| 1287612 | 34 | 62 | 48 | 40 |
| 1287613 | 65 | 63 | 48 | 48 |
| 1287614 | 37 | 55 | 54 | 31 |
| 1287615 | 42 | 45 | 48 | 29 |
| 1287617 | 48 | 26 | 39 | 18 |
| 1287618 | 41 | 55 | 53 | 38 |
| 1287619 | 42 | 42 | 61 | 50 |
| 1287620 | 58 | 76 | 64 | 55 |
| 1287621 | 33 | 27 | 51 | 29 |

TABLE 12
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 1288220 | 38 | 21 | 62 | 41 |
| 1288221 | 34 | 28 | 64 | 42 |
| 1288222 | 63 | 61 | 90 | 83 |
| 1288223 | 35 | 31 | 75 | 39 |
| 1288287 | 48 | 20 | 63 | 46 |
| 1288288 | 23 | 14 | 55 | 26 |
| 1288289 | 46 | 35 | 82 | 47 |

TABLE 13
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 650528 | 70 | 43 | 84 | 41 |
| 1304855 | 80 | 58 | 81 | 53 |
| 1304856 | 104 | 102 | 99 | 98 |
| 1304857 | 105 | 61 | 83 | 64 |
| 1304858 | 82 | 59 | 86 | 65 |
| 1304859 | 94 | 64 | 88 | 77 |
| 1304860 | 98 | 82 | 95 | 75 |
| 1304861 | 42 | 27 | 61 | 38 |
| 1304862 | 22 | 17 | 39 | 21 |
| 1304863 | 66 | 46 | 81 | 52 |
| 1304864 | 49 | 45 | 70 | 44 |
| 1304865 | 108 | 94 | 106 | 80 |
| 1304866 | 127 | 106 | 118 | 105 |
| 1304867 | 72 | 72 | 107 | 59 |
| 1304868 | 127 | 98 | 122 | 99 |
| 1304869 | 96 | 65 | 113 | 75 |
| 1304870 | 117 | 93 | 118 | 95 |
| 1304871 | 106 | 107 | 120 | 100 |
| 1304872 | 115 | 109 | 107 | 103 |
| 1304873 | 53 | 42 | 85 | 47 |
| 1304874 | 89 | 103 | 105 | 94 |
| 1304875 | 75 | 66 | 94 | 72 |
| 1304876 | 129 | 114 | 107 | 107 |
| 1304877 | 90 | 84 | 94 | 84 |
| 1304878 | 86 | 87 | 97 | 80 |

TABLE 14
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 650528 | 45 | 40 | 42 | 86 |
| 1295851 | 31 | 40 | 40 | 72 |
| 1295852 | 40 | 49 | 47 | 75 |
| 1295853 | 33 | 42 | 42 | 74 |
| 1295854 | 21 | 36 | 40 | 71 |
| 1295855 | 33 | 38 | 36 | 65 |
| 1295856 | 32 | 37 | 44 | 68 |
| 1295857 | 41 | 47 | 46 | 68 |
| 1295858 | 29 | 31 | 31 | 60 |
| 1295859 | 25 | 29 | 32 | 65 |
| 1295860 | 27 | 25 | 24 | 61 |
| 1295861 | 26 | 32 | 27 | 64 |
| 1295862 | 18 | 17 | 18 | 50 |
| 1295863 | 19 | 30 | 33 | 54 |
| 1295864 | 23 | 41 | 44 | 66 |
| 1295865 | 43 | 44 | 42 | 68 |
| 1295866 | 33 | 41 | 48 | 66 |
| 1295867 | 32 | 37 | 40 | 81 |
| 1295868 | 33 | 41 | 42 | 78 |
| 1295869 | 54 | 51 | 60 | 80 |
| 1295870 | 26 | 23 | 26 | 59 |
| 1295871 | 31 | 34 | 33 | 70 |
| 1295872 | 25 | 24 | 27 | 64 |
| 1295873 | 23 | 26 | 27 | 72 |
| 1295874 | 14 | 17 | 18 | 62 |
| 1295875 | 27 | 28 | 30 | 55 |
| 1295876 | 21 | 32 | 33 | 55 |
| 1295877 | 55 | 28 | 45 | 86 |
| 1295878 | 23 | 50 | 32 | 64 |
| 1295879 | 24 | 35 | 33 | 74 |
| 1295880 | 39 | 56 | 59 | 89 |
| 1295881 | 53 | 60 | 56 | 84 |
| 1295882 | 20 | 28 | 30 | 69 |
| 1295883 | 39 | 42 | 41 | 71 |

TABLE 15
Reduction of human ATXN3 RNA in transgenic mice

| | hATXN3 Expression (% control) | | | |
|---|---|---|---|---|
| Compound | Spinal | | | Brain |
| Number | cord | Cortex | Cerebellum | stem |
| PBS | 100 | 100 | 100 | 100 |
| 650528 | 41 | 44 | 82 | 45 |
| 1299087 | 37 | 22 | 68 | 35 |
| 1299088 | 30 | 28 | 64 | 29 |
| 1299089 | 46 | 44 | 74 | 43 |
| 1299090 | 31 | 20 | 63 | 33 |
| 1299091 | 55 | 53 | 75 | 57 |
| 1299092 | 43 | 40 | 76 | 49 |
| 1299093 | 52 | 51 | 81 | 59 |

### Example 4

### Potency of Modified Oligonucleotides Complementary to Human ATXN3 in Transgenic Mice

Modified oligonucleotides were tested in the ATXN3 YAC transgenic mouse model which contains the full-length human ATXN3 disease gene harboring an expanded CAG repeat (CAG₈₄, Q84). The hemizygous SCA3-Q84.2 mice are designated as wt/Q84 and were described in Costa Mdo C., et al., Toward RNAi Therapy for the Polyglutamine Disease Machado-Joseph Disease. Mol Ther, 2013. 21 (10): 1898-908.

### Treatment

The ATXN3 transgenic mice were divided into groups of 4 mice each. Each mouse received a single ICV bolus of modified oligonucleotide at the doses indicated in tables below. A group of 4 mice received PBS as a negative control.

### RNA Analysis

Two weeks post treatment, mice were sacrificed, and RNA was extracted from cortical brain tissue, brain stem, and spinal cord for real-time qPCR analysis of RNA expression of ATXN3 using primer probe set RTS43981 (described herein above). The expression level of ATXN3 RNA was normalized to that of the house keeping gene cyclophilin-A mRNA using mouse primer probe set m_cyclo24 (described herein above), and this was further normalized to the group mean of vehicle control treated animals Expression data are reported as percent mean vehicle-treated control group (%control). ED₅₀ were calculated from log transformed dose and individual animal ATXN3 RNA levels using the built in GraphPad formula “log(agonist) vs. response—Find ECanything.

As shown in the table below, treatment with modified oligonucleotides resulted in dose-responsive reduction of ATXN3 RNA in comparison to the PBS control. Each of Tables 16-18 represents a different experiment.

TABLE 16
Reduction of human ATXN3 RNA in transgenic mice

| | | Spinal Cord | | Cortex | | Brainstem | |
|---|---|---|---|---|---|---|---|
| | | hATXN3 | | hATXN3 | | hATXN3 | |
| Compound | Dose | Expression | ED₅₀ | Expression | ED₅₀ | Expression | ED₅₀ |
| Number | (μg) | (% control) | (μg) | (% control) | (μg) | (% control) | (μg) |
| 1248274 | 10 | 84 | 38.4 | 93 | 86.9 | 94 | 111.2 |
| | 30 | 63 | | 82 | | 71 | |
| | 100 | 36 | | 48 | | 51 | |
| | 300 | 25 | | 27 | | 51 | |
| | 700 | 22 | | 22 | | 37 | |

TABLE 17
Reduction of human ATXN3 RNA in transgenic mice

| | | Spinal Cord | | Cortex | | Brainstem | |
|---|---|---|---|---|---|---|---|
| | | hATXN3 | | hATXN3 | | hATXN3 | |
| Compound | Dose | Expression | ED₅₀ | Expression | ED₅₀ | Expression | ED₅₀ |
| Number | (μg) | (% control) | (μg) | (% control) | (μg) | (% control) | (μg) |
| 12694555 | 10 | 71 | 15.7 | 81 | 61.2 | 75 | 20.2 |
| | 30 | 39 | | 83 | | 48 | |
| | 100 | 19 | | 38 | | 25 | |
| | 300 | 15 | | 19 | | 17 | |
| | 700 | 14 | | 11 | | 15 | |
| 1287089 | 10 | 80 | 19.6 | 87 | 58.3 | 76 | 27.8 |
| | 30 | 41 | | 64 | | 53 | |
| | 100 | 24 | | 48 | | 31 | |
| | 300 | 18 | | 19 | | 22 | |
| | 700 | 14 | | 12 | | 19 | |
| 1287090 | 10 | 71 | 14.5 | 88 | 39.8 | 70 | 19.8 |
| | 30 | 33 | | 63 | | 50 | |
| | 100 | 21 | | 32 | | 31 | |
| | 300 | 14 | | 15 | | 18 | |
| | 700 | 10 | | 10 | | 15 | |
| 1287621 | 10 | 82 | 23.1 | 69 | 36.7 | 79 | 34.8 |
| | 30 | 45 | | 64 | | 61 | |
| | 100 | 31 | | 42 | | 40 | |
| | 300 | 19 | | 21 | | 22 | |
| | 700 | 15 | | 15 | | 18 | |

TABLE 18
Reduction of human ATXN3 RNA in transgenic mice

| | | Spinal Cord | | Cortex | | Brainstem | |
|---|---|---|---|---|---|---|---|
| | | hATXN3 | | hATXN3 | | hATXN3 | |
| Compound | Dose | Expression | ED₅₀ | Expression | ED₅₀ | Expression | ED₅₀ |
| Number | (μg) | (% control) | (μg) | (% control) | (μg) | (% control) | (μg) |
| 1269635 | 10 | 72 | 32.1 | 77 | 56.8 | 69 | 33.4 |
| | 30 | 71 | | 72 | | 68 | |
| | 100 | 27 | | 46 | | 39 | |
| | 300 | 22 | | 26 | | 28 | |
| | 700 | 18 | | 15 | | 24 | |
| 1287091 | 10 | 52 | 10.4 | 79 | 57.4 | 61 | 18.2 |
| | 30 | 56 | | 82 | | 59 | |
| | 100 | 22 | | 35 | | 29 | |
| | 300 | 34 | | 22 | | 42 | |
| | 700 | 14 | | 12 | | 18 | |
| 1287095 | 10 | 69 | 14.5 | 84 | 28.9 | 71 | 21.2 |
| | 30 | 37 | | 68 | | 51 | |
| | 100 | 21 | | 37 | | 32 | |
| | 300 | 18 | | 21 | | 24 | |
| | 700 | 15 | | 11 | | 19 | |
| 1287103 | 10 | 80 | 30.2 | 88 | 72.2 | 71 | 23.5 |
| | 30 | 58 | | 85 | | 52 | |
| | 100 | 34 | | 40 | | 39 | |
| | 300 | 20 | | 27 | | 26 | |
| | 700 | 15 ^{Δ} | | 11 ^{Δ} | | 20 ^{Δ} | |

^{Δ} Indicates that the group had less than 4 animals

### Example 5

### Effect of 5-10-5 Gapmers with Mixed Internucleoside Linkages on Human ATXN3 In Vitro, Multiple Doses

Modified oligonucleotides selected from the examples above were tested at various doses in A431 cells by free uptake. Cells were plated at a density of 11,000 cells per well, and treated with 109.4 nM, 437.5 nM, 1,750.0 nM, and 7,000.0 nM concentrations of modified oligonucleotide, as specified in the tables below. After a treatment period of approximately 48 hours, total RNA was isolated from the cells and ATXN3 RNA levels were measured by RT-qPCR. Human primer probe set RTS38920 (forward sequence CTATCAGGACAGAGTTCACATCC, designated herein as SEQ ID NO: 173; reverse sequence GTTTCTAAAGACATGGTCACAGC, designated herein as SEQ ID NO: 174; probe sequence AAAGGCCAGCCACCAGTTCAGG, designated herein as SEQ ID: 175) was used to measure RNA levels. Comparator Compound No. 650528 was also tested in this assay. ATXN3 RNA levels were adjusted according to total RNA content, as measured by RiboGreen®. Results are presented in the table below as percent ATXN3 RNA levels relative to untreated control cells. IC₅₀ was calculated using the “log(inhibitor) vs. normalized response—variable slope” formula using Prism7.01 software.

TABLE 19
Dose-dependent reduction of human ATXN3 RNA by modified oligonucleotides

| Compound | % control | | | | |
|---|---|---|---|---|---|
| Number | 109.4 nM | 437.5 nM | 1750.0 nM | 7000.0 nM | IC₅₀ (μM) |
| 650528 | 38 | 48 | 67 | 84 | 2.03 |
| 1269455 | 5 | 9 | 19 | 47 | 0.09 |
| 1269635 | 8 | 15 | 33 | 55 | 0.15 |
| 1287095 | 8 | 10 | 17 | 32 | 0.02 |
| 1287621 | 20 | 35 | 58 | 85 | 0.8 |

### Example 6

### Tolerability of Modified Oligonucleotides Complementary to Human ATXN3 in Wild-Type Mice

Modified oligonucleotides described above were tested in wild-type female C57/B16 mice to assess the tolerability of the oligonucleotides. Wild-type female C57/B16 mice each received a single ICV dose of 700 μg of modified oligonucleotide listed in the table below. Each treatment group consisted of 4 mice. A group of 4 mice received PBS as a negative control. At 3 hours post-injection, mice were evaluated according to 7 different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB score). After all 7 criteria were evaluated, the scores were summed for each mouse and averaged within each treatment group. The results are presented in the table below.

TABLE 20
FOB scores in wild-type mice

| Compound | 3 hour |
|---|---|
| Number | FOB |
| PBS | 0.00 |
| 1248263 | 0.00 |
| 1248274 | 0.00 |
| 1248283 | 2.25 |
| 1248284 | 0.00 |
| 1248287 | 1.50 |
| 1248288 | 0.00 |
| 1248290 | 2.25 |
| 1248292 | 0.00 |

TABLE 21
FOB scores in wild-type mice

| Compound | 3 hour |
|---|---|
| Number | FOB |
| PBS | 0.00 |
| 1247564 | 2.50 |
| 1247565 | 3.00 |
| 1247566 | 3.00 |
| 1247568 | 5.50 |

TABLE 22
FOB scores in wild-type mice

| Compound | 3 hour |
|---|---|
| Number | FOB |
| PBS | 0.00 |
| 1269485 | 1.00 |
| 1269486 | 0.25 |
| 1269493 | 0.00 |
| 1269494 | 0.00 |
| 1269495 | 0.00 |
| 1269632 | 2.00 |
| 1269633 | 1.25 |
| 1269634 | 6.00 |
| 1269635 | 0.00 |
| 1269636 | 1.00 |
| 1269637 | 2.50 |
| 1269639 | 4.00 |
| 1269640 | 0.25 |

TABLE 23
FOB scores in wild-type mice

| Compound | 3 hour |
|---|---|
| Number | FOB |
| PBS | 0.00 |
| 1287089 | 1.00 |
| 1287091 | 0.25 |
| 1287092 | 2.75 |
| 1287093 | 1.00 |
| 1287094 | 0.00 |
| 1287095 | 3.00 |
| 1287096 | 0.50 |
| 1287098 | 1.00 |
| 1287099 | 1.00 |
| 1287100 | 0.00 |
| 1287101 | 1.00 |
| 1287102 | 3.00 |
| 1287103 | 0.00 |
| 1287104 | 1.00 |
| 1287569 | 2.00 |
| 1287570 | 0.00 |
| 1287612 | 5.25 |
| 1287613 | 1.00 |
| 1287614 | 0.50 |
| 1287615 | 2.50 |
| 1287617 | 1.00 |
| 1287618 | 1.00 |
| 1287619 | 1.75 |
| 1287620 | 0.50 |
| 1287621 | 0.00 |

TABLE 24
FOB scores in wild-type mice

| Compound | 3 hour |
|---|---|
| Number | FOB |
| PBS | 0.00 |
| 1269442 | 1.00 |
| 1269448 | 1.25 |
| 1269449 | 2.75 |
| 1269454 | 1.00 |
| 1269455 | 1.00 |
| 1269456 | 3.00 |
| 1269457 | 3.50 |
| 1269458 | 4.50 |
| 1269466 | 0.00 |
| 1269468 | 0.00 |
| 1269469 | 0.00 |
| 1269470 | 0.50 |
| 1269471 | 0.00 |

TABLE 25
FOB scores in wild-type mice

| Compound | 3 hour |
|---|---|
| Number | FOB |
| PBS | 0.00 |
| 1287090 | 0.00 |
| 1287096 | 0.00 |
| 1287099 | 0.25 |
| 1287612 | 5.50 |
| 1287613 | 1.25 |
| 1287614 | 0.00 |
| 1287615 | 0.00 |
| 1287617 | 2.50 |
| 1287618 | 1.75 |
| 1287619 | 0.75 |
| 1287620 | 0.25 |
| 1287750 | 0.00 |

TABLE 26
FOB scores in wild-type mice

| Compound | 3 hour |
|---|---|
| Number | FOB |
| PBS | 0.00 |
| 1288220 | 1.00 |
| 1288221 | 0.75 |
| 1288222 | 0.00 |
| 1288223 | 1.50 |
| 1288287 | 1.00 |
| 1288288 | 0.25 |
| 1288289 | 1.25 |

TABLE 27
FOB scores in wild-type mice

| Compound | 3 hour |
|---|---|
| Number | FOB |
| PBS | 0.00 |
| 1295851 | 4.00 |
| 1295854 | 1.00 |
| 1295855 | 1.75 |
| 1295856 | 4.00 |
| 1295858 | 1.50 |
| 1295859 | 1.00 |
| 1295860 | 1.00 |
| 1295861 | 1.00 |
| 1295862 | 1.50 |
| 1295863 | 1.25 |
| 1295864 | 1.00 |
| 1295866 | 1.00 |
| 1295867 | 1.25 |
| 1295868 | 3.50 |
| 1295870 | 2.50 |
| 1295871 | 1.00 |
| 1295872 | 1.00 |
| 1295873 | 3.25 |
| 1295874 | 1.00 |
| 1295875 | 1.00 |
| 1295876 | 1.00 |
| 1295878 | 1.00 |
| 1295879 | 1.50 |
| 1295882 | 1.00 |
| 1304861 | 1.00 |
| 1304862 | 1.00 |

1. An oligomeric compound comprising a modified oligonucleotide consisting of 12 to 50 linked nucleosides wherein the nucleobase sequence of the modified oligonucleotide is at least 90% complementary to an equal length portion of an ATXN3 nucleic acid, and wherein the modified oligonucleotide comprises at least one modification selected from a modified sugar moiety and a modified internucleoside linkage.

2. An oligomeric compound comprising a modified oligonucleotide consisting of 12 to 50 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of any of the nucleobase sequences of SEQ ID NOs: 11-172, wherein the modified oligonucleotide comprises at least one modification selected from a modified sugar moiety and a modified internucleoside linkage.

3. The oligomeric compound of claim 1 or claim 2, wherein the modified oligonucleotide consists of 15, 16, 17, 18, 19, or 20 linked nucleosides and has a nucleobase sequence comprising at least 15, at least 16, at least 17, at least 18, at least 19, or 20 contiguous nucleobases of any of the nucleobase sequences of SEQ ID NOs: 11-172.

4. The oligomeric compound of claim 3, wherein the modified oligonucleotide consists of 18, 19, or 20 linked nucleosides.

5. The oligomeric compound of any of claims 1-4, wherein the modified oligonucleotide has a nucleobase sequence that is at least 90%, at least 95%, or 100% complementary to an equal length portion of an ATXN 3 nucleic acid when measured across the entire nucleobase sequence of the modified oligonucleotide.

6. The oligomeric compound of any of claims 1-5, wherein the modified oligonucleotide has a nucleobase sequence comprising a portion of at least 8, at least 9, at least 10, at least 11, at least 12, at least 13, at least 14, at least 15, at least 16, at least 17, at least 18, at least 19, or 20 contiguous nucleobases, wherein the portion is complementary to:
an equal length portion of nucleobases 6,597-6,619 of SEQ ID NO: 2;
an equal length portion of nucleobases 15,664-15,689 of SEQ ID NO: 2;
an equal length portion of nucleobases 19,451-19,476 of SEQ ID NO: 2;
an equal length portion of nucleobases 30,448-30,473 of SEQ ID NO: 2;
an equal length portion of nucleobases 32,940-32,961 of SEQ ID NO: 2;
an equal length portion of nucleobases 34,013-34,039 of SEQ ID NO: 2;
an equal length portion of nucleobases 37,151-37,172 of SEQ ID NO: 2;
an equal length portion of nucleobases 43,647-43,674 of SEQ ID NO: 2;
an equal length portion of nucleobases 46,389-46,411 of SEQ ID NO: 2;
an equal length portion of nucleobases 46,748-46,785 of SEQ ID NO: 2; or
an equal length portion of nucleobases 47,594-47,619 of SEQ ID NO: 2.

7. The oligomeric compound of any one of claims 1-6, wherein the ATXN3 nucleic acid has the nucleobase sequence of any of SEQ ID NOs: 1, 2, or 3.

8. The oligomeric compound of any of claims 1-7, wherein the modified oligonucleotide comprises at least one modified sugar moiety.

9. The oligomeric compound of any of claims 8-10, wherein the modified oligonucleotide comprises at least one bicyclic sugar moiety.

10. The oligomeric compound of claim 9, wherein the bicyclic sugar moiety has a 4′-2′ bridge, wherein the 4′-2′ bridge is selected from —CH₂—O—-; and —CH(CH₃)—O—.

11. The oligomeric compound of claim 8, wherein the modified oligonucleotide comprises at least one non-bicyclic modified sugar moiety.

12. The oligomeric compound of claim 11, wherein the non-bicyclic modified sugar moiety is any of a 2′-MOE sugar moiety or a 2′-OMe sugar moiety.

13. The oligomeric compound of claim 12, wherein each modified nucleoside of the modified oligonucleotide comprises a modified non-bicyclic sugar moiety comprising a 2′-MOE sugar moiety or a 2′-OMe sugar moiety.

14. The oligomeric compound of claim 12, wherein each modified sugar moiety is a 2′-MOE sugar moiety.

15. The oligomeric compound of any of claims 8-12, wherein the modified oligonucleotide comprises at least one sugar surrogate.

16. The oligomeric compound of claim 15, wherein the sugar surrogate is any of morpholino, modified morpholino, PNA, THP, and F-HNA.

17. The oligomeric compound of any of claims 1-12 and 15-16, wherein the modified oligonucleotide is a gapmer or an altered gapmer.

18. The oligomeric compound of any of claims 1-12 and 15-17, wherein the modified oligonucleotide has a sugar motif comprising:
a 5′-region consisting of 1-6 linked 5′-nucleosides;
a central region consisting of 6-10 linked central region nucleosides; and
a 3′-region consisting of 1-5 linked 3′-nucleosides; wherein each of the 5′-region nucleosides and each of the 3′-region nucleosides comprises a modified sugar moiety and each of the central region nucleosides comprises a 2′-β-D—deoxyribosyl sugar moiety.

19. The oligomeric compound of claim 18, wherein the modified sugar moiety is a 2′-MOE sugar moiety.

20. The oligomeric compound of any of claims 1-12 and 15-17, wherein the modified oligonucleotide has a sugar motif comprising:
a 5′-region consisting of 1-6 linked 5′-nucleosides, each comprising a 2′-MOE sugar moiety;
a 3′-region consisting of 1-5 linked 3′-nucleosides, each comprising a 2′-MOE sugar moiety; and
a central region consisting of 6-10 linked central region nucleosides, wherein one of the central region nucleosides comprises a 2′-O-methyl sugar moiety and the remainder of the central region nucleosides each comprise a 2′-β-D-deoxyribosyl sugar moiety.

21. The oligomeric compound of claim 20, wherein the central region has the following formula (5′-3′):
(N_{d})(N_{y})(N_{d})ₙ, wherein N_{y} is a nucleoside comprising a 2′-O-methyl sugar moiety and each N_{d} is a nucleoside comprising a 2′-β-D-deoxyribosyl sugar moiety, and n is 10.

22. The oligomeric compound of any of claims 1-21, wherein the modified oligonucleotide comprises at least one modified internucleoside linkage.

23. The oligomeric compound of claim 22, wherein each internucleoside linkage of the modified oligonucleotide is a modified internucleoside linkage.

24. The oligomeric compound of claim 22 or claim 23, wherein at least one internucleoside linkage is a phosphorothioate internucleoside linkage.

25. The oligomeric compound of claim 22 or claim 24 wherein the modified oligonucleotide comprises at least one phosphodiester internucleoside linkage.

26. The oligomeric compound of any of claim 22 or 24-25, wherein each internucleoside linkage is either a phosphodiester internucleoside linkage or a phosphorothioate internucleoside linkage.

27. The oligomeric compound of claim 23, wherein each internucleoside linkage is a phosphorothioate internucleoside linkage.

28. The oligomeric compound of claim 1-22 or 24-25, wherein the modified oligonucleotide has an internucleoside linkage motif (5′ to 3′) selected from among: sooooossssssssssoss, soooossssssssssooos, soooossssssssssooss, sooosssssssssooss, sooossssssssssooss, sooosssssssssssooos, sooosssssssssssooss, sossssssssssssssoss, and ssoosssssssssssooss; wherein,
s=a phosphorothioate internucleoside linkage, and
o=a phosphodiester internucleoside linkage.

29. The oligomeric compound of any of claims 1-28, wherein the modified oligonucleotide comprises at least one modified nucleobase.

30. The oligomeric compound of claim 29, wherein the modified nucleobase is a 5-methyl cytosine.

31. The oligomeric compound of any one of claims 1-30, wherein the modified oligonucleotide consists of 12-22, 12-20, 14-20, 16-20, 18-20, or 18-22 linked nucleosides.

32. The oligomeric compound of any one of claims 1-30, wherein the modified oligonucleotide consists of 16, 17, 18, 19, or 20 linked nucleosides.

33. An oligomeric compound comprising a modified oligonucleotide according to the following chemical notation:
AₑₛGₑₒ^{m}Cₑₒ^{m}CₑₒAₑₛA_{ds}T_{ds}A_{ds}T_{ds}T_{ds}T_{ds}A_{ds}T_{ds}A_{ds}G_{ds}GₑₒTₑₒGₑₛ^{m}CₑₛTₑ (SEQ ID NO: 117), wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

34. An oligomeric compound comprising a modified oligonucleotide according to the following chemical notation:
Gₑₛ^{m}Cₑₒ^{m}CₑₒAₑₒTₑₒTₑₒA_{ds}A_{ds}T_{ds}^{m}C_{ds}T_{ds}A_{ds}T_{ds}A_{ds}^{m}C_{ds}T_{ds}GₑₒAₑₛAₑₛTₑ (SEQ ID NO: 137), wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

35. An oligomeric compound comprising a modified oligonucleotide according to the following chemical notation:
Ges^{m}CₑₒAₑₒTₑₒAₑₒTₑₒT_{ds}G_{ds}G_{ds}T_{ds}T_{ds}T_{ds}T_{ds}^{m}C_{ds}T_{ds}^{m}C_{ds}AₑₒTₑₛTₑₛTₑ (SEQ ID NO: 50), wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

36. The oligomeric compound of any of claims 1-35, wherein the oligomeric compound is a singled-stranded oligomeric compound.

37. The oligomeric compound of any of claims 1-36 consisting of the modified oligonucleotide.

38. The oligomeric compound of any of claims 1-37 comprising a conjugate group comprising a conjugate moiety and a conjugate linker.

39. The oligomeric compound of claim 38, wherein the conjugate group comprises a GalNAc cluster comprising 1-3 GalNAc ligands

40. The oligomeric compound of claim 38 or claim 39, wherein the conjugate linker consists of a single bond.

41. The oligomeric compound of claim 38, wherein the conjugate linker is cleavable.

42. The oligomeric compound of claim 38, wherein the conjugate linker comprises 1-3 linker-nucleosides.

43. The oligomeric compound of any of claims 38-42, wherein the conjugate group is attached to the modified oligonucleotide at the 5′-end of the modified oligonucleotide.

44. The oligomeric compound of any of claims 38-42, wherein the conjugate group is attached to the modified oligonucleotide at the 3′-end of the modified oligonucleotide.

45. The oligomeric compound of any of claim 1-36 or 38-44 comprising a terminal group.

46. The oligomeric compound of any of claim 1-41 or 43-45, wherein the oligomeric compound does not comprise linker-nucleosides.

47. A modified oligonucleotide according to the following chemical structure: 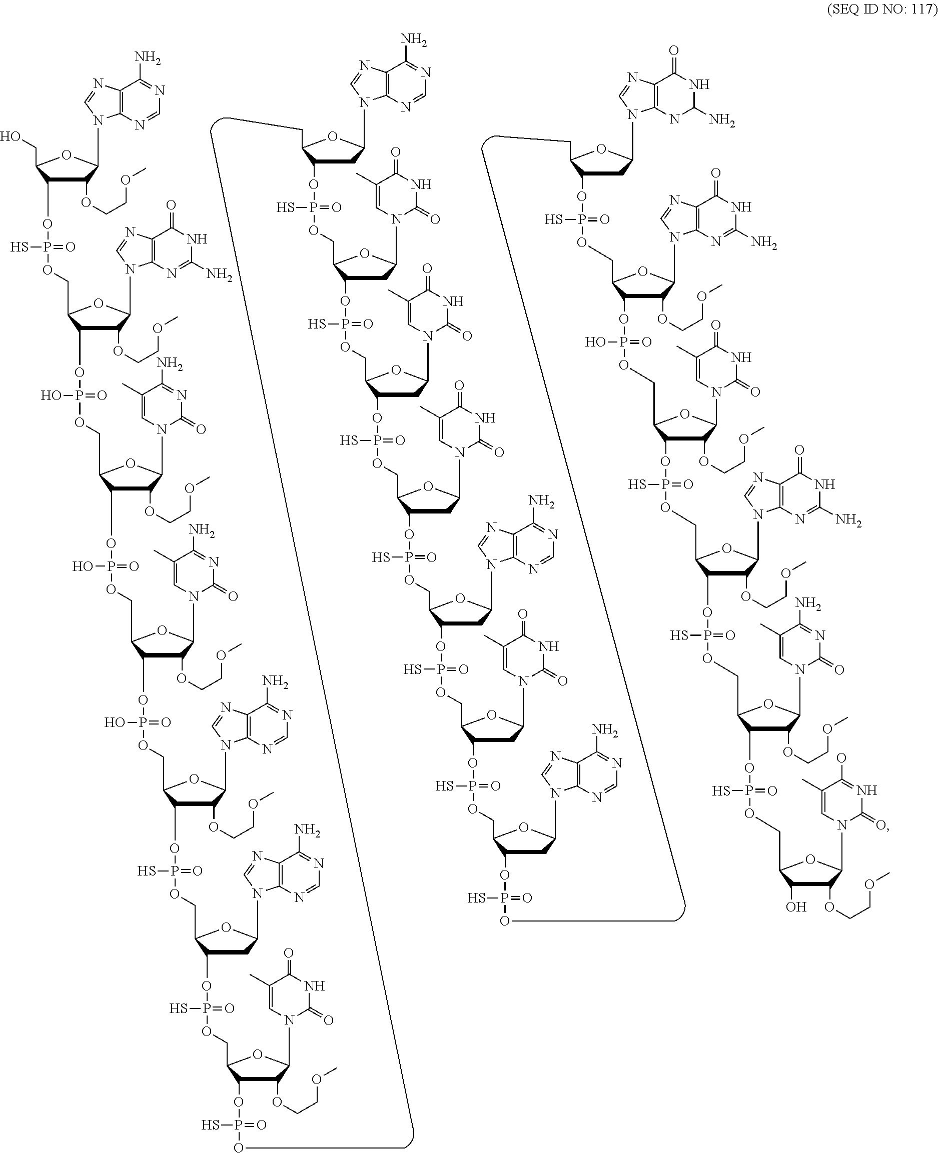
or a salt thereof.

48. The modified oligonucleotide of claim 47, which is the sodium salt or the potassium salt.

49. A modified oligonucleotide according to the following formula:

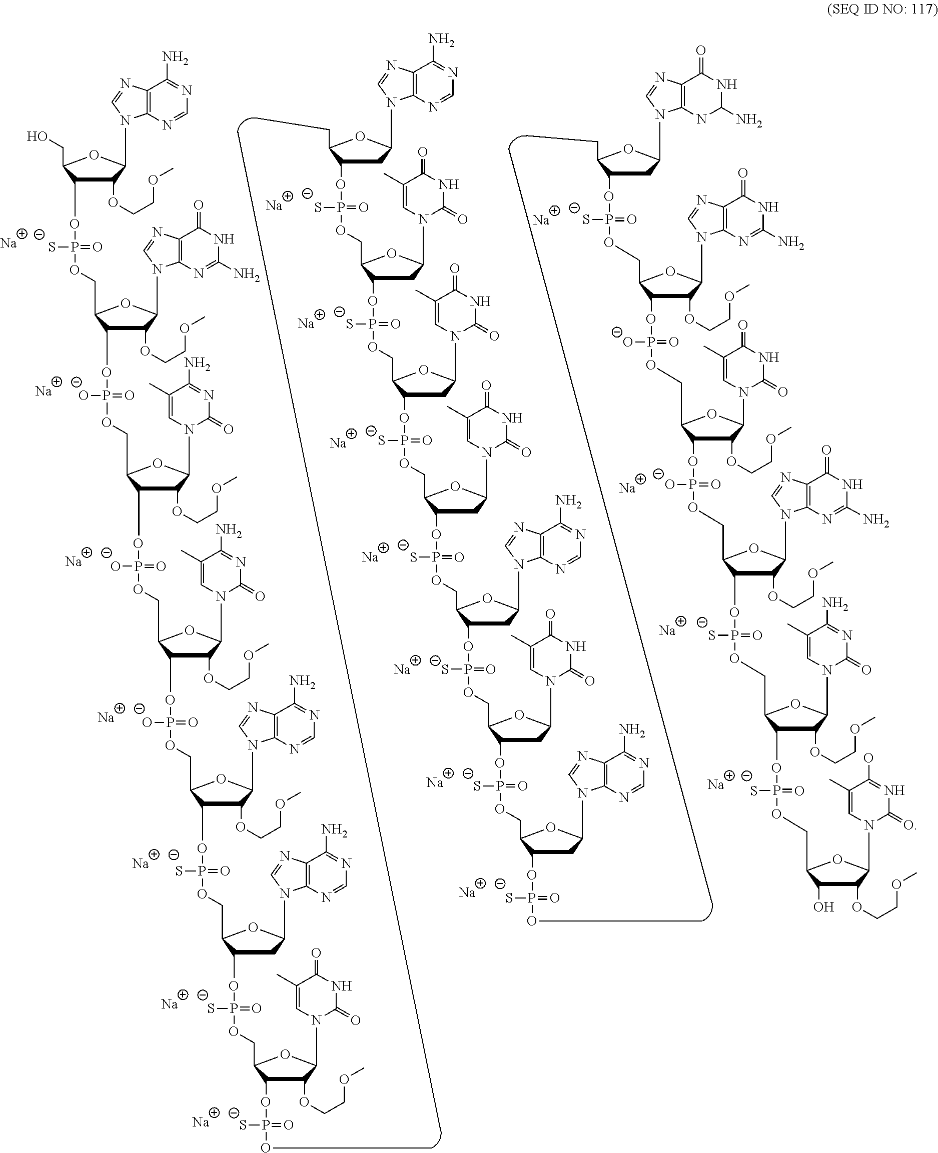

50. A modified oligonucleotide according to the following formula: 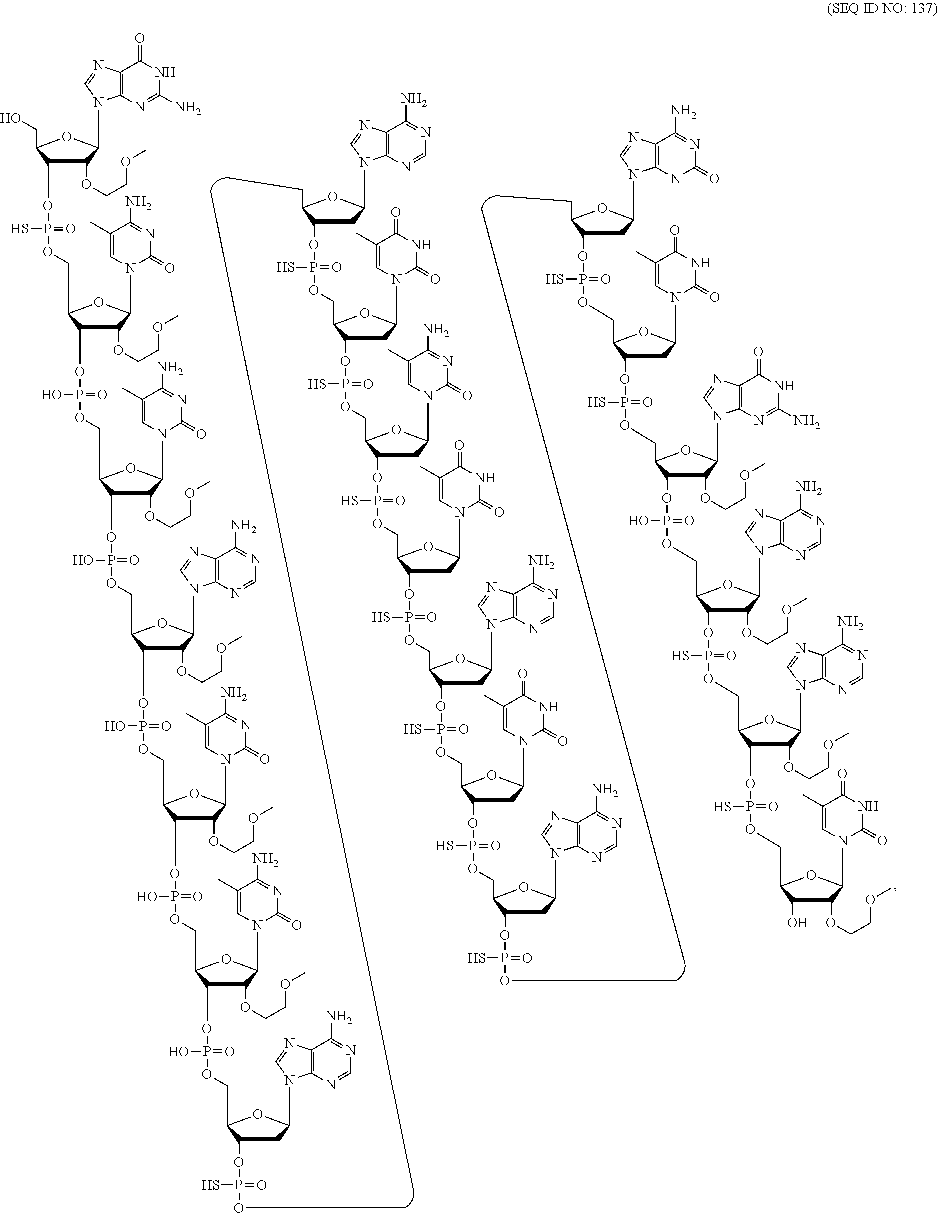
or a salt thereof.

51. The modified oligonucleotide of claim 50, which is the sodium salt or the potassium salt.

52. A modified oligonucleotide according to the following formula:

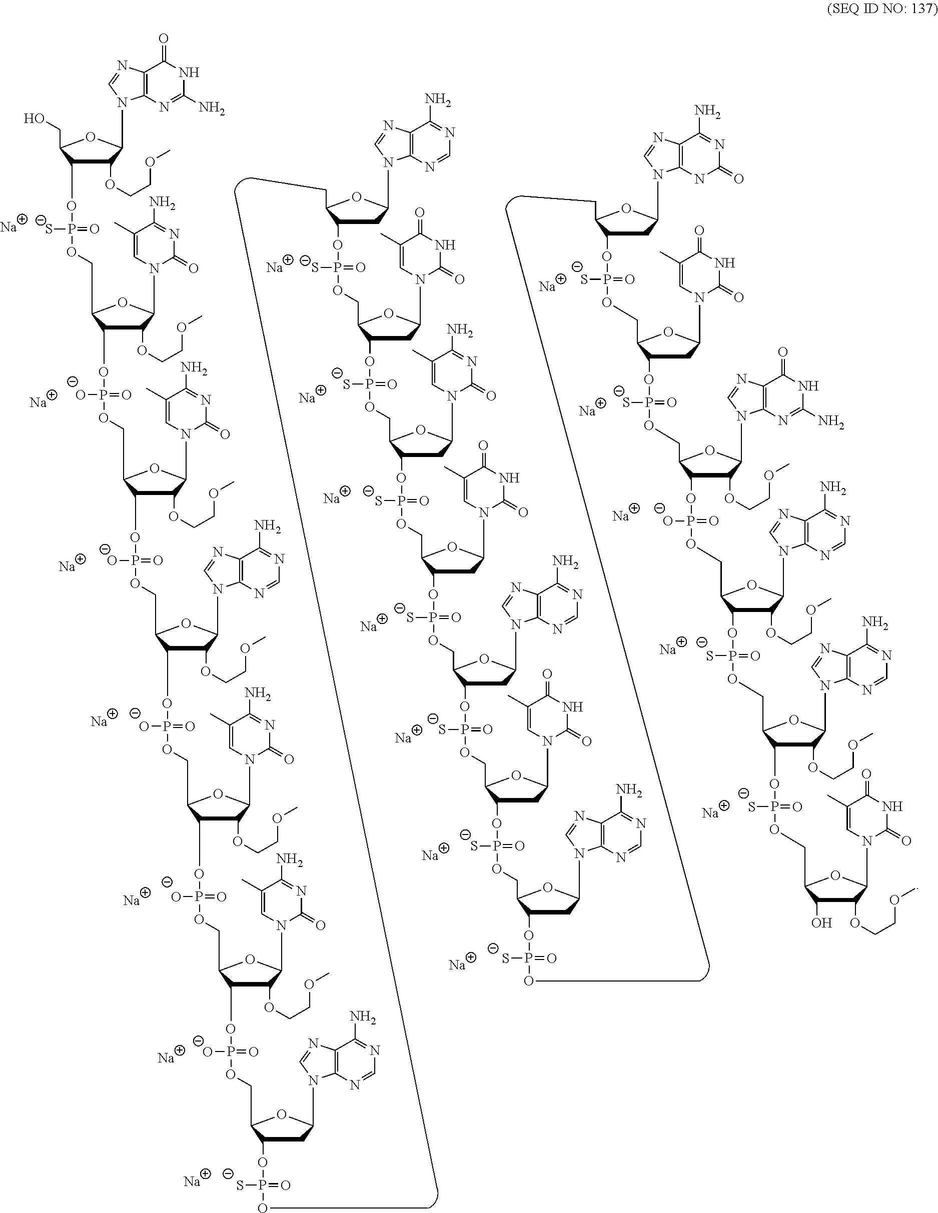

53. A modified oligonucleotide according to the following formula: 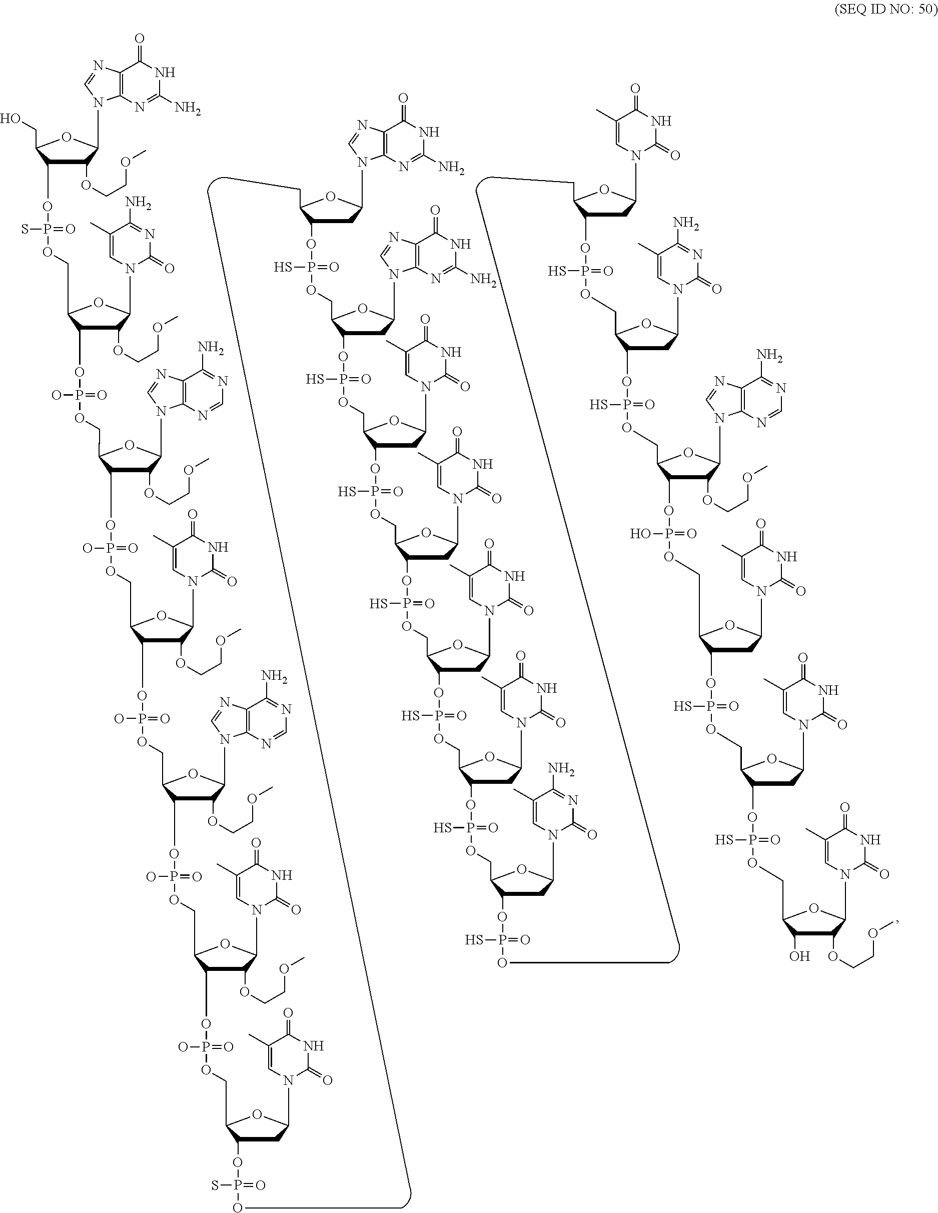
or a salt thereof.

54. The modified oligonucleotide of claim 53, which is the sodium salt or the potassium salt.

55. A modified oligonucleotide according to the following formula:

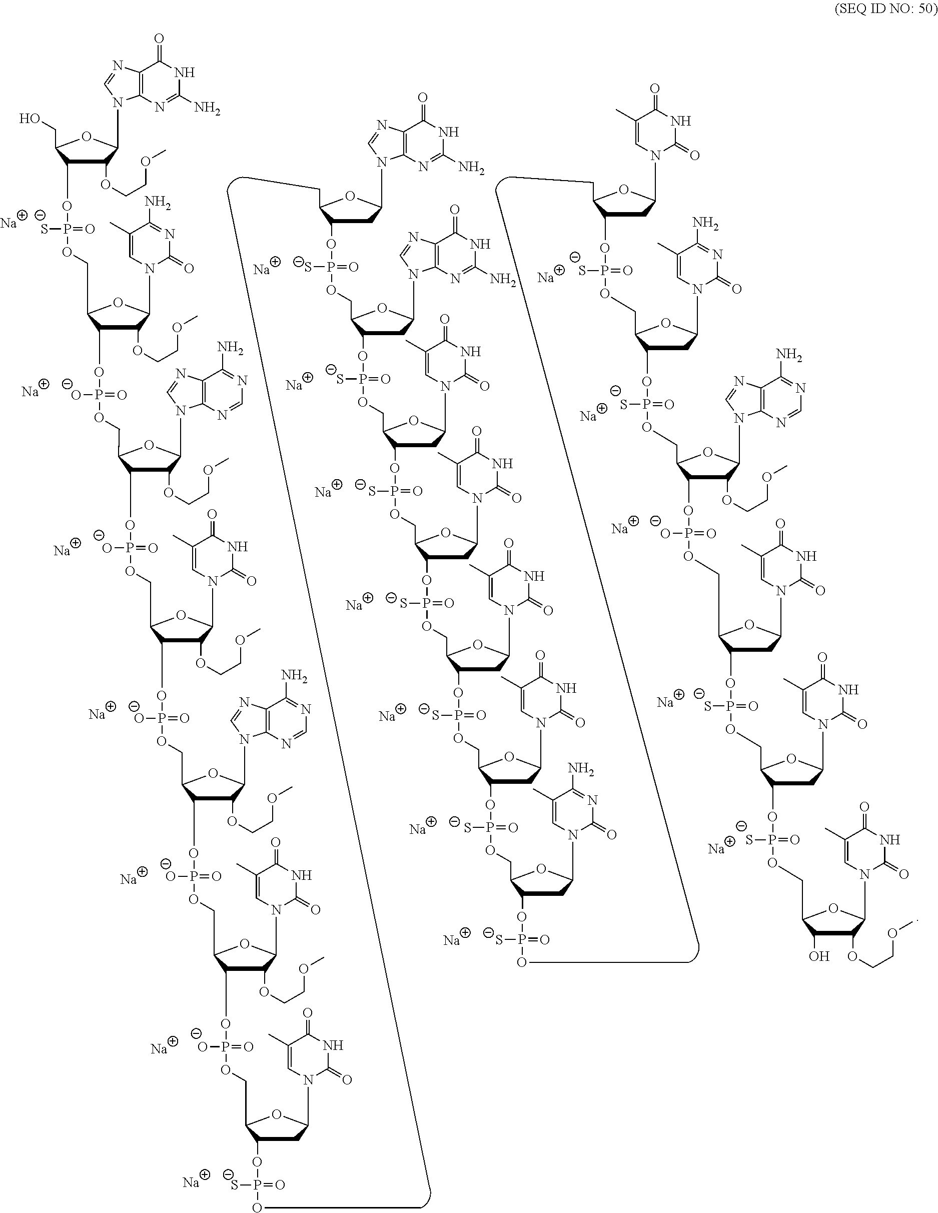

56. A pharmaceutical composition comprising the oligomeric compound of any of claims 1-46 or the modified oligonucleotide of any of claims 47-55, and a pharmaceutically acceptable diluent or carrier.

57. The pharmaceutical composition of claim 56, comprising a pharmaceutically acceptable diluent and wherein the pharmaceutically acceptable diluent is artificial CSF (aCSF) or PBS.

58. The pharmaceutical composition of claim 57, wherein the pharmaceutical composition consists essentially of the modified oligonucleotide and artificial CSF (aCSF).

59. The pharmaceutical composition of claim 57, wherein the pharmaceutical composition consists essentially of the modified oligonucleotide and PBS.

60. A chirally enriched population of modified oligonucleotides of any of claims 56-59, wherein the population is enriched for modified oligonucleotides comprising at least one particular phosphorothioate internucleoside linkage having a particular stereochemical configuration.

61. The chirally enriched population of claim 60, wherein the population is enriched for modified oligonucleotides comprising at least one particular phosphorothioate internucleoside linkage having the (Sp) configuration.

62. The chirally enriched population of claim 60, wherein the population is enriched for modified oligonucleotides comprising at least one particular phosphorothioate internucleoside linkage having the (Rp) configuration.

63. The chirally enriched population of claim 60, wherein the population is enriched for modified oligonucleotides having a particular, independently selected stereochemical configuration at each phosphorothioate internucleoside linkage.

64. The chirally enriched population of claim 63, wherein the population is enriched for modified oligonucleotides having the (Sp) configuration at each phosphorothioate internucleoside linkage or for modified oligonucleotides having the (Rp) configuration at each phosphorothioate internucleoside linkage.

65. The chirally enriched population of claim 63, wherein the population is enriched for modified oligonucleotides having the (Rp) configuration at one particular phosphorothioate internucleoside linkage and the (Sp) configuration at each of the remaining phosphorothioate internucleoside linkages.

66. The chirally enriched population of claim 63, wherein the population is enriched for modified oligonucleotides having at least 3 contiguous phosphorothioate internucleoside linkages in the Sp, Sp, and Rp configurations, in the 5′ to 3′ direction.

67. A population of modified oligonucleotides of any of claims 47-55, wherein all of the phosphorothioate internucleoside linkages of the modified oligonucleotide are stereorandom.

68. A method of reducing expression of Ataxin 3 in a cell, comprising contacting the cell with an oligomeric compound of any of claims 1-46 or a modified oligonucleotide of any of claims 47-55.

69. The method of claim 68, wherein the level of Ataxin 3 RNA is reduced.

70. The method of any of claims 68-69, wherein the level of Ataxin 3 protein is reduced.

71. The method of any of claims 68-69, wherein the cell is in vitro.

72. The method of any of claims 68-69, wherein the cell is in an animal

73. A method comprising administering to an animal the pharmaceutical composition of any of claims 56-59.

74. The method of claim 73, wherein the animal is a human.

75. A method of treating a disease associated with ATXN3 comprising administering to an individual having or at risk for developing a disease associated with ATXN3 a therapeutically effective amount of a pharmaceutical composition of claims 56-59, and thereby treating the disease associated with ATXN3.

76. The method of claim 75, wherein the disease associated with ATXN3 is a neurodegenerative disease.

77. The method of claim 76, wherein the neurodegenerative disease is SCA3.

78. The method of claim 76, wherein at least one symptom or hallmark of the neurodegenerative disease is ameliorated.

79. The method of claim 77, wherein the symptom or hallmark is ataxia, neuropathy, and aggregate formation.

80. The method of any of claims 73-79, wherein the pharmaceutical composition is administered to the central nervous system or systemically.

81. The method of claim 80, wherein the pharmaceutical composition is administered to the central nervous system and systemically.

82. The method of any of claim 73-79, wherein the pharmaceutical composition is administered any of intrathecally, systemically, subcutaneously, or intramuscularly.

83. Use of an oligomeric compound of any of claims 1-46 or a modified oligonucleotide of any of claims 47-55 for reducing Ataxin 3 expression in a cell.

84. The use of claim 83, wherein the level of Ataxin 3 RNA is reduced.

85. The use of claim 83, wherein the level of Ataxin 3 protein is reduced.
